(19) United States
(12) Patent Application Publication
(10) Pub. No.: US 2012/0058133 A1
(43) Pub. Date: Mar. 8, 2012

(54) INHIBITION OF TRNA SYNTHETASES AND THERAPEUTIC APPLICATIONS THEREOF

(75) Inventors: Malcolm Whitman, Jamaica Plain, MA (US); Tracy Keller, Jamaica Plain, MA (US)

(73) Assignee: President and Fellows of Harvard College, Cambridge, MA (US)

(21) Appl. No.: 13/201,933

(22) PCT Filed: Feb. 17, 2010
(86) PCT No.: PCT/US10/00460

§ 371 Date: Oct. 11, 2011

Related U.S. Application Data
(60) Provisional application No. 61/153,867, filed on Feb. 19, 2009.

(51) Int. Cl. A61K 35/00; A61K 31/7076; A61P 29/00; A61P 31/00; A61P 11/06; C12N 5/0783; C12Q 1/25; G01N 21/64; C12Q 1/68; G01N 33/566; C40B 30/04; A61K 31/519; A61K 31/473; A61K 31/47; A61K 31/4704; A61K 31/5375; A61K 31/137; A61P 37/06

(52) U.S. Cl. 424/184.1; 435/377; 435/29; 435/7.1; 435/6.12; 435/7.24; 506/9

(57) ABSTRACT

The present invention provides novel methods for modulating Th 17-mediated immune responses using aminoacyl tRNA synthetase inhibitors. Inhibition of aminoacyl tRNA synthetase inhibitors activates an amino acid starvation response (AAR) and can produce beneficial therapeutic effects. In some embodiments, aminoacyl tRNA synthetase inhibitors are used to treat disorders such as autoimmune diseases, graft rejection, infections, fibrosis, and inflammatory diseases.

### RELATED APPLICATIONS

The present application claims priority under 35 U.S.C. §119(e) to U.S. provisional application, U.S. Ser. No. 61/153,867, filed Feb. 19, 2009, which is incorporated herein by reference.

### BACKGROUND OF THE INVENTION

Naïve CD4⁺ T helper cells differentiate into diverse sets of effector and regulatory T cells to coordinate protective immune responses against foreign pathogens and provide tolerance to self antigens and commensal organisms. The classical T helper cell effector subsets, Th1 and Th2 cells, produce interferon-γ (IFNγ) or interleukin-4 (IL-4), IL-5 and IL-13, respectively. Naïve T cells can also differentiate into pro-inflammatory Th17 cells that produce IL-17, or into tissue-protective iTreg cells (Dong, Nat. Rev. Immunol. 8:337, 2008; Bettelli et al., Nature 453(7198):1051-7, 2008). Differentiation of T cells toward a particular phenotype is influenced by the local cytokine milieu. Transforming growth factor-β (TGFβ) has been shown to have obligate roles in mediating both iTreg and Th17 differentiation. TGFβ cooperates with IL-2 and retinoic acid to induce expression of the winged-helix forkhead transcription factor Foxp3 and direct cells toward the iTreg lineage. However, when present in combination with the STAT3-activating cytokines, IL-6 or IL-21, TGFβ initiates Th17 differentiation (Zhou et al., Nat. Immunol. 8:967, 2007). Yet another cytokine, IL-23, is dispensable for Th17 differentiation but is important for maintaining the inflammatory effector function of differentiated Th17 cells in vivo (McGeachy et al., Nat. Immunol. 8:1390, 2007; Kastelein et al., Annu. Rev. Immunol. 25:221, 2007). Th17 cells play a critical role in inflammatory functions associated with host defense against pathogens, and are implicated in the development of tissue inflammation and autoimmune diseases (Bettelli et al., Nature 453(7198):1051-7, 2008).

### SUMMARY OF THE INVENTION

The present invention arises from the recognition that activation of an AAR through inhibition of tRNA synthetases can be beneficial for treating various conditions. Agents that inhibit aminoacyl tRNA synthetases modulate immune responses in a subject by inhibiting the differentiation and activity of pro-inflammatory Th17 cells. Accordingly, agents that inhibit aminoacyl tRNA synthetases are useful in the treatment of disorders associated with the activity of Th17 cells such as autoimmune diseases, inflammation, infectious diseases, graft rejection, and graft versus host disease. Agents that inhibit aminoacyl tRNA synthetases can also inhibit fibrosis, scar formation, cardiovascular disease, angiogenesis (e.g., angiogenesis associated with cancer, macular degeneration, or choroidal neovascularization), cellulite formation, or cellulite progression are provided. Thus, compositions comprising an inhibitor of a eukaryotic aminoacyl tRNA synthetases and methods of using such compositions for the treatment of various diseases and/or for modulating T cell differentiation and/or activity are provided herein.

In one aspect, the invention features a method of inhibiting an immune response mediated by IL-17 expressing T cells in a subject, the method comprising administering to the subject an agent that inhibits a eukaryotic aminoacyl tRNA synthetase, wherein the agent is administered in an amount effective to inhibit the aminoacyl tRNA synthetase in T cells in the subject. In some embodiments, the agent induces an amino acid starvation response (AAR) in T cells of the subject. The agent can be an agent that inhibits Th17 differentiation in vitro. In some embodiments, the agent inhibits Th17 differentiation in vitro at a concentration below the concentration at which the agent inhibits cell proliferation. In some embodiments, the agent is administered in an amount below that which causes non-specific biological effects (e.g., generalized immunosuppression) in the subject.

In some embodiments, the agent inhibits a eukaryotic aminoacyl tRNA synthetase selected from: a prolyl tRNA synthetase, a cysteinyl tRNA synthetase, a methionyl tRNA synthetase, a leucyl tRNA synthetase, a tryptophanyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, a threonyl tRNA synthetase, a lysyl tRNA synthetase, an arginyl tRNA synthetase, a histidyl tRNA synthetase, a phenylalanyl tRNA synthetase, a tyrosyl tRNA synthetase, and a glutamyl-prolyl-tRNA synthetase (EPRS).

In some embodiments, the agent inhibits a eukaryotic aminoacyl tRNA synthetase of an essential amino acid.

In some embodiments, the agent inhibits a eukaryotic aminoacyl tRNA synthetase of a non-essential amino acid. For example, in some embodiments, the agent inhibits a eukaryotic aminoacyl tRNA synthetase selected from a prolyl tRNA synthetase, a cysteinyl tRNA synthetase, a glycyl tRNA synthetase, an alanyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS). For example, in some embodiments, the agent inhibits a glutamyl-prolyl-tRNA synthetase (EPRS).

The agent can be an active site inhibitor of a eukaryotic aminoacyl tRNA synthetase or a noncompetitive inhibitor of a eukaryotic aminoacyl tRNA synthetase. In various embodiments, the agent comprises a compound shown in FIG. 1. In various embodiments, the agent comprises a compound shown in Appendix A.

In some embodiments of the method, a second agent that inhibits a second eukaryotic aminoacyl tRNA synthetase, or the same eukaryotic aminoacyl tRNA synthetase, is administered to the subject. In some embodiments of the method, a second agent which inhibits expression or activity of a proinflammatory cytokine is administered to the subject. In some embodiments, the proinflammatory cytokine is selected from one or more of TNFα, IFNγ, GM-CSF, MIP-2, IL-12, IL-1α, IL-1β, and IL-23. In some embodiments of the method, a second agent which is an agent that inhibits expression or activity of IL-6 and/or IL-21 is administered to the subject. In some embodiments, a second agent which is an agent that inhibits TNFα is administered to the subject. In some embodiments, the agent that inhibits TNFα comprises an anti-TNFα antibody. In some embodiments, the agent that inhibits TNFα comprises a soluble TNF receptor.

In some embodiments, the first agent (i.e., the agent that inhibits an aminoacyl tRNA synthetase) inhibits an inflammatory immune response in the subject. For example, in some embodiments, the agent inhibits an activity (e.g., proliferation, differentiation, and/or cytokine production) of IL-17-expressing T cells in the subject. the agent inhibits proliferation of IL-17-expressing T cells in the subject. In some embodiments, the agent inhibits production of a cytokine in cells of the subject, wherein the cytokine is selected from IL-17, IL-6, IL-21, TNFα, and GM-CSF.

In some embodiments, the subject is a subject at risk for, or suffering from, an IL-17-mediated disorder. In some embodiments, the IL-17-mediated disorder is an autoimmune disease, e.g., a disease selected from rheumatoid arthritis, multiple sclerosis, Crohn's disease, inflammatory bowel disease, systemic lupus erythematosus, rheumatoid arthritis, scleroderma, dry eye disease, and insulin dependent diabetes mellitus type I. In some embodiments, the autoimmune disease is psoriasis. In some embodiments, the IL-17-mediated disorder is an infectious disease. In some embodiments, the IL-17-mediated disorder is graft rejection. In some embodiments, the IL-17-mediated disorder is graft versus host disease. In some embodiments, the IL-17-mediated disorder is asthma. In some embodiments, the IL-17-mediated disorder is chronic inflammation. In some embodiments, the IL-17-mediated disorder is inflammation associated with a microbial infection. In some embodiments, the microbial infection is a viral infection, protozoal infection, or fungal infection. In some embodiments, the microbial infection is a viral infection.

In some embodiments, a subject is identified as at risk for, or suffering from an IL-17-mediated disorder, prior to the administering.

In another aspect, the invention features a method of inhibiting one or more of fibrosis, angiogenesis, scar formation, cellulite formation or cellulite progression in a subject, the method comprising administering to the subject an agent that inhibits a eukaryotic aminoacyl tRNA synthetase, wherein the agent is administered in an amount effective to inhibit the aminoacyl tRNA synthetase in the subject. In some embodiments, the agent induces an amino acid starvation response (AAR) in cells of the subject. In some embodiments, the agent inhibits maturation of fibroblasts. In some embodiments, the agent inhibits one or more biological activities of fibroblasts. In some embodiments, the agent inhibits extracellular matrix deposition. In some embodiments, the agent is administered in an amount below that which causes non-specific effects in the subject.

The agent can be an agent that inhibits a eukaryotic aminoacyl tRNA synthetase selected from: a prolyl tRNA synthetase, a cysteinyl tRNA synthetase, a methionyl tRNA synthetase, a leucyl tRNA synthetase, a tryptophanyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, a threonyl tRNA synthetase, a lysyl tRNA synthetase, an arginyl tRNA synthetase, a histidyl tRNA synthetase, a phenylalanyl tRNA synthetase, a tyrosyl tRNA synthetase, and a glutamyl-prolyl-tRNA synthetase (EPRS).

The agent can inhibit a eukaryotic aminoacyl tRNA synthetase of an essential amino acid, or a eukaryotic aminoacyl tRNA synthetase of a non-essential amino acid. In some embodiments, the agent inhibits a eukaryotic aminoacyl tRNA synthetase selected from a prolyl tRNA synthetase, a cysteinyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS). In some embodiments, the agent inhibits a glutamyl-prolyl-tRNA synthetase (EPRS). The agent can be an active site inhibitor of a eukaryotic aminoacyl tRNA synthetase or a noncompetitive inhibitor of a eukaryotic aminoacyl tRNA synthetase. In various embodiments, the agent comprises a compound shown in FIG. 1. In various embodiments, the agent comprises a compound shown in Appendix A.

In some embodiments of the method, a second agent that inhibits a second eukaryotic tRNA synthetase is administered to the subject.

In another aspect, the invention features a method of modulating differentiation of a T cell, the method comprising contacting a T cell with an agent that inhibits a eukaryotic tRNA synthetase under conditions in which differentiation occurs, thereby modulating differentiation of the T cell. The T cell can be contacted with the agent in vitro. In other embodiments, the T cell is contacted with the agent in a subject in vivo. In some embodiments, the method inhibits Th17 differentiation. In some embodiments, the agent inhibits a eukaryotic aminoacyl tRNA synthetase selected from: a prolyl tRNA synthetase, a cysteinyl tRNA synthetase, a methionyl tRNA synthetase, a leucyl tRNA synthetase, a tryptophanyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, a threonyl tRNA synthetase, a lysyl tRNA synthetase, an arginyl tRNA synthetase, a histidyl tRNA synthetase, a phenylalanyl tRNA synthetase, a tyrosyl tRNA synthetase, and a glutamyl-prolyl-tRNA synthetase (EPRS). The agent can inhibit a eukaryotic aminoacyl tRNA synthetase of an essential amino acid or a non-essential amino acid. In some embodiments, the agent inhibits a eukaryotic aminoacyl tRNA synthetase selected from a prolyl tRNA synthetase, a cysteinyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and EPRS.

In another aspect, the invention features a method of identifying an agent that modulates T cell differentiation, the method comprising (a) contacting a T cell with an inhibitor of a eukaryotic aminoacyl tRNA synthetase under conditions in which T cell differentiation occurs, and (b) evaluating a marker of T cell differentiation, wherein a change in the marker of T cell differentiation, relative to a control, indicates that the inhibitor of the aminoacyl tRNA synthetase is an agent that modulates T cell differentiation. In some embodiments, the marker of T cell differentiation includes one or more of IL-17 expression, STAT3 phosphorylation, Foxp3 expression, expression of amino acid starvation response (AAR) genes, ATF4 expression, and eIF2 alpha phosphorylation. In some embodiments, an increase in one or more of Foxp3 expression, AAR gene expression, ATF4 expression, and eIF2 alpha phosphorylation indicates that the agent inhibits Th17 cell differentiation. In some embodiments, a decrease in one or more of IL-17 expression and STAT3 phosphorylation indicates that the agent inhibits Th17 differentiation.

In another aspect, the invention features a method for inducing an amino acid starvation response (AAR) in a subject, the method comprising administering to the subject an agent that inhibits a eukaryotic aminoacyl tRNA synthetase, wherein the agent is administered in an amount effective to inhibit the aminoacyl tRNA synthetase in T cells in the subject.

In some embodiments, the agent inhibits a eukaryotic aminoacyl tRNA synthetase selected from a prolyl tRNA synthetase, a cysteinyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS).

The invention also features a pharmaceutical composition comprising an agent that inhibits a eukaryotic aminoacyl tRNA synthetase in a pharmaceutically acceptable carrier. The agent can inhibit a eukaryotic aminoacyl tRNA synthetase selected from a prolyl tRNA synthetase, a cysteinyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS).

In still another aspect, the invention features a kit for modulating differentiation of a T cell, the kit comprising an agent that inhibits a eukaryotic aminoacyl tRNA synthetase. The agent can inhibit a eukaryotic aminoacyl tRNA synthetase selected from a prolyl tRNA synthetase, a cysteinyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS).

In some embodiments, the kit includes a second agent that inhibits a eukaryotic aminoacyl tRNA synthetase.

In some embodiments, the kit includes a second agent, wherein the second agent inhibits expression or activity of a proinflammatory cytokine. In some embodiments, the proinflammatory cytokine is selected from one or more of TNFα, IFNγ, GM-CSF, MIP-2, IL-12, IL-1α, IL-1β, and IL-23. In some embodiments, the second agent inhibits expression or activity of IL-6 or IL-21. In some embodiments, the second agent inhibits TNFα. In some embodiments, the second agent that inhibits TNFα is an anti-TNFα antibody. In some embodiments, the second agent that inhibits TNFα comprises a soluble TNF receptor.

### DEFINITIONS

The following definitions are more general terms used throughout the present application:

The terms “administer,” “administering,” or “administration,” as used herein refers to implanting, absorbing, ingesting, injecting, or inhaling an agent.

The terms “amino acid starvation response” and “AAR” refer to a cellular response pathway normally induced by insufficient amino acid levels. An agent is said to induce an AAR in a cell if the cell exhibits one or more characteristics of an AAR response. In some embodiments, an AAR is characterized by phosphorylation of eukaryotic translation initiation factor 2A (eIF2α) and/or increased expression of the transcription factor ATF4. Gene expression patterns associated with induction of the AAR pathway are described herein and, e.g., in Fafournoux et al., Biochem J. 351:1, 2000.

The terms “effective amount” and “therapeutically effective amount,” as used herein, refer to the amount or concentration of an agent, that, when administered to a subject, is effective to at least partially treat a condition from which the subject is suffering (e.g., an autoimmune disease).

The term “immune response,” as used herein, refers to a biological response by a cell of the immune system. In some embodiments, an immune response includes production of one or more soluble factors (e.g., a cytokine, such as an interleukin). In some embodiments, an immune response is mediated by T cells (e.g., IL-17 producing T cells). An immune response can be determined by any available means. In some embodiments, an immune response is determined by evaluating one or more of cytokine secretion, immune cell proliferation, immune cell phenotype (e.g., expression of activation markers), antibody secretion, or an indirect measure of immune activation, such as inflammation.

The terms “interleukin-17” or “IL-17” refer to any member of the IL-17 family, such as IL-17A, IL-17B, IL-17C, IL-17D, IL-17E, and IL-17F (see, e.g., Kolls and Linden, Immunity 21:467-76, 2004; and GenBank Acc. Nos. AAF28104, AAF28105, and NP_{—}002181).

The term “subject,” as used herein, refers to any animal. In certain embodiments, the subject is a mammal (e.g., a rodent, dog, cat, horse, cow, non-human primate, or human). In certain embodiments, the term “subject”, as used herein, refers to a human (e.g., a man, a woman, or a child).

“Th17 differentiation” refers to the differentiation of a T cell towards a Th17 phenotype. Th17 cells express IL-17. In some embodiments, a Th17 cell is IL-17⁺ IFNγ⁻.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

### BRIEF DESCRIPTION OF THE DRAWINGS

FIGS. 1A-1E show exemplary inhibitors of Ile tRNA synthetases.

FIGS. 1F-1K show exemplary inhibitors of Leu tRNA synthetases.

FIGS. 1L-1AP show exemplary inhibitors of Pro tRNA synthetases.

FIGS. 1AP-1AX show exemplary inhibitors of Asn tRNA synthetases.

FIGS. 1AY-1BX show exemplary inhibitors of Met tRNA synthetases.

FIG. 2. Selective inhibition of Th17 cell development by halofuginone.

FIG. 2A, Left, is a graph showing dose response analyses performed on activated carboxyfluorescein succinimidyl ester (CFSE)-labeled CD4⁺CD25⁻ T cells in the presence of DMSO, 40 nM MAZ1310, or increasing concentrations of halofuginone (HF) (1.25-40 nM). CFSE dilution and cell surface CD25 expression were determined 48 hours after activation. Intracellular cytokine production was determined on day 4 or 5 following restimulation with phorbol myristate acetate (PMA) and ionomycin. CFSE dilution and percentages of cells expressing CD25, IFN-γ⁺ IL4⁻ (Th1 cells), IL-4⁺ IFNγ⁻ (Th2 cells) or IL-17⁺ IFNγ⁻ (Th17 cells) cells are displayed and the values are normalized to T cells treated with 40 nM MAZ1310±SD. FIG. 2A, Right is a graph showing dose-response analyses of HF effects on CD8⁺ T cell or B cell function. Cells were activated in the presence of DMSO, 40 nM MAZ1310, or increasing concentrations of HF (1.25-40 nM). CFSE dilution, cell surface CD25 expression, and intracellular cytokine production were determined as above 2-5 days after activation. CFSE dilution and percentages of CD8⁺ T cells expressing CD25, IFNγ⁺ granzyme B⁺ (cytotoxic T lymphocytes) or IL-6⁺ B cells are displayed and the values are normalized to cells treated with 40 nM MAZ1310±SD.

FIG. 2B is a table showing IC₅₀ values calculated for the effects of HF on CD4⁺ CD25⁻ T cell functions as indicated.

FIG. 2C is a graph showing data for experiments in which a racemic mixture of HF (HF) or HPLC-purified D- or L-enantiomers of HF (HF-D, or HF-L) were added to CD4⁺ CD25⁻ T cells activated in the presence of TGFβ plus IL-6 and the percent of Th17 cells (IL-17⁺ IFNγ⁻) was determined by intracellular cytokine staining on day 4. Values are normalized to cells treated with 40 nM MAZ1310±SD.

FIG. 2D is a graph showing the percent of Th17 cells (IL-17⁺ IFNγ⁻) determined by intracellular staining 4 days after activation as above and values are presented as mean percent of Th17 cells ±SD. CD4⁺ CD25⁻ T cells were activated in the indicated cytokine conditions, and 10 nM HF was added at the indicated times following activation. Asterisks indicate statistical significance (p<0.005) relative to T cells treated with 10 nM MAZ1310 at the time of activation.

FIG. 2E is a set of FACS analyses of CFSE-labeled T cells activated in the indicated cytokine conditions in the presence of DMSO, 5 nM HF, 10 nM HF, 10 nM MAZ1310, or 10 μM SB-431542. Foxp3 intracellular staining was performed 3 days after T cell activation and intracellular cytokine staining was performed on day 4. Cells with equivalent CFSE fluorescence are gated on as indicated and intracellular Foxp3 or cytokine expression is shown within each gated population.

FIG. 2F is a set of FACS analyses of purified primary human memory T cells (CD4⁺ CD45RO⁺) activated in co-culture with CD14⁺ monocytes and treated with DMSO, 100 nM HF or 100 nM MAZ1310. T cells were expanded for 6 days and intracellular cytokine expression was determined following restimulation with PMA plus ionomycin.

FIG. 2G is a graph depicting the percent of IL-17⁻ (black bars) or IFNγ⁻ (white bars) expressing T cells upon treatment with the indicated additives. The data were normalized to T cells treated with MAZ1310 and are displayed as mean values ±SD. Asterisk indicates statistical significance (p<0.05). All data represent at least three similar experiments.

FIG. 3. HF inhibits Th17 differentiation through effects on STAT3 phosphorylation. FIG. 3A is a set of representative histograms displaying the kinetics of STAT3 phosphorylation in developing Th17 cells treated with or without HF. Resting naïve T cells (grey, shaded peak), T cells activated in the presence of TGFβ plus IL-6 (TGFβ/IL-6) treated with 10 nM MAZ1310 (light gray trace), 5 nM HF (medium gray trace), or 10 nM HF (dark gray trace). T cells were fixed at the indicated times and intracellular phospho-STAT3 staining was performed.

FIG. 3B depicts western blot analysis of CD4⁺ CD25⁻ T cells treated with 10 nM HF or 10 nM MAZ1310 and activated in the presence or absence of TGFβ plus IL-6. Whole cell lysates were generated at the indicated times following activation.

FIG. 3C is a set of FACS analyses of CD4⁺ CD25⁻ T cells from YFPfl/fl or STAT3C-GFPfl/fl mice treated with recombinant TAT-Cre which were activated in the presence or absence of TGFβ plus IL-6 and treated with DMSO, 5 nM HF, 10 nM HF, or 10 nM MAZ1310 as indicated. T cells were restimulated after 4 days and intracellular cytokine staining was performed. T cells expressing YFP or GFP are gated on as shown.

FIG. 3D is a bar graph displaying the percent of Th17 cells (IL-17⁺ IFNγ⁻) within YFP⁻ cells (black bars), YFP⁺ cells (grey bars), STAT3CGFP⁻ cells (white bars) or STAT3C⁻GFP⁺ (etched bars). The data are normalized to DMSO-treated cultures and are presented as mean values ±SD on duplicate samples. Asterisks indicate statistical differences between STAT3C-GFP⁺ cells and YFP⁺ cells (p<0.05).

FIG. 3E is a set of FACS analyses of CD4⁺ CD25⁻ T cells activated in medium or TGFβ plus IL-6, treated with DMSO, 10 nM HF, 10 nM MAZ1310, or 10 nM HF plus 10 μM SB-431542. Foxp3 expression was determined on day 3 by intracellular staining. All experiments were performed at least three times with similar results.

FIG. 4. HF activates the amino acid starvation response pathway in T cells.

FIG. 4A shows dot plot analyses of microarray data from CD4⁺ CD25⁻ T cells treated with 10 nM HF or 10 nM MAZ1310 and activated in Th17 polarizing cytokine conditions for 3 or 6 hours. Gray dots indicate transcripts increased at least 2-fold by HF treatment at both 3 and 6 hours. Hallmark amino acid starvation response genes are identified by text and arrowheads.

FIG. 4B is a graph showing chi-squared analysis of microarray data from FIG. 4A, which shows the expression distribution of genes previously found to be regulated by ATF4 in tunicamycin-treated mouse embryonic fibroblasts (dark dots—see the table in FIG. 14).

FIG. 4C is a graph depicting results of quantitative real-time PCR performed on cDNA generated from unstimulated naïve T cells or those activated for 4 hours in the presence of 10 nM MAZ1310 or 10 nM HF. Asns, Gpt2 or eIF4Ebp1 mRNA expression was normalized to Hprt levels and data are presented as mean values ±SD in duplicate samples.

FIG. 4D depicts western blot analysis of purified CD4⁺ CD25⁻ T cells either unstimulated, or TCR-activated without exogenous cytokines in the presence of DMSO, 40 nM MAZ1310 or titrating concentrations of HF (1.25-40 nM). Whole cell lysates were prepared 4 hours-post TCR activation and immunoblotting was performed with the indicated antibodies. ATF4 protein is indicated by arrowhead. NS—non-specific band.

FIG. 4E depicts western blot analysis of purified CD4⁺ CD25⁻ T cells activated through the TCR for the indicated times without exogenous cytokines in the presence of either 10 nM MAZ1310 or 10 nM HF as indicated. Whole cell lysates were prepared during the timecourse and immunoblotting was performed.

FIG. 4F depicts western blot analysis of CD4⁺ CD25⁻ T cells either left unstimulated or were TCR-activated in the absence or presence of the indicated polarizing cytokine conditions and 10 nM MAZ1310 or 10 nM HF as indicated. Whole cell lysates were generated 4 hours after activation and immunoblotting was performed. Microarray data were generated from three independent experiments and all other data are representative of at least two similar experiments.

FIG. 5. Amino acid deprivation inhibits Th17 differentiation. FIG. 5A depicts western blot and Xbp1 splicing analysis of CD4⁺ CD25⁻ T cells left unstimulated, or activated through the TCR for 4 hours in complete medium (complete—200 μM Cys/100 μM Met), medium lacking Cys/Met (−Cys/Met) or complete medium containing 1 μg/ml tunicamycin, 10 nM HF or 10 nM MAZ1310. Western blotting was performed on whole cell extracts with the indicated antibodies. Xbp-1 splicing assay was performed on cDNA synthesized from T cell cultures.

FIG. 5B is a graph depicting dose-response analyses of the effects of limiting Cys/Met concentrations on T cell activation and differentiation. Activated CD4⁺ CD25⁻ T cells were cultured in the absence or presence of polarizing cytokines to induce Th1, Th2, iTreg or Th17 differentiation. Titrating concentrations of Cys/Met are indicated. CD25 and Foxp3 expression was determined 3 days post activation, cytokine production determined by intracellular staining on day 4 or 5. Percentages of cells expressing CD25, Foxp3, IFNγ⁺ IL4⁻ (Th1 cells), IL-4⁺ IFNγ⁻ (Th2 cells), or IL-17⁺ IFNγ⁻ (Th17 cells) are displayed, and the values are normalized to T cells cultured in complete medium (200 μM Cys/100 μM Met).

FIG. 5C is a set of representative histograms show the kinetics of STAT3 phosphorylation in CD4⁺ CD25⁻ T cells activated in the presence of TGFβ plus IL-6. Resting naïve T cells (grey, shaded peak), T cells cultured in complete medium (200 μM Cys/100 μM Met—dark grey trace), low Cys/Met concentrations (10 μM Cys/5 μM Met—medium gray trace) or complete medium with 10 nM HF (light gray trace). T cells were fixed at the indicated times and intracellular phospho-STAT3 staining was performed.

FIG. 5D is a graph depicting quantification of the intracellular phospho-STAT3 data. Data are presented as the percent of phospho-STAT3⁺ T cells in each condition multiplied by mean fluorescence intensity (MFI). Mean values from duplicate samples are displayed ±SD.

FIG. 5E is a set of FACS analyses of activated T cells cultured in the indicated cytokine condition in complete medium (complete—200 μM Cys/100 μM Met/4 mM Leucine), medium containing 0.1× cysteine and methionine (Cys/Met), medium containing 0.1× leucine (Leu) or complete medium plus 0.2 mM L-tryptophanol. Cells were expanded for 4 days and restimulated with PMA and ionomycin for intracellular cytokine staining.

FIG. 5F is a graph depicting analyses of CD4⁺ CD25⁻ T cells cultured in the presence of titrating concentrations of tunicamycin as indicated. These cells were analyzed for CD25 upregulation or differentiation into Th1, Th2, iTreg or Th17 cells. All experiments were performed 3 times with similar results.

FIG. 6 shows the molecular structures of halofuginone (FIG. 6A), MAZ1310 (FIG. 6B), and SB-431542 (FIG. 6C).

FIG. 7. Effects of HF on T cell activation and effector function. FIG. 7A is a set of FACS analyses of CFSE labeled CD4⁺ CD25⁻ T cells activated in the absence or presence of polarizing cytokines. DMSO, 5 nM HF, or 5 nM MAZ1310 was added to the cells at the time of T cell activation. Intracellular Foxp3 staining was performed on expanded cells 3 days after activation. Cytokine expression was determined by intracellular staining after PMA and ionomycin restimulation on day 4-5.

FIG. 7B is a set of FACS and graphical analyses of CFSE labeled CD4⁺ CD25⁻ T cells treated with DMSO, 5 nM HF, or 5 nM MAZ1310 activated in the absence of polarizing cytokines. CFSE dilution and CD25 cell surface expression was determined on day 2 by FACS analyses. T cells were activated as above without exogenous cytokines and supernatants were harvested at the indicated time-points following activation. Cytokine secretion was determined using a cytometric bead array (CBA) on duplicate samples. Cytokine concentrations were determined by comparison to standard curves and data are presented as the mean cytokine concentrations ±SD.

FIG. 7C is a set of graphs depicting HF effects on IL-17 and IL-17f mRNA expression in Th cells. CD4⁺ CD25⁻ T cells were differentiated under Th17 cytokine conditions in the presence of DMSO, 10 nM HF or 10 nM MAZ1310 for 4 days as above. Cells were harvested and restimulated with PMA and ionomycin as above, and cDNA was generated for Sybrgreen real-time PCR analysis. Data indicate fold changes in mRNA expression normalized to Hprt and are presented as mean expression ±SD. Asterisks indicate statistical significance for IL-17 mRNA (p<0.001) and IL-17f mRNA (p<0.05) for HF-treated T cells relative to those treated with MAZ1310. All data are representative of at least three independent experiments.

FIG. 8. HF does not regulate TGFβ signaling in T and B cells. FIG. 8A is a set of FACS analyses of CD4⁺ CD25⁻ T cells activated in Th1 or Th2 polarizing conditions, either in the presence or absence of TGFβ. DMSO, 10 nM HF, 10 nM MAZ1310, or 10 μM SB-431542 added as indicated at the time of activation. Intracellular cytokine staining was performed on expanded T cells on day 5.

FIG. 8B is a set of FACS analyses of CD8⁺ T cells activated in the presence or absence of TGFβ and cultured with DMSO, 10 nM HF, 10 nM MAZ1310, or 10 μM SB-431542. Expanded cells were restimulated on day 5 and intracellular staining was performed.

FIG. 8C is a set of FACS analyses of CFSE-labeled B cells activated by LPS stimulation in the presence or absence of TGFβ plus DMSO, 10 nM HF, 10 nM MAZ1310, or 10 μM SB-431542. Intracellular IL-6 production in B cells restimulated with PMA plus ionomycin, or cell-surface IgA expression was determined 4 days after activation.

FIG. 8D depicts western blot analyses of purified CD4⁺ CD25⁻ T cells treated with DMSO, 40 nM MAZ1310, titrating concentrations of HF (2.5-40 nM) or 10 μM SB-431542 for 30 minutes in complete medium supplemented with 0.1% fetal calf serum. T cells were then activated in the presence or absence of 3 ng/ml TGFβ. Whole cell extracts were prepared after 1 hour of stimulation and western blot analyses were performed using the indicated antibodies. These data are representative of three similar experiments.

FIG. 9. HF inhibits RORγt function, not expression. FIG. 9A is a graph depicting analyses of CD4⁺ CD25⁻ T cells treated with DMSO (if no indication), 10 nM HF, or 10 nM MAZ1310 as indicated and activated in the presence of cytokines as noted. T cells were harvested at the indicated times following activation, RNA was isolated and quantitative real-time PCR was performed on cDNA. RORγt expression was normalized to Gapdh levels and the data are presented as fold changes relative to unstimulated T cells.

FIG. 9B is a set of FACS analyses of CD4⁺ CD25⁻ T cells activated in the presence or absence of TGFβ plus IL-6, which were transduced with empty (MIG) or RORγt-expressing (MIG.RORγt) retroviruses 12 hours-post activation. Infected T cells were expanded and restimulated on day 4 for intracellular staining. MIG and MIG.RORγt-transduced cells were gated based on GFP fluorescence.

FIG. 9C is a graph depicting the percent of Th17 cells (IL-17⁺ IFNγ⁻) in cultures of MIG-transduced (black bars) or MIG.RORγt-transduced (white bars) T cells as determined by intracellular cytokine staining were normalized to DMSO-treated cultures. The data are presented as mean values ±SD on duplicate samples. These data are representative of three similar experiments.

FIG. 10. HF-enforced Foxp3 expression is not necessary or sufficient for the inhibition of Th17 differentiation. FIG. 10A is a set of FACS analyses of CD4⁺ CD25⁻ T cells activated in the presence or absence of TGFβ plus IL-6 which were transduced with empty (pRV) or FOXP3-expressing (pRV.FOXP3) retroviruses 12 hours after activation. Intracellular FOXP3 and cytokine expression was determined 3 days after infection (4 days after activation). IFNγ and IL-17 expression in pRV- and pRV.FOXP3-transduced cells was determined by FACS analyses after gating on GFP⁺ cells.

FIG. 10B is a set of FACS analyses of FACS sorted naïve CD4⁺ T cells from wild-type (WT) or Foxp3-deficient (Foxp3 KO) male mice, treated with DMSO, 10 nM HF, or 10 nM MAZ1310 as indicated and activated in the absence or presence of TGFβ plus IL-6. T cells were expanded and were restimulated on day 4 for intracellular cytokine staining. These results are representative of cells purified from two pairs of WT and Foxp3 KO mice.

FIG. 11. HF induces a stress response in fibroblasts. SV-MES mesangial cells were stimulated for 2 hours with DMSO, 20 nM MAZ1310, or 20 nM HF. Whole cell lysates were analyzed for expression of phosphorylated or total eIF2α by western blotting. These data represent at least two similar experiments.

FIG. 12. Amino acid deprivation mimics the effects of HF on T cell differentiation. FIG. 12A depicts western blot analyses of CD4⁺ CD25⁻ T cells activated through the TCR for the indicated times without polarizing cytokines in the presence or absence of cysteine and methionine (Cys/Met). Whole cell lysates were prepared and immunoblotting was performed.

FIG. 12B is a graph depicting results of quantitative real-time PCR performed on cDNA generated from naïve T cells, either left unstimulated or activated through the TCR for 4 hours without exogenous cytokines in the presence or absence of Cys/Met as indicated. Asns, Gpt2 or eIF4Ebp1 mRNA expression was normalized to Hprt levels, and data are presented as mean expression values ±SD in duplicate samples.

FIG. 12C is a set of FACS analyses of CD4⁺ CD25⁻ T cells cultured in complete medium (200 μM Cys/100 μM Met), medium containing limiting concentrations of Cys/Met (0.1×-20 μM Cys/10 μM Met), or complete medium plus 31.25 ng/ml tunicamycin. Cells were activated through the TCR in the absence or presence of polarizing cytokines to induce Th1, Th2, iTreg, or Th17 differentiation. Foxp3 intracellular staining was performed on day 3-post activation, and intracellular cytokine expression was determined on cells restimulated with PMA plus ionomycin on day 4-5.

FIG. 12D is a set of FACS analyses of CD4⁺ CD25⁻ T cells labeled with CFSE, cultured in medium containing the indicated concentrations of Cys/Met and activated in the absence or presence of TGFβ plus IL-6. Cells were expanded until day 4 when CFSE dilution and intracellular cytokine production was determined on restimulated cells. Cells with equivalent CFSE fluorescence are gated on as indicated, and intracellular cytokine expression is shown within each gated population.

FIG. 13. Genes induced by HF treatment in T cells. Gene symbols and names of transcripts increased at least 2-fold by HF treatment at both 3 and 6 hours. Mean fold increases ±SD from triplicate samples of HF-versus MAZ1310-treated T cells is shown at 3 and 6 hours.

FIGS. 14A-14C. Probe IDs of known stress response genes. Affymetrix probe IDs and gene names previously identified as ATF4 responsive during tunicamycin-induced ER stress in mouse embryonic fibroblasts.

FIG. 15 depicts western blot analysis showing that halofuginone stimulates the amino acid response (AAR) in a fibroblastic cell line.

FIG. 16 depicts western blot analysis showing that phosphorylation of eIF2alpha by halofuginone is GCN2-dependent.

FIG. 17 is a graph showing that proline rescues translational inhibition by halofuginone.

FIG. 18A depicts western blot analysis showing that halofuginone induced eIF2alpha phosphorylation.

FIG. 18B is a graph showing that halofuginone-inhibited Th17 differentiation is blocked by added proline.

FIG. 19 is a set of FACS analyses showing that depletion of amino acids or tRNA synthetase inhibition with L-tryptophanol inhibits Th17 differentiation. T cells were cultured in the presence of medium containing 0.1×, 0.2×, and 1× cysteine and methionine (Cys/Met), medium containing 0.1×, 0.2×, and 1× leucine (Leu), or complete medium plus 0.1 mM, 0.2 mM, 0.4 mM, or 0.8 mM tryptophanol. Cells were activated and cultured under Th17 differentiating conditions, restimulated, and assayed for IL-17 and IFNγ expression.

FIG. 20. Inhibition of IL-17-associated autoimmune inflammation in vivo.

FIG. 20A is a set of FACS analyses of CNS-infiltrating mononuclear cells which were isolated from myelin oligodendrocyte glycoprotein (MOG)-immunized mice during active disease (day 19−clinical score=2) and stimulated ex vivo with PMA and ionomycin. Expression of IFNγ (left panel) and IL-17 (right panel) was determined in CD4⁺ TCRβ⁺ T cells.

FIG. 20B is a graph depicting the effect of systemic HF administration on adjuvant-driven experimental autoimmune encephalomyelitis (EAE). Control mice were immunized with an emulsion of PBS in Complete Freund's Adjuvant (CFA) and treated with 2 mg HF daily (no MOG+HF (n=10)). Other mice were immunized with MOG₃₃₋₅₅ in CFA and treated daily with either DMSO (MOG+DMSO (n=12)), or 2 mg HF (MOG+HF (n=14)). Disease was monitored daily.

FIG. 20C is a set of FACS analyses of leukocytes isolated from CNS tissue of mice with active EAE following transfer of PLP-specific T cells. Cells were stimulated ex vivo with PMA and ionomycin and expression of IFNγ (left panel) or IL-17 (right panel) was determined in PLP-reactive (TCRVβ6 gated) CD4⁺ T cells by intracellular staining.

FIG. 20D is a graph depicting the effect of HF in a passive EAE model. Following the transfer of PLP-specific T cells, recipient mice were treated daily either with 2 mg HF (n=6) or vehicle control (n=5) and disease was monitored daily. Data are shown as mean EAE scores.

FIG. 20E is a set of FACS analyses of cells from lymph node or CNS of HF treated animals or control animals in an adjuvant-driven EAE model. For FIG. 20E, left panels, paraaortic lymph nodes were harvested from MOG-immunized mice treated with DMSO or HF after 6 days. Cells were cultured in the absence (resting—top panels) or presence (P+I—bottom panels) of PMA and ionomycin and stained for intracellular cytokine expression. For FIG. 20E, right panels, mononuclear cells were isolated from CNS tissue of DMSO-treated (clinical score=2) or HF-treated (clinical score=0) mice 17 days after immunization with MOG. Intracellular staining was performed on cells following PMA and ionomycin stimulation as above. Cytokine production is shown in TCRβ⁺ CD4⁺ gated cells and the percentages of IL-17-expressing cells are indicated.

FIG. 20F, left panel, depicts western blot analysis of protein from cells of wild-type mice injected i.p. with vehicle (DMSO) or 2.5 mg HF. Spleens were harvested 6 hours post injection, red blood cells were removed by NH₄Cl lysis buffer and immunoblotting for phosphorylated or total eIF2a was performed on whole cell extracts. FIG. 20F, right panel is a graph depicting levels of AAR-associated gene expression (Asns, Gpt2, eIF4Ebp1) analyzed by quantitative real-time PCR using cDNA from splenocytes of mice treated with DMSO or HF as above. Expression of AAR-associated transcripts were normalized to Hprt levels and data are presented as mean relative expression from duplicate samples ±SD. All data represent 2-3 similar experiments.

FIG. 21. Regulation of T cell differentiation by halofuginone during adjuvant-driven EAE. FIG. 21A is a set of FACS analyses of T cells from paraaortic lymph nodes (day 6). FIG. 21B, left panel, is a set of FACS analyses of T cells from CNS tissue (day 18). T cells analyzed in FIGS. 21A and 21B were from control- or HF-treated mice analyzed for CD44 and CD62L expression following induction of EAE. CD44 and CD62L expression in shown on cells gated for CD4 and TCR expression as shown. FIG. 21B, right panel, is a graph depicting cell numbers of CNS infiltrates in DMSO-treated mice (clinical score=2) or HF-treated mice (clinical score=0), which were determined during active EAE disease (day 18). Total mononuclear cells, CD4⁺ TCRβ⁺ T cells, Th1 cells (IFNγ⁺) or Th17 cells (IL-17⁺) present within CNS preparations were quantified following FACS analyses and are displayed as mean numbers +SD. Asterisks indicate statistical significance. These data are representative of at least 2 independent experiments analyzing at least 3 mice per group.

FIG. 22. Structure of Prolyl Adenylate and Febrifugine derivatives. Stereospecific structure and nomenclature are shown for enantiomers of HF.

FIG. 23. HF and FF inhibit prolyl tRNA synthetase activity in vitro. FIG. 23A is a graph showing that proline rescues inhibition of translation by HF in RRL. Rabbit reticulocyte lysate (RRL) was incubated with luciferase mRNA and translation quantitated in a luminescence assay. A high concentration of mixed or individual amino acids (1 mM of each) was added to rescue translational inhibition. AA Mix 1: Asn, Arg, Val, Glu, Gly; AA Mix2: Lys, Ile, Tyr, Asp, Trp; Mix 3: His, Met, Leu, Ala, Thr; Mix 4: Ser, Phe, Pro, Gln. HF had no direct effect on luciferase activity in a standard luciferase assay (not shown). Note log scale of y axis.

FIG. 23B is a graph showing structural specificity of HF/FF derivative-inhibition of translation in RRL. Inhibitors (FIG. 22) and proline were assayed as described in FIG. 23A. Note log scale at Y axis. Error bars reflect standard deviation of triplicate determinations.

FIG. 23C is a gel showing that HF and FF do not inhibit translation of a polypeptide lacking proline. Short myc-tagged polypeptides of identical sequence (see Example 9, “Materials and Methods”) with the exception that NoPro lacks proline, while ProPro contains a proline dipeptide, were translated in RRL in the presence of indicated inhibitors. Translation was examined by anti-myc Western blot.

FIG. 23D is a graph showing that HF inhibits prolyl tRNA synthetase activity in RRL. ¹⁴C Pro or ³⁵S Met were incubated with RRL and total bovine tRNA in the presence or absence of HF or MAZ1310, and incorporation of radioactivity into tRNA was measured by liquid scintillation counting. Error bars reflect standard deviation of triplicate determinations. Data are representative of two separate experiments.

FIG. 23E is a graph showing that HF inhibits purified EPRS activity. EPRS was purified from rat liver and assayed for prolyl-tRNA charging activity as previously described (Ting, S. M., Bogner, P. & Dignam, J. D. Isolation of prolyl-tRNA synthetase as a free form and as a form associated with glutamyl-tRNA synthetase. J Biol Chem 267, 17701-17709, (1992)). Data are representative of three separate experiments.

FIG. 23F is a plot showing that EPRS rescues HF inhibition of translation. Inhibition of translation in RRL was measured as in FIG. 23A in the absence or presence of low (80 ng) or high (0.5 μg) concentrations of added EPRS purified from rat liver. Note log scale of Y axis.

FIG. 24. Activation of the AAR in cells by HF/FF occurs through inhibition of proline utilization. FIG. 24A is a gel showing HF-induction of the AAR pathway is reversed by proline. MEFs were treated with the indicated concentration of HF or FF in the presence or absence of 2 mM Proline for 2 hours (left) or treated with 1 μM borrelidin in the presence or absence of 2 mM Threonine for 2 hours (right). Cells were lysed and assessed by anti-pGCN2 or anti-GCN2 Western blot. Data are representative of three separate experiments.

FIG. 24B is a gel showing HF induction of eIF2a phosphorylation and CHOP expression is GCN2 dependent. Wild Type or GCN2−/− MEFs were treated with 50 nM HF in the presence or absence of 2 mM proline. Cells were lysed at 2 hours post-HF addition (peIF2a) or 6 hours post-HF addition (CHOP) and analyzed for phosphoprotein or total protein levels by Western blot.

FIG. 24C is a graph showing incubation with proline does not change intracellular accumulation of HF. MEFs were incubated with the indicated concentration of HF in the presence or absence of 2 mM proline for 2 hours and then lysed. Lysates were then tested for HF levels in comparison to a standard curve using known concentrations of HF with an anti-HF antibody based ELISA assay (see Example 9, “Materials and Methods”). Error bars reflect standard deviation of triplicate determinations. Similar results were obtained in three separate experiments.

FIG. 25. HF does not directly inhibit downstream targets of the mTOR pathway in fibroblasts. MEFs growing in DME/10% FCS were treated with 100 nM HF or 0.5 μM Rapamycin and analyzed by Western blot for phosphorylation of component of the mTor signaling pathway. Lane 1: 100 nM MAZ1310 1 hr; Lane 2: 100 nM HF 8 hr; Lane 3: 100 nM HF 4 hr; Lane 4: 100 nM HF 2 hr; Lane 5: 100 nM HF 1 hr; Lane 6: 0.5 μM Rapamycin 1 hr.

FIG. 26. Functional effects of HF are mediated by inhibition of proline utilization. FIG. 26A is a graph and gel showing that proline rescues HF-inhibition of TH17 differentiation. Top—Primary murine CD4+ CD25− T cells were activated through the TCR in Th17 polarizing conditions in the presence of either 10 nM MAZ1310 or HF. HF-treated T cell cultures were further supplemented with amino acids as follows: 10× concentration of essential (EAA) or non-essential (NEAA) amino acids mixtures (Biowhittaker or Invitrogen, respectively), or 10× concentrations (1 mM) of indicated individual amino acids. Th17 differentiation was determined on day 4-post TCR activation by intracellular cytokine staining (see Example 9, “Materials and Methods”). Data are presented as mean percentage of Th17 (IL-17+ IFNg−) cells +/− SD from triplicate wells. Bottom—Murine CD4+ CD25− T cells activated through the TCR in the absence of polarizing cytokines were treated with 10 nM MAZ1310 or HF for 4 hours. HF-treated T cells were supplemented with control water or 10× concentrations (1 mM) of individual amino acids as indicated. Whole T cell lysates were analyzed for phosphorylated or total eIF2a by western blotting.

FIG. 26B. Inhibition of TH17 differentiation by the threonyl tRNA synthetase inhibitor borrelidin is rescued by its cognate amino acid. Primary murine CD4+ CD25− T cells were activated through the TCR in non-polarizing (ThN), or Th17 polarizing conditions and treated with DMSO, 10 nM MAZ1310, 10 nM HF, or 6 nM (3 ng/mL) borrelidin in the presence or absence proline or threonine (0.5 mM). Th17 differentiation was determined as in FIG. 26A. Data are presented as mean percentage of Th17 (IL-17+ IFNg−) cells +/− SD from triplicate wells. These data show that a threonyl tRNA synthetase inhibitor, like the prolyl tRNA synthetase inhibitor halofuginone, selectively inhibits Th17 differentiation, and that this inhibition can be rescued by the cognate amino acid for the synthetase, in this case threonine.

FIG. 26C. Proline rescues both HF stimulated and HF inhibited gene expression. MEFs were treated with or without HF (50 nM) and/or Proline (2 mM) for 4 hours (CHOP, S100A4) or 24 hours (CoLIA1, ColIA2). DMSO was used as vehicle control. Changes in mRNA levels are expressed as fold-change relative to control and normalized to expression of TBP. Error bars reflect standard deviation of determinations from plates of cells treated in triplicate, confidence interval (p-value) for the effect of HF alone versus HF+Pro was determined using a two-tailed Student's t test. Data are representative of two separate experiments.

FIG. 26D. HF-inhibition of collagen production is rescued by proline and doesn't affect overall protein synthesis. Cells were pre-incubated with HF with or without proline for 4 hours, and ³⁵S-methionine and HF with or without proline was added. 24 hours later, conditioned medium and total cell lysate was harvested. New production of Type I procollagen was measured in conditioned medium by Western blot, and bands quantitated using Image J quantitation software. The original blot is shown in FIG. 27; total protein synthesis was measured as TCA-precipitable ³⁵S in a scintillation counter. Error bars reflect standard deviation of triplicate determinations.

FIG. 27. HF effect on Type I procollagen production. Cells were treated with HF and conditioned medium analyzed for Type I procollagen as described in FIG. 26D.

FIG. 28. Borrelidin Selectively Inhibits Th17 Differentiation. Primary T-cells were differentiated and analyzed under different polarization conditions as described in FIG. 26B and in Sundrud, M. S. et al. (Halofuginone inhibits TH17 cell differentiation by activating the amino acid starvation response. Science 324, 1334-1338, (2009)), in the presence or absence of 3 ng/ml Borrelidin or 10 nM HF. Cells were analyzed for effector T-cell subtype differentiation as in Sundrud, M. S. et al. (Halofuginone inhibits TH17 cell differentiation by activating the amino acid starvation response. Science 324, 1334-1338, (2009)). These data show that inhibition of threonyl tRNA synthetase, like inhibition of prolyl tRNA synthetase by halofuginone, exerts a selective effect on Th17 differentiation rather than a general immunosuppressive effect on all T cells.

FIG. 29. HF effect on cellular metabolic activity. MEFs were incubated at indicated dose of HF with or without 2 mM proline for 24 hours and tested for metabolic activity using an Alamar blue assay (Invitrogen).

FIG. 30. Proline addition to fibroblasts rescues HF-inhibition of TGFβ Signaling. MEFs were treated with HF for 12 hours in the presence or absence of 2 mM proline, treated with 2 ng/mL TGFβ for 1 hour, and assayed for total Smad2 or pSmad2 by Western blot. Total and pSmad2 levels were quantitated using Image J software (bottom panel). Data are representative of three separate experiments.

FIG. 31. HF Dose response for pGCN2-induction and pSmad2-inhibition. Fibroblasts were treated for 12 hours with the indicated dose of HF and analyzed for Smad2 or GCN2 phosphorylation by Western blot. Data are representative of three separate experiments.

FIG. 32. pSmad2 inhibition by HF develops slowly over time. Fibroblasts were treated for the indicated time with 20 nM HF, then treated for 1 hr with 2 ng/mL TGFβ and analyzed for phosphorylation of Smad2 or GCN2 by Western blot. Data are representative of three separate experiments. Bottom panel: Western data in top panel were quantitated using Image J software.

FIG. 33. Proline rescues HF-suppression of ECM protein production. MEFs were incubated for 4 hours in HF with or without 2 mM Pro, to measure new collagen production in conditioned medium (cells were washed into fresh DMEM/0.2% FBS with fresh HF and proline re-added, and either conditioned medium (for Type I procollagen) or total cell lysate (for fibronectin, c-actin) harvested twenty four hours later. Protein levels were assayed by Western blot.

FIG. 34. The tryptophanyl tRNA synthetase inhibitor tryptophanol inhibits Th17 differentiation. Primary T-cells were differentiated and analyzed under Th17 polarization conditions as described in FIG. 28, in medium lacking leucine (−Leu), lacking Cysteine and Methionine (−Cys/−Met), or in complete medium supplemented with 1 mM Tryptophanol. These data show that the inhibition of tryptophanyl tRNA synthetase with tryptophanol, like inhibition of prolyl tRNA synthetase or threonyl tRNA synthetase, inhibits Th17 differentiation.

### DETAILED DESCRIPTION OF CERTAIN EMBODIMENTS OF THE INVENTION

The present invention provides novel methods and compositions for activating an amino acid starvation response (AAR) in cells using agents that inhibit eukaryotic aminoacyl tRNA synthetases. It has been discovered that activation of an AAR through inhibition of tRNA synthetases can be beneficial for treating various conditions. In some embodiments, agents that inhibit aminoacyl tRNA synthetases modulate immune responses in a subject by modulating differentiation and/or activity of T helper type 17 (Th17) cells. Therefore, agents that inhibit aminoacyl tRNA synthetases are useful in the treatment of disorders associated with the activity of pro-inflammatory Th17 cells such as autoimmune diseases, inflammation, infectious diseases, graft rejection, and graft versus host disease. In some embodiments, methods of using agents that inhibit aminoacyl tRNA synthetases to inhibit fibrosis, scar formation, cardiovascular disease, angiogenesis (e.g., angiogenesis associated with cancer, macular degeneration, or choroidal neovascularization), cellulite formation, or cellulite progression are provided. The present invention also provides compositions comprising an inhibitor of a eukaryotic aminoacyl tRNA synthetases and methods of using such compositions for the treatment of various diseases and/or for modulating T cell differentiation and/or activity.

Inhibition of Aminoacyl tRNA Synthetases

Aminoacyl tRNA synthetases catalyze the acylation of tRNAs with their cognate amino acids. The present invention encompasses the discovery that inhibition of aminoacyl tRNA synthetases has certain effects on biological processes in immune and non-immune cell types. The selectivity of aminoacyl tRNA synthetase inhibition for certain processes can provide beneficial therapeutic effects. For example, inhibition of aminoacyl tRNA synthetases, e.g., via activation of an amino acid starvation response, selectively suppresses differentiation of Th17 cells. Inhibition of aminoacyl tRNA synthetases can also suppress pro-fibrotic gene expression, viral gene expression, viral replication, and viral maturation, and organ stress. Accordingly, agents that inhibit eukaryotic aminoacyl tRNA synthetases can be used to modulate a Th17-mediated immune response, e.g., by suppressing the differentiation of Th17 cells and functions associated with Th17 cells such as IL-17 production, IL-6 production, nitric oxide production, prostaglandin E₂ production, and promotion of inflammation. Agents that inhibit aminoacyl tRNA synthetases can be used to suppress fibrosis, angiogenesis, cardiovascular disease, and cellulite formation. Any eukaryotic tRNA synthetase can be inhibited in accordance with the present invention. It has been discovered that inhibition of aminoacyl tRNA synthetases for multiple amino acids mediate selective, potent effects on biological processes such as Th17 differentiation. As non-limiting examples of the generality of this discovery, it is shown in the Examples and Figures that halofuginone inhibits prolyl-tRNA synthetase, borrelidin inhibits threonyl tRNA synthetase, and tryptophanol inhibits tryptophanyl tRNA synthetase. It is further shown that inhibition of these aminoacyl tRNA synthetases can elicit an amino acid starvation response, modulate IL-17 levels, and modulate Th17 differentiation. Similar effects can be achieved by inhibiting any aminoacyl tRNA synthetase. Furthermore, as discussed herein, inhibition of an aminoacyl tRNA synthetase is not limited to any particular inhibitor or group of inhibitors. Any agent, known, unknown, or to be discovered, that can inhibit an aminoacyl tRNA synthetase may be utilized in the present invention.

In some embodiments of methods provided herein, a tRNA synthetase of a non-essential amino acid (e.g., Ala, Asp, Asn, Cys, Glu, Gln, Gly, Pro, Ser, Tyr, Arg, His) is inhibited. In some embodiments, a tRNA synthetase of an essential amino acid (e.g., Phe, Val, Thr, Trp, Ile, Met, Leu, or Lys) is inhibited. In certain embodiments, glutamyl-prolyl tRNA synthetase (EPRS) is inhibited. In certain embodiments, a prolyl (Pro) tRNA synthetase is inhibited. In certain embodiments, cysteinyl (Cys) tRNA synthetase is inhibited. In certain embodiments, methionyl (Met) tRNA synthetase is inhibited. In certain embodiments, leucyl (Leu) tRNA synthetase is inhibited. In certain embodiments, tryptophanyl (Trp) tRNA synthetase is inhibited. In certain embodiments, glycyl (Gly) tRNA synthetase is inhibited. In certain embodiments, alanyl (Ala) tRNA synthetase is inhibited. In certain embodiments, valyl (Val) tRNA synthetase is inhibited. In certain embodiments, isoleucyl (Ile) tRNA synthetase is inhibited. In certain embodiments, aspartyl (Asp) tRNA synthetase is inhibited. In certain embodiments, glutamyl (Glu) tRNA synthetase is inhibited. In certain embodiments, asparagyl (Asn) tRNA synthetase is inhibited. In certain embodiments, glutaminyl (Gln) tRNA synthetase is inhibited. In certain embodiments, seryl (Ser) tRNA synthetase is inhibited. In certain embodiments, threonyl (Thr) tRNA synthetase is inhibited. In certain embodiments, lysyl (Lys) tRNA synthetase is inhibited. In certain embodiments, arginyl (Arg) tRNA synthetase is inhibited. In certain embodiments, histidyl (His) tRNA synthetase is inhibited. In certain embodiments, phenylalanyl (Phe) tRNA synthetase is inhibited. In certain embodiments, tyrosyl (Tyr) tRNA synthetase is inhibited.

Any agent that inhibits a eukaryotic aminoacyl tRNA synthetase can be used in a method described herein (e.g., a method of modulating a Th17-mediated immune response). In certain embodiments, an aminoacyl tRNA synthetase inhibitor used in a method described herein is an active site inhibitor (i.e., a competitive inhibitor) of a tRNA synthetase. In certain embodiments, an aminoacyl tRNA synthetase inhibitor used in a method described herein is a noncompetitive inhibitor of a tRNA synthetase. In certain embodiments, an aminoacyl tRNA synthetase inhibitor used in a method described herein comprises an amino acid alcohol. In certain embodiments, an aminoacyl tRNA synthetase inhibitor used in a method described herein is specific for a eukaryotic aminoacyl tRNA synthetase (i.e., the inhibitor does not significantly inhibit activity of a prokaryotic aminoacyl tRNA synthetase).

In certain embodiments, an inhibitor of a glutamyl-prolyl tRNA synthetase (EPRS) is employed in a method provided herein. Halofuginone is an example of an EPRS inhibitor.

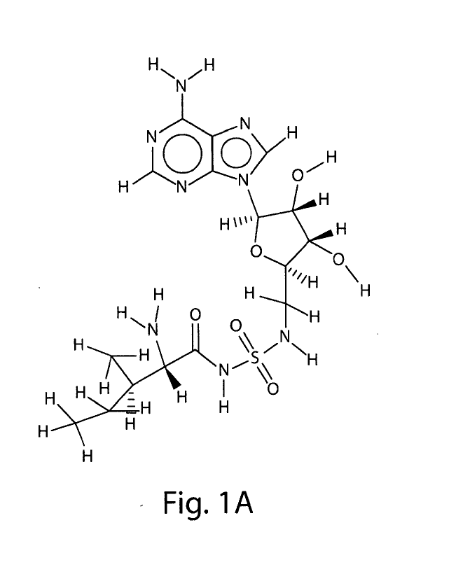

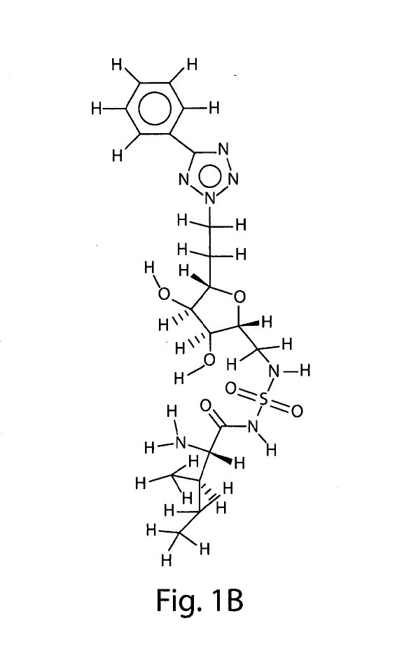

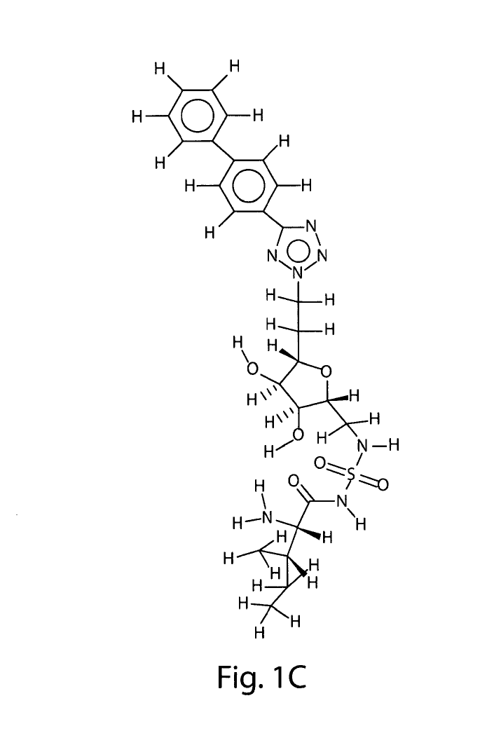

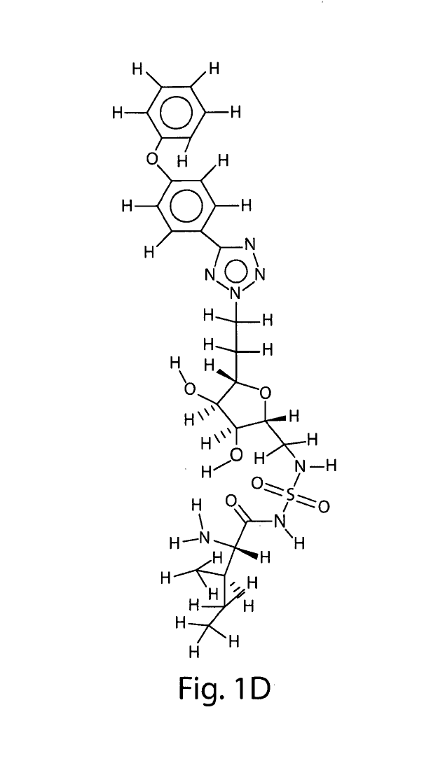

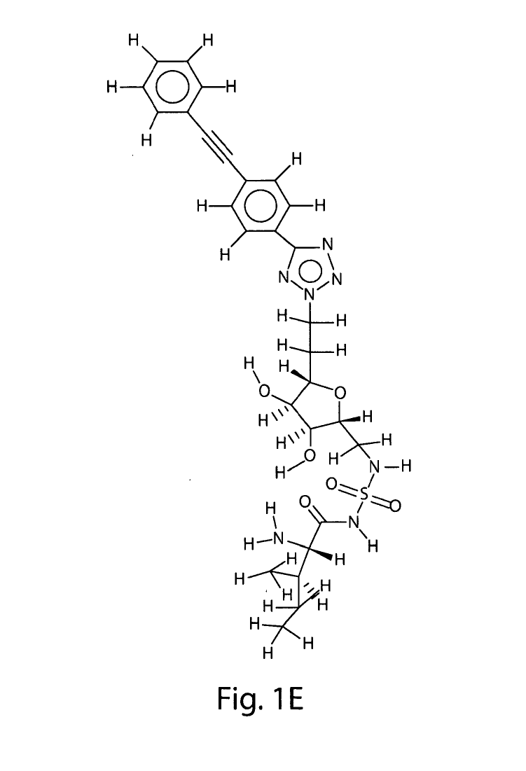

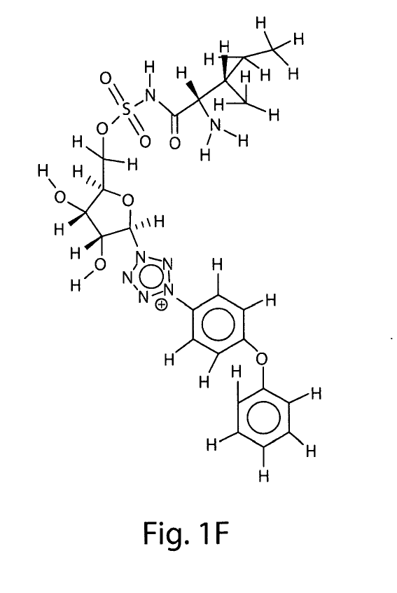

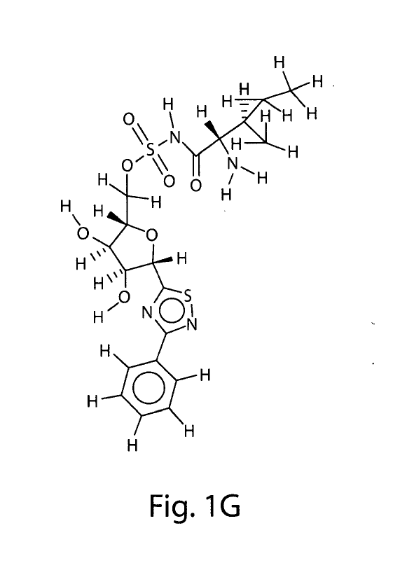

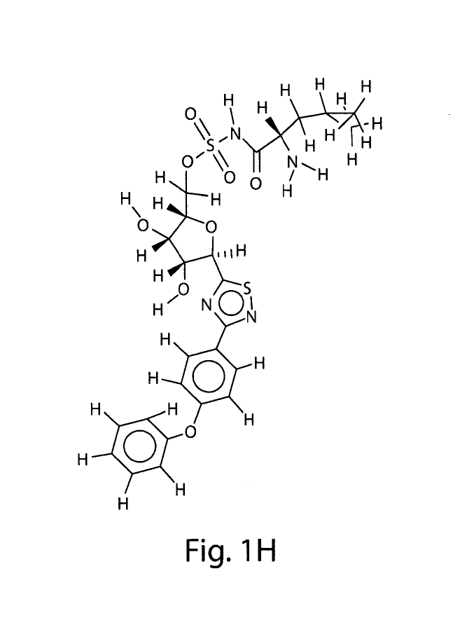

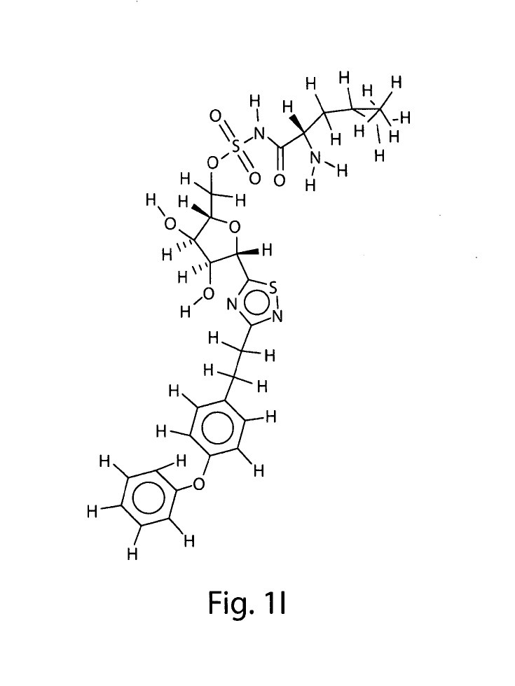

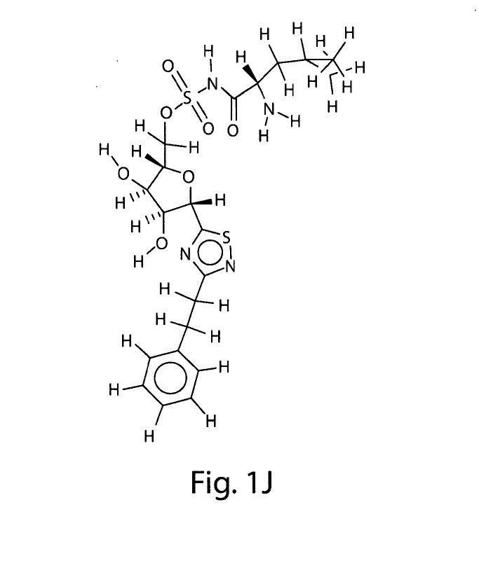

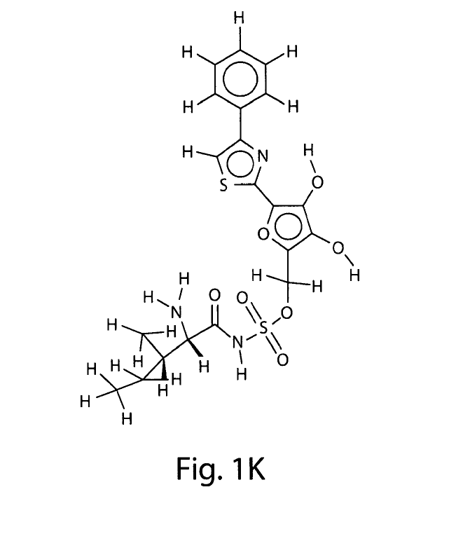

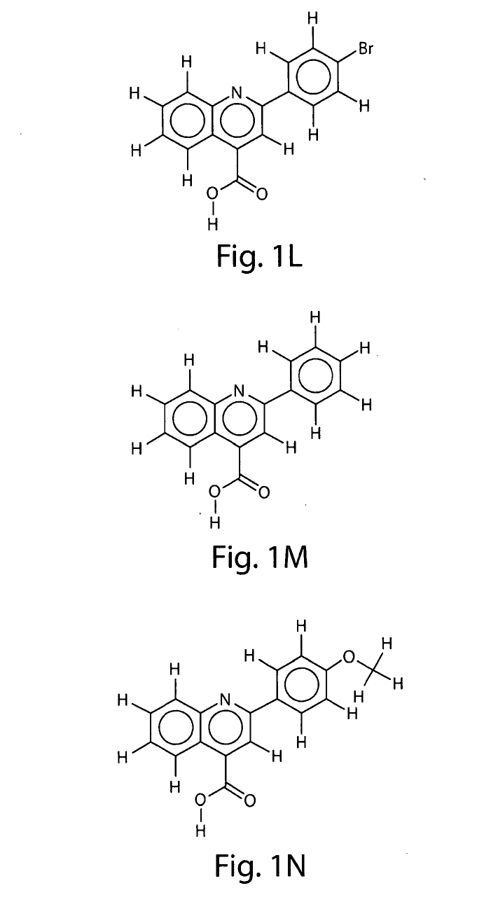

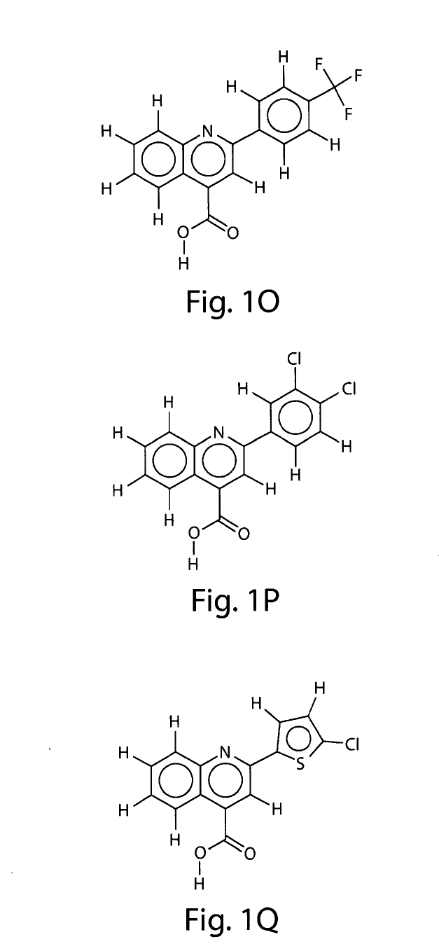

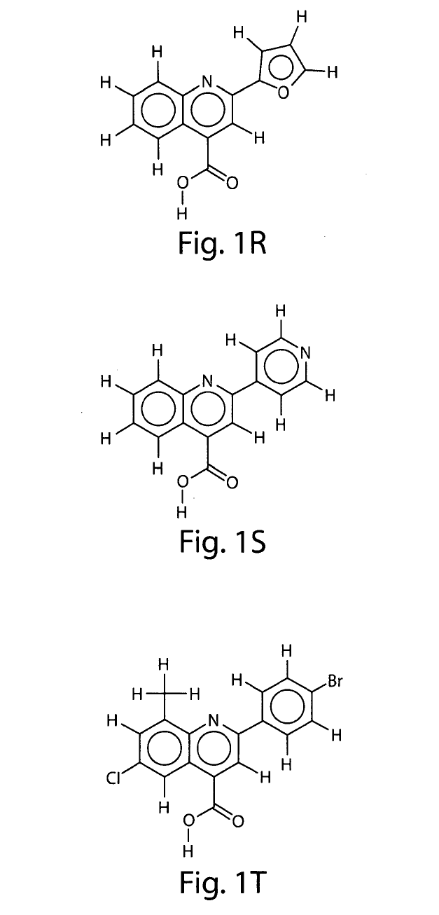

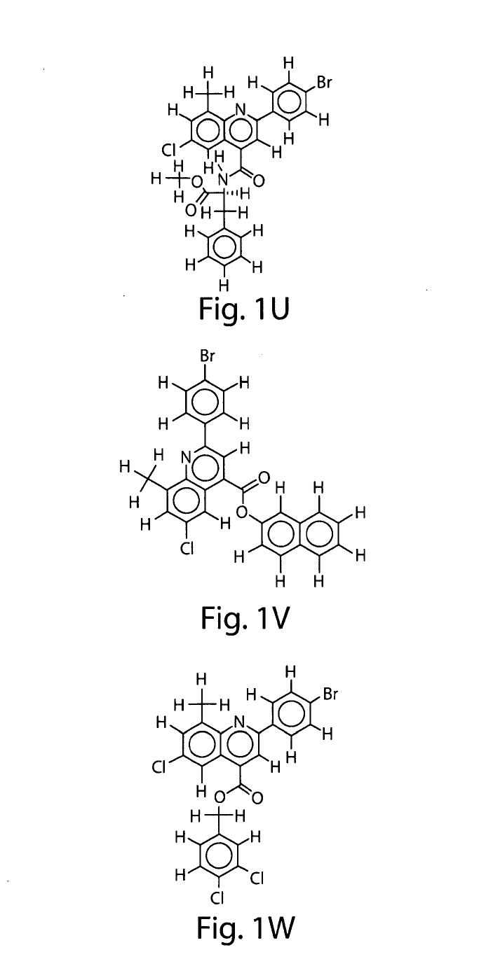

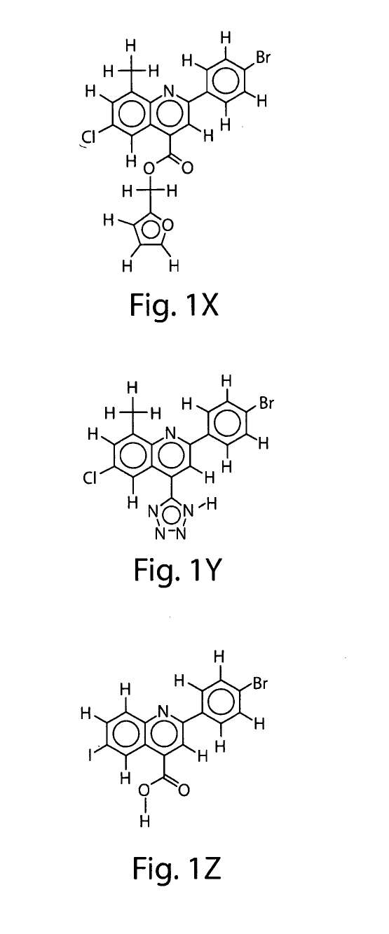

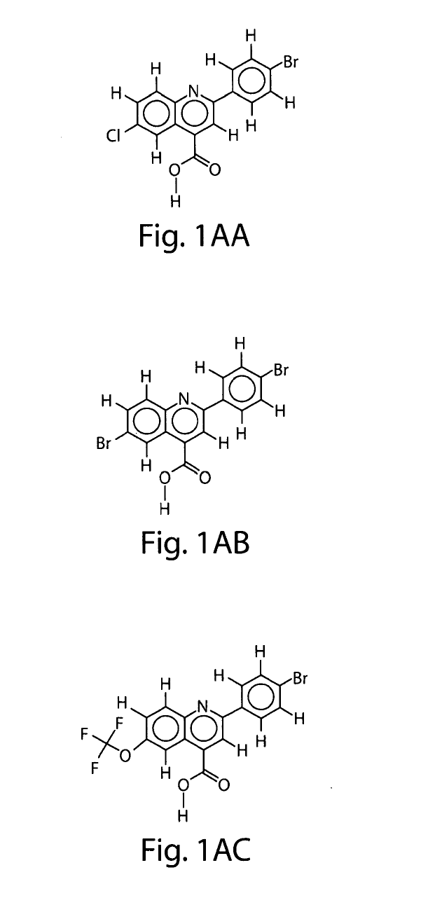

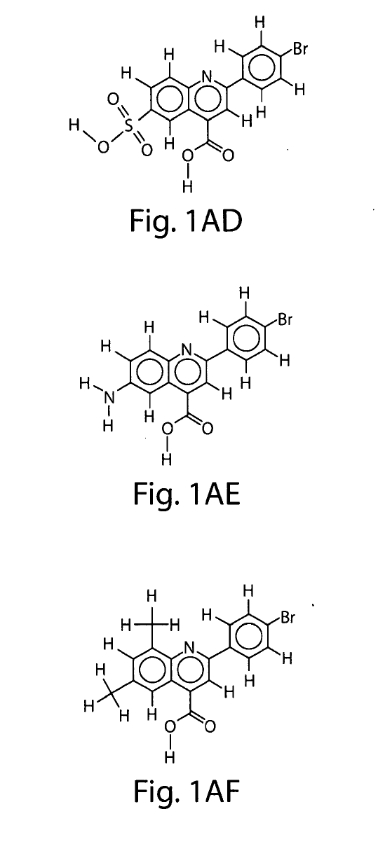

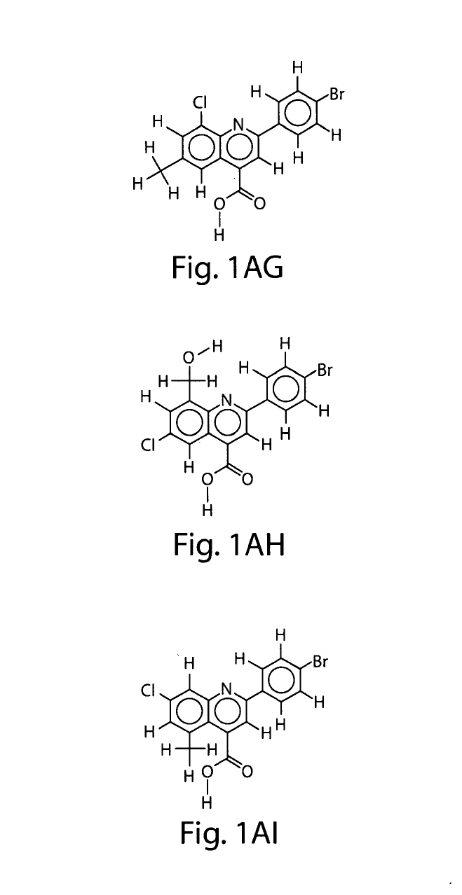

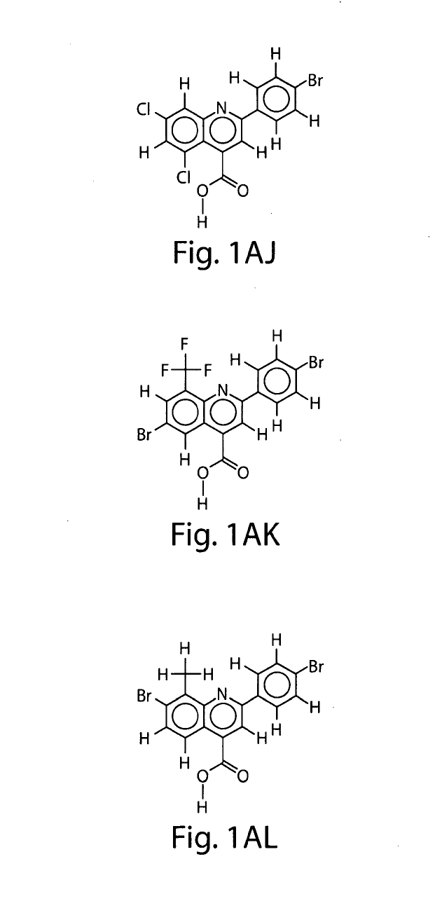

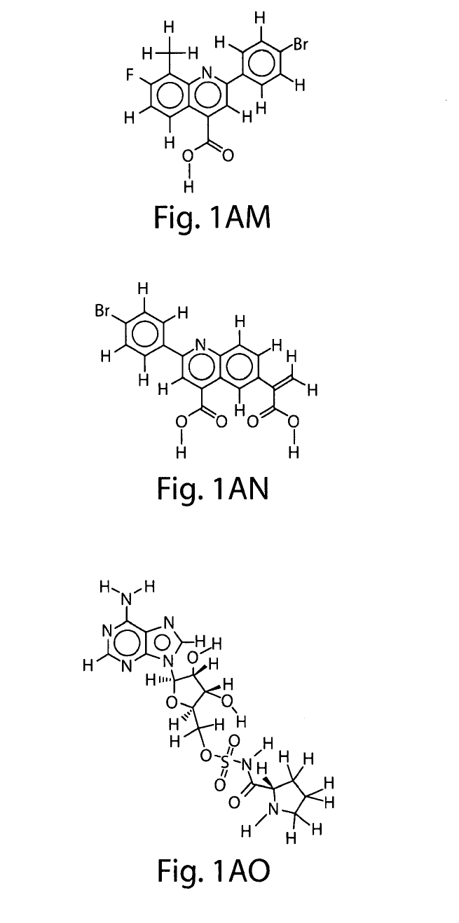

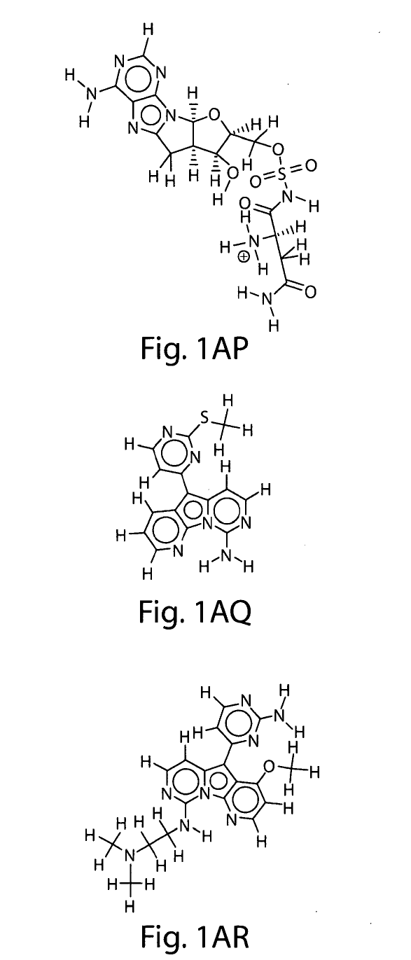

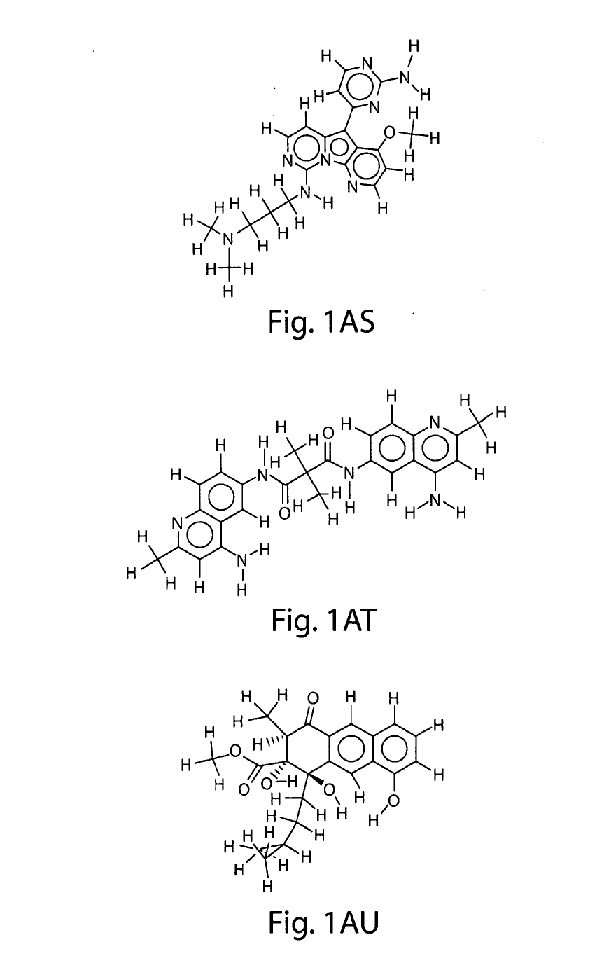

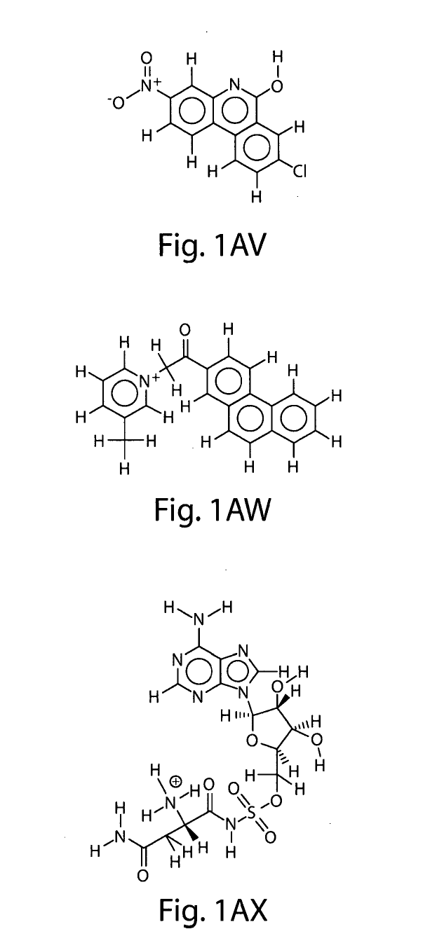

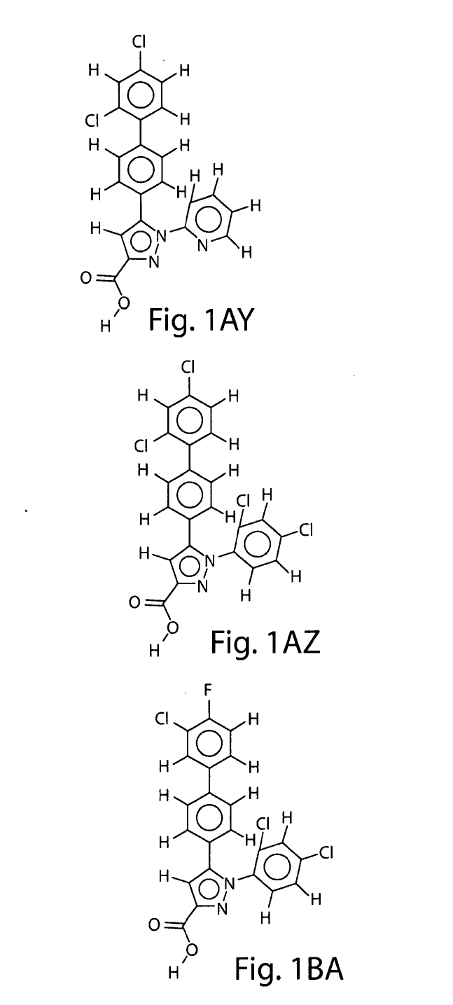

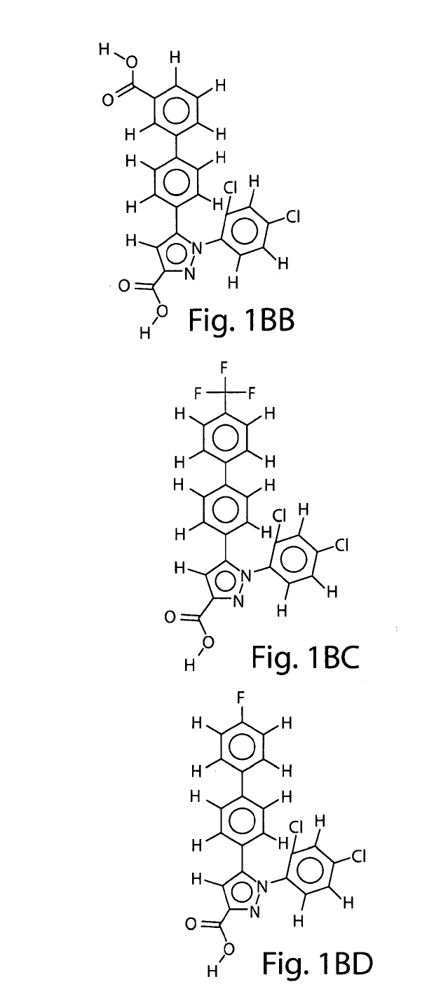

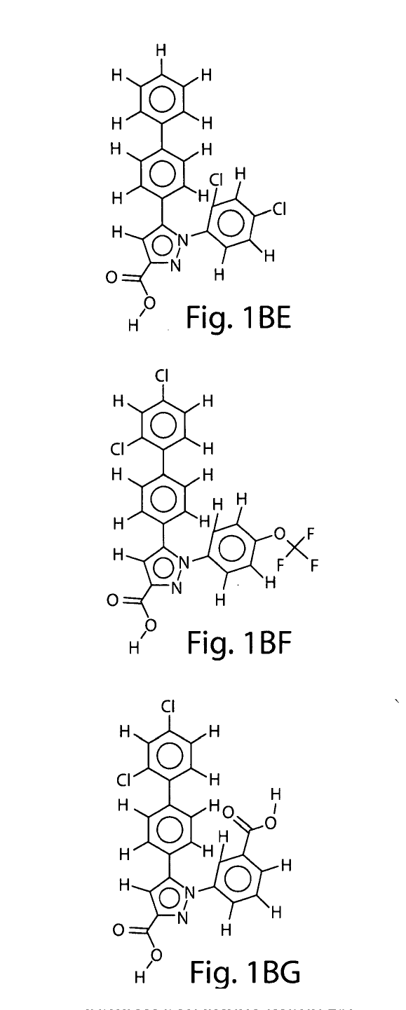

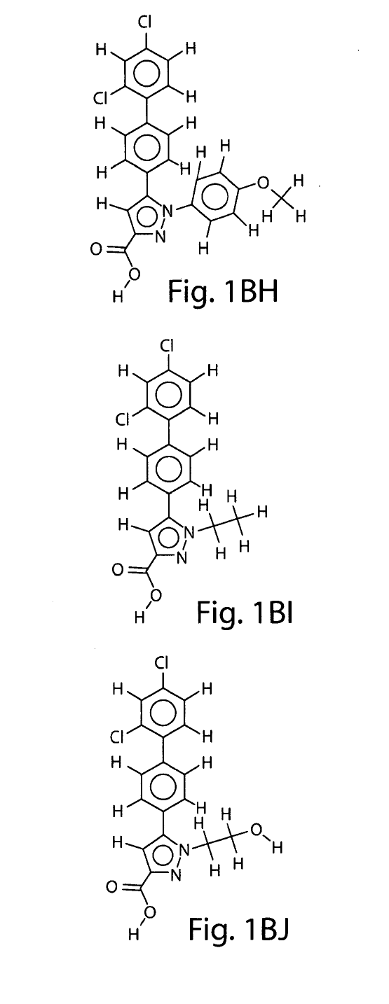

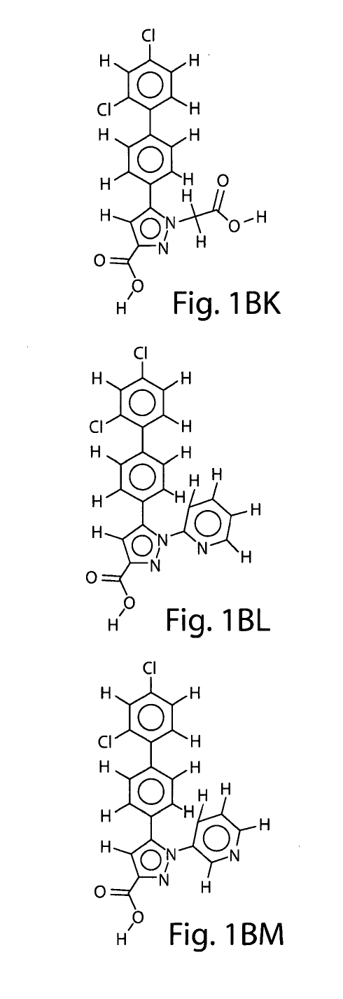

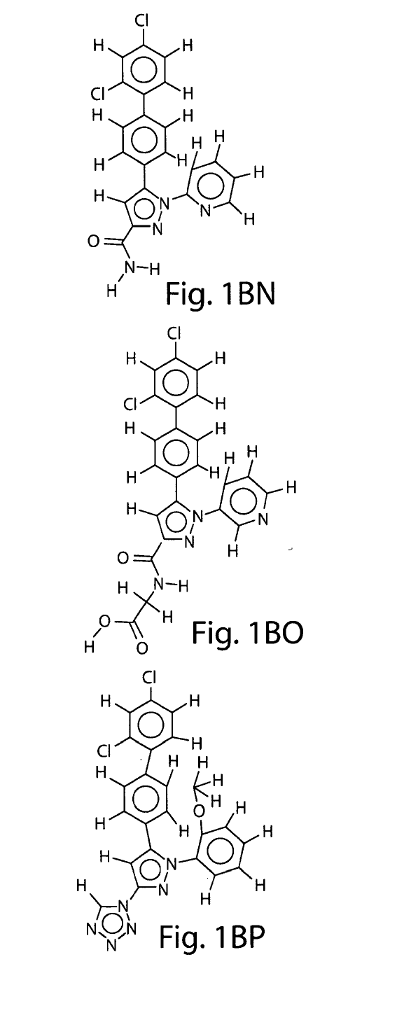

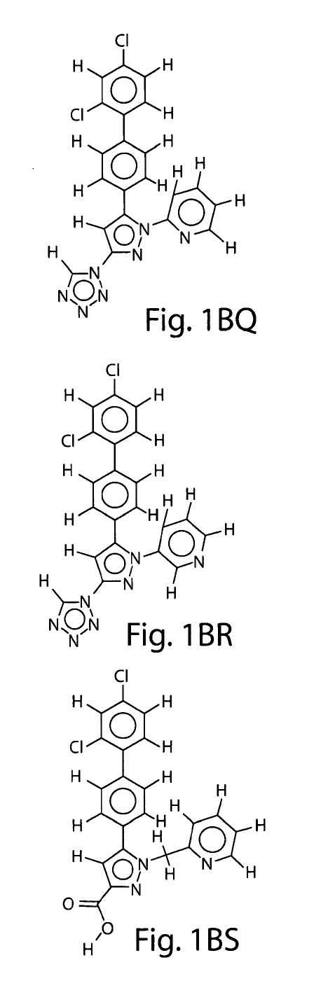

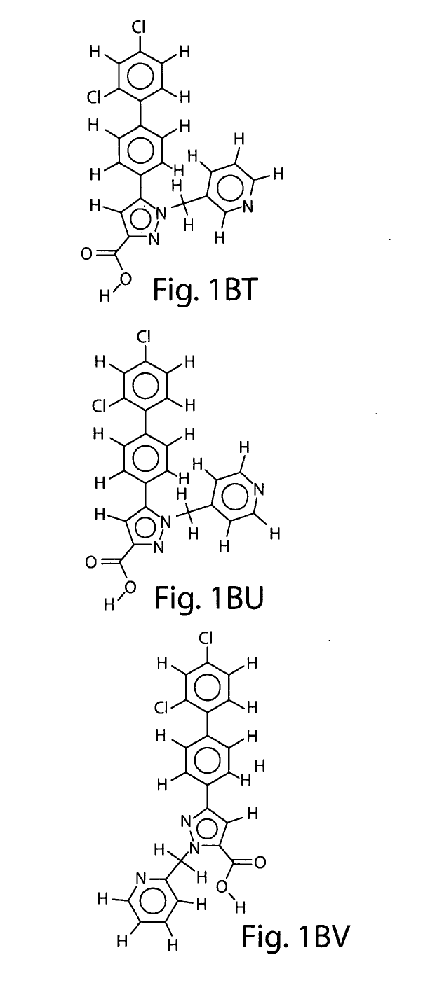

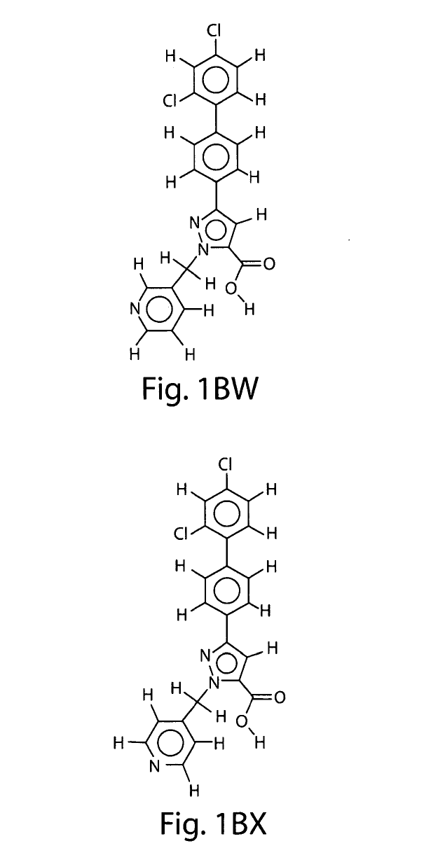

In certain embodiments, an isoleucyl tRNA synthetase inhibitor is employed. Examples of isoleucyl tRNA synthetase inhibitors include cispentacin, mupirocin, Icofungipen, reveromycin A, isoleucinol, and the compounds shown in FIGS. 1A-1E. See also Schimmel, et al., FASEB J., 12(15):1599-609, 1998; and Hurdle et al., Antimicrob. Agents Chemother., 49(12):4821-33, 2005). Reveromycin A is an isoleucyl tRNA synthetase inhibitor produced by Actinomycetes.

In certain embodiments, a tryptophanyl tRNA synthetase inhibitor is employed. Tryptophanol is an example of a tryptophanyl tRNA synthetase inhibitor.

In certain embodiments, a histidinyl tRNA synthetase inhibitor is employed. Histidinol is an example of a histindinyl tRNA synthetase inhibitor.

In certain embodiments, a leucyl tRNA synthetase inhibitor is employed. Examples of leucyl tRNA synthetase inhibitors are AN2690, leucinol, and the compounds shown in FIGS. 1F-1K. See also Winum et al., Med. Res. Rev., 25(2):186-228, 2005; and Kim et al., Appl. Microbiol. Biotechnol., 61(4):278-88, 2003.

In certain embodiments, a prolyl tRNA synthetase inhibitor is employed. Prolinol is a prolyl tRNA synthetase inhibitor. In addition, FIGS. 1L-1AP show examples of inhibitors of prolyl tRNA synthetase. See also Yu et al., Bioorg. Med. Chem. Lett., 11(4):541-4, 2001; and Hurdle et al., Antimicrob. Agents Chemother., 49(12):4821-33, 2005.

In certain embodiments, an asparagyl tRNA synthetase inhibitor is employed. Asparaginol is an example of an asparagyl tRNA synthetase inhibitor. FIGS. 1AP-1AX show examples of inhibitors of asparagyl tRNA synthetase. See also Sukuru et al., J. Comput. Aided Mol. Des., 20(3):159-78, 2006.

In certain embodiments, a methionyl tRNA synthetase inhibitor is employed. Examples of inhibitors of methionyl tRNA synthetase include methionol and compounds shown in FIGS. 1AY-BX. See also Finn et al., Bioorg. Med. Chem. Lett., 13(13):2231-4, 2003.

In certain embodiments, a threonyl tRNA synthetase inhibitor is employed. Threoninol and borrelidin are examples of threonyl tRNA synthetase inhibitors. Borrelidin is a macrolide polyketide produced by Streptomyces species and acts as a noncompetitive inhibitor of threonyl tRNA synthetase.

In certain embodiments, a tyrosyl tRNA synthetase inhibitor is employed. Tyrosinol is an example of a tyrosyl tRNA synthetase inhibitor.

In certain embodiments, a glycyl tRNA synthetase inhibitor is employed. Glycinol is an example of a glycyl tRNA synthetase inhibitor.

In certain embodiments, a valyl tRNA synthetase inhibitor is employed. Valinol is an example of a valyl tRNA synthetase inhibitor.

In certain embodiments, a glutaminyl tRNA synthetase inhibitor is employed. Glutaminol is an example of a glutaminyl tRNA synthetase inhibitor.

In certain embodiments, a cysteinyl tRNA synthetase inhibitor is employed. Cysteinol is an example of a cysteinyl tRNA synthetase inhibitor.

In certain embodiments, an alanyl tRNA synthetase inhibitor is employed. Alaninol is an example of an alanyl tRNA synthetase inhibitor.

In certain embodiments, an aspartyl tRNA synthetase inhibitor is employed. Aspartanol is an example of an aspartyl tRNA synthetase inhibitor.

In certain embodiments, a glutamyl tRNA synthetase inhibitor is employed. Glutamol is an example of a glutamyl tRNA synthetase inhibitor.

In certain embodiments, a seryl tRNA synthetase inhibitor is employed. Serinol is an example of a seryl tRNA synthetase inhibitor.

In certain embodiments, a lysyl tRNA synthetase inhibitor is employed. Lysinol is an example of a lysyl tRNA synthetase inhibitor.

In certain embodiments, an arginyl tRNA synthetase inhibitor is employed. Arginol is an example of an arginyl tRNA synthetase inhibitor.

In certain embodiments, a phenylalanyl tRNA synthetase inhibitor is employed. Phenylalanol is an example of a phenylalanyl tRNA synthetase inhibitor.

Additional tRNA synthetase inhibitors are found in a database found on the interne at the following address: ia.bioinfo.pl/download.php (Torchala and Hoffmann, I A, Database of known ligands of aminoacyl-tRNA synthetases, J. Comp-Aid. Mol. Des. 21:523-525, 2007) and are described in the following references, which are herein incorporated by reference in their entireties: Szymanski et al., The new aspects of aminoacyl-tRNA synthetases. Acta Biochim. Pol., 47(3): 821-34, 2000; Sukuru et al., Discovering new classes of Brugia malayi asparaginyl-tRNA synthetase inhibitors and relating specificity to conformational change. J. Comput. Aided Mol. Des., 20(3): 159-78, 2006; Kim et al., Deoxyribosyl analogues of methionyl and isoleucyl sulfamate adenylates as inhibitors of methionyl-tRNA and isoleucyl-tRNA synthetases. Bioorg. Med. Chem. Lett., 15(14):3389-93, 2005; Farhanullah et al., Design and synthesis of quinolinones as methionyl-tRNA synthetase inhibitors. Bioorg. Med. Chem., 14:7154-9, 2006; Jarvest et al., Discovery and optimisation of potent, selective, ethanolamine inhibitors of bacterial phenylalanyl tRNA synthetase. Bioorg. Med. Chem. Lett., 15(9):2305-9, 2005; Critchley et al., Antibacterial activity of REP8839, a new antibiotic for topical use. Antimicrob. Agents Chemother., 49(10): 4247-52, 2005; Petraitis et al., Efficacy of PLD-118, a novel inhibitor of candida isoleucyl-tRNA synthetase, against experimental oropharyngeal and esophageal candidiasis caused by fluconazole-resistant C. albicans. Antimicrob. Agents Chemother., 48(10): 3959-67, 2004; Kanamaru et al., In vitro and in vivo antibacterial activities of TAK-083, an agent for treatment of Helicobacter pylori infection. Antimicrob. Agents Chemother., 45(9):2455-9, 2001; Winum et al., Sulfamates and their therapeutic potential. Med. Res. Rev., 25(2):186-228, 2005; Schimmel et al., Aminoacyl tRNA synthetases as targets for new anti-infectives. FASEB J., 12(15):1599-609, 1998; Yu et al., A series of quinoline analogues as potent inhibitors of C. albicans prolyl tRNA synthetase. Bioorg. Med. Chem. Lett., 11(4):541-4, 2001; Kim et al., Aminoacyl-tRNA synthetases and their inhibitors as a novel family of antibiotics. Appl. Microbiol. Biotechnol., 61(4):278-88, 2003; Banwell et al., Analogues of SB-203207 as inhibitors of tRNA synthetases. Bioorg. Med. Chem. Lett., 10(20): 2263-6, 2000; Jarvest et al., Inhibitors of bacterial tyrosyl tRNA synthetase: synthesis of carbocyclic analogues of the natural product SB-219383. Bioorg. Med. Chem. Lett., 11(18):2499-502, 2001; Yu et al., A series of heterocyclic inhibitors of phenylalanyl-tRNA synthetases with antibacterial activity. Bioorg. Med. Chem. Lett., 14(5):1343-6, 2004; Tandon et al., Potent and selective inhibitors of bacterial methionyl tRNA synthetase derived from an oxazolone-dipeptide scaffold. Bioorg. Med. Chem. Lett., 14(8):1909-11, 2004; Hurdle et al., Prospects for aminoacyl-tRNA synthetase inhibitors as new antimicrobial agents. Antimicrob. Agents Chemother., 49(12):4821-33, 2005; Pohlmann and Brotz-Oesterhelt, New aminoacyl-tRNA synthetase inhibitors as antibacterial agents. Curr. Drug. Targets Infect. Disord., 4(4):261-72, 2004; Jarvest et al., Definition of the heterocyclic pharmacophore of bacterial methionyl tRNA synthetase inhibitors: potent antibacterially active non-quinolone analogues. Bioorg. Med. Chem. Lett., 14(15):3937-41, 2004; Jarvest et al., Conformational restriction of methionyl tRNA synthetase inhibitors leading to analogues with potent inhibition and excellent gram-positive antibacterial activity. Bioorg. Med. Chem. Lett., 13(7):1265-8, 2003; Qiu et al., Crystal structure of Staphylococcus aureus tyrosyl-tRNA synthetase in complex with a class of potent and specific inhibitors. Protein Sci., 10(10):2008-16, 2001; Finn et al., Discovery of a potent and selective series of pyrazole bacterial methionyl-tRNA synthetase inhibitors. Bioorg. Med. Chem. Lett., 13(13):2231-4, 2003; Lee et al., N-Alkoxysulfamide, N-hydroxysulfamide, and sulfamate analogues of methionyl and isoleucyl adenylates as inhibitors of methionyl tRNA and isoleucyl tRNA synthetases. Bioorg. Med. Chem. Lett., 13(6):1087-92, 2003. Structures of 480 tRNA synthetase inhibitors are shown in Appendix A, all of which may be useful in the present invention.

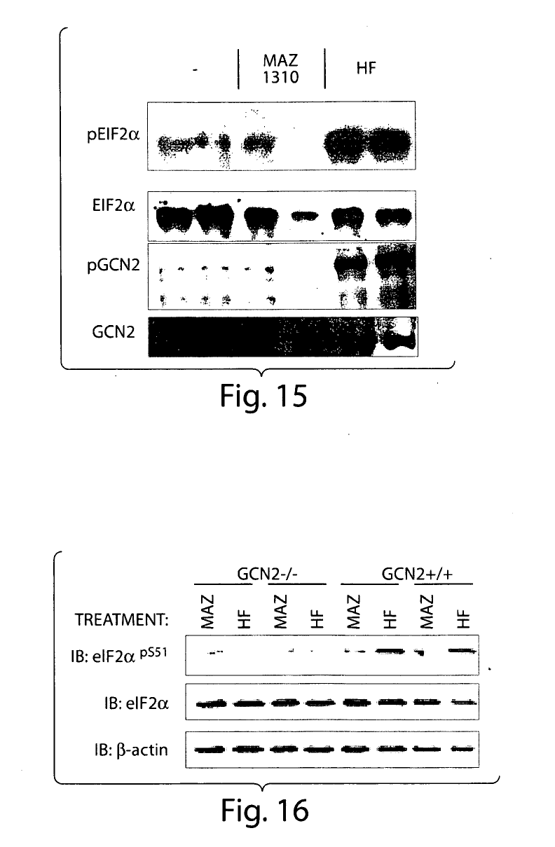

Data herein show that inhibition of tRNA synthetases leads to the accumulation of uncharged prolyl tRNAs, which in turn activate the amino acid starvation response (AAR, FIG. 15). Activation of the AAR in T-cells suppresses the differentiation of a subset of effector T-cells (i.e., Th17 cells). Suppressing the production of Th17 cells has been found to lead to autoimmunity. The AAR also suppresses pro-fibrotic gene expression as well as viral gene expression, replication, and maturation. AAR may contribute to the protection of organs from stress (e.g., ER stress in the pancreas during the development of diabetes). Based on these findings, inhibition of tRNA synthetases may be used in the treatment of a variety of diseases and conditions.

Accordingly, in certain embodiments, an agent that inhibits an aminoacyl tRNA synthetase suppresses the differentiation of a subset of effector T-cells (i.e., Th17 cells). In certain embodiments, an agent that inhibits an aminoacyl tRNA synthetase suppresses IL-17 production. In some embodiments, an agent that inhibits an aminoacyl tRNA synthetase is useful in the treatment of a disease associated with IL-17 production, such as arthritis, inflammatory bowel disease, psoriasis, multiple sclerosis, lupus, asthma, scleroderma, graft rejection, graft versus host disease, chronic inflammation, asthma, and other autoimmune and inflammatory disease.

In some embodiments, an agent that inhibits an aminoacyl tRNA synthetase inhibits viral gene expression, replication, and/or maturation.

In some embodiments, an agent that inhibits an aminoacyl tRNA synthetase inhibits fibrosis. In certain embodiments, an agent that inhibits an aminoacyl tRNA synthetase inhibits angiogenesis. In certain embodiments, an agent that inhibits an aminoacyl tRNA synthetase may be used to inhibit scar formation. In certain embodiments, an agent that inhibits an aminoacyl tRNA synthetase may be used to treat or inhibit cellulite.

In some embodiments, inhibition of an aminoacyl tRNA synthetase can lead to accumulation of uncharged tRNAs, which in turn can activate an AAR.

### In Vitro Methods

Agents that inhibit aminoacyl tRNA synthetases can be used to inhibit cells in vitro. In some embodiments, agents are used to inhibit the development and/or proliferation of Th17 cells, e.g., IL-17 secreting cells, in vitro. An in vitro method for employing an agent that is a tRNA synthetase inhibitor to suppress development and/or proliferation of IL-17 expressing effector T-cells can include contacting a naïve T-cell population with the agent under conditions that allow Th17 cell development and/or proliferation in the absence of the agent, and culturing the cell population. In some embodiments, the level of IL-17 expression and/or the number of Th17 cells in the cell population is assessed. Lack of a change or a decrease in IL-17 expression in the cell population indicates that the agent inhibits Th17 differentiation.

In some embodiments, conditions that allow Th17 cell development include contacting a population of naïve T cells with one or more agents that activate the T cells and incubating the cells in a culture that includes cytokines that drive Th17 differentiation. In some embodiments, T cells are activated with anti-CD3 and anti-CD28 antibodies. As is known to one of skill in the art, other reagents can be used to activate T cells in vitro. Activated T cells can be differentiated into Th17 cells by culture in the presence of TGFβ and IL-6 (see, e.g., Veldhoen et al., Immunity 24:179, 2006; Ivanov et al., Cell 126:1121, 2006; Bettelli et al., Nature 441:235, 2006). In some embodiments, Th17 cell cultures are maintained in the absence of exogenous IL-2. In some embodiments, T cells are restimulated (e.g., with PMA and ionomycin) prior to examination of phenotype.

Determining the level of IL-17 expression and/or the number of Th17 cells in the cell population can be accomplished, for example, by using a detection agent that binds to IL-17 or other marker for Th17 cells, for example, the Th17-specific transcription factors RORγt and RORα, or by detecting STAT3 activation. The detection agent is, for example, an antibody. The detection agent can be coupled with a detectable moiety (e.g., a radioisotope, a fluorescent tag, peptide tag, or enzyme) such that binding of the detection agent to IL-17 or other Th17 marker can be determined by detecting the detectable moiety. In some embodiments, Th17 differentiation is evaluated by determining the percentage of IL-17⁺ T cells (e.g., the percentage of IL-17⁺IFNγ⁻ cells) in a T cell culture following restimulation (e.g., 2-8 days following restimulation). In some embodiments, IL-17 expression is determined by examining IL-17 mRNA expression. In some embodiments, expression of multiple genes is examined (e.g., using gene expression profiling, e.g., microarray analysis).

Selectivity of a tRNA synthetase inhibitor for inhibition of Th17 cell differentiation or proliferation can be examined. In some embodiments, a tRNA synthetase inhibitor reduces Th17 differentiation at a concentration at least 2, 5, 10, or 20 times lower than the concentration at which the inhibitor reduces one or more of general T cell proliferation, CD25 expression, Th1 differentiation, Th2 differentiation, or protein synthesis. In some embodiments, a tRNA synthetase inhibitor inhibits Th17 differentiation at a concentration at least 2, 5, 10, or 20 times lower than the concentration at which the inhibitor modulates cell proliferation in a mixed lymphocyte reaction. In some embodiments, a tRNA synthetase inhibitor modulates Th17 differentiation with an IC₅₀ below 1×10⁻⁶M, e.g., below 1×10⁻⁷M, 1×10⁻⁸M, or 1×10⁻⁹M.

In some embodiments, selectivity of an agent for inhibiting Th17 differentiation is examined, e.g., by comparing the effect of the agent on Th17 differentiation to its effect on one or more of Th1, Th2, and iTreg differentiation. Conditions for directing T cells down Th1, Th2, and iTreg differentiation pathways are known (see, e.g., Djuretic et al., Nat. Immunol. 8:145, 2007). iTreg differentiation can be directed by culturing T cells in the presence of TGFβ. In some embodiments, selectivity of an agent for modulating Th17 differentiation is examined, e.g., by comparing the effect of the agent on Th17 differentiation to its effect on one or more of T cell proliferation, CD25 upregulation, IL-2 production, TNF production, or IFNγ production.

Methods of Modulating Th17 Cell Differentiation and/or Proliferation and Other Cellular Functions Using tRNA Synthetase Inhibitors

Aminoacyl tRNA synthetase inhibitors have been found to specifically alter the development of T cells away from the Th17 lineage, which is associated with cell-mediated damage, persistent inflammation, and autoimmunity.

Th17 cells secrete several cytokines that may have a role in promoting inflammation and fibrosis, including IL-17, IL-6, IL-21, and GM-CSF. Of these cytokines, IL-17 is a specific product of Th17 cells, and not other T cells. Whether Th17 cells are the only source of IL-17 during inflammatory response is not clear, but elevated IL-17 levels are in general thought to reflect expansion of the Th17 cell population.

Diseases that have been associated with expansion of a Th17 cell population or increased IL-17 production include, but are not limited to, rheumatoid arthritis, multiple sclerosis, Crohn's disease, inflammatory bowel disease, Lyme disease, airway inflammation, transplantation rejection, graft versus host disease, lupus, psoriasis, scleroderma, periodontitis, systemic sclerosis, coronary artery disease, myocarditis, atherosclerosis, diabetes, and inflammation associated with microbial infection (e.g., viral, protazoal, fungal, or bacterial infection).

Agents that inhibit a tRNA synthetase can be useful for treatment of any of these diseases by suppressing the chronic inflammatory activity of IL-17 expressing cells, such as IL-17 expressing effector T-cells, e.g., Th17 cells. In some instances, this may address the root cause of the disease (e.g., self-sustaining inflammation in rheumatoid arthritis); in other cases (e.g., diabetes, periodontitis) it may not address the root cause but may ameloriate the symptoms associated with the disease.

IL-17 expressing effector T-cells, e.g., Th17 cells, and their associated cytokine IL-17 provide a broad framework for predicting or diagnosing diseases potentially treatable by agents that inhibit tRNA synthetases. Specifically, pre-clinical fibrosis and/or transplant/graft rejection could be identified and treated with an agent that inhibits a tRNA synthetase, or with a tRNA synthetase inhibitor in combination with other Th17 antagonists. Additionally, diseases that are not currently associated with Th17 cell damage and persistence of inflammation may be identified through the measurement of Th17 cell expansion, or of increased IL-17 levels (e.g., in serum or synovial fluid). Alternatively, or in addition, the use of gene profiling to characterize sets of genes activated subsequent to Th17 differentiation may allow detection of Th17-affected tissues, prior to histological/pathologic changes in tissues.

Agents that inhibit tRNA synthetases could be used in combination with other agents that act to suppress Th17 development to achieve synergistic therapeutic effects. Current examples of potential synergistic agents would include anti-IL-21 antibodies or antigen binding fragments thereof, retinoic acid, or anti-IL-6 antibodies or antigen binding fragments thereof, all of which can reduce Th17 differentiation.

Agents that inhibit tRNA synthetases could be used in combination with other agents that act to suppress inflammation and/or immunological reactions, such as steroids (e.g., cortisol (hydrocortisone), dexamethasone, methylprednisolone, and/or prednisolone), non-steroidal anti-inflammatory drugs (NSAIDs; e.g., ibuprofen, acetominophin, aspirin, celecoxib, valdecoxib, etoricoxib, lumiracoxib, parecoxib, rofecoxib, nimesulide, and/or naproxen), or immunosuppressants (e.g., cyclosporine, rapamycin, and/or FK506). In some embodiments, an agent that inhibits a tRNA synthetase is used in combination with an inhibitor of a proinflammatory cytokine. Proinflammatory cytokines that can be targeted (in addition to IL-6 and IL-21, discussed above) include TNFα, IFNγ, GM-CSF, MIP-2, IL-12, IL-1α, IL-1β, and IL-23. Examples of such inhibitors include antibodies that bind to the cytokine or that bind to a receptor of the cytokine and block its activity, agents that reduce expression of the cytokine (e.g., small interfering RNA (siRNA) or antisense agents), soluble cytokine receptors, and small molecule inhibitors (see, e.g., WO 2007/058990).

In some embodiments, agents that inhibit tRNA synthetases are used in combination with an inhibitor of TNFα. In some embodiments, an inhibitor of TNFα comprises an anti-TNFα antibody or antigen binding fragment thereof. In some embodiments, the anti-TNFα antibody is adalimumab (Humira™). In some embodiments, the anti-TNFα antibody is infliximab (Remicade™). In some embodiments, the anti-TNFα antibody is CDP571. In some embodiments, an inhibitor of TNFα comprises a TNFα receptor. For example, in some embodiments, the TNFα inhibitor is etanercept (Enbrel™), which is a recombinant fusion protein having two soluble TNF receptors joined by the Fc fragment of a human IgG1 molecule. In some embodiments, an inhibitor of TNFα comprises an agent that inhibit expression of TNFα, e.g., such as nucleic acid molecules that mediate RNA interference (RNAi) (e.g., a TNFα selective siRNA or shRNA) or antisense oligonucleotides. For example, a TNFα inhibitor can include, e.g., a short interfering nucleic acid (siNA), a short interfering RNA (siRNA), a double-stranded RNA (dsRNA), or a short hairpin RNA (shRNA) (see, e.g., U.S. Patent Application No. 20050227935).

Aminoacyl tRNA synthetase inhibitors can be evaluated in animal models. To determine whether a particular aminoacyl tRNA synthetase inhibitor suppresses graft rejection, allogeneic or xenogeneic grafting (e.g., skin grafting, organ transplantation, or cell implantation) can be performed on an animal such as a rat, mouse, rabbit, guinea pig, dog, or non-human primate. Strains of mice such as C57B1-10, B10.BR, and B10.AKM (Jackson Laboratory, Bar Harbor, Me.), which have the same genetic background but are mismatched for the H-2 locus, are well suited for assessing various organ grafts.

In another example, heart transplantation is performed, e.g., by performing cardiac grafts by anastomosis of the donor heart to the great vessels in the abdomen of the host as described by Ono et al., J. Thorac. Cardiovasc. Surg. 57:225, 1969. See also Corry et al., Transplantation 16:343, 1973. Function of the transplanted heart can be assessed by palpation of ventricular contractions through the abdominal wall. Rejection is defined as the cessation of myocardial contractions. A tRNA synthetase inhibitor would be considered effective in reducing organ rejection if animals treated with the inhibitor experience a longer period of myocardial contractions of the donor heart than do untreated hosts.

In another example, effectiveness of an aminoacyl tRNA synthetase inhibitor at reducing skin graft rejection is assessed in an animal model. To perform skin grafts on a rodent, a donor animal is anesthetized and a full thickness skin is removed from a part of the tail. The recipient animal is also anesthetized, and a graft bed is prepared by removing a patch of skin (e.g., 0.5×0.5 cm) from the shaved flank. Donor skin is shaped to fit the graft bed, positioned, covered with gauze, and bandaged. Grafts are inspected daily beginning on the sixth post-operative day and are considered rejected when more than half of the transplanted epithelium appears to be non-viable. A tRNA synthetase inhibitor that causes a host to experience a longer period of engraftment than seen in an untreated host would be considered effective in this type of experiment.

In another example, a tRNA synthetase inhibitor is evaluated in a pancreatic islet cell allograft model. DBA/2J islet cell allografts can be transplanted into rodents, such as 6-8 week-old B6 AFl mice rendered diabetic by a single intraperitoneal injection of streptozotocin (225 mg/kg; Sigma Chemical Co., St. Louis, Mo.). As a control, syngeneic islet cell grafts can be transplanted into diabetic mice. Islet cell transplantation can be performed by following published protocols (for example, see Emamaullee et al., Diabetes 56(5):1289-98, 2007). Allograft function can be followed by serial blood glucose measurements (Accu-Check III™; Boehringer, Mannheim, Germany). A rise in blood glucose exceeding normal levels (on each of at least 2 successive days) following a period of primary graft function is indicative of graft rejection. The NOD (non-obese diabetic) mouse model is another model that can be used to evaluate ability of an agent to treat or prevent type I diabetes.

In another example, a tRNA synthetase inhibitor is evaluated in a model of dry eye disease (DED). In one such model, DED is induced in mice in a controlled environment chamber by administering scopolamine hydrobromide into the skin four times daily. Chamber conditions include a relative humidity <30%, airlflow of 15 L/min, and constant temperature (21-23° C.). Induction of dry eye can be confirmed by measuring changes in corneal integrity with corneal fluorescein staining (see, e.g., Chauhan et al., J. Immunol. 182:1247-1252, 2009; Barabino et al., Invest. Ophthamol. Visual Sci. 46:2766-2771, 2005; and Rashid et al., Arch. Ophthamol. 126: 219-225, 2008).

Numerous autoimmune diseases have been modeled in animals, including rheumatic diseases, such as rheumatoid arthritis and systemic lupus erythematosus (SLE), type I diabetes, and autoimmune diseases of the thyroid, gut, and central nervous system. For example, animal models of SLE include MRL mice, BXSB mice, and NZB mice and their Fl hybrids. The general health of the animal as well as the histological appearance of renal tissue can be used to determine whether the administration of a tRNA synthetase inhibitor can effectively suppress the immune response in an animal model of one of these diseases.

Animal models of intestinal inflammation are described, for example, by Elliott et al. (Elliott et al., 1998, Inflammatory Bowel Disease and Celiac Disease. In: The Autoimmune Diseases, Third ed., N. R. Rose and I. R. MacKay, eds. Academic Press, San Diego, Calif.). Some mice with genetically engineered gene deletions develop chronic bowel inflammation similar to IBD. See, e.g., Elson et al., Gastroenterology 109:1344, 1995; Ludviksson et al., J. Immunol. 158:104, 1997; and Mombaerts et al., Cell 75:274, 1993). One of the MRL strains of mice that develops SLE, MRL-lpr/lpr, also develops a form of arthritis that resembles rheumatoid arthritis in humans (Theofilopoulos et al., Adv. Immunol. 37:269, 1985).

Models of autoimmune disease in the central nervous system (CNS), such as experimental allergic encephalomyelitis (EAE), can also be experimentally induced, e.g., by injection of brain or spinal cord tissue with adjuvant into the animal (see, e.g., Steinman and Zamvil, Ann Neurol. 60:12-21, 2006). In one EAE model, C57B/6 mice are injected with an immunodominant peptide of myelin basic protein in Complete Freund's Adjuvant. EAE disease correlates such as limp tail, weak/altered gait, hind limb paralysis, forelimb paralysis, and morbidity are monitored in animals treated with a tRNA synthetase inhibitor as compared to controls.

In addition to T cell differentiation processes, aminoacyl tRNA synthetase inhibitors can specifically alter processes such as fibrosis and angiogenesis. Fibrosis can be assayed in vitro by observing the effect of a tRNA synthetase inhibitor on fibroblast behavior. In one exemplary assay for use in evaluating tRNA synthetase inhibitors, primary dermal fibroblasts are cultured in a matrix of Type I collagen, which mimics the interstitial matrix of the dermis and hypodermis, such that fibroblasts attach to the substratum and spread. Inhibition of fibroblast attachment and spreading in the presence of an inhibitor indicates that the inhibitor has anti-fibrotic properties. Biological effects of tRNA synthetase inhibitors on non-immune cell functions can also be evaluated in vivo. In some embodiments, an agent that inhibits an aminoacyl tRNA synthetase reduces extracellular matrix deposition (e.g., in an animal model of wound healing; see, e.g., Pines et al., Biol. Bone Marrow Transplant 9:417-425, 2003). In some embodiments, an aminoacyl tRNA synthetase inhibitor reduces extracellular matrix deposition at a concentration lower than the concentration at which it inhibits another cellular function, such as cell proliferation or protein synthesis.

The invention further provides methods of treating a disease using an agent that inhibits tRNA synthetases. The inventive method involves the administration of a therapeutically effective amount of an agent that inhibits a tRNA synthetase to a subject (including, but not limited to a human or other animal).

Compounds and compositions described herein are generally useful for the inhibition of the activity of one or more eukaryotic aminoacyl tRNA synthetases. Examples of tRNA synthetases that can be inhibited include tRNA synthetases of an essential amino acid (e.g., Phe, Val, Thr, Trp, Ile, Met, Leu, or Lys) and tRNA synthetases of a non-essential amino acid. In certain embodiments, a glutamyl-prolyl tRNA synthetase (EPRS) is inhibited. In certain embodiments, a prolyl tRNA synthetase is inhibited. In certain embodiments, a cysteinyl tRNA synthetase is inhibited. In certain embodiments, a methionyl tRNA synthetase is inhibited. In certain embodiments, a leucyl tRNA synthetase is inhibited. In certain embodiments, a tryptophanyl tRNA synthetase is inhibited. In certain embodiments, a glycyl tRNA synthetase is inhibited. In certain embodiments, an alanyl tRNA synthetase is inhibited. In certain embodiments, a valyl tRNA synthetase is inhibited. In certain embodiments, an isoleucyl tRNA synthetase is inhibited. In certain embodiments, an aspartyl tRNA synthetase is inhibited. In certain embodiments, a glutamyl tRNA synthetase is inhibited. In certain embodiments, an asparagyl tRNA synthetase is inhibited. In certain embodiments, a glutaminyl tRNA synthetase is inhibited. In certain embodiments, a seryl tRNA synthetase is inhibited. In certain embodiments, a threonyl tRNA synthetase is inhibited. In certain embodiments, a lysyl tRNA synthetase is inhibited. In certain embodiments, an arginyl tRNA synthetase is inhibited. In certain embodiments, a histidyl tRNA synthetase is inhibited. In certain embodiments, a phenylalanyl tRNA synthetase is inhibited. In certain embodiments, a tyrosyl tRNA synthetase is inhibited.

Inhibition of an aminoacyl tRNA synthetase leads to the accumulation of uncharged tRNAs, which in turn activate the amino acid starvation response (AAR). Activation of this response suppresses 1) pro-fibrotic gene expression; 2) the differentiation of naïve T-cells into Th17 cells that promote autoimmunity; 3) viral gene expression, replication, and maturation; and/or 4) stress to organs (e.g., during transplantation).

In some embodiments, an aminoacyl tRNA synthetase inhibitor has anti-fibrotic properties in vivo. For example, an EPRS inhibitor, halofuginone, potently reduces dermal extracellular matrix (ECM) deposition (Pines, et al., Biol. Blood Marrow Transplant 9: 417-425, 2003). Halofuginone inhibits the transcription of a number of components and modulators of ECM function, including Type I collagen, fibronectin, the matrix metallopeptidases MMP-2 and MMP-9, and the metalloprotease inhibitor TIMP-2 (Li, et al., World J. Gastroenterol. 11: 3046-3050, 2005; Pines, et al., Biol. Blood Marrow Transplant 9: 417-425, 2003). The major cell types responsible for altered ECM deposition, tissue thickening, and contracting during fibrosis are fibroblasts and myofibroblasts. Myofibroblasts mature/differentiate from their precursor fibroblasts in response to cytokine release, often following tissue damage and mechanical stress, and can be distinguished from fibroblasts in a wide range of organs and pathological conditions (Border, et al., New Eng. J. Med. 331: 1286-1292, 1994; Branton et al., Microbes Infect. 1: 1349-1365, 1999; Flanders, Int. J. Exp. Pathol. 85: 47-64, 2004). Halofuginone has been studied extensively as a potential anti-fibrotic therapeutic and has progressed to phase 2 clinical trials for applications stemming from these properties.

In animal models of wound healing and fibrotic disease, halofuginone reduces excess dermal ECM deposition when introduced intraperitoneally, added to food, or applied locally (Pines, et al., Biol. Blood Marrow Transplant 9: 417-425, 2003). Halofuginone is currently in phase 2 clinical trials as a treatment for scleroderma (Pines, et al., Biol. Blood Marrow Transplant 9: 417-425, 2003), bladder cancer (Elkin, et al., Cancer Res. 59: 4111-4118, 1999), and angiogenesis during Kaposi's sarcoma, as well as in earlier stages of clinical investigation for a wide range of other fibrosis-associated disorders (Nagler, et al., Am. J. Respir. Crit. Care Med. 154: 1082-1086, 1996; Nagler, et al., Arterioscler. Thromb. Vasc. Biol. 17: 194-202, 1997; Nagler, et al., Eur. J. Cancer 40: 1397-1403, 2004; Ozcelik, et al., Am. J. Surg. 187: 257-260, 2004). The results presented herein indicate that the inhibition of fibrosis may be due at least in part to the inhibition of glutamyl-prolyl tRNA synthetase (EPRS).

In some embodiments, an agent that inhibits an aminoacyl tRNA synthetase inhibits pro-fibrotic activities of fibroblasts. Thus, in certain embodiments, the present invention provides a method for treating a fibroblast-associated disorder comprising the step of administering to a patient in need thereof an agent that inhibits an aminoacyl tRNA synthetase or pharmaceutically acceptable composition thereof.

As used herein, the term “fibroblast-associated” disorders means any disease or other deleterious condition in which fibroblasts are known to play a role. Accordingly, another embodiment of the present invention relates to treating or lessening the severity of one or more diseases in which fibroblasts are known to play a role including, but not limited to, fibrosis.

While halofuginone at high concentrations (between 20-40 nM) does generally inhibit CD4⁺ T cell, CD8⁺ T cell, and B220⁺ B cell activation, halofuginone also specifically inhibits the development of Th17 cells, i.e., the T helper subset that exclusively expresses high levels of the pro-inflammatory cytokine interleukin IL-17, at low concentrations (PCT/US08/09774, filed Aug. 15, 2008, which claims priority to U.S. Ser. No. 60/964,936, filed Aug. 15, 2007, the entirety of each of which is incorporated herein by reference). Th17 cells, as a function of their IL-17 secretion, play causal roles in the pathogenesis of two important autoimmune diseases in the mouse, experimental autoimmune encephalomyelitis (EAE) and type II collagen-induced arthritis (CIA). EAE and CIA are murine models of the human autoimmune pathologies, multiple sclerosis (MS) and rheumatoid arthritis (RA). Halofuginone has been shown to be active in these models. Halofuginone-mediated specific inhibition of IL-17 expressing cell development, such as IL-17 expressing effector T cell development, e.g., Th17 cell development, takes place at remarkably low concentrations, with 50% inhibition being achieved around 3 nM. Therefore, halofuginone treatment specifically inhibits the development of Th17-mediated and/or IL-17 related diseases, including autoimmune diseases, persistent inflammatory diseases, and infectious diseases, while not leading to profound T cell dysfunction, either in the context of delayed-type hypersensitivity or infection. Other agents that inhibit aminoacyl tRNA synthetases can also be used to inhibit the development of Th17-mediated and/or IL-17 related diseases.

Agents that inhibit aminoacyl tRNA synthetases interfere with the differentiation of naïve T-cells into IL-17-expressing Th17 cells. Thus, in certain embodiments, the present invention provides a method for treating a Th17-mediated or IL-17-mediated disorder comprising the step of administering to a patient in need thereof an agent that inhibits an aminoacyl tRNA synthetase or a pharmaceutically acceptable composition thereof.

As used herein, the terms “Th17-mediated” disorder and “IL-17-mediated” disorder means any disease or other deleterious condition in which Th17 or IL-17 is known to play a role. Accordingly, another embodiment of the present invention relates to treating or lessening the severity of one or more diseases in which Th17 or IL-17 is known to play a role including, but not limited to, autoimmune diseases, inflammatory diseases, infectious diseases, angiogenesis, and organ protection during transplantation.

The compounds and pharmaceutical compositions of the present invention may be used in treating or preventing diseases or conditions including, but not limited to, asthma, arthritis, inflammatory diseases (e.g., Crohn's disease, rheumatoid arthritis, psoriasis), proliferative diseases (e.g., cancer, benign neoplasms, diabetic retinopathy), cardiovascular diseases, and autoimmune diseases (e.g., rheumatoid arthritis, lupus, multiple sclerosis). Agents that inhibit tRNA synthetases and pharmaceutical compositions thereof may be administered to animals, preferably mammals (e.g., domesticated animals, cats, dogs, mice, rats), and more preferably humans. Any method of administration may be used to deliver the agent or pharmaceutical composition to the animal. In certain embodiments, the agent or pharmaceutical composition is administered orally. In other embodiments, the agent or pharmaceutical composition is administered parenterally.

In certain embodiments, the present invention provides methods for treating or lessening the severity of autoimmune diseases including, but not limited to, acute disseminated encephalomyelitis, alopecia universalis, alopecia greata, Addison's disease, ankylosing spondylosis, antiphospholipid antibody syndrome, aplastic anemia, arthritis, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, celiac disease, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, dry eye disease, endometriosis, dysautonomia, essential mixed cryoglobulinemia, fibromyalgia-fibromyositis, glomerulonephritis, idiopathic pulmonary fibrosis, Goodpasture's syndrome, Graves' disease, Guillain-Barre syndrome, Hashimoto's thyroiditis, IgA neuropathy, inflammatory bowel disease, interstitial cystitis, juvenile arthritis, lichen planus, Meniere's disease, mixed connective tissue disease, type 1 or immune-mediated diabetes mellitus, juvenile arthritis, multiple sclerosis, myasthenia gravis, neuromyotonia, opsoclonus-myoclonus syndrome, optic neuritis, Ord's thyroiditis, osteoarthritis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, rheumatoid arthritis, sarcoidosis, scleroderma, Sjogren's syndrome, stiff-man syndrome, Still's disease, systemic lupus erythematosus, takayasu arteritis, temporal arteritis/giant cell arteritis, idiopathic thrombocytopenic purpura, ulcerative colitis, uveitis, vasculitides such as dermatitis herpetiformis vasculitis, vitiligo, vulvodynia, warm autoimmune hemolytic anemia, and Wegener's granulomatosis.

In some embodiments, the present invention provides a method for treating or lessening the severity of one or more diseases and conditions, wherein the disease or condition is selected from immunological conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the present invention provides a method for treating or lessening the severity of an inflammatory disease including, but not limited to, asthma, appendicitis, Blau syndrome, blepharitis, bronchiolitis, bronchitis, bursitis, cervicitis, cholangitis, cholecystitis, chronic obstructive pulmonary disease (COPD), chronic recurrent multifocal osteomyelitis (CRMO), colitis, conjunctivitis, cryopyrin associated periodic syndrome (CAPS), cystitis, dacryoadenitis, dermatitis, dermatomyositis, encephalitis, endocarditis, endometritis, enteritis, enterocolitis, epicondylitis, epididymitis, familial cold-induced autoinflammatory syndrome, familial Mediterranean fever (FMF), fasciitis, fibrositis, gastritis, gastroenteritis, hepatitis, hidradenitis suppurativa, laryngitis, mastitis, meningitis, mevalonate kinase deficiency (MKD), Muckle-Well syndrome, myelitis myocarditis, myositis, nephritis, oophoritis, orchitis, osteitis, inflammatory osteolysis, otitis, pancreatitis, parotitis, pericarditis, peritonitis, pharyngitis, pleuritis, phlebitis, pneumonitis, pneumonia, proctitis, prostatitis, pulmonary fibrosis, pyelonephritis, pyoderma gangrenosum and acne syndrome (PAPA), pyogenic sterile arthritis, rhinitis, salpingitis, sinusitis, stomatitis, synovitis, systemic juvenile rheumatoid arthritis, tendonitis, TNF receptor associated periodic syndrome (TRAPS), tonsillitis, undifferentiated spondyloarthropathy, undifferentiated arthropathy, uveitis, vaginitis, vasculitis, vulvitis, or chronic inflammation resulting from chronic viral or bacteria infections, psoriasis (e.g., plaque psoriasis, pustular psoriasis, erythrodermic psoriasis, guttate psoriasis or inverse psoriasis).

In certain embodiments, the present invention provides methods for treating or lessening the severity of arthropathies and osteopathological diseases including, but not limited to, rheumatoid arthritis, osteoarthitis, gout, polyarthritis, and psoriatic arthritis.

In certain embodiments, the present invention provides methods for treating or lessening the severity of hyperproliferative diseases including, but not limited to, psoriasis or smooth muscle cell proliferation including vascular proliferative disorders, atherosclerosis, and restenosis. In certain embodiments, the present invention provides methods for treating or lessening the severity of endometriosis, uterine fibroids, endometrial hyperplasia, and benign prostate hyperplasia.

In certain embodiments, the present invention provides methods for treating or lessening the severity of acute and chronic inflammatory diseases including, but not limited to, ulcerative colitis, inflammatory bowel disease, Crohn's disease, allergic rhinitis, allergic dermatitis, cystic fibrosis, chronic obstructive bronchitis, and asthma.

In some embodiments, the present invention provides a method for treating or lessening the severity of a cardiovascular disorder including, but not limited to, myocardial infarction, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis, ischemic stroke, cardiac hypertrophy, and heart failure.

The present invention further includes a method for the treatment of mammals, including humans, which are suffering from one of the above-mentioned conditions, illnesses, disorders, or diseases. The method comprises that a pharmacologically active and therapeutically effective amount of one or more of the agents according to this invention is administered to the subject in need of such treatment.

The invention further relates to the use of the agents according to the present invention for the production of pharmaceutical compositions which are employed for the treatment and/or prophylaxis and/or amelioration of the diseases, disorders, illnesses, and/or conditions as mentioned herein.

The invention further relates to the use of the agents according to the present invention for the production of pharmaceutical compositions that inhibit an aminoacyl tRNA synthetase.

The invention further relates to the use of the agents according to the present invention for the production of pharmaceutical compositions for inhibiting or treating fibrosis.

The invention further relates to the use of the agents according to the present invention for the production of pharmaceutical compositions which can be used for treating, preventing, or ameliorating of diseases responsive to inhibiting IL-17 production, such as autoimmune or inflammatory diseases, such as any of those diseases mentioned herein.

The exact amount required will vary from subject to subject, depending on the species, age, and general condition of the subject, the particular agent, its mode of administration, its mode of activity, and the like. The compounds of the invention are preferably formulated in dosage unit form for ease of administration and uniformity of dosage. It will be understood, however, that the total daily usage of the agents of the present invention will be decided by the attending physician within the scope of sound medical judgment. The specific therapeutically effective dose level for any particular patient or organism will depend upon a variety of factors including the disorder being treated and the severity of the disorder; the specific agent employed; the age, body weight, general health, sex, and diet of the patient; the time of administration, route of administration, and rate of excretion of the specific agent employed; the duration of the treatment; drugs used in combination or coincidental with the specific agent employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, an agent of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, an agent that inhibits a tRNA synthetase is administered at a dose that is below the dose at which the agent causes non-specific effects. In certain embodiments, an agent that inhibits a tRNA synthetase is administered at a dose that does not cause generalized immunosuppression in a subject.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL (polyethoxylated castor oil), alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as poly(lactide-co-glycolide). Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active agent.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the agents and pharmaceutical compositions of the present invention can be employed in combination therapies, that is, the agents and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an agent that inhibits a tRNA synthetase may be administered concurrently with another agent), or they may achieve different effects (e.g., control of any adverse effects).

In still another aspect, the present invention also provides a pharmaceutical pack or kit comprising one or more containers filled with one or more of the ingredients of a pharmaceutical composition (e.g., one or more inhibitors of an aminoacyl tRNA synthetase), and in certain embodiments, includes an additional approved therapeutic agent for use as a combination therapy (e.g., one or more immunosuppressive agents). In certain embodiments, a kit comprises an aminoacyl tRNA synthetase and an inhibitor of a proinflammatory cytokine, e.g., an inhibitor of one or more of IL-6, IL-21, TNFα, IFNγ, GM-CSF, MIP-2, IL-12, IL-1α, IL-Iβ, or IL-23. In some embodiments, a cytokine inhibitor comprises an antibody that binds to the cytokine or that binds to a receptor of the cytokine, an agent that reduces expression of the cytokine (e.g., a small interfering RNA (siRNA) or antisense oligonucleotide), a soluble cytokine receptor, or a small molecule inhibitor. In some embodiments, a cytokine inhibitor comprises an inhibitor of TNFα. In some embodiments, an inhibitor of TNFα comprises an anti-TNFα antibody or antigen binding fragment thereof. In some embodiments, the anti-TNFα antibody is adalimumab (Humira™). In some embodiments, the anti-TNFα antibody is infliximab (Remicade™). In some embodiments, the anti-TNFα antibody is CDP571. In some embodiments, an inhibitor of TNFα comprises a TNFα receptor, e.g., wherein the TNFα inhibitor is etanercept (Enbrel™). In some embodiments, an inhibitor of TNFα comprises an agent that inhibit expression of TNFα (e.g., a short interfering nucleic acid (siNA), a short interfering RNA (siRNA), a double-stranded RNA (dsRNA), a short hairpin RNA (shRNA), or an antisense oligonucleotide).

Optionally associated with such container(s) can be a notice in the form prescribed by a governmental agency regulating the manufacture, use or sale of pharmaceutical products, which notice reflects approval by the agency of manufacture, use or sale for human administration.

### Methods of Identifying Subjects in Need of Th17 Modulation

In various embodiments of the invention, suitable in vitro or in vivo studies are performed to determine whether administration of a specific therapeutic agent (i.e., tRNA synthetase inhibitor) that modulates the development of IL-17 expressing cells, such as IL-17 expressing effector T-cells, e.g., Th17 cells is indicated for treatment of a given subject or population of subjects. For example, subjects in need of treatment using a compound that modulates IL-17 expressing cell development, such as IL-17 expressing effector T-cell development, e.g., Th17 cell development, are identified by obtaining a sample of IL-17 expressing cells, such as IL-17 expressing effector T-cells, e.g., Th17 cells from a given test subject and expanding the sample of cells. If the concentration of any of a variety of inflammatory cytokine markers, including IL-17, IL-17F, IL-6, IL-21, IL-2, and TNFα, also increases as the cell population expands, then the test subject is a candidate for treatment using any of the agents, compositions, and methods described herein.

Subjects in need of treatment are also identified by detecting an elevated level of IL-17 expressing cells, such as IL-17 expressing effector T-cells, e.g., Th17 cells or a Th17 cell-associated cytokine or a cytokine that is secreted by a Th17 cell. Cytokine levels to be evaluated include IL-17, IL-17F, IL-6, IL-21, TNFα, and GM-CSF. The cytokine IL-17, as well as other cytokines such as IL-6, IL-21, IL-2, TNFα, and GM-CSF, are typically induced during inflammation and/or infection. Thus, any elevated level of expression of these cytokines in a subject or biological sample as compared to the level of expression of these cytokines in a normal subject is useful as an indicator of a disease state of other situation where treatment with a tRNA synthetase inhibitor is desirable. Studies have shown that the levels of IL-17 in healthy patient serum is less than 2 pg/mL (i.e., below the detection limit of the assay used), while patients with liver injury had levels of IL-17 expression in the range of 2-18 pg/mL and patients with rheumatoid arthritis had levels greater than 100 pg/mL (see Yasumi, et al., Hepatol Res. 37: 248-254, 2007; and Ziolkowska, et al., J. Immunol. 164: 2832-2838, 2000, each of which is incorporated herein by reference). Thus, detection of an expression level of IL-17 greater than 2 pg/mL in a subject or biological sample is useful in identifying subjects in need of treatment.

A subject suffering from or at risk of developing a Th17-related and/or IL-17-related disease such as an autoimmune disease, a persistent inflammatory disease, or an infectious disease is identified by methods known in the art. For example, subjects suffering from an autoimmune disease, persistent inflammatory disease, or an infectious disease are diagnosed based on the presence of one or more symptoms associated with a given autoimmune, persistent inflammatory, or infectious disease. Symptoms may include, for example, inflammation, fever, loss of appetite, weight loss, abdominal symptoms such as, for example, abdominal pain, diarrhea or constipation, joint pain or aches (arthralgia), fatigue, rash, anemia, extreme sensitivity to cold (Raynaud's phenomenon), muscle weakness, muscle fatigue, change in skin or tissue tone, shortness of breath or other abnormal breathing patterns, chest pain or constriction of the chest muscles, abnormal heart rate (e.g., elevated or lowered), light sensitivity, blurry or otherwise abnormal vision, and reduced organ function.

Subjects suffering from an autoimmune disease such as, e.g., multiple sclerosis, rheumatoid arthritis, Crohn's disease, are identified using any of a variety of clinical and/or laboratory test such as physical examination, radiological examination, and blood, urine, and stool analysis, to evaluate immune status.

Determination of Biological Effects of tRNA Synthetase Inhibition

In various embodiments of the invention, suitable in vitro or in vivo studies are performed to determine the effect of a specific therapeutic agent that modulates the development of IL-17 expressing cells, such as IL-17 expressing effector T-cells, e.g., Th17 cells, and whether its administration is indicated for treatment of a given subject or population of subjects. For example, the biological effect of a tRNA synthetase inhibitor is monitored by measuring level of IL-17 production and/or the number of IL-17 expressing cells, such as IL-17 expressing effector T-cells, e.g., Th17 cells in a patient-derived sample. The biological effect of a therapeutic agent is also measured by physical and/or clinical observation of a patient suffering from, or at risk of developing, a Th17-related and/or Il-17-related disease such as an autoimmune disease, persistent inflammatory disease, and/or an infectious disease. For example, administration of a specific Th17 inhibitor to a patient suffering from a Th17-related disease and/or an IL-17-related disease is considered successful if one or more of the signs or symptoms (e.g., fever, pain, swelling, redness) associated with the disorder is alleviated, reduced, inhibited, or does not progress to a further, i.e., worse, state.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Inhibition of Th17 Cell Development Through Activation of an Amino Acid Starvation Response

This example shows that the aminoacyl tRNA synthetase inhibitor, halofuginone (HF), imparts a selective block of Th17 differentiation in both human and mouse T cells by inducing the AAR response.

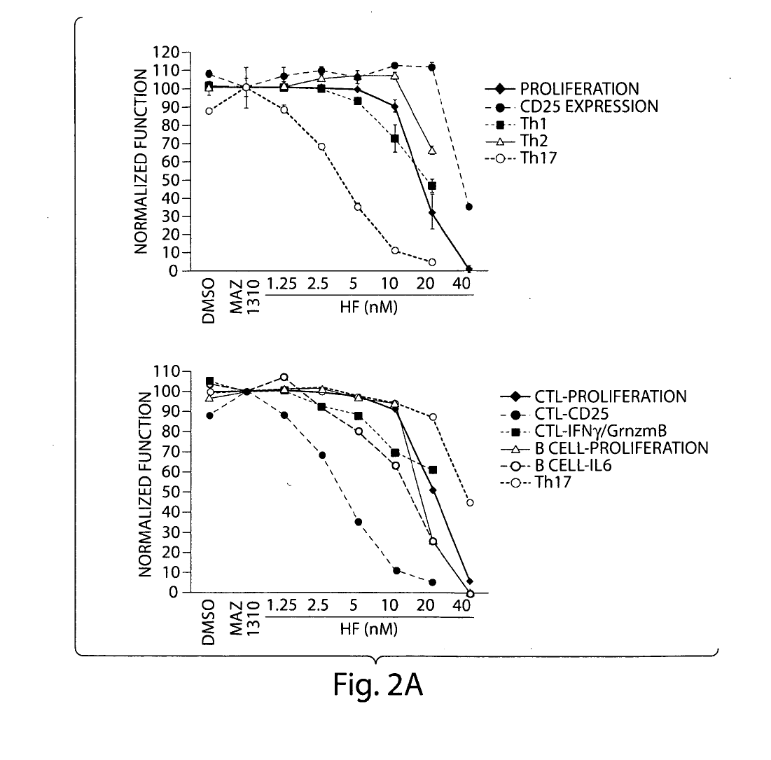

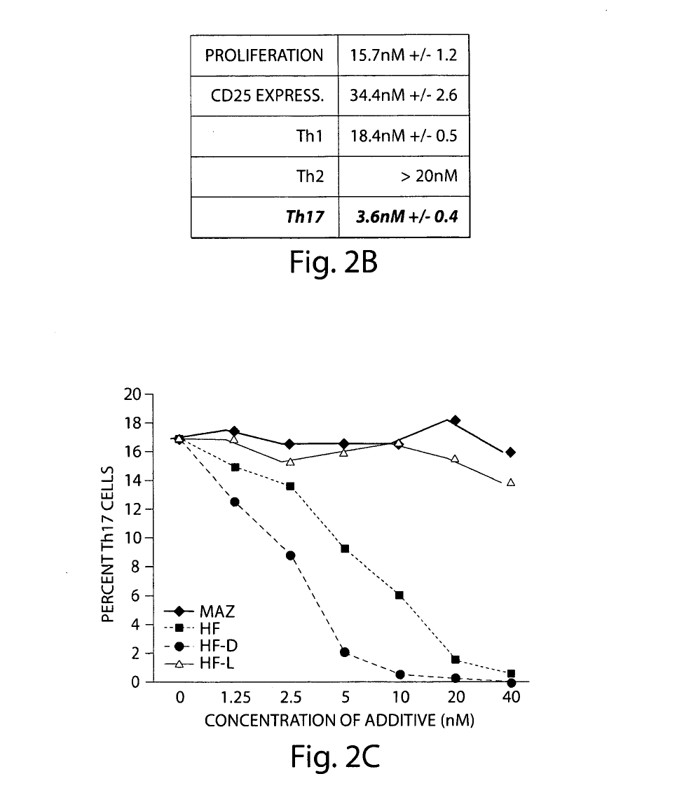

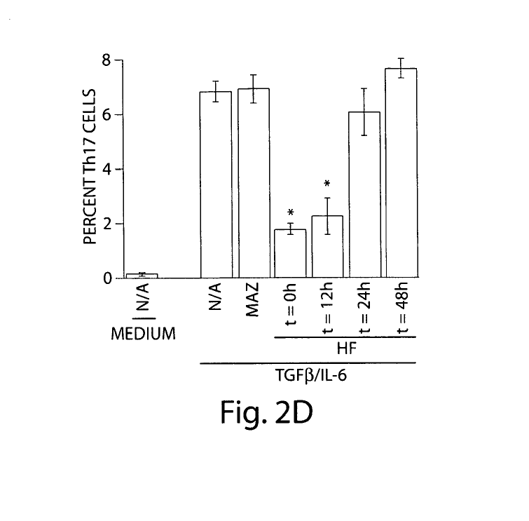

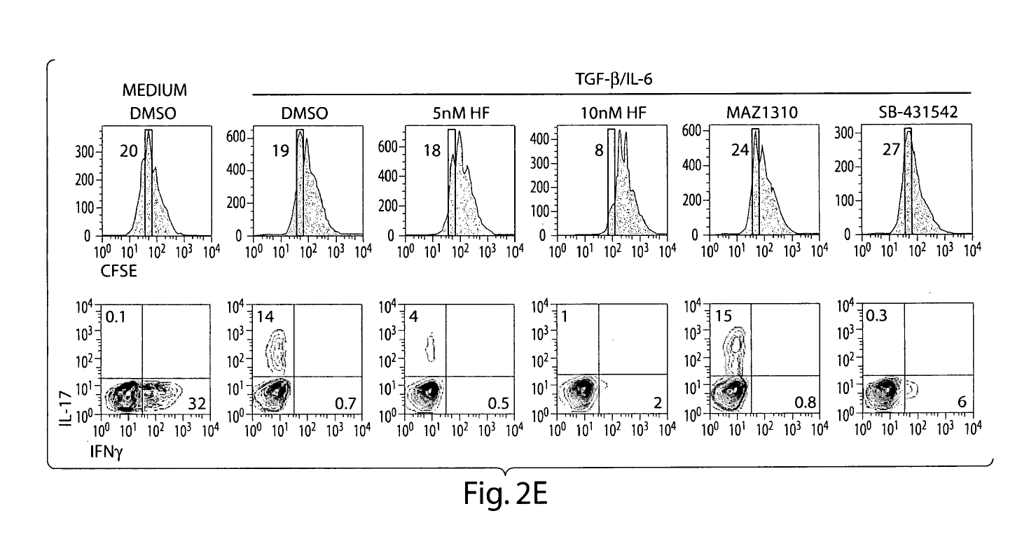

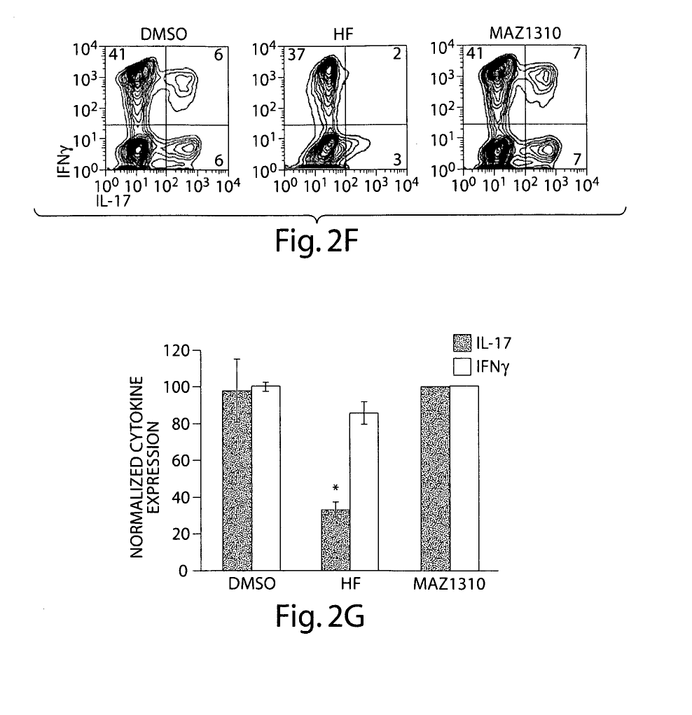

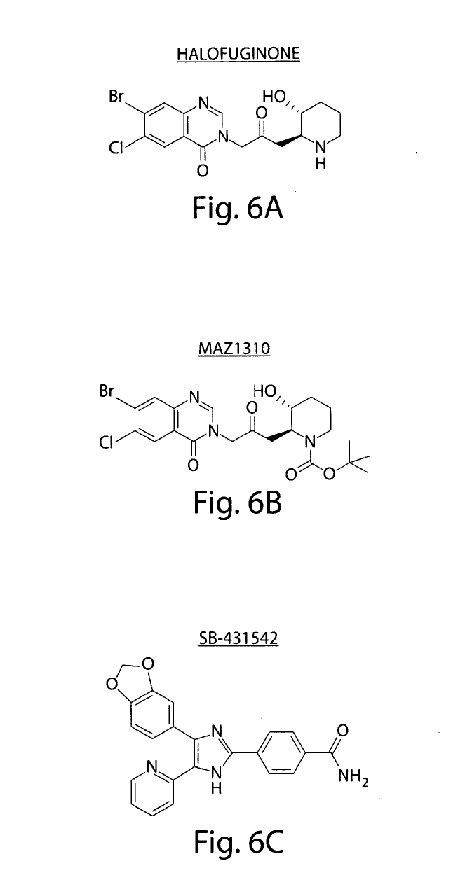

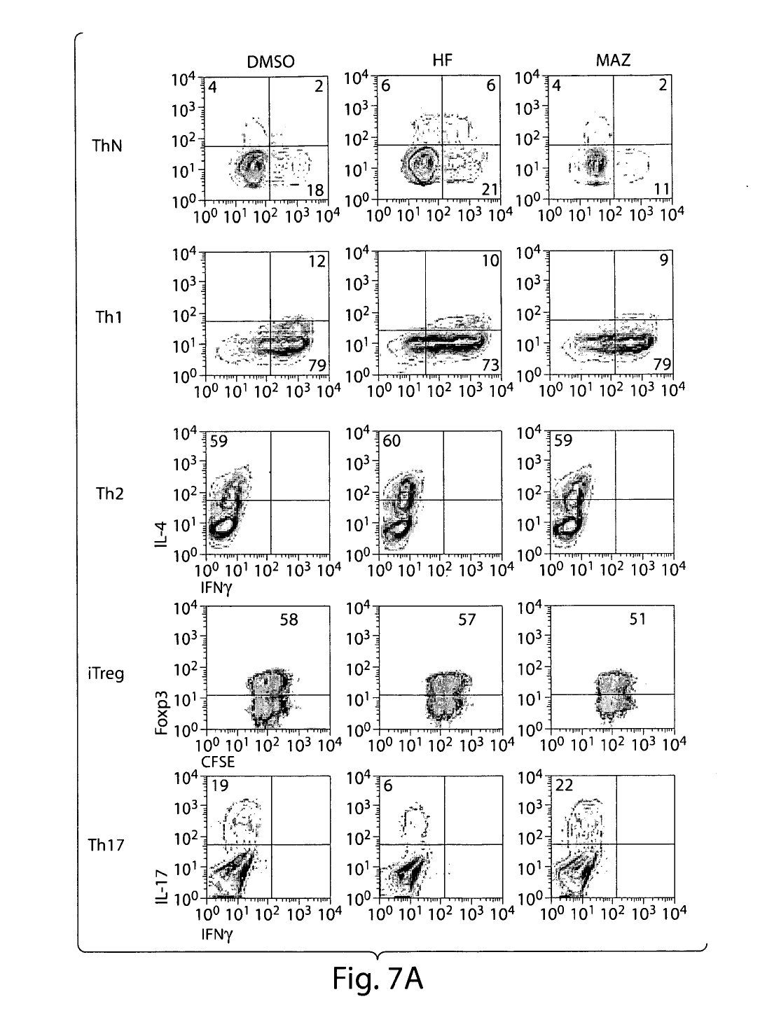

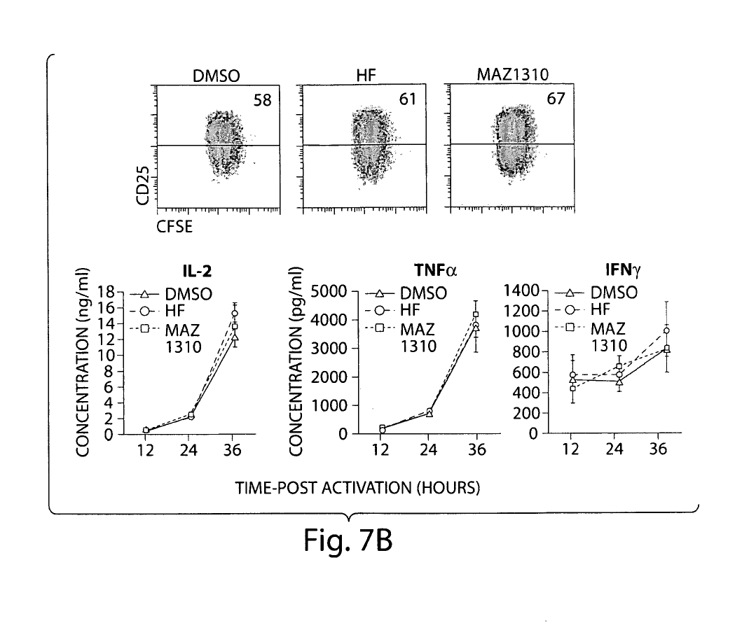

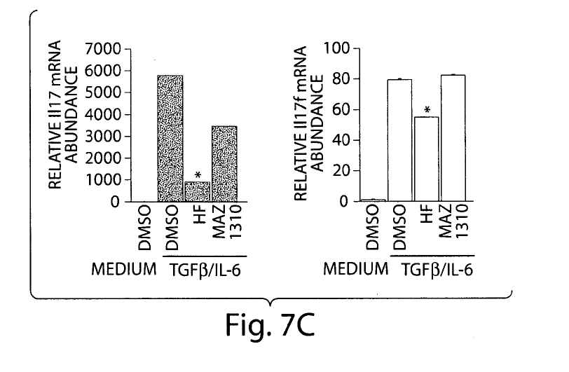

To investigate whether HF can modulate T cell differentiation or effector function, purified murine CD4⁺ CD25⁻ T cells were treated with HF or its inactive derivative MAZ1310 (FIG. 6) and stimulated in the absence or presence of polarizing cytokines to induce Th1, Th2, iTreg, or Th17 differentiation. Dose-response experiments revealed a remarkably selective effect of HF on Th17 differentiation (defined here as the percentage of IL-17⁺ IFNγ⁻ cells following restimulation on day 4-5). HF repressed Th17 differentiation in a dose-dependent manner with an IC₅₀ of 3.6 nM±0.4 nM (FIG. 2A, 2B). Low concentrations of HF (1-10 nM) that strongly reduced IL-17 production (FIG. 2A, 2B, FIG. 7A) did not affect T cell proliferation, CD25 upregulation, or production of IL-2, TNF, or IFNγ (FIG. 7B). Low-dose HF also failed to modulate Th1, Th2 or iTreg differentiation as assessed by IFNγ, IL-4, or Foxp3 expression, respectively (FIG. 1A, FIG. 7A). At approximately 10-fold higher concentrations (>20 nM), HF induced a general inhibition of T and B cell activation, proliferation, and effector function (FIG. 2A, 2B).

The selective inhibition of Th17 differentiation by low-dose HF was stereospecific: the HPLC-purified D-enantiomer of HF inhibited IL-17 expression more potently than a racemic mixture, whereas the L-enantiomer was completely inactive (FIG. 2C). Inhibition of IL-17 expression was most pronounced when HF was added during a 12-hour window at the start of the culture period (FIG. 2D) and HF treatment impaired expression of both IL-17 and IL-17f mRNA (FIG. 7C). These results suggest that HF regulates early events, possibly involved in Th17 lineage commitment, rather than influencing the expansion of Th17 cells or preventing acute cytokine expression upon restimulation. Inhibition by HF was not due to perturbation of cell cycle progression or selective survival; HF inhibited IL-17 expression in a dose-dependent manner even when considering only cells that had completed an equivalent number of cell divisions based on CFSE dilution (FIG. 2E). HF also reduced IL-17 expression in cultures where IFNγ and IL-4, cytokines known to inhibit Th17 differentiation (Park et al., Nat. Immunol. 6:1133, 2005), were blocked by addition of neutralizing antibodies. Thus, HF-mediated inhibition of Th17 cell development is not secondary to effects on T cell proliferation or auxiliary cytokine production.

In light of reports that IL-17 expression may be differentially regulated in murine versus human T cells (Manel et al., Nat. Immunol. 9:641, 2008; Wilson et al., Nat. Immunol. 8:950, 2007; Acosta-Rodriguez et al., Nat. Immunol. 8:942, 2007), HF modulation of IL-17 expression by human CD4⁺ T cells was investigated. These experiments showed that HF treatment greatly reduced both the percentage of human T cells expressing IL-17 and the amount of IL-17 produced (FIG. 2F, 2G). In striking contrast, IFNγ, expression was essentially unaffected by HF treatment (FIG. 2F, 2G). Therefore, HF selectively limits IL-17 expression in both human and mouse T cells.

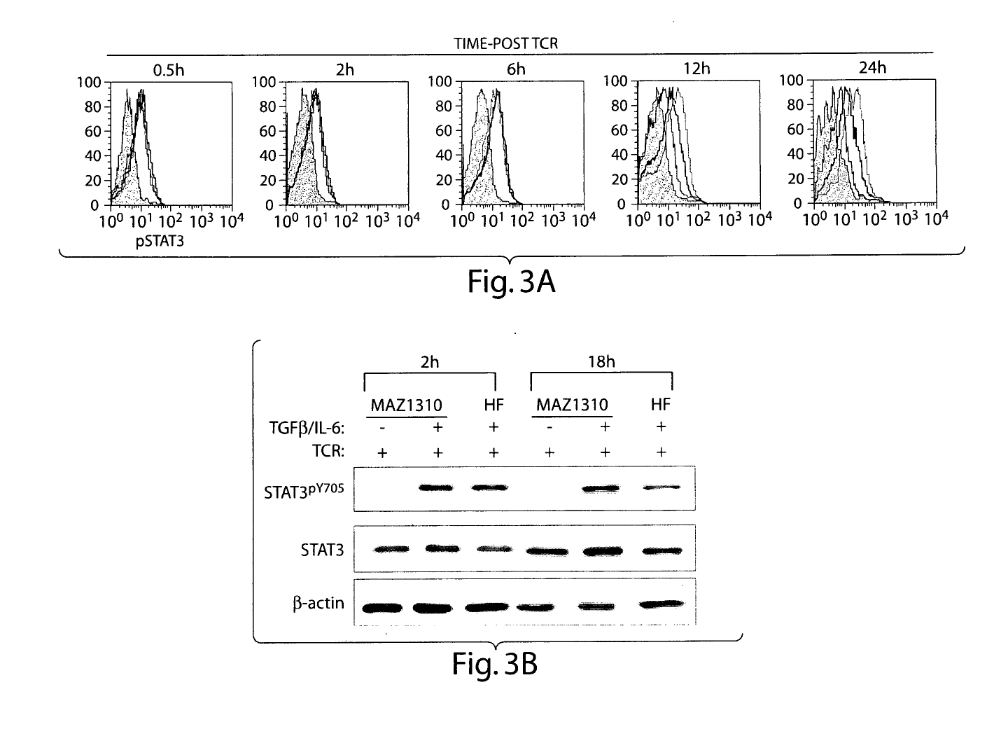

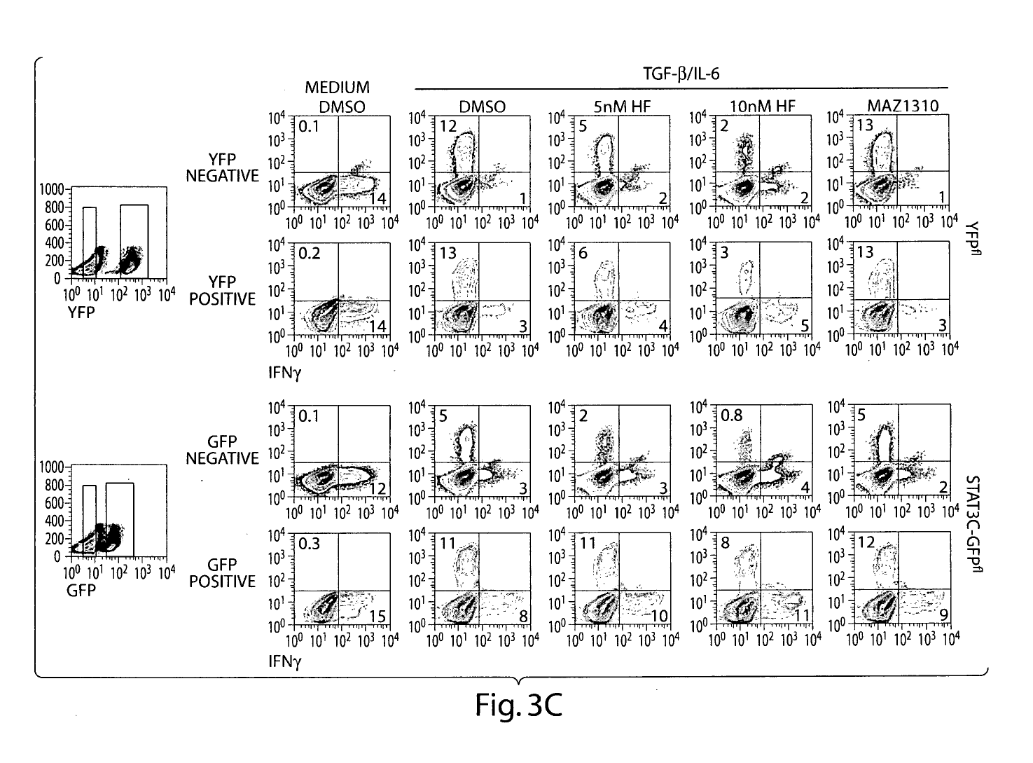

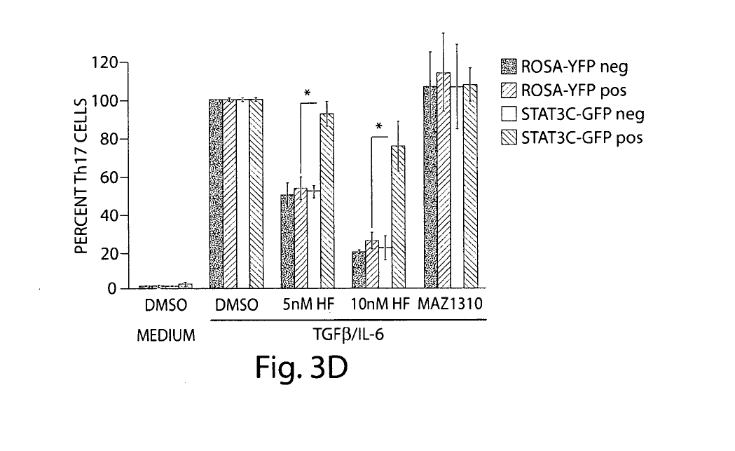

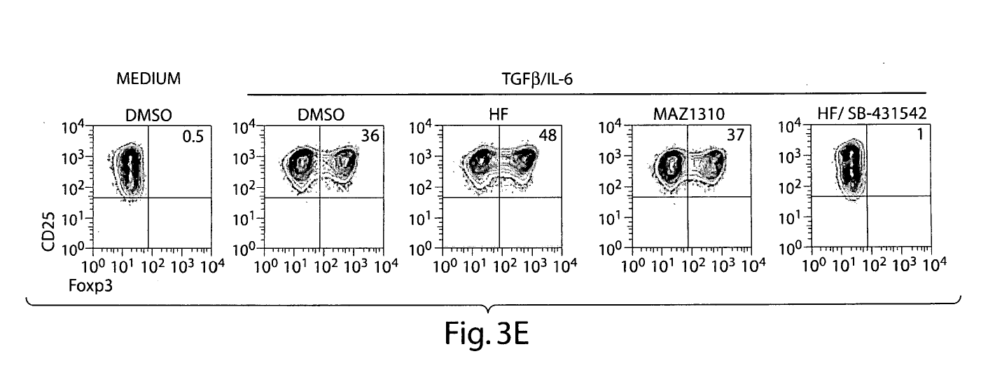

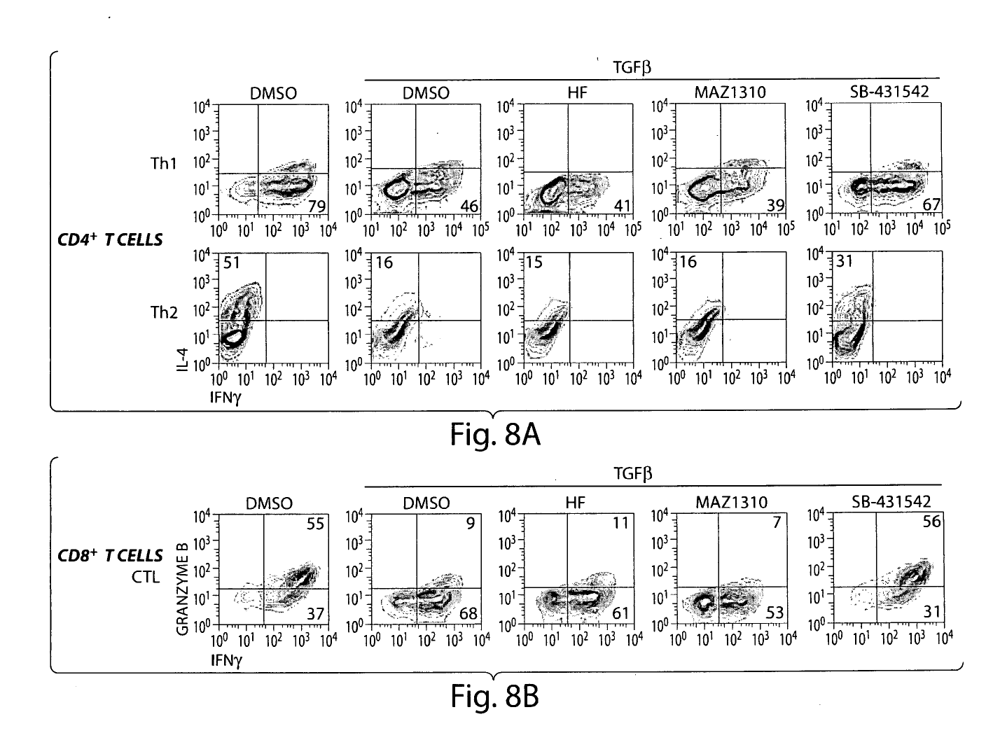

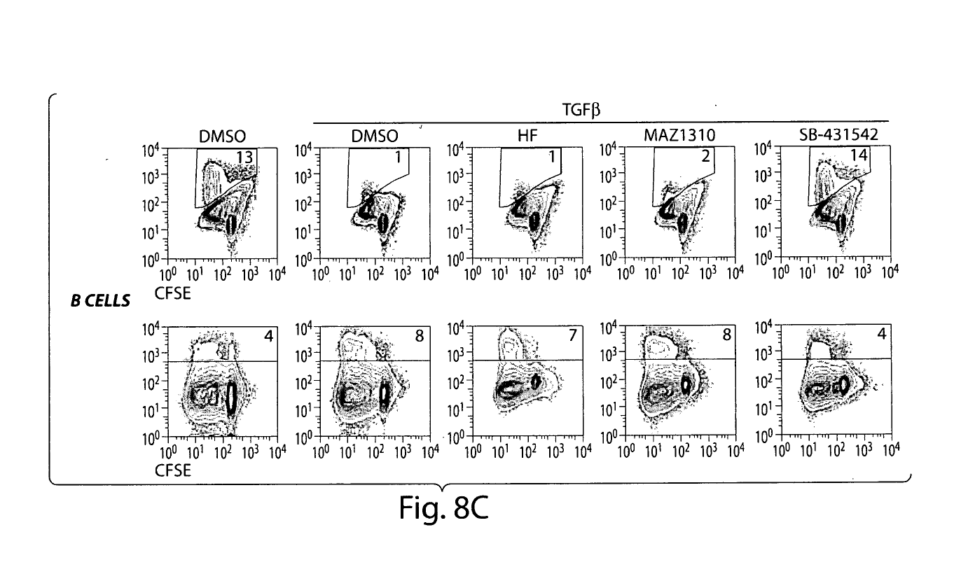

Th17 differentiation is synergistically regulated by TGFβ and the pro-inflammatory cytokines IL-6 and IL-21. Although reports had indicated that HF can attenuate TGFβ signaling at high concentrations (>50 nM) (Gnainsky et al., Cell Tiss. Res. 328:153, 200; Flanders, Int. J. Exp. Pathol. 85:47, 2004), it was discovered that low dose HF inhibited neither TGFβ-induced Smad phosphorylation nor a variety of other lymphocyte responses to TGFβ (Li et al., Ann. Rev. Immunol. 24:99, 2006; Glimcher et al., Nat. Rev. Immunol. 4:900, 2004; van Vlasselaer et al., J. Immunol. 148:2062, 1992), in contrast to the type 1 TGFβ receptor kinase inhibitor SB-431542, which abrogated all responses to TGFβ (FIG. 8). Since STAT3 is the major transducer of IL-6 and IL-21 signaling, the kinetics of STAT3 phosphorylation in HF-treated T cells were examined. HF did not interfere with STAT3 activation during the first 6 hours of Th17 differentiation, but rather decreased the maintenance of STAT3 phosphorylation, beginning around 12 hours post activation (FIG. 3A, 3B).

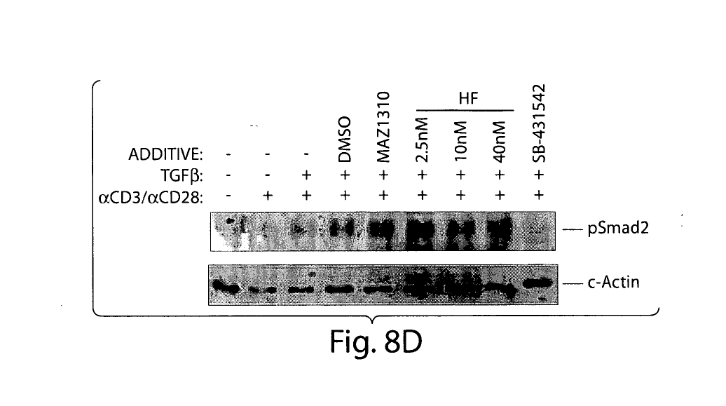

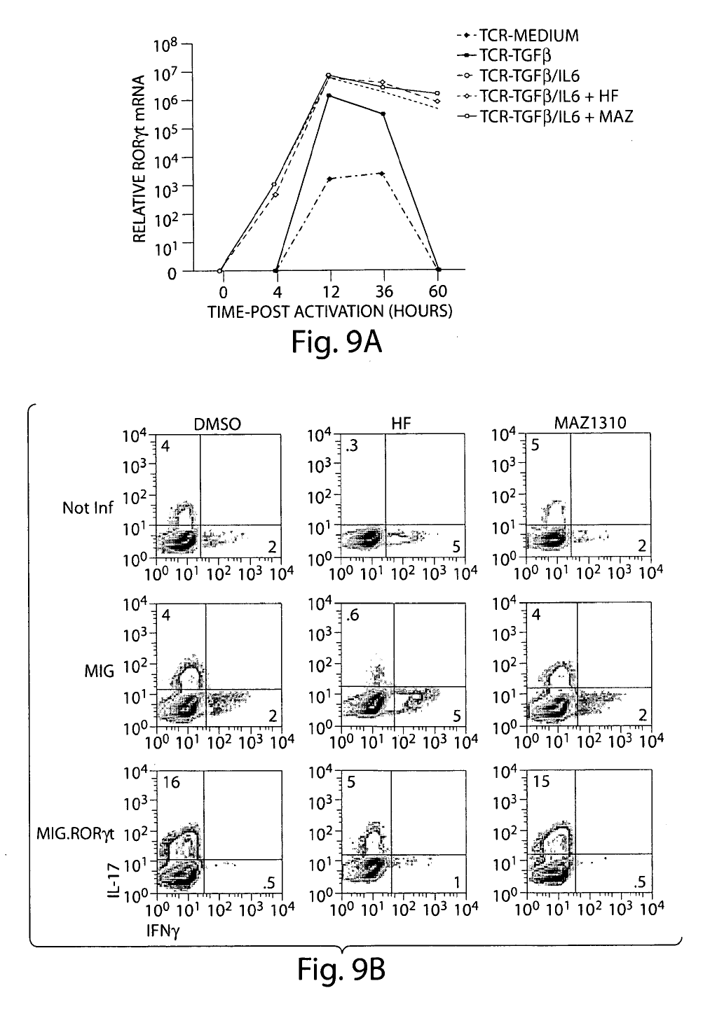

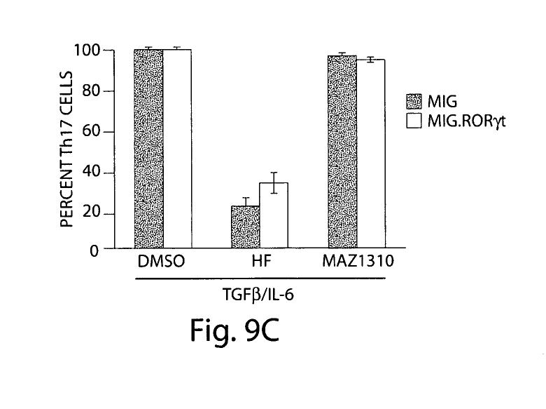

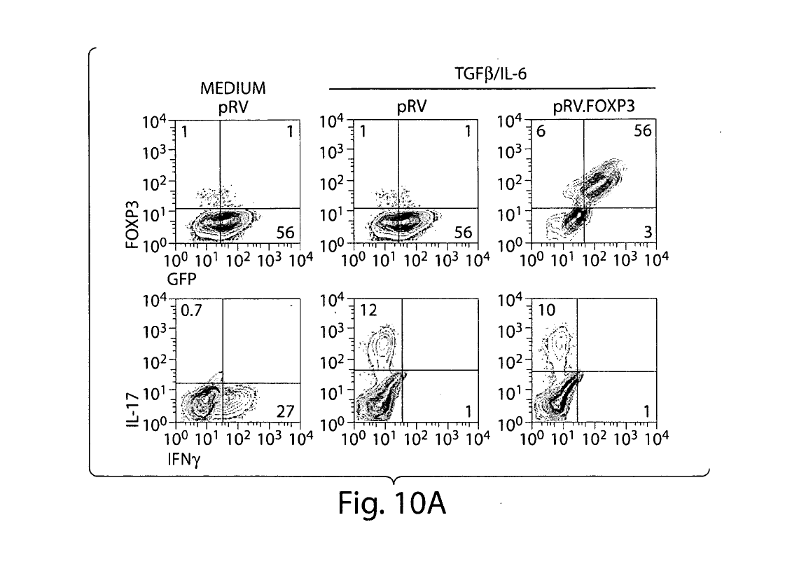

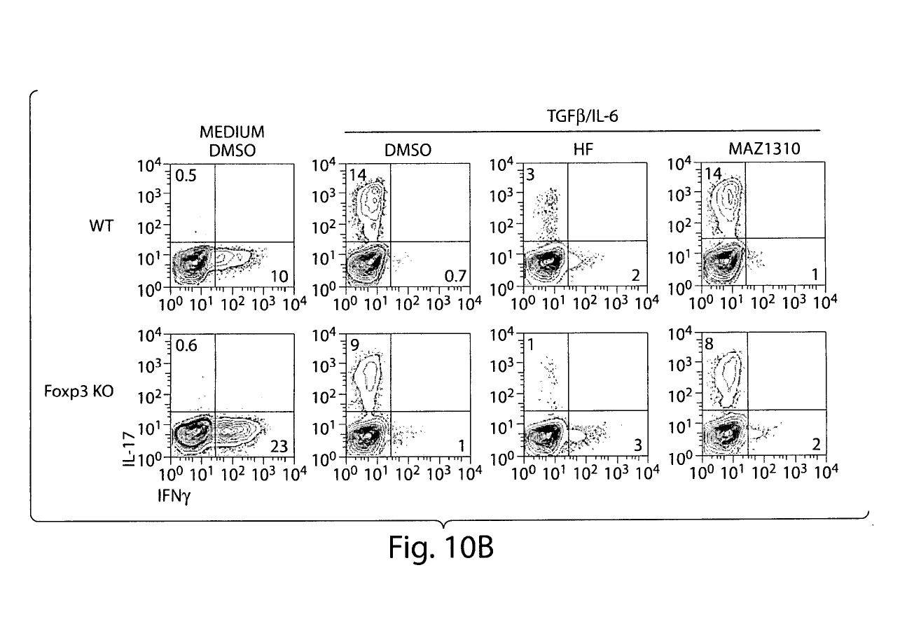

Next, it was investigated whether inhibition of Th17 differentiation by HF could be restored by transgenic expression of a hyperactive STAT3 protein (STAT3C) (Bromberg et al., Cell. 98:295, 1999). T cells isolated from homozygous mice containing a floxed stop-STAT3C-IRES-EGFP (STAT3C-GFPfl/fl) or stop-YFP (YFPfl/fl) cassette inserted into the ROSA26 locus were transduced with a cell-permeant TAT-Cre fusion protein to delete the stop cassette and these cells were activated in the presence of TGFβ plus IL-6, with either HF or MAZ1310. As expected, HF strongly impaired Th17 differentiation of cells expressing YFP or those not expressing a transgene (FIG. 3C, top three panels); in contrast, T cells expressing STAT3C (defined by their concomitant expression of GFP) remained capable of differentiating into Th17 cells even in the presence of 10 nM HF (FIG. 3C, bottom panel). Data from a number of similar experiments are quantified and summarized in FIG. 3D. Collectively, these results suggest that HF inhibits Th17 differentiation through its ability to prevent sustained activation of STAT3. STAT3 promotes Th17 lineage commitment through the induction of the orphan nuclear receptors RORγt and RORα (Yang et al., J. Biol. Chem. 282:9358, 2007; Ivanov et al., Cell 126:1121, 2006; Yang et al., Immunity 28:29, 2008). Consistent with the finding that HF did not affect STAT3 phosphorylation during the first 12 hours of stimulation, HF did not interfere with the upregulation of RORγt or RORα during Th17 differentiation (FIG. 9A). Moreover, HF inhibited Th17 differentiation as effectively in T cells retrovirally transduced with RORγt-expressing retroviruses as in those transduced with empty retroviruses (FIG. 9B, 9C). T cells differentiated in the presence of HF showed enhanced Foxp3 expression (FIG. 3E), as expected from the observations that HF inhibits STAT3 signaling and Th17 differentiation (Yang et al., J. Biol. Chem. 282:9358, 2007). This result suggested that HF redirects developing Th17 cells to the iTreg lineage rather than simply blocking their effector function. However, upregulation of Foxp3 by HF was neither necessary nor sufficient to inhibit Th17 differentiation; retroviral expression of FOXP3 in T cells did not decrease IL-17 expression induced by TGFβ plus IL-6 (FIG. 10A), though it markedly reduced IL-2 and IFNγ production in T cells cultured under non-polarizing conditions. Moreover, HF strongly repressed IL-17 expression in T cells lacking Foxp3 (FIG. 10B). Therefore, the inhibitory effects of HF on Th17 differentiation are not exerted indirectly through the upregulation of Foxp3. Rather, HF impairs the maintenance of STAT3 phosphorylation in developing Th17 cells, resulting in a reciprocal increase in iTreg cell development.

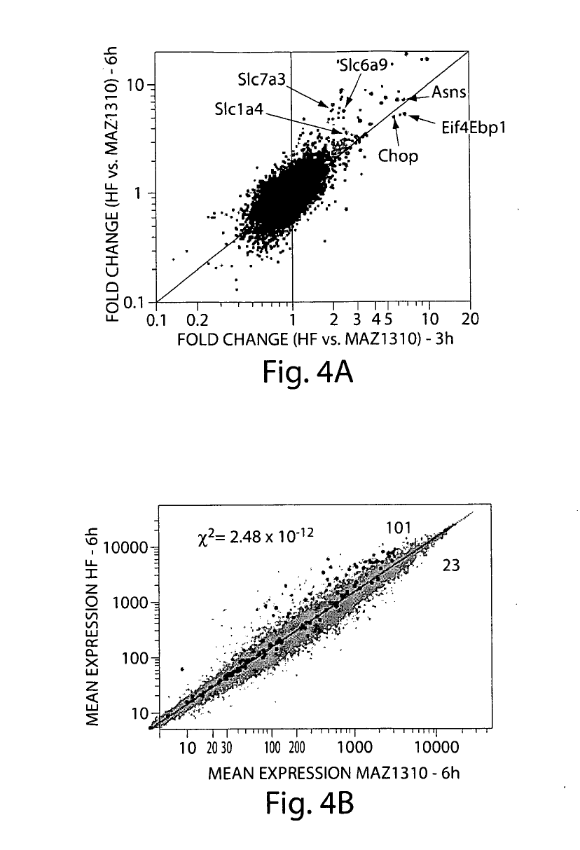

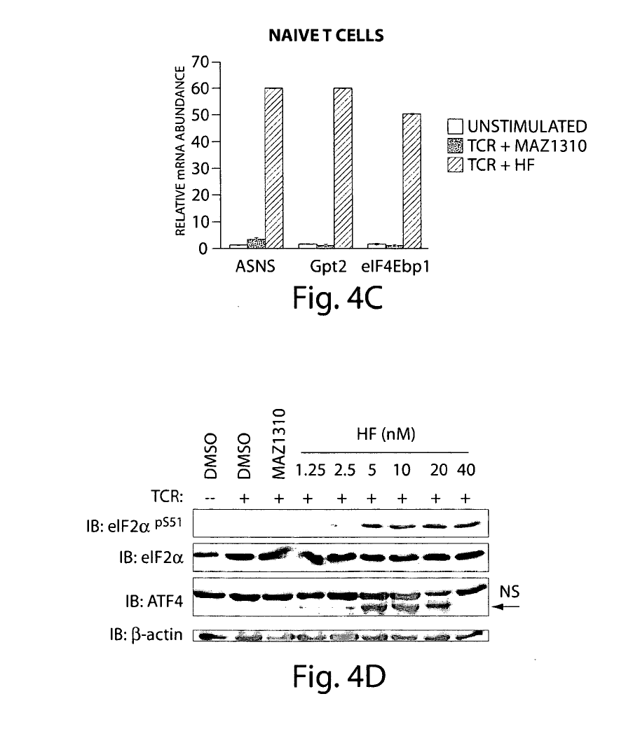

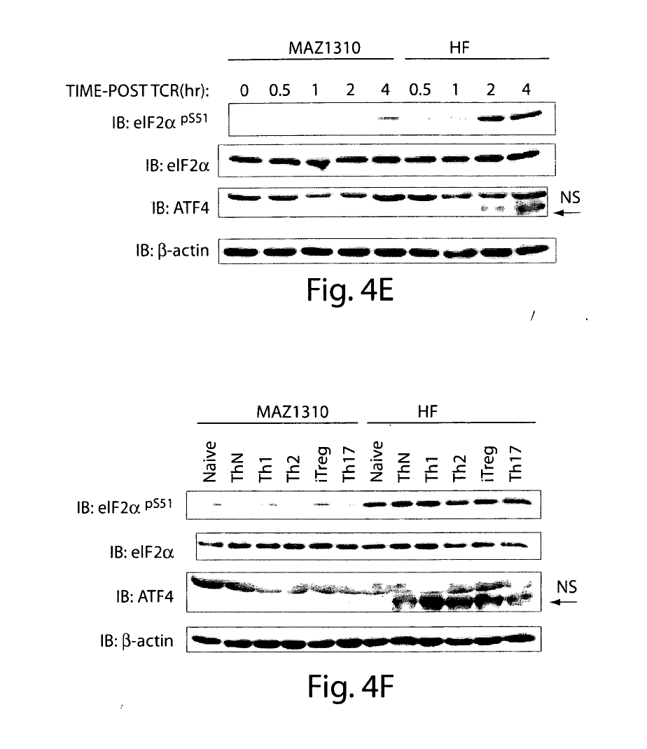

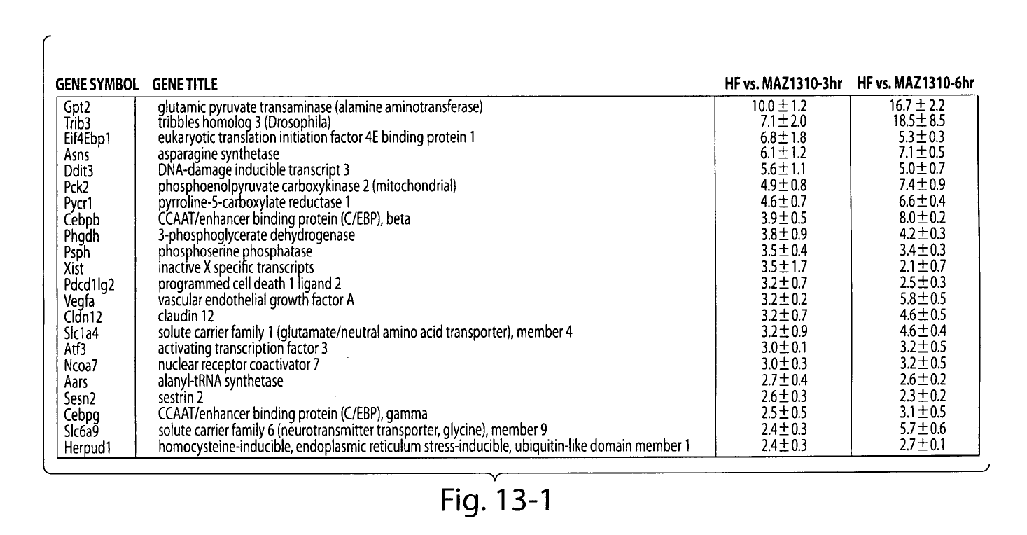

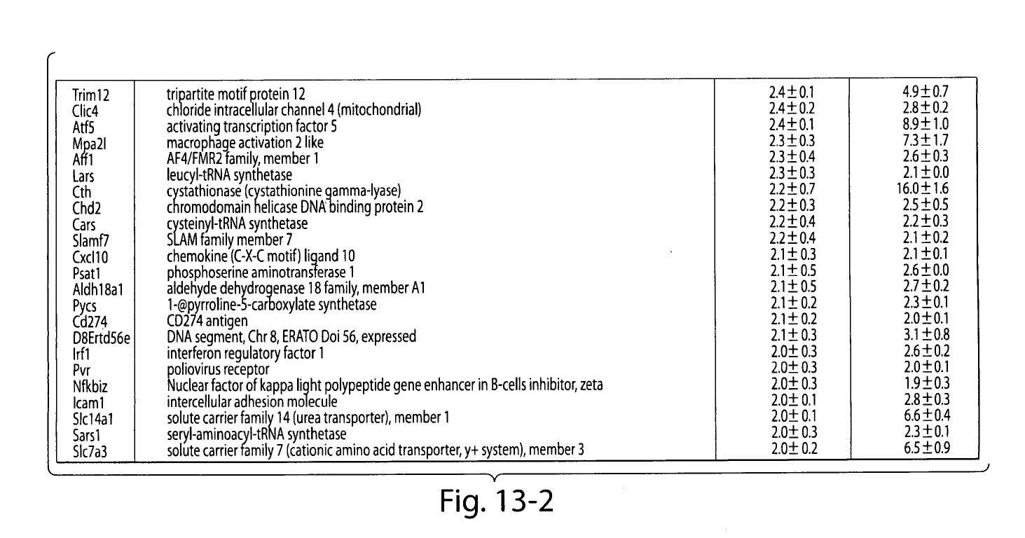

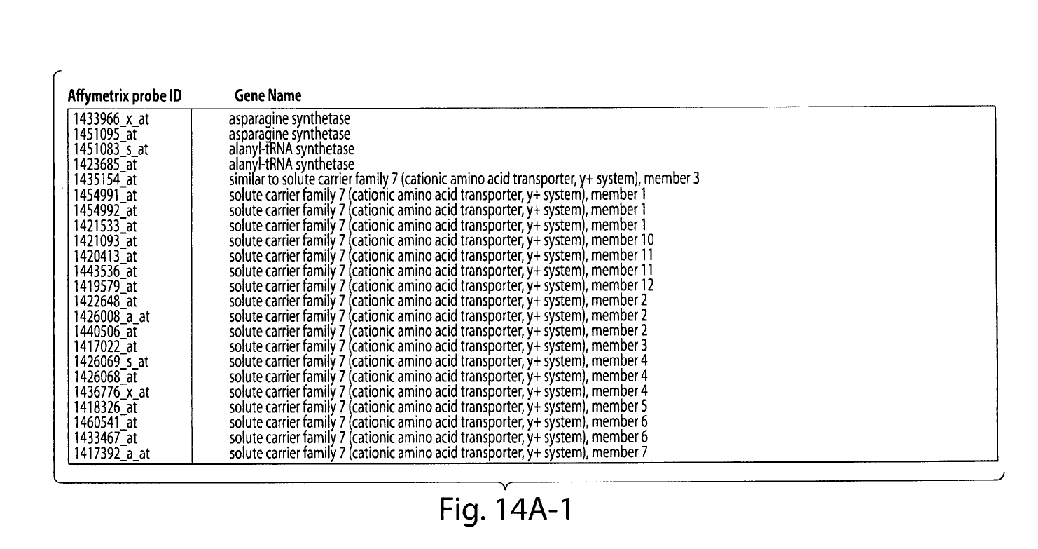

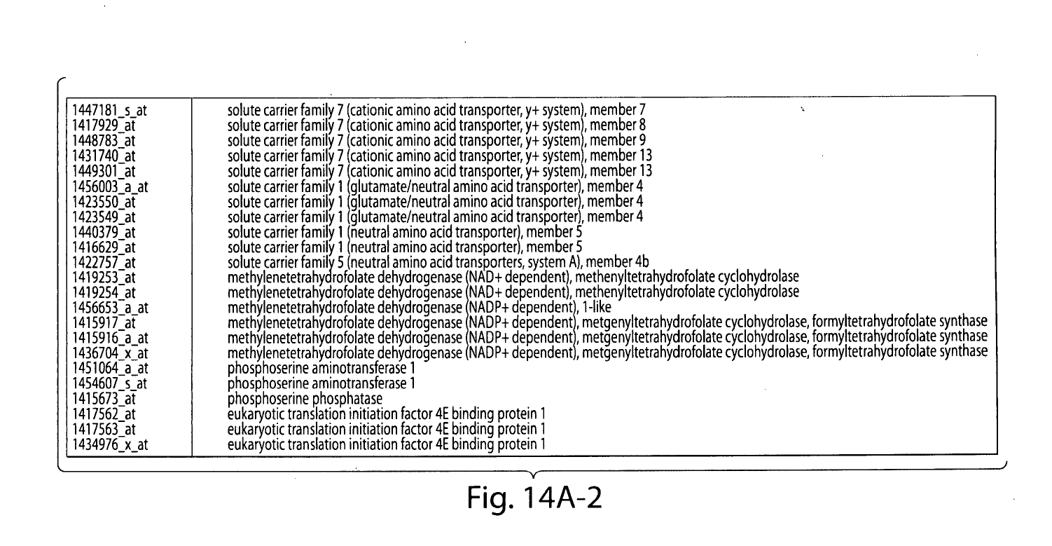

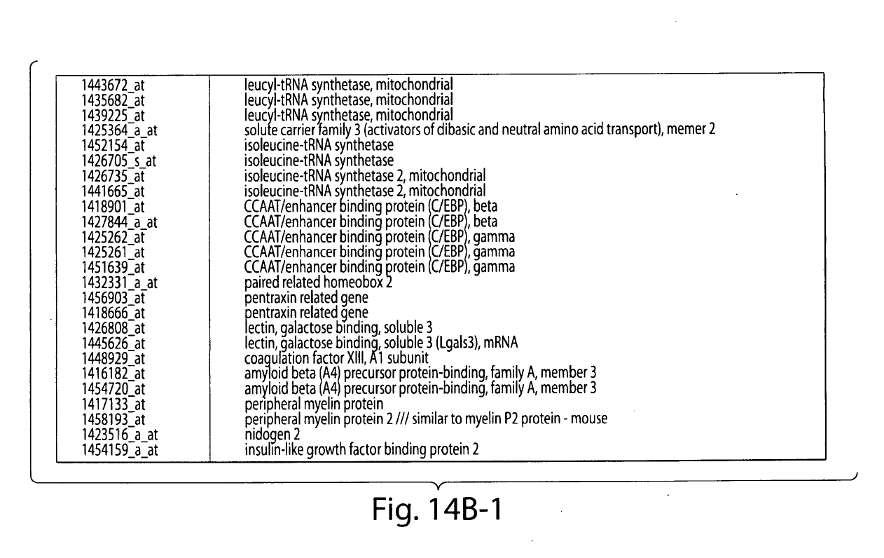

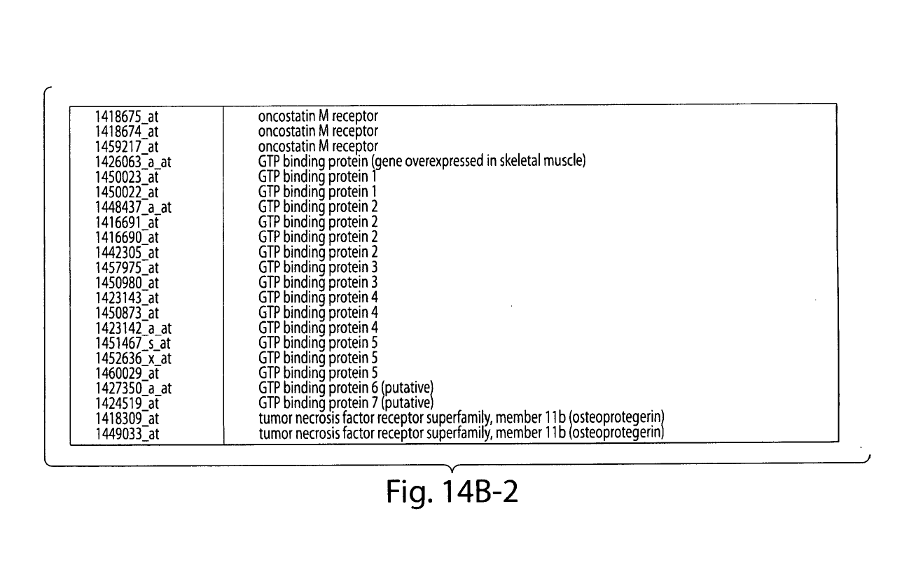

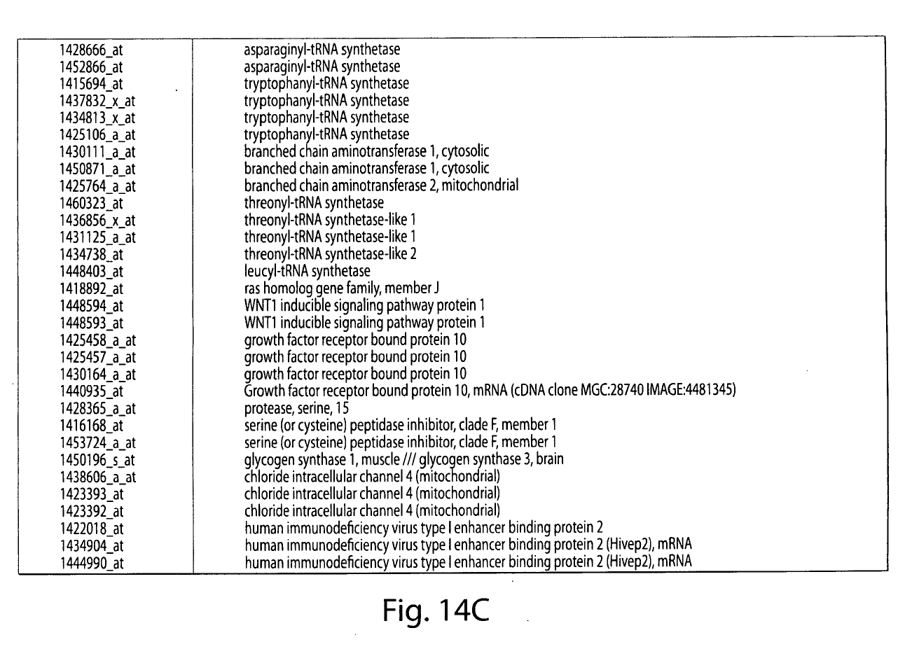

The 12-hour lag period between the addition of HF to T cell cultures and the ensuing effect on STAT3 phosphorylation strongly suggested an indirect effect. To identify more proximal cellular effects of HF treatment, we used DNA microarrays to define the transcriptional profiles of HF- and MAZ1310-treated T cells activated in Th17-priming conditions for 3 or 6 hours. Eighty one annotated genes that were differentially expressed at both time points in HF-versus MAZ1310-treated cells were identified, the majority of which were upregulated following HF treatment (FIG. 4A, FIG. 13). Among the HF-inducible transcripts, a large number of genes functionally associated with amino acid synthesis and transport, as well as protein synthesis, were observed (FIG. 4A, FIG. 13). Similar gene expression profiles have been observed during cellular responses to amino acid starvation (Fafournoux et al., Biochem. J. 351:1, 2000; Peng et al., Mol. Cell. Biol. 22:5575, 2002). Insufficient cellular levels of amino acids lead to the accumulation of uncharged tRNAs that, in turn, activate the amino acid response (AAR) pathway via the protein kinase GCN2. Activated GCN2 phosphorylates and inhibits eukaryotic translation initiation factor 2A (eIF2α), thereby reducing overall protein translation, while specifically enhancing translation of the transcription factor ATF4 (Harding et al., Mol. Cell. 11:619, 2003; Harding et al., Mol. Cell. 6:1099, 2000). Indeed, a number of stress-induced genes reportedly regulated by ATF4 in mouse embryonic fibroblasts (Harding et al., Mol. Cell. 11:619, 2003) were over-represented among the genes induced by HF treatment in T cells (FIG. 4B, FIG. 14). These analyses suggest that at least a portion of the transcriptional response to HF is mediated by ATF4. Furthermore, quantitative real-time PCR (qPCR) experiments confirmed that at least three known AAR-associated genes (Asns, Gpt2, eIF4Ebp1) were induced by HF treatment within 4 hours of T cell activation (FIG. 4C).

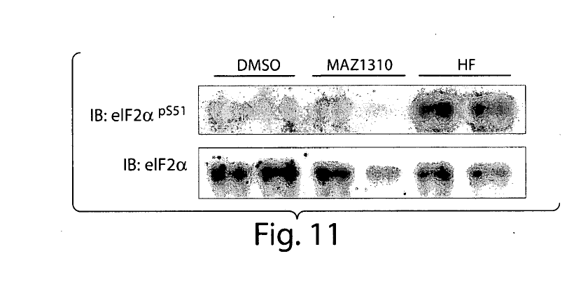

To directly address whether HF activates the AAR pathway, eIF2α phosphorylation and ATF4 protein levels in HF-treated T cells was examined. HF induced detectable eIF2α phosphorylation at 2.5 nM, and this effect plateaued at 5-10 nM HF (FIG. 4D). ATF4 expression levels were highest in T cells treated with 5-10 nM HF and were reduced in cells treated with higher concentrations of HF (20-40 nM) (FIG. 4D), demonstrating a positive correlation between the concentrations of HF that induce ATF4 expression and those that selectively inhibit Th17 differentiation (FIG. 2A). In kinetic analyses, eIF2α phosphorylation in HF-treated cells reached maximum levels by 2 hours and ATF4 protein continued to accumulate until 4 hours (FIG. 4E), indicating that HF activates the AAR pathway before any detectable effects on STAT3 phosphorylation or IL-17 production are observed. AAR activation was a general consequence of HF treatment; HF induced eIF2α phosphorylation, not only in T cells activated in Th17-priming conditions, but also in resting naïve T cells and T cells activated in ThN, Th1, Th2, and iTreg polarizing conditions (FIG. 4F). HF treatment also increased eIF2α phosphorylation in cultured fibroblasts (FIG. 11) and microarray analyses of fibroblasts revealed that HF induced a pattern of early gene induction similar to that seen in T cells. These data demonstrate that activation of the AAR pathway by HF is not a cell type-specific effect. HF treatment induced ATF4 expression in all differentiated T cells, but not in naïve T cells (FIG. 4F). This result most likely reflects the low metabolic rate and relatively inefficient translation capacity of naïve T cells (Rathmell et al., Eur. J. Immunol. 33:2223, 2003). Thus, the rapid activation of the AAR pathway by HF could underlie both its selective inhibition of Th17 differentiation and its effects on fibroblasts (Pines and Nagler, Gen. Pharmacol. 30:445, 1998).

A variety of other cellular stresses (ER stress, oxidative stress, viral infection) also result in eIF2α phosphorylation and ATF4 translation, a phenomenon termed the integrated stress response (ISR) (Harding et al., Mol. Cell. 11:619, 2003; Harding et al., Mol. Cell. 6:1099, 2000). Individual stressors, however, can also activate stress type-specific pathways. For instance, the unfolded protein response (UPR), which is activated by ER stress, results in expression of the transcription factor Xbp-1 through a mechanism involving IRE-1-dependent splicing, as well as nuclear translocation of the ER-sequestered transcription factor ATF6 in addition to eIF2α phosphorylation catalyzed by the protein kinase Perk (Ron and Walter, Nat. Rev. Mol. Cell. Biol. 8:519, 2007; Brunsing et al., J. Biol. Chem. 283, 17954, 2008; Lin et al., Science 318:944, 2007). Xbp-1 and ATF6, in turn, upregulate ER chaperones such as GRP78/BiP and calreticulin, whose expression is specific to the UPR and independent of the eIF2α/ATF4 ISR pathway (Ron and Walter, Nat. Rev. Mol. Cell. Biol. 8:519, 2007; Lee et al., Mol. Cell. Biol. 23: 7448, 2003). However, HF did not induce the expression of these and other hallmark ER stress response genes.

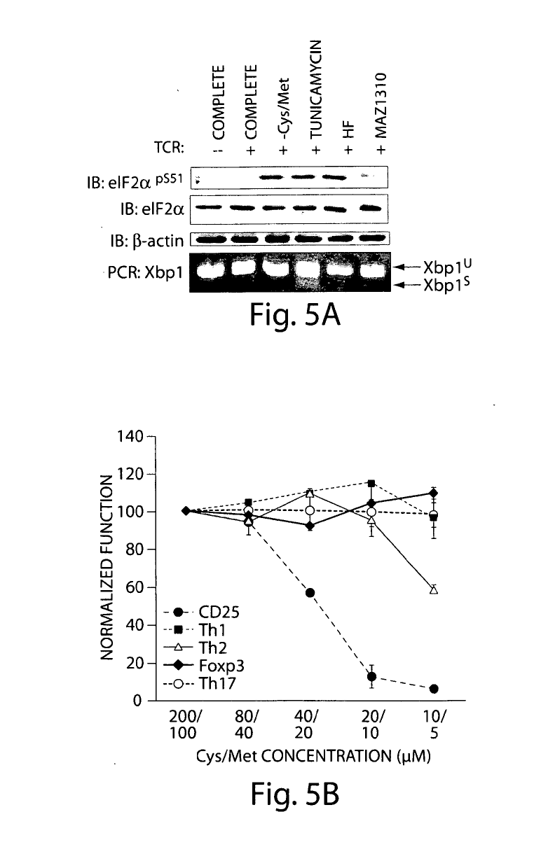

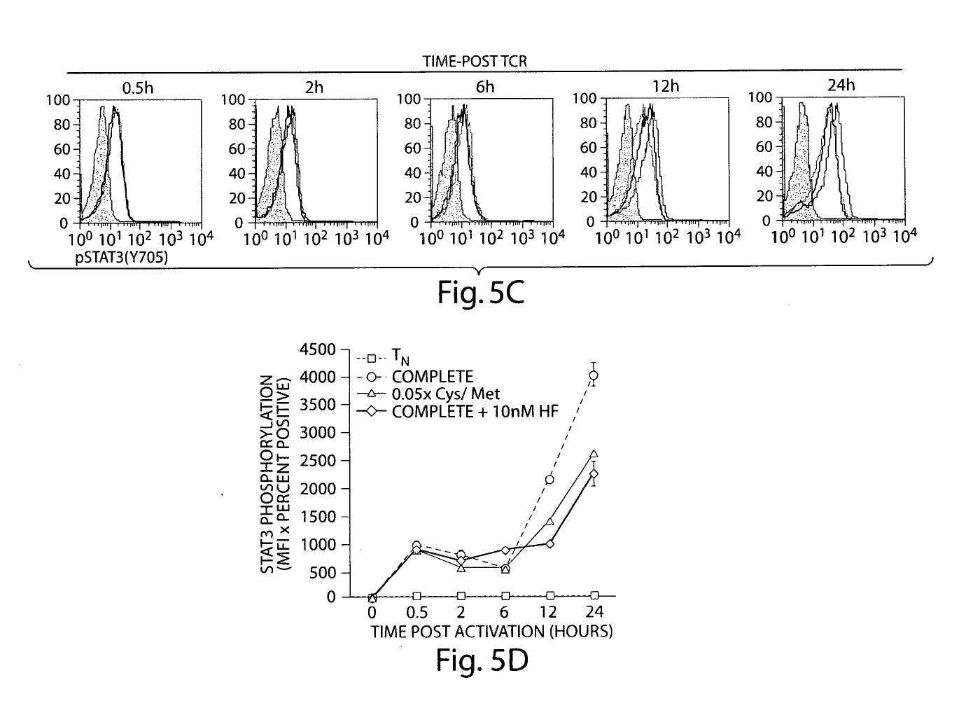

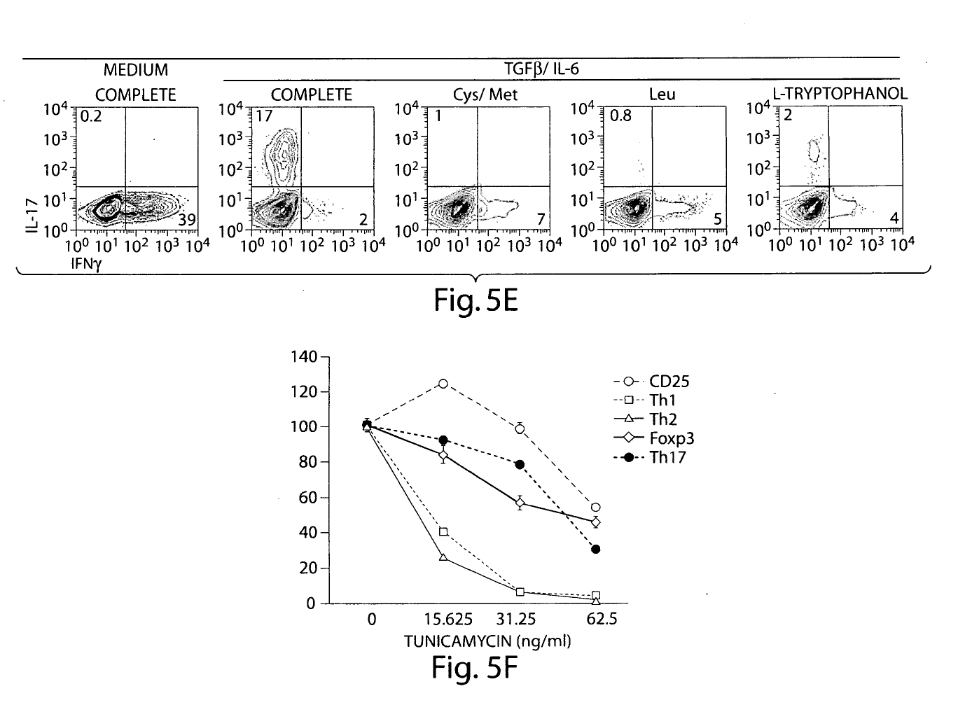

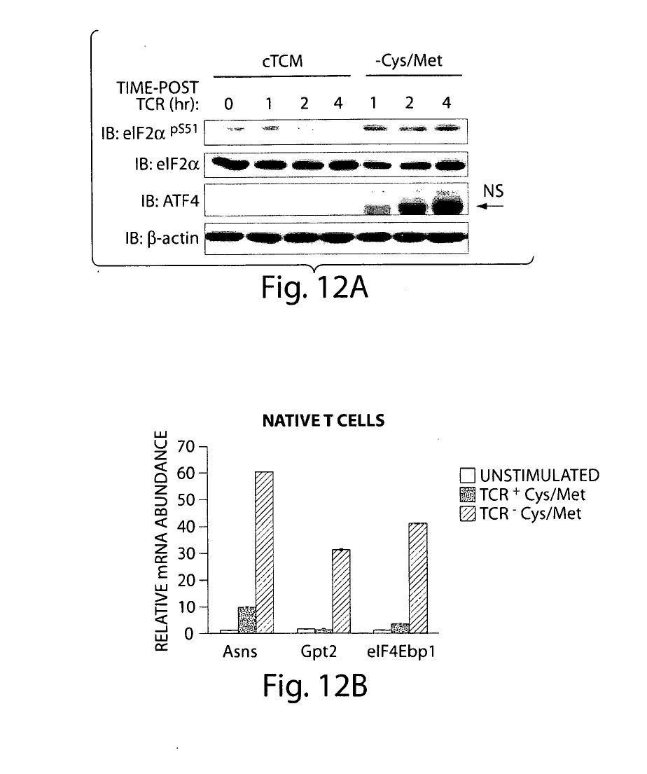

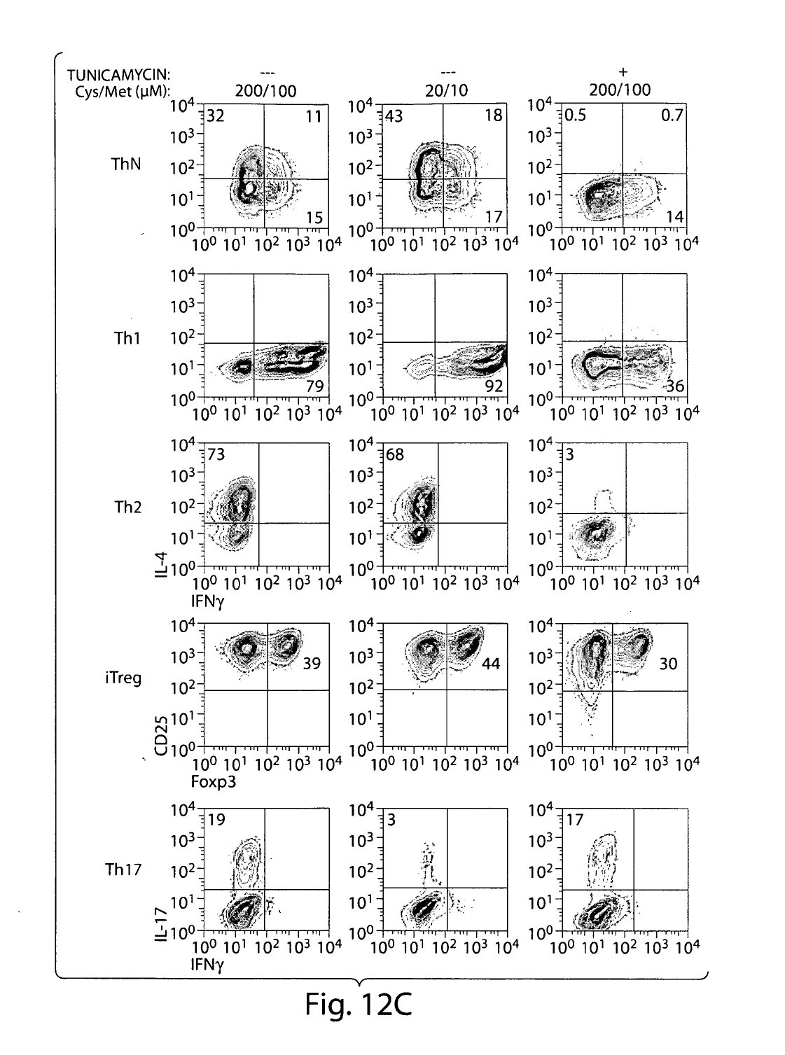

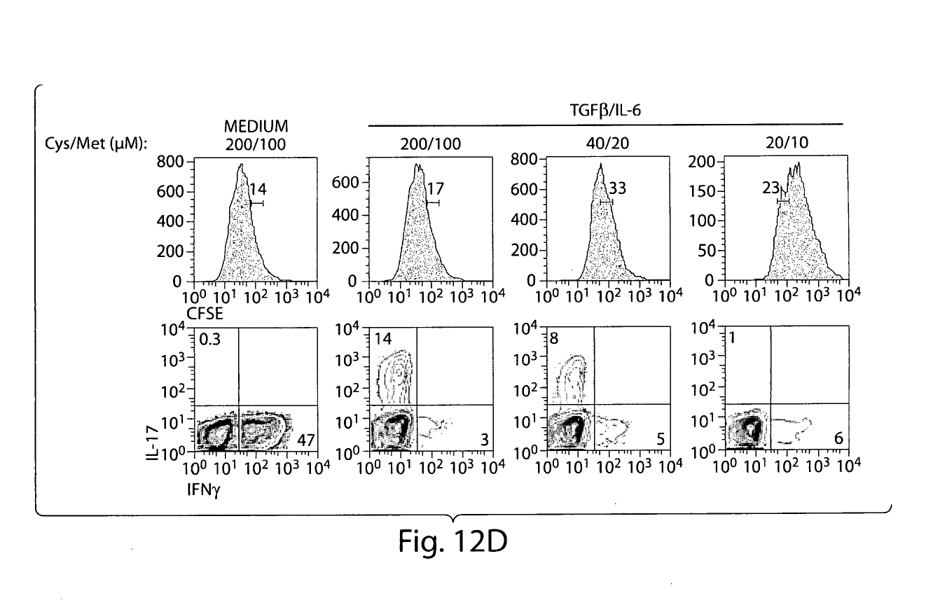

To delineate the stress response pathway activated by HF, the effects of amino acid starvation with those of tunicamycin (an inducer of ER stress) or HF treatment during T cell activation were compared. As expected, cells deprived of cysteine (Cys) and methionine (Met) displayed eIF2α phosphorylation, ATF4 expression, and upregulation of AAR-associated genes but did not induce Xbp-1 splicing (FIG. 5A, FIG. 12A, 12B). In contrast, tunicamycin treatment induced eIF2α phosphorylation and ATF4 expression together with Xbp-1 splicing (FIG. 5A), as characteristic of the UPR. The effects of HF treatment closely resembled those of amino acid starvation, inducing eIF2α phosphorylation without promoting Xbp-1 splicing (FIG. 5A). Taken together, these data indicate that HF specifically induces an AAR.

Next, the effects of amino acid starvation on Th17 differentiation and STAT3 activation were investigated. It was discovered that the functional consequences of Cys/Met-deprivation were remarkably similar to those of HF treatment in T cells. Cys/Met deprivation profoundly and selectively impaired Th17 differentiation in a manner directly related to the concentration of these amino acids in the culture medium. T cells cultured under limiting Cys/Met concentrations showed greatly diminished Th17 differentiation but upregulated CD25 expression and differentiated into Th1, Th2, and iTreg subsets as effectively as T cells cultured in complete medium (FIG. 5B, FIG. 12C). As shown for HF (FIG. 2E), inhibition of IL-17 expression by amino acid starvation was unrelated to cell survival or proliferation (FIG. 12D). Further similar to the effects of HF, Cys/Met-deprivation did not affect the early phase of STAT3 phosphorylation but impaired the maintenance of STAT3 phosphorylation (FIG. 5C, 5D). Moreover, L-tryptophanol, a tryptophan derivative that competitively inhibits tryptophanyl-tRNA loading, or limiting concentrations of a different amino acid, leucine, also impaired IL-17 production (FIG. 5E), suggesting that inhibition of Th17 differentiation is a general consequence of amino acid starvation. The mammalian target of rapamycin (mTOR) pathway represents a second, complementary mechanism through which cells respond to amino acid availability (Fingar and Blenis, Oncogene 23:3151, 2004). However, early transcriptional responses induced by HF and the mTOR inhibitor rapamycin are distinct (Peng et al., Mol. Cell. Biol. 22:5575, 2002 and FIG. 13), and HF did not inhibit signaling downstream of mTOR in fibroblasts.

To test whether inhibition of IL-17 expression was specific to stress induced by amino acid starvation, the influence of tunicamycin on T cell activation and differentiation was tested. Surprisingly, low concentrations of tunicamycin had little influence on IL-17 expression in T cells (FIG. 5F, FIG. 12C) but instead preferentially impaired Th1 and Th2 differentiation (FIG. 5F, FIG. 12C). These data suggest that individual stress response pathways can regulate distinct aspects of T cell differentiation and effector function but also indicate that eIF2α phosphorylation and ATF4 translation (shared consequences of both AAR and UPR) are not sufficient to explain the selective regulation of Th17 differentiation by HF or amino acid deprivation. The impact of cellular stress on the immune system is complex. Data herein show here that Th17 differentiation is particularly susceptible to stress induced by amino acid deprivation, whereas ER stress blunts Th1 and Th2 differentiation. In addition to these effects on T cell effector function, eIF2α phosphorylation induced during ER stress may have cytoprotective effects in oligodendrocytes and pancreatic β cells during acute inflammation associated with autoimmune encephalomyelitis and diabetes (Puccetti and Grohmann, Nat. Rev. Immunol. 7:817, 2007; Lin et al., J. Clin. Invest. 117:448, 2007). Diverse cellular responses to stress may regulate both T cell function and the downstream cellular targets of inflammatory cytokine signaling during tissue inflammation.

The distinctive sensitivity of Th17 cells to AAR pathway activation may have a role during adaptive immune responses in vivo. For example, indoleamine 2,3-dioxygenase (IDO), an IFNγ-induced enzyme that breaks down tryptophan, has been shown to cause local depletion of tryptophan at sites of inflammation and activate the AAR pathway in resident T cells (Puccetti and Grohmann, Nat. Rev. Immunol. 7:817, 2007; Munn et al., Immunity 22:633, 2005). While local IDO accumulation is most often associated with proliferative impairment in T cells, expansion or conversion of Foxp3+ T cells also has been reported following upregulation of IDO (Puccetti and Grohmann, Nat. Rev. Immunol. 7:817, 2007; Park et al., Arthritis Res. 10:R11, 2008). Given the reciprocal relationship between the development of pro-inflammatory Th17 cells and tissueprotective iTreg cells, it is postulated that IDO-mediated immune tolerance involves local AAR mediated inhibition of Th17 differentiation and consequent skewing of the Th17: iTreg balance in favor of iTreg cells (Romani et al., J. Immunol. 180:5157, 2008).

Materials and Methods

Mice

Mice were housed in specific pathogen-free barrier facilities and were used in accordance with protocols approved by the animal care and use committees of the Immune Disease Institute and Harvard Medical School. Wild-type C57B/6 mice were purchased from Jackson laboratories (Bar Harbor, Me.) and were used for all in vitro culture experiments unless otherwise noted. ROSA26-YFPfl/fl (Srinivas et al., BMC Dev. Biol. 1:4, 2001) and ROSA26- STAT3C-GFPfl/fl (Mesaros et al., Cell. Metab. 7:236, 2008) mice have been described. Dr. Alexander Rudensky provided lymphoid organs from Foxp3gfp and Foxp3ko mice (Gavin et al., Nature 445:771, 2007).

Cell Isolation

Primary murine T and B cells were purified by cell sorting. CD4⁺ CD25⁻ T cells were positively selected using CD4 dynabeads and detachabeads (Dynal—Oslo, Norway) per manufacturers instructions followed by nTreg depletion using a CD25 microbead kit (Miltenyi biotech—Auburn, Calif.). Naïve (CD4⁺ CD62Lhi CD44lo Foxp3gfp- or CD4⁺ CD62Lhi CD44lo CD25⁻) T cells were purified from Foxp3gfp or Foxp3ko mice, respectively, by FACS sorting. CD8⁺ T cells or B cells were isolated from CD4⁻ fractions using CD8 negative isolation kit (Dynal) or CD43 negative isolation kit (Miltenyi biotech), respectively. Resting human CD4⁺ T cells were isolated from PBMC of healthy human donors using Dynal CD4 Positive Isolation Kit (Invitrogen—Carlsbad, Calif.) as previously described (Sundrud et al., Blood 106:3440, 2005). CD4⁺ cells were further purified to obtain memory T cells by staining with PE-conjugated anti-human CD45RO-PE antibodies (BD Biosciences), and sorting on a FACSAria cytometer (BD Biosciences). Following purification, cells were greater than 99% CD4⁺ CD45RO⁺. CD14⁺ monocytes were isolated from autologous PBMC by MACS sorting using a magnetic separator (AutoMACS, Miltenyi Biotech) and were more then 99% pure following isolation.

Cytokines, Antibodies and Cell Culture

Purified CD4⁺ CD25⁻ T cells were activated in vitro as previously described (Djuretic et al., Nat. Immunol. 8:145, 2007) using 0.3 μg/ml hamster anti-mouse CD3 (clone 145-2C11) (ATCC—Manassas, Va.) and 0.5 μg/ml hamster anti-mouse CD28 (BD Pharmingen—San Jose, Calif.). Activated cell cultures were differentiated using the following combinations of cytokines and antibodies: iTreg-recombinant human TGFβ1 (3 ng/ml—R&D systems, Minneapolis, Minn.), Th17-TGFβ1 (3 ng/ml) plus recombinant mouse IL-6 (30 ng/ml—R&D systems). Th1 and Th2 differentiation was performed as previously described (Djuretic et al., Nat. Immunol. 8:145, 2007). Human IL-2 supernatant (National Cancer Institute) was used in culture at 0.01 U/ml and was added at 48 hours-post activation when T cells were split into tissue culture wells lacking CD3 and CD28 antibodies, with the exception of Th17 cultures that were maintained in the absence of exogenous IL-2. CD8⁺ T cells were activated with 1 μg/ml anti-CD3 and 1 μg/ml anti-CD28 and were expanded in 0.1 U/ml IL-2 until day 6 post activation. CD43-depleted B cells were activated in vitro by culturing with 25 μg/ml LPS (Sigma—St. Louis, Mo.) for 3-4 days in the presence or absence of TGFβ. All reagents (see below) were added at the time of T cell activation and again at 48 hours post activation unless indicated otherwise. For some experiments, purified CD4⁺ CD25⁻ T cells, CD8⁺ T cells or B cells were labeled with 1 μM CFSE (Invitrogen) prior to activation in accordance with manufacturer's instructions. Human T cell activation was performed by plating purified monocytes in a 96-well flat bottom plate at a concentration of 2×10⁴ cells per well in complete medium overnight. 10⁵ purified human memory T cells were added to monocyte cultures in the presence of soluble anti-CD3/anti-CD28 beads (Dynabeads, Invitrogen). T cells were expanded in the presence HF or MAZ1310 for up to 6 days.

Inhibitors and Amino Acid Starvation

1 kg of 10% pure HF was received as a gift from Hangpoon Chemical Co. (Seoul, Korea), which was further purified via HPLC to >99% purity and used for experiments. MAZ1310 (Kamberov, Ph.D. Dissertation, Harvard University, 2008) was generated by chemical derivatization of halofuginone and was used at equal concentrations as a negative control. HF and MAZ1310 were prepared as 100 mM stock solutions in DMSO and diluted to the indicated concentrations. SB-431542 (Inman et al., Mol. Pharmacol. 62:65, 2002) (Tocris bioscience—Ellisville, Mo.) was prepared as a 10 mM stock solution in DMSO and was used in culture at 10 μM. L-tryptophanol was prepared as a 20 mM stock solution in 0.1 M NaOH, pH 7.4 and was used at 0.2 mM. For amino acid starvation experiments, T cells were activated and differentiated as above in D-MEM medium without L-cysteine and L-methionine (Invitrogen—Carlsbad, Calif.), or D-MEM medium without L-leucine. Stocks containing 20 mM L-cysteine (Sigma—St. Louis, Mo.) plus 10 mM L-methionine (Sigma), or 400 mM L-leucine (Sigma) were prepared in ddH2O, pH 1.0 and were added to medium at the indicated concentrations.

Tat-Cre Transduction

6×His-TAT-NLS-Cre (HTNC—herein called TAT-Cre) was prepared as previously described (Peitz et al., Proc. Nat. Acad. Sci. USA 99:4489, 2002). Purified T cells where rested in complete medium for 30 minutes, washed 3 times in ADCF-Mab serum free medium (Hyclone—Logan, Utah) and resuspended in pre-warmed serum free medium supplemented with 50 μg/ml of TATCre. Following a 45 minute incubation at 37° C., transduction was stopped using media containing 10% FCS and T cells were rested for 4-6 hours in complete medium prior to activation.

Retroviral Transductions

MIG and MIG.RORγt retroviral cDNA were gifts from Dr. Dan Littman. pRV and pRV.FOXP3 retroviral constructs have been described previously (Wu et al., Cell 126:375, 2006). Retroviral particles were generated using the phoenix-Eco system (ATCC). Supernatants were concentrated by centrifugation and stored at −80° C. prior to use in culture. Thawed retroviral supernatants were added to T cell cultures 12 hours after T cell activation in the presence of 8 μg/ml polybrene (American bioanalytical—Natick, Mass.) and centrifuged for 1 hour at room temperature to enhance infections.

Detection of Cytokine Production

Cytokines secreted into media supernatant were measured using the mouse Th1/Th2 cytometric bead array (CBA—BD Pharmingen) in accordance with manufacturers instructions. Briefly, CD4+ CD25− T cells were activated in anti-CD3/anti-CD28-coated tissue culture wells (see above) and supernatants were collected at the indicated times.

For detection of intracellular cytokines in murine cells, cultured T or B cells were stimulated with 10 nM PMA (Sigma) and 1 mM ionomycin (Sigma) for 4-5 hours in the presence of 10 mM brefeldin A (Sigma). Stimulated cells were harvested, washed with PBS and fixed with PBS plus 4% paraformaldehyde at room temperature for 20 minutes. Cells were then washed with PBS, permeabilized with PBS supplemented with 1% BSA and 0.5% saponin (Sigma) at room temperature for 10 minutes before cytokine-specific antibodies were added and incubated with cells for an additional 20 minutes at room temperature. Human T cells were restimulated with PMA (20 ng/ml) (Sigma) and lonomycin (500 ng/ml) (Sigma) for 6 hours in the presence of golgi plug (BD Biosciences) and intracellular staining was performed using cytofix/cytoperm kit (BD Biosciences) per manufacturers instructions. All stained cells were stored at 4° C. in PBS plus 1% paraformaldehyde prior to FACS analyses.

FACS Analyses and Sorting

All cell surface staining was performed in FACS buffer (PBS/2% FBS/0.1% NaN₃) and antibodies were incubated with cells on ice for 20-30 minutes. Cells were washed with FACS buffer and fixed with FACS buffer plus 1% paraformaldehyde prior to data acquisition. For phospho-STAT3 intracellular staining, stimulated T cells cultured with or without TGFβ plus IL-6 for the indicated times were harvested on ice and fixed in PBS plus 2% paraformaldehyde for 10 minutes at 37° C. Fixed cells were washed twice with staining buffer (PBS/1% BSA/0.1% NaN₃) and then permeabilized with perm buffer III (BD Pharmingen) on ice for 30 minutes. Cells were then washed twice with staining buffer and PE-conjugated anti-STAT3 (pY705) (BD Pharmingen) was added per the manufacturer's instructions and incubated with cells at room temperature for 45-60 minutes. Cells were then washed and stored in staining buffer prior to data acquisition. Foxp3 intracellular staining was performed using a Foxp3 intracellular staining kit (eBioscience—San Diego, Calif.) in accordance with the manufacturer's instructions. Fluorescent-conjugated antibodies purchased from BD Pharmingen were percp-Cy5.5-conjugated anti-CD4, PE-conjugated anti-CD25, PE-conjugated anti-IL-17, PE-conjugated anti-phospho-STAT3 and APC-conjugated anti-human IFNγ. Fluorescent conjugated antibodies purchased from eBioscience include FITC-conjugated anti-CD8, APC-conjugated anti-mouse/rat Foxp3, PE-conjugated anti-IL-4, APC-conjugated anti-IFNγ, PE-conjugated anti-granzyme B, APC-conjugated streptavidin, PE-conjugated anti-IL-6, and PE-conjugated anti-human IL-17. Biotin-conjugated anti-IgA antibody was purchased from Southern biotech (Birmingham, Ala.). All FACS data was acquired on a FACSCalibur flow cytometer (BD Pharmingen) and analyzed using FlowJo software (Treestar, Inc.—Ashland, Oreg.). FACS sorting was performed on a FACS-Diva cytometer (BD Pharmingen).

Quantitative Real-Time PCR

T cells were activated as described above, collected at the indicated times and pellets were flash-frozen in liquid nitrogen. Total RNA was obtained by RNeasy (Quiagen—Valencia, Calif.) column purification per manufacturers instructions. RORγt expression was determined after reverse transcription using the message sensor kit (Ambion—Austin, Tex.) per the manufacturer's instructions and taqman primers and probe as described elsewhere (Ivanov et al., Cell 126:1121, 2006). Sybrgreen quantitative real-time PCR was performed on T cell RNA samples following reverse transcription via SuperScript II first-strand cDNA synthesis kit (Invitrogen—Carlsbad, Calif.). All PCR data was collected on an iCycler thermal cycler (Biorad—Hercules, Calif.). Primer sequences used for detecting stress response genes are listed below.

Asns forward:
(SEQ ID NO: _)
5′-TGACTGCCTTTCCGTGCAGTGTCTGAG-3′
Asns reverse:
(SEQ ID NO: _)
5′-ACAGCCAAGCGGTGAAAGCCAAAGCAGC-3′
Gpt2 forward:
(SEQ ID NO: _)
5′-TAGTCACAGCAGCGCTGCAGCCGAAGC-3′
Gpt2 reverse:
(SEQ ID NO: _)
5′-TACTCCACCGCCTTCACCTGCGGGTTC-3′
elF4Ebp1 forward:
(SEQ ID NO: _)
5′-ACCAGGATTATCTATGACCGGAAATTTC-3′
elF4Ebp1 reverse:
(SEQ ID NO: _)
5′-TGGGAGGCTCATCGCTGGTAGGGCTAG-3′
Hprt forward:
(SEQ ID NO: _)
5′-GGGGGCTATAAGTTCTTTGCTGACC-3′
Hprt reverse:
(SEQ ID NO: _)
5′-TCCAACACTTCGAGAGGTCCTTTTCAC-3′

Western Blotting

Whole cell lysates were generated from T cells activated for the indicated times. For STAT3 and Smad2/3 western blots cells were harvested, washed in PBS and lysed in 50 mM Tris, pH 7.4, 0.1% SDS, 1% Triton-X 100, 140 mM NaCl, 1 mM EDTA, 1 mM EGTA supplemented with protease inhibitors tablets (Roche—Germany), 1 mM NaF and 1 mM Na₃VO₄. For eIF2α and ATF4 western blots, cells were harvested as above and lysed in 50 mM Tris, pH 7.4, 2% SDS, 20% glycerol and 2 mM EDTA supplemented with protease and phosphatase inhibitors as above. All lysates were cleared via centrifugation and 15-30 μg of protein was resolved by SDS-PAGE. Protein was transferred to nitrocellulose membranes, blocked and blotted using specific antibodies. Antibodies used for western blot analysis were anti-phospho-Smad2, anti-STAT3 (pY705), anti-STAT3, anti-eIF2α^{ps51}, anti-eIF2α (all from cell signaling technology—Danvers, Mass.). Anti-ATF4/CREB2 and anti-β-actin were purchased from Santa Cruz Biotechnology (Santa Cruz, Calif.). HRP-conjugated secondary antibodies were all purchased from Sigma, with the exception of HRP-conjugated anti-armenian hamster antibody (Jackson Immunoresearch—West Grove, Pa.).

Microarrays, Data Analyses and Statistics

RNA prepared from activated T cells treated with 10 nM HF or MAZ1310 for either 3 or 6 hours, was amplified, biotin-labeled (MessageAmp II Biotin-Enhanced kit, Ambion—Austin, Tx), and purified using the RNeasy Mini Kit (Qiagen—Valencia, Calif.). Resulting cRNAs were hybridized to M430 2.0 chips (Affymetrix, Inc.). Raw data were normalized using the RMA algorithm implemented in the “Expression File Creator” module from the GenePattern software package (Reich et al., Nat. Gen. 38:500, 2006) (available on the internet at the following address: broad.mit.edu/cancer/software/genepattern/). Data were visualized using the GenePattern “Multiplot” modules. Gene expression distribution analyses were performed using Chi-squared statistical tests. For all other statistical comparisons, p values were generated using one-tailed student T-tests on duplicate or triplicate samples.

### Example 2

### The Amino Acid Starvation Response (AAR) is Activated by HF in Cultured Fibroblastic Cells

SV-MES mesangial cells were stimulated for 2 hours with halofuginone (20 nM), or an inactive derivative of halofuginone (MAZ1310, 20 nM) or control buffer, lysed, and analyzed by SDS PAGE/Western blot for total or Ser51 phosphorylated eIF2alpha, and total or Thr 898 phosphorylated GCN2. FIG. 15 shows the results of the experiment. Duplicate cell samples are shown. Phosphorylation of GCN2 at Thr898 is a defining characteristic of AAR activation, therefore the activation of GCN2 phosphorylation at this site following HF treatment indicates that HF activates the AAR. Activated GCN2 phosphorylates eif2alpha at Ser 51; therefore, this is an expected downstream outcome of AAR activation.

### Example 3

### GCN-2 Dependency of Halofuginone Stimulated eIF2Alpha Phosphorylation

CD4⁺ CD25⁻ T cells purified from wild type of GCN2^{-/-} mice were activated through TCR for 4 hours in the presence of halofuginone (10 nM) or (10 nM). Results are shown in FIG. 16. Whole cell lysates were analyzed by SDS PAGE/Western blot and antibodies indicated. Treatment with Halofuginone, but not the inactive derivative, leads to phosphorylation of Ser51 of eif2alpha only in wild type cells and not in GCN2^{-/-} cells, establishing the eif2alpha phosphorylation following halofuginone stimulation occurs through activation of the AAR/GCN2 pathway.

### Example 4

### Proline Rescue of Translation Inhibition by Halofuginone

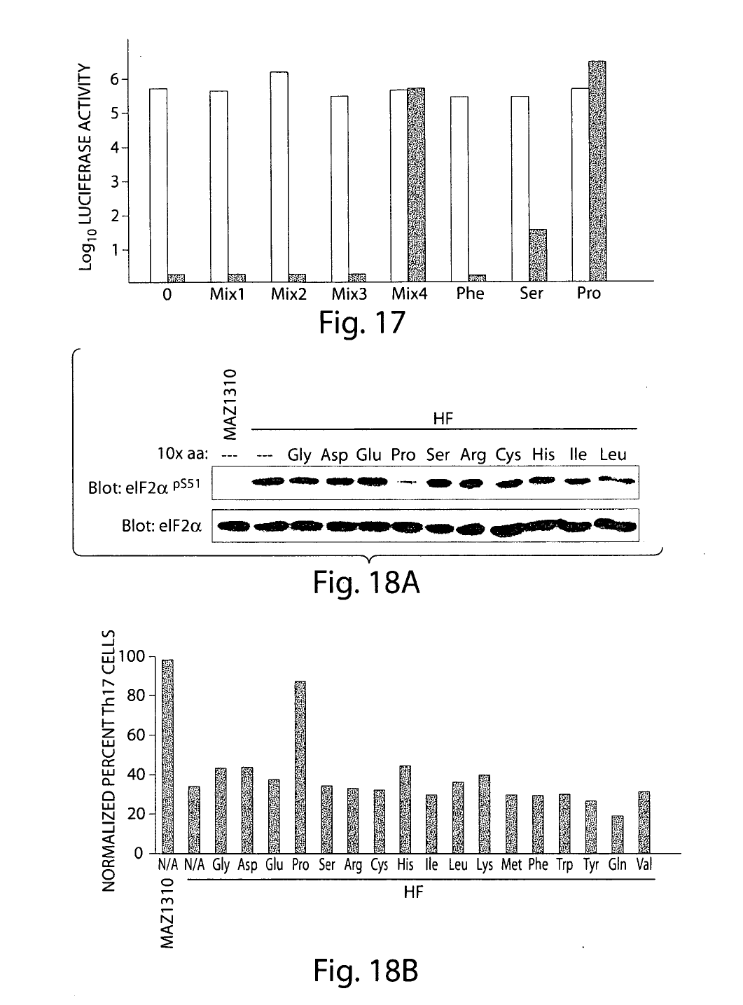

Translation in vitro was performed using rabbit reticulocyte lysates and luciferase mRNA as template. Translation was measured as arbitrary units of luciferase activity using a luminometer based luminescence assay. Results are shown in FIG. 17. Log scale presentation of background-subtracted data is shown. Translations were performed without (dark bars) or with (light bars) 400 nM halofuginone, in the absence of amino acids (O), or with the following additions: Mix 1:1 mM Asn, 1 mM Arg; Mix 2: 1 mM Lys, 1 mM Ile, 1 mM Tyr; Mix 3: 1 mM His, 1 mM Met, 1 mM Leu; Mix 4: 1 mM Ser, 1 mM Phe, 1 mM Pro, Phe: 2 mM Phe; Pro: 2 mM Pro; Ser: 2 mM Ser. Addition of proline, either alone or in combination with phenylaline and serine, rescues inhibition of translation by halofuginone, establishing that proline utilization for translation (by glutamyl prolyl tRNA synthetase) is the critical target of halofuginone action.

### Example 5

### Halofuginone-Induced eIF2Alpha Phosphorylation is Rescued by Proline Addition

Naïve T-cells were treated to stimulate the T-cell receptor (TCR) in the presence or absence of 10 nM halofuginone in the presence of 1 mM added amino acid, and then assayed for eIF2alpha activity phosphorylation by SDS PAGE/Western blot. Results are shown in FIG. 18A. Phosphorylation induced by halofuginone is blocked by added proline. These data establish that utilization of proline is inhibited by halofuginone, leading to activation of the AAR.

### Example 6

### Rescue of Halofuginone Inhibited Th17 Differentiation by Proline

Naïve T-cells were stimulated to differentiate in the presence or absence of 10 nM halofuginone, with 1 mM of the indicated amino acids added to the medium, and stained for Th17 differentiation on day 4. Results are shown in FIG. 18B. Naïve murine T cells were activated in the presence or absence of TGFβ plus IL-6 as indicated, expanded in for 4 days and restimulated with PMA and ionomycin for intracellular cytokine staining. For intracellular cytokine staining, fixed cells were washed twice with staining buffer (PBS/1% BSA/0.1% NaN₃) and then permeabilized with perm buffer III (BD Pharmingen) on ice for 30 minutes. Cells were then washed and stored in staining buffer prior to data acquisition. All FACS data was acquired on a FACSCalibur flow cytometer (BD Pharmingen) and analyzed using FlowJo software (Treestar, Inc.—Ashland, Oreg.). FACS sorting was performed on a FACS-Diva cytometer (BD Pharmingen). Bars indicate percentage of cells differentiation as Th17 as indicated by IL17 expression. Proline, and no other added amino acid, rescues the inhibition of Th17 differentiation by halofuginone, confirming that proline utilization is the critical target for halofuginone inhibition of Th17 differentiation.

### Example 7

### Depletion of Amino Acids or tRNA Synthetase Inhibition with L-Tryptophanol Inhibits Th17 Differentiation

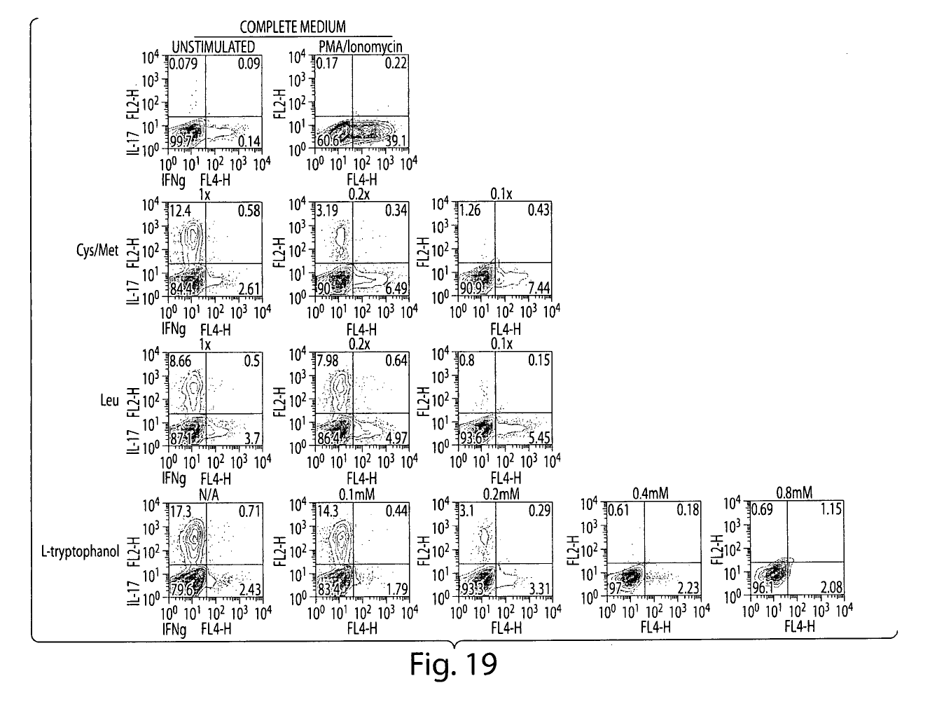

T cells were cultured in complete medium (complete—200 μM Cys/100 μM Met/4 mM Leu), medium containing 0.1×, 0.2×, or 1× cysteine and methionine (Cys/Met), medium containing 0.1× leucine (Leu), or complete medium plus 0.2 mM L-tryptophanol. Cells were activated in the presence or absence of TGFβ plus IL-6, expanded for 4 days and restimulated with PMA and ionomycin for intracellular cytokine staining. For intracellular cytokine staining, fixed cells were washed twice with staining buffer (PBS/1% BSA/0.1% NaN₃) and permeabilized with perm buffer III (BD Pharmingen) on ice for 30 minutes. Cells were then washed and stored in staining buffer prior to data acquisition. All FACS data were acquired on a FACSCalibur flow cytometer (BD Pharmingen) and analyzed using FlowJo software (Treestar, Inc., Ashland Oreg.). FACS sorting was performed on a FACS-Diva cytometer (BD Pharmingen). The results, depicted in FIG. 19, show that depletion of Cys/Met, depletion of Leu, and treatment with tryptophanol all inhibited Th17 differentiation.

### Example 8

### Modulation of Th17-Mediated Effects In Vivo

The ability of systemic HF treatment to block IL-17 expression and associated autoimmune inflammation in vivo was examined using two distinct models of experimental autoimmune encephalomyelitis (EAE). The first model used is referred to as adjuvant-driven EAE and is actively induced by immunization of wild-type mice with the immunodominant myelin-derived peptide antigen MOG₃₃₋₅₅ emulsified in Complete Freund's Adjuvant (CFA). The second model, a passive model of EAE induction, is initiated by the transfer of myelin proteolipid protein (PLP)-reactive T cells into lymphopenic hosts.

Adjuvant-driven EAE was induced in 8 week-old wild-type B6 mice purchased from Charles River laboratories (Kingston, N.Y.) by subcutaneous injection of MOG₃₃₋₅₅ peptide emulsified in Incomplete Freund's Adjuvant (IFA) plus 5 mg/ml heat-killed M. tuburculosis (BD Biosciences) in both dorsal flanks as described in Veldhoen et al. (Nat. Immunol. 7(11):1151-1156, 2006).

Passive EAE was induced by intravenous transfer of purified CD3⁺ splenic T cells isolated from PLP TCR transgenic B10.S mice into syngeneic RAG2-deficient mice (3×10⁶ cells/mouse) (Waldner et al., J. Clin. Invest. 113(7):990-997, 2004).

Mice were injected daily with HF (2 μg/mouse) or vehicle control (DMSO) i.p. Clinical signs of EAE were assessed according to the following score: 0, no signs of disease; 1, flaccid tail; 2, weak gait/hind limb paresis; 3, hind limb paralysis; 4, tetraplegia; 5, moribund. Cytokine production during EAE was determined either in peripheral T cells isolated from spleen or lymph nodes of mice prior to disease onset (day 6-10) or in mononuclear cells isolated from the brain and spinal cords of mice with severe disease (clinical score ≧2) between days 15-20. Briefly, splenocytes were stained for intracellular cytokines following erythrocyte lysis with ammonium chloride buffer. T cells were isolated from brain and spinal cords of mice with active EAE following perfusion with cold PBS. Minced CNS tissue was digested with liberase C1 (0.33 mg/ml, Roche Diagnostics) or collagenase D (10 mg/ml, Roche Diagnostics) at 37° C. for 30-45 minutes. Cell suspensions were passed through 70 μm cell strainers (VWR) and fractionated by 70%/30% Percoll gradient centrifugation. Mononuclear cells were collected from the interphase, washed and used for intracellular cytokine analysis.

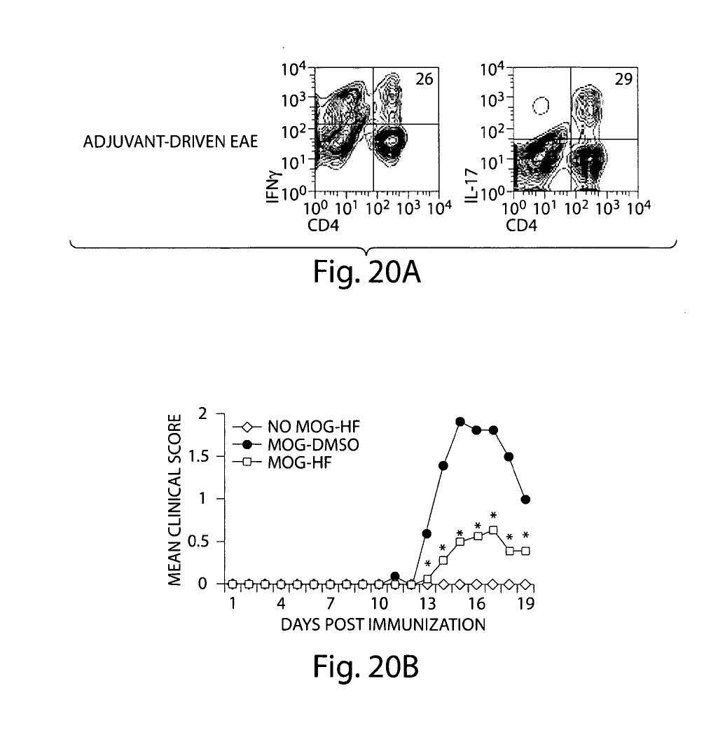

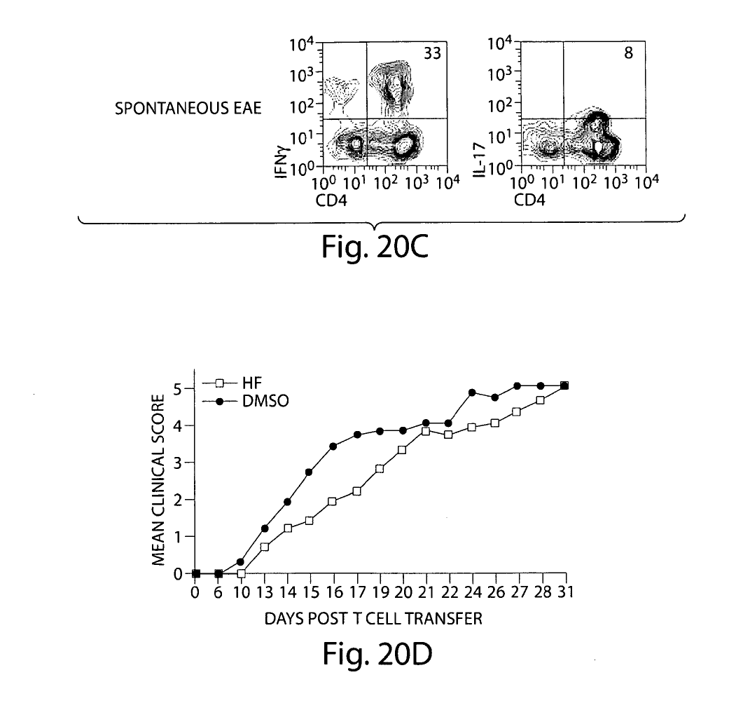

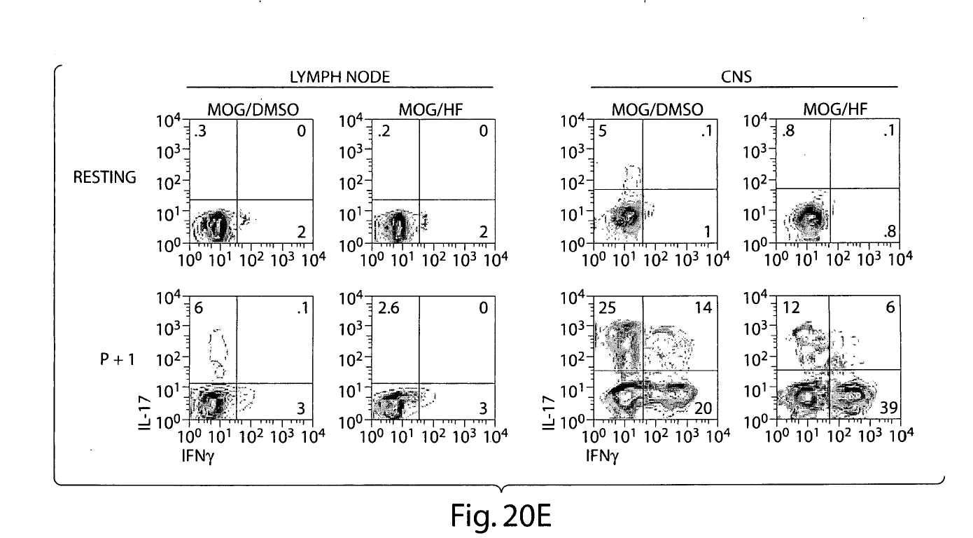

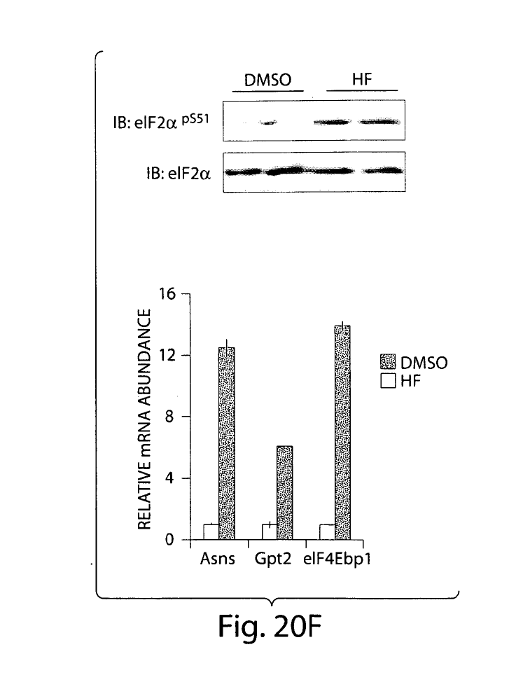

The adjuvant-driven EAE model is associated with infiltration of both IL-17- and IFNγ-expressing CD4⁺ T cells into the CNS (FIG. 20A). Low-dose HF treatment (2 μg HF daily, ˜0.1 mg/kg) significantly reduced both the severity of adjuvant-driven EAE disease and frequency of disease onset (FIG. 20B). The second, passive model of EAE induction leads to a predominant Th1 response, rather than Th17 response, within CNS infiltrates (FIG. 20C). In marked contrast to the adjuvant-driven EAE model, HF-treated mice in the passive EAE model developed disease symptoms with kinetics and severity similar to control treated animals (FIG. 20D). The contrasting effects of HF in these two models of EAE support the notion that HF selectively inhibits IL-17-associated inflammatory T cell function without inducing general T cell hyporesponsiveness. Taken together, these data suggest that HF can modulate autoimmune inflammation associated with Th17, but not Th1, responses.

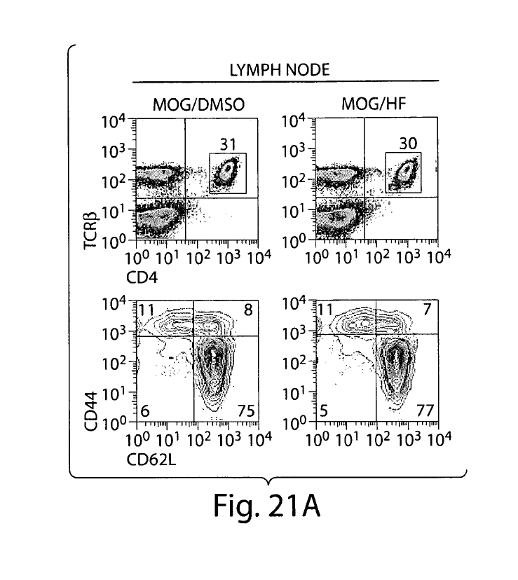

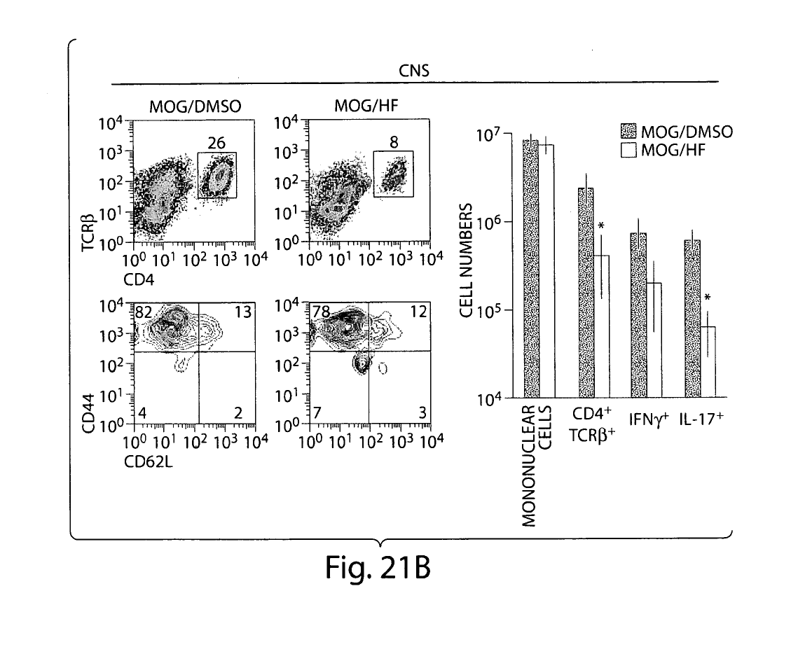

HF-mediated protection from adjuvant-driven EAE was accompanied by a reduction in T cell-derived IL-17-expression, both in peripheral lymph nodes prior to disease onset and in CNS tissue during active disease (FIG. 20E), as well as an overall reduction in CD4⁺ T cell infiltrates into the CNS (FIG. 21). Consistent with in vitro results, HF impaired IL-17 production but did not affect IFNγ expression in the same T cell populations. Moreover, splenocytes isolated ex vivo from HF-injected mice displayed increased eIF2α phosphorylation and expression of AAR-associated transcripts (FIG. 20F). Thus, systemic administration of low doses of HF activates the AAR, leading to a selective impairment of Th17 differentiation, and concomitant blunting of IL-17 associated inflammatory responses in vivo.

Thus, consistent with in vitro data, it was discovered that HF protects mice from adjuvant-driven EAE through in vivo activation of the AAR. HF selectively reduced the number of IL-17 expressing T cells in vivo, but had no effect on the number of IFNγ T-cells. These data are consistent with reports showing that adjuvant-driven EAE disease is particularly sensitive to modulation of IL-17 expression. Notably, HF had no effect on an independent, passive model of EAE that develops in the absence of a Th17 response, demonstrating that HF is neither globally immunosuppressive nor generically protective against CNS inflammation. Both Th1 and Th17 cells can drive EAE pathogenesis when transferred into mice. In the adjuvant-driven EAE model described above, a roughly equal induction of Th1 and Th17 cells was observed, whereas in the passive model of EAE, encephalitogenic T cells were biased towards a Th1 response. Thus, the lack of an effect of HF in the passive model, in comparison to the adjuvant-driven EAE is likely due to the distinctive inflammatory T cell responses in the two models.

### Example 9

### Inhibition of Prolyl-tRNA Synthetase Underlies the Bioactivity of Halofuginone

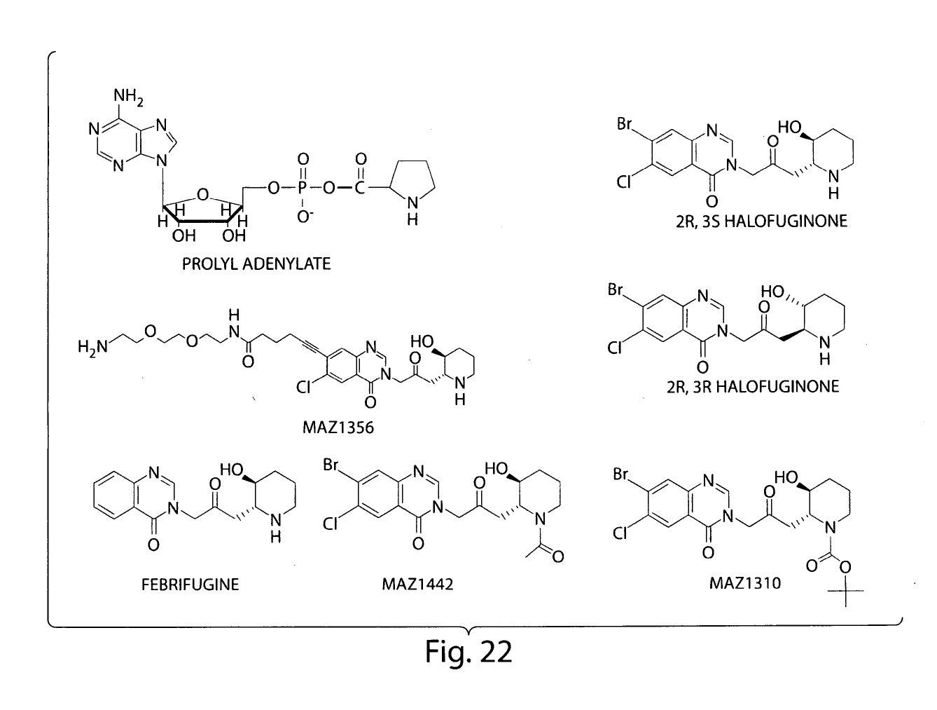

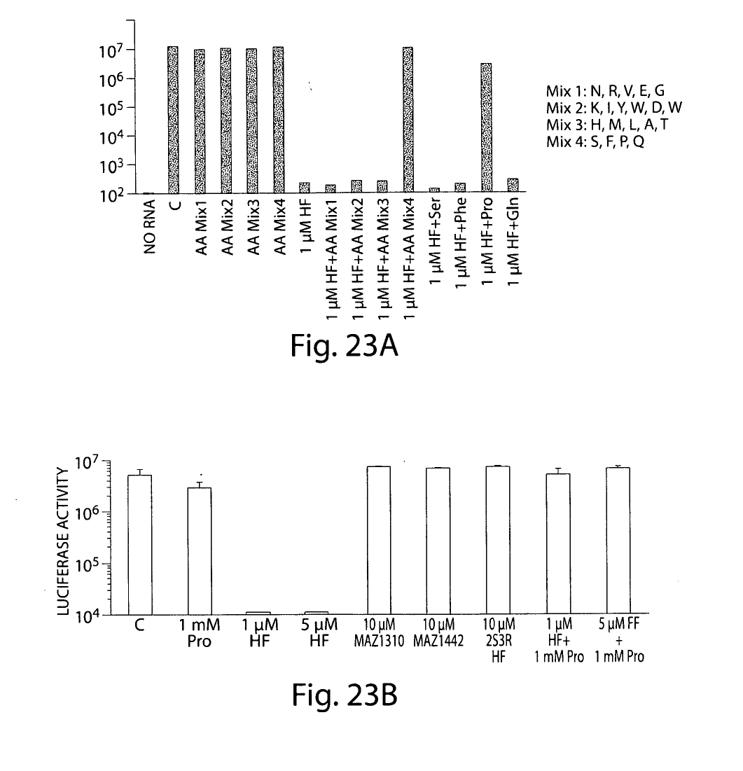

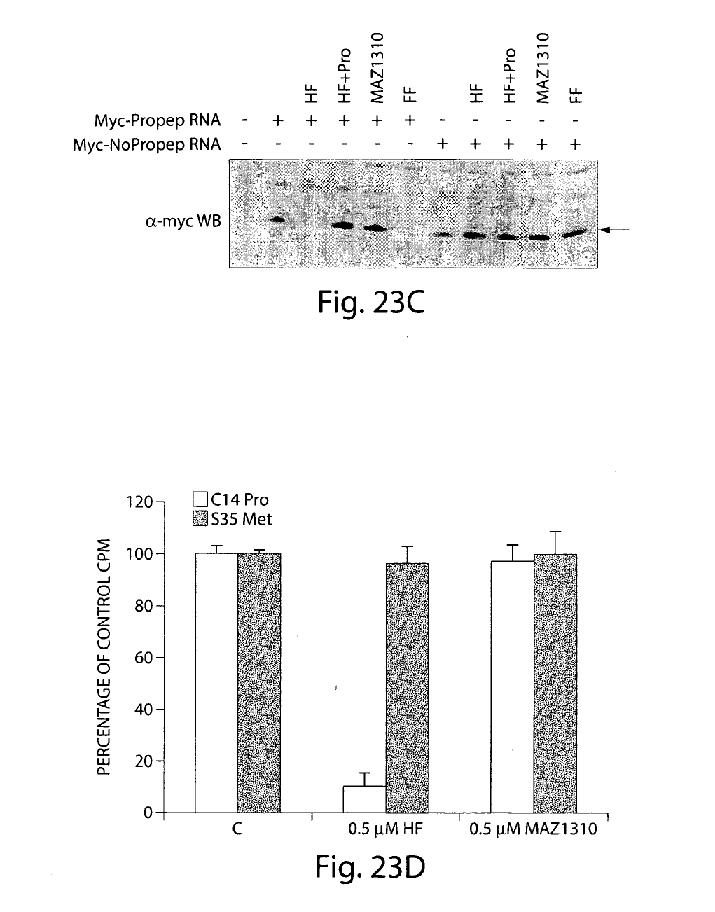

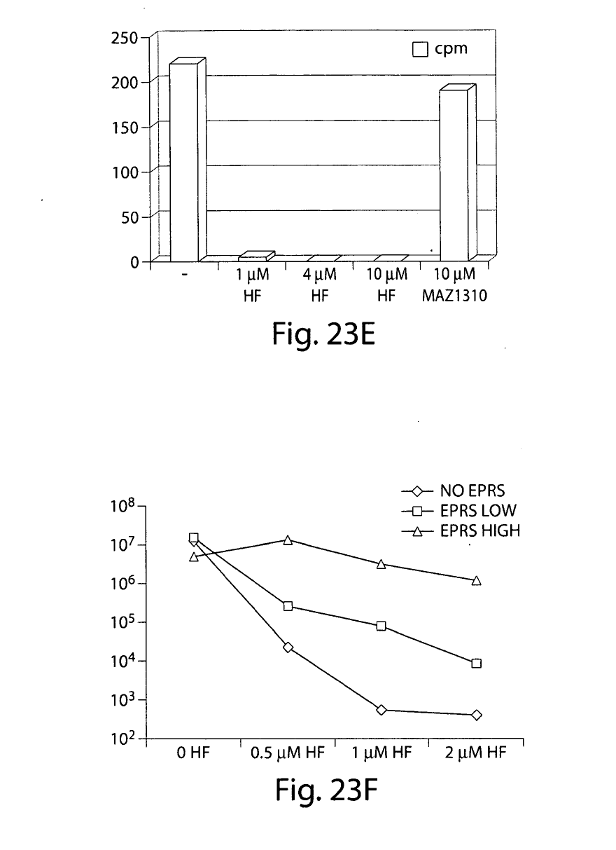

In intact cells, amino acid incorporation into tRNA can be limited, for example, by inhibiting the enzymes responsible for tRNA charging or by altering the intracellular levels of amino acid through effects on transport, synthesis, or catabolism. To distinguish amongst these possibilities, the effects of halofuginone (HF) and febrifugine (FF) (FIG. 22) were tested in a cell free in vitro translation system (rabbit reticulocyte lysate, RRL) where amino acid availability for translation can be controlled directly. Both HF and FF inhibited the translation of luciferase RNA in RRL in the presence of a standard amino acid mix (FIG. 23A). Supplementation of RRL with excess amino acids established that only proline could restore translation inhibited by HF and FF (FIG. 23A), indicating that these compounds act to limit proline utilization by the translational apparatus. The activities of FF and HF as antimalarials (Kobayashi, S. et al. Catalytic Asymmetric Synthesis of Antimalarial Alkaloids Febrifugine and Isofebrifugine and Their Biological Activity. J Org Chem 64, 6833-6841, (1999)) and HF as an inhibitor of Th17 cell differentiation (Sundrud, M. S. et al. Halofuginone inhibits TH17 cell differentiation by activating the amino acid starvation response. Science 324, 1334-1338, (2009)) previously have been shown to be stereospecific, with the 2R3S isomer showing no biological activity (FIG. 23B). Consistent with these observations, the 2R3S isomer has no activity in the RRL assay. Additionally, derivatives of HF that lack activity in cell-based assays, MAZ1310 and MAZ1442, also have no activity in the RRL assay (FIG. 23B). These data suggest that the ability of FF and HF to inhibit proline utilization is functionally linked to the bioactivities of these compounds.

To confirm that HF/FF-inhibition specifically targets the utilization of proline for translation, how these compounds affect the translation of a pair of small synthetic polypeptides that differ only with respect to the presence of proline was examined. The NoPro polypeptide completely lacks proline, whereas ProPro contains a proline dipeptide. HF and FF prevented translation of ProPro, but had no effect on the translation of NoPro (FIG. 23C), suggesting that proline utilization may be the sole target for the inhibitory effect of these compounds on translation in RRL. RRL lacks detectable GCN2, and showed no increase in eIF2-alpha phosphorylation following HF addition (data not shown). Inhibition of translation by limiting amino acid utilization in this system therefore likely may be the result of limitation of a charged tRNA species rather than of indirect regulation of the translational apparatus, and the lack of inhibition of NoPro by HF and FF supports this interpretation.

The effects of HF on prolyl-tRNA charging was examined. RRL were supplemented with ¹⁴C-Pro or ³⁵S-Met in the presence or absence of HF, and total tRNA was isolated (FIG. 23D). HF inhibited the incorporation of ¹⁴C-Pro, but not ³⁵S-Met, into tRNA at doses comparable to those necessary to inhibit translation, indicating that inhibition of amino acid utilization by HF was specific to proline, a reconstituted prolyl-tRNA charging reaction was set up using purified EPRS; HF inhibited this reaction (FIG. 23E), as it did the RRL prolyl-tRNA charging. Moreover, addition of purified EPRS to RRL rescued HF/FF-inhibition of protein translation (FIG. 23F), suggesting that EPRS may be a critical target for inhibition of translation by these compounds in RRL.

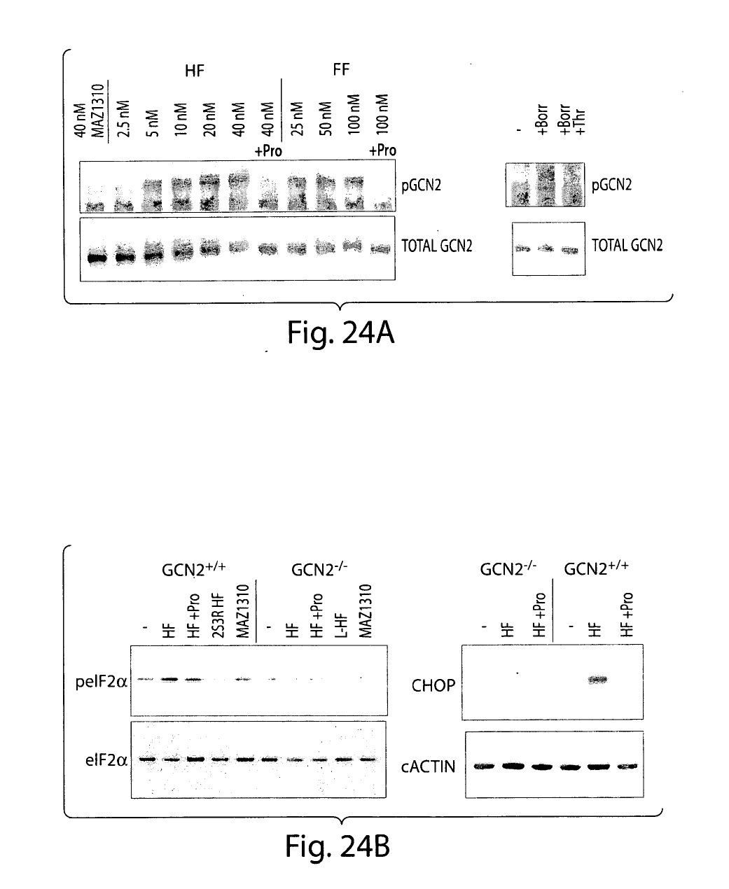

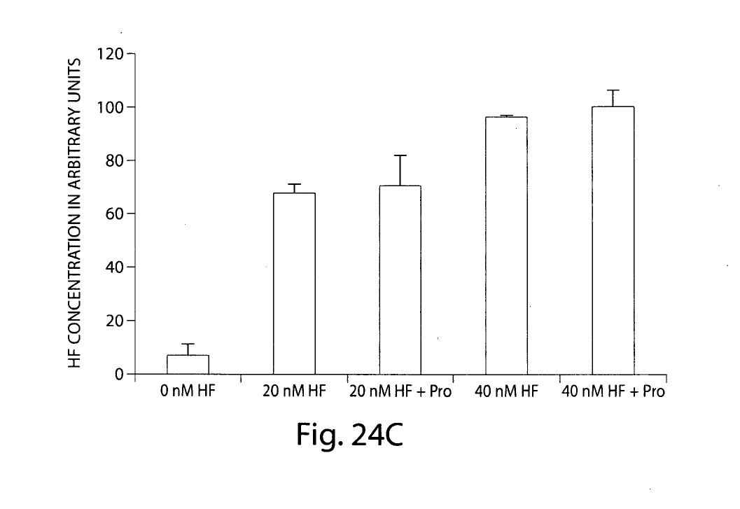

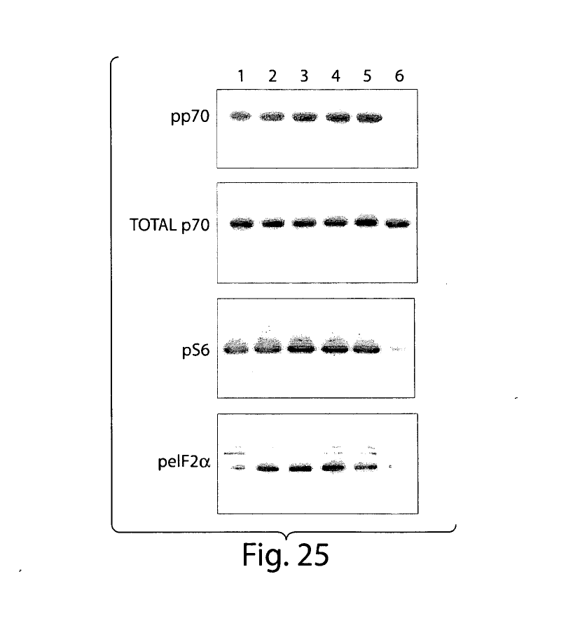

It was examined whether the observed activation of the AAR in intact cells by HF proceeds through inhibition of proline utilization. Stimulation of GCN2 phosphorylation by HF/FF in fibroblasts was abrogated by the addition of 2 mM proline (FIG. 24A, left). Likewise, the ability of borrelidin, a known threonyl tRNA synthetase inhibitor, to stimulate GCN2 phosphorylation was prevented by addition of an excess of its cognate amino acid threonine (FIG. 24A, right). Addition of proline also prevented HF-dependent activation of AAR pathway components downstream of GCN2 phosphorylation, including eIF2-alpha phosphorylation and CHOP induction. These downstream AAR responses to HF were absent in GCN2−/− fibroblasts (FIG. 24B), suggesting that proline utilization may be the principal target for HF action in intact cells. The mTOR pathway, like the AAR, acts as a cellular sensor for amino acid availability, but, unlike the AAR, mTOR signaling was not blocked by tRNA synthetase-inhibitors. HF-treatment of T cells and fibroblasts activated the AAR pathway without concomitant inhibition of mTOR signaling (Sundrud, M. S. et al. Halofuginone inhibits TH17 cell differentiation by activating the amino acid starvation response. Science 324, 1334-1338, (2009)) (FIG. 25). It was concluded that HF was not exerting a direct effect on mTOR signaling, consistent with a model in which HF acts to limit tRNA charging rather than altering amino acid levels in intact cells. To exclude the possibility that proline blocks the action of HF by preventing its uptake or accumulation in intact cells, An anti-HF antibody was used in an ELISA assay to measure intracellular HF levels directly in the presence or absence of excess proline. The intracellular accumulation of HF was not affected by proline addition (FIG. 24C), supporting the interpretation that proline reverses the effect of HF on AAR activation by enhancing intracellular proline utilization.

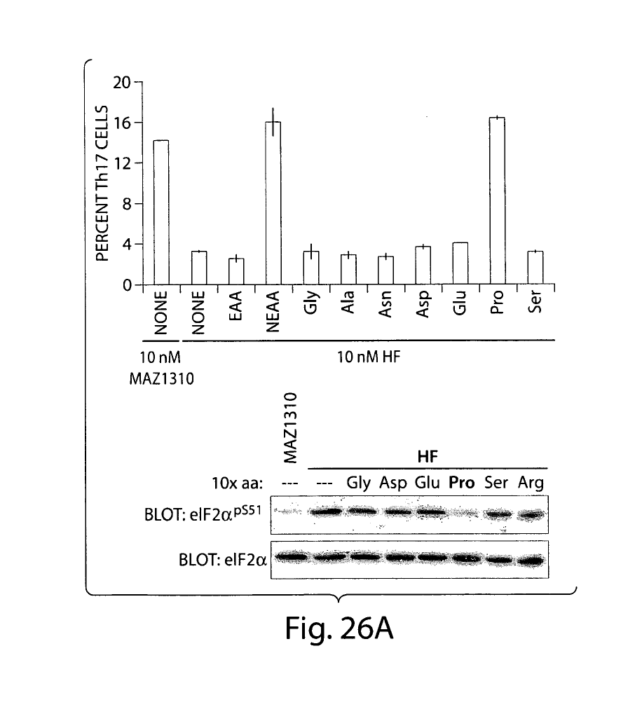

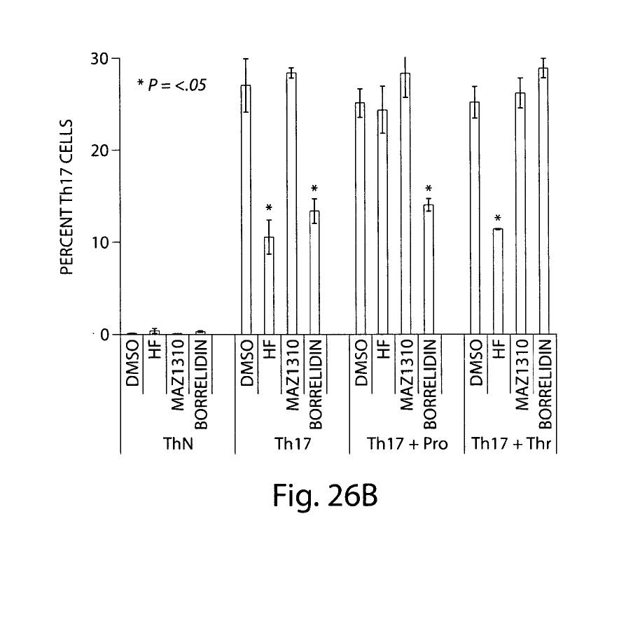

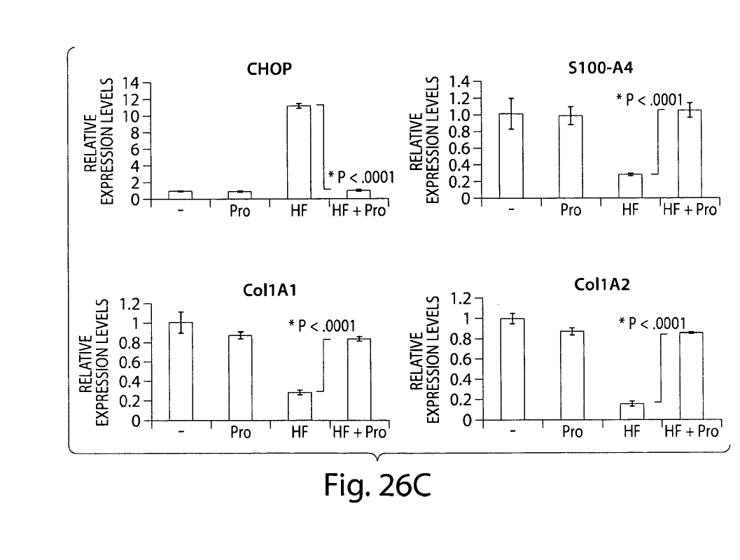

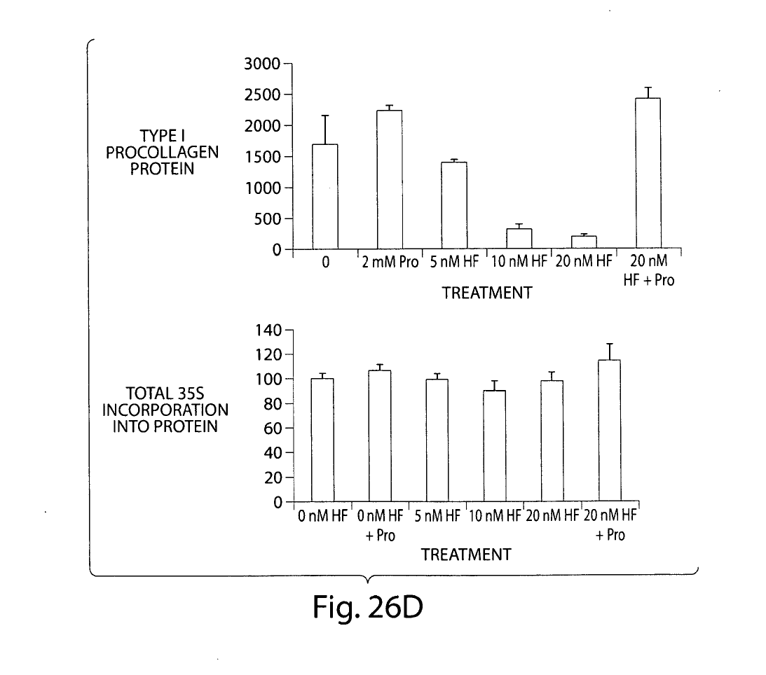

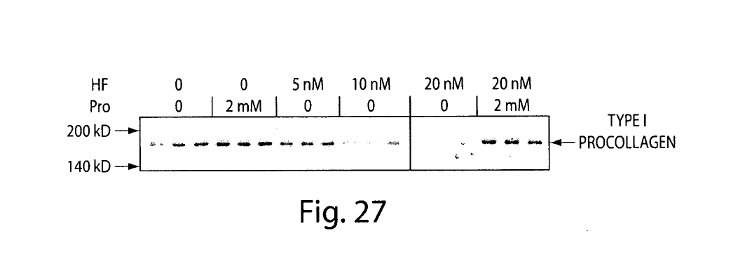

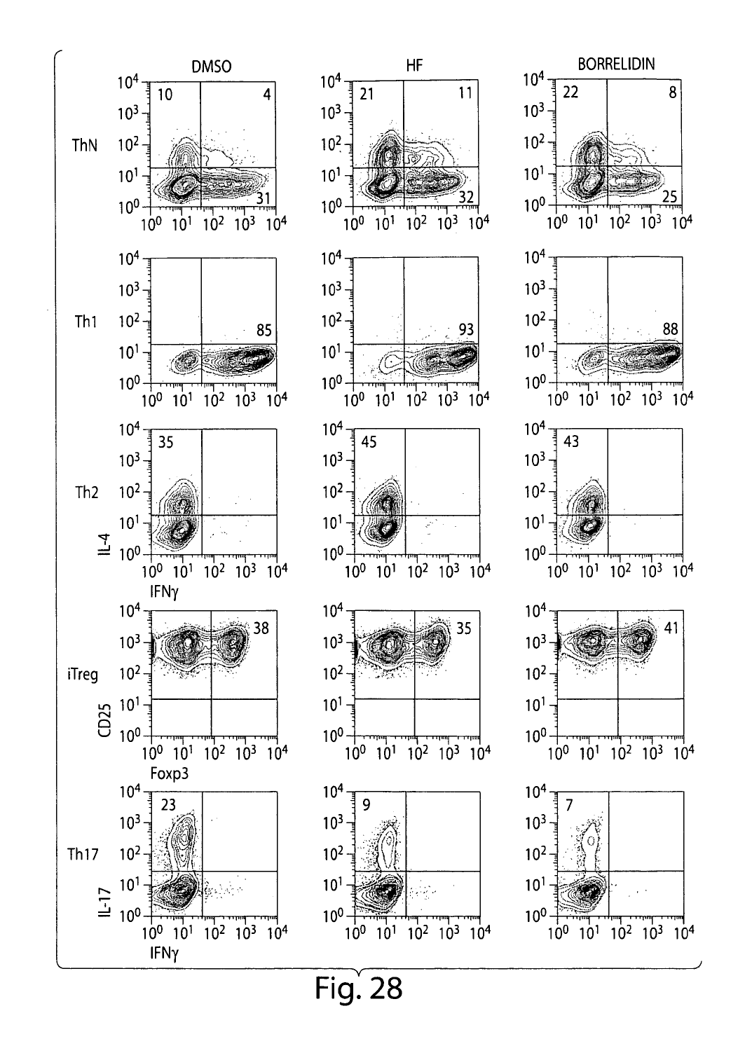

It was previously shown that HF selectively inhibits Th17 cell differentiation through AAR activation, and that media-supplementation with excess, pooled amino acids effectively reverses these effects (Sundrud, M. S. et al. Halofuginone inhibits TH17 cell differentiation by activating the amino acid starvation response. Science 324, 1334-1338, (2009)). Comparison of the effects of non-essential versus essential amino acid pools established that only non-essential amino acids restored Th17 cell differentiation, or prevented eIF2-alpha phosphorylation in the presence of 10 nM HF (FIG. 26A). Testing of individual non-essential amino acids established that only proline rescued Th17 differentiation in HF-treated T cells (FIG. 26A). It was next tested whether the threonyl tRNA synthetase inhibitor borrelidin could recapitulate the effects of HF on Th17 differentiation. Borrelidin inhibited Th17 cell differentiation and, like HF, these effects were reversed by the addition of threonine, borrelidin's cognate amino acid (FIG. 26B). As in the case of HF-inhibition of Th17 cell differentiation, borrelidin's effects were selective; borrelidin acted without perturbing the differentiation of Th1, Th2, or iTreg cells (FIG. 28). These results demonstrated that AAR activation by tRNA synthetase inhibitors provides a general approach to the selective inhibition of Th17 cell differentiation.

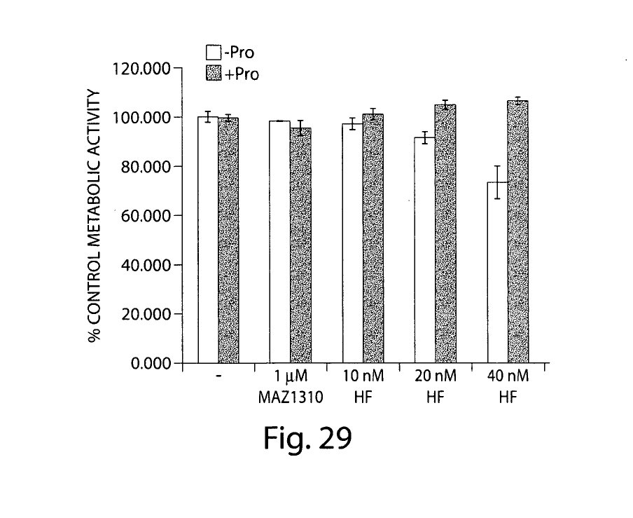

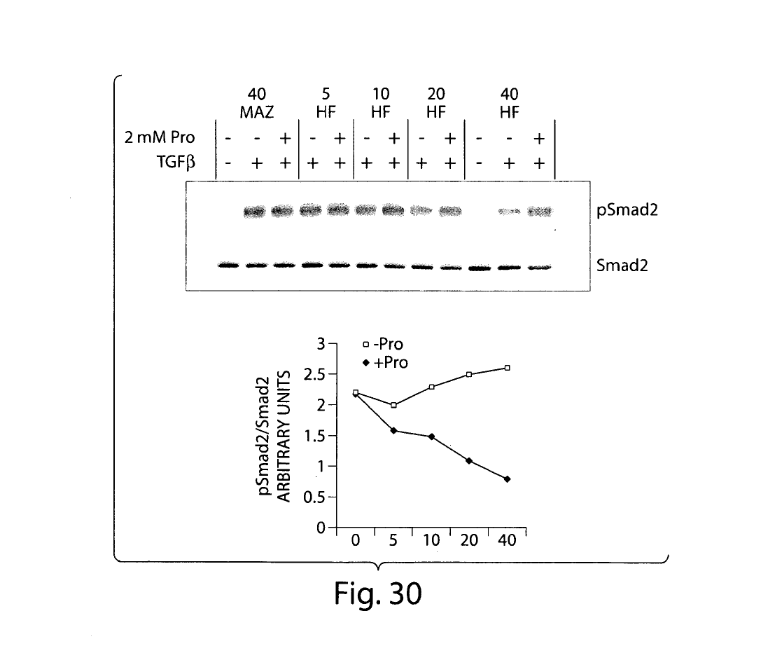

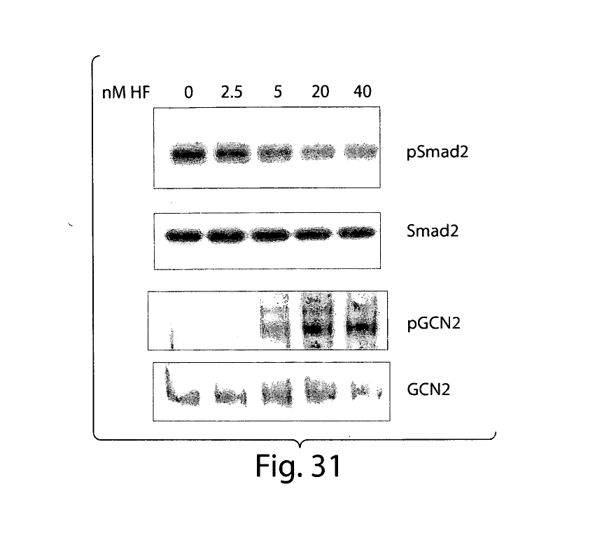

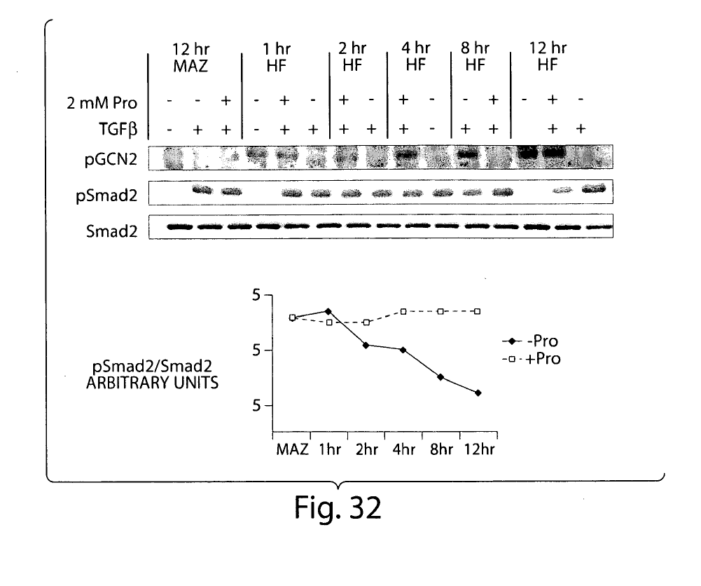

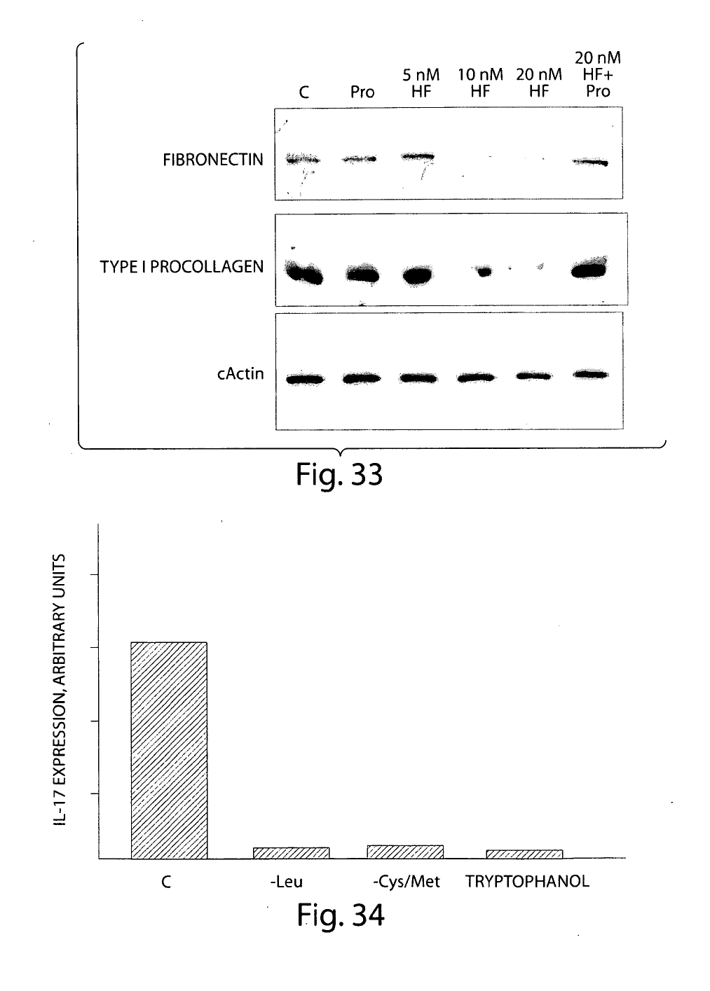

The ability of HF to inhibit tissue remodeling in vivo is evidenced by its potent suppression of tissue fibrosis (Pines, M. & Nagler, A. Halofuginone: a novel antifibrotic therapy. Gen Pharmacol 30, 445-450, (1998); McGaha, T. et al. Effect of halofuginone on the development of tight skin (TSK) syndrome. Autoimmunity 35, 277-282, (2002)) and tumor progression (Elkin, M. et al. Inhibition of bladder carcinoma angiogenesis, stromal support, and tumor growth by halofuginone. Cancer Res 59, 4111-4118, (1999)). As an antifibrotic agent, HF inhibits the overproduction and deposition of extracellular matrix (ECM) components, such as Type I collagen and fibronectin, both in vivo and in cultured fibroblasts. Herein, it was shown that HF inhibited mRNA levels, and proline rescued expression, for ColIA1, ColIA2, and S100A4 in mouse embryo fibroblasts (MEFs) (FIG. 26C). S100A4, which is produced and secreted from tumor-activated stromal cells, has been implicated in fibrosis and tumor metastasis, as well as in tissue invasion by synoviocytes during rheumatoid arthritis (Boye, K. & Maelandsmo, G. M. S100A4 and Metastasis: A Small Actor Playing Many Roles. Am J Pathol, (2009); Schneider, M., Hansen, J. L. & Sheikh, S. P. S100A4: a common mediator of epithelial-mesenchymal transition, fibrosis and regeneration in diseases? J Mol Med 86, 507-522, (2008); Oslejskova, L. et al. Metastasis-inducing S100A4 protein is associated with the disease activity of rheumatoid arthritis. Rheumatology (Oxford) 48, 1590-1594, (2009); Oslejskova, L., Grigorian, M., Gay, S., Neidhart, M. & Senolt, L. The metastasis associated protein S100A4: a potential novel link to inflammation and consequent aggressive behaviour of rheumatoid arthritis synovial fibroblasts. Ann Rheum Dis 67, 1499-1504, (2008)). Expression of mRNA encoding the AAR-responsive factor CHOP was stimulated by HF, concomitant with inhibition of the expression of ECM genes. Consistent with prior reports, HF-treatment of cells for 24 hours also dramatically inhibited the production of secreted Type I procollagen protein (Pines, M. & Nagler, A. Halofuginone: a novel antifibrotic therapy. Gen Pharmacol 30, 445-450, (1998); Huebner, K. D., Jassal, D. S., Halevy, O., Pines, M. & Anderson, J. E. Functional resolution of fibrosis in mdx mouse dystrophic heart and skeletal muscle by halofuginone. Am J Physiol Heart Circ Physiol 294, H1550-1561, (2008)) and the production of fibronectin (Sato, S. et al. Halofuginone prevents extracellular matrix deposition in diabetic nephropathy. Biochem Biophys Res Commun 379, 411-416, (2009)), at doses that did not significantly change ³⁵S-methionine incorporation into total protein (FIG. 26D). HF-inhibition of these ECM proteins, like the HF-induced modulation of gene transcription, was reversed by the addition of 2 mM proline to cells. The total metabolic activity of fibroblasts also was unaffected over this time period (FIG. 29). Additionally, in mouse and human fibroblasts the production of collagen protein was inhibited by borrelidin-treatment and rescued by the addition of threonine (data not shown). These data indicated that the antifibrotic effects of HF on ECM production: 1) were mediated through EPRS inhibition, 2) were not exclusive to proline-rich proteins, such as collagen, and 3) were mediated by AAR pathway activation, rather than complete blockage of proline utilization during translation. HF-inhibition of tissue remodeling also has been associated with affects on TGF-beta signaling both in vitro (Sato et al. Halofuginone prevents extracellular matrix deposition in diabetic nephropathy. Biochem Biophys Res Commun 379, 411-416, (2009)) and in vivo (Huebner et al. Functional resolution of fibrosis in mdx mouse dystrophic heart and skeletal muscle by halofuginone. Am J Physiol Heart Circ Physiol 294, H1550-1561, (2008)). HF reduced TGF-beta-stimulated Smad2 phosphorylation in fibroblasts over the same dose range that it upregulated GCN2 phosphorylation, and this inhibition was reversed by the addition of proline (FIGS. 30 and 31). The time course for HF-inhibition of Smad2 phosphorylation is much slower than that for activation of GCN2 phosphorylation (FIG. 32), indicating that the inhibition of TGF-beta signaling is a secondary, indirect effect of HF-treatment. In summary, HF-induced inhibition of signaling and gene expression related to tissue remodeling, and HF-inhibition of ECM protein production was reversed by proline supplementation, suggesting that these effects resulted from suppression of EPRS activity and subsequent activation of the AAR pathway.

Materials and Methods

Protein Sequence of ProPro and NoPro Polypeptides

ProPro:
(SEQ ID NO: _)
MEQKLISEEDLNEMEQKLISEEDLNEMEQKLISEEDLNEMEQKLIS
EEDLNEMEQKLISEEDLNEMESLGDLTMEQKLISEEDLNSSSQSLY
RGAFVYDCSPPKFKASRASRTIVSRIT
(the location of the proline dipeptide is
indicated by the bold “PP”)
NoPro:
(SEQ ID NO: _)
MEQKLISEEDLNEMEQKLISEEDLNEMEQKLISEEDLNEMEQKLIS
EEDLNEMEQKLISEEDLNEMESLGDLTMEQKLISEEDLNSSSQSLY
RGAFVYDCSKFKASRASRTIVSRIT

FACS Analysis

All FACS data was acquired on a FACSCalibur flow cytometer (BD Pharmingen) and analyzed using FlowJo software (Treestar, Inc.). Protocols and antibodies used for FACS staining of T cells have been described previously. Briefly, Th17 differentiation (percentage of IL-17+ IFNg− cells) was determined on day 4-cultured T cells following restimulation with phorbol myristate acetate (PMA; 10 nM) and ionomycin (1 mM), in the presence of brefeldin A (10 mg/ml) for 4-5 hours. Cytokine expression in restimulated cells was determined by intracellular cytokine staining as detailed in (Sundrud et al). In some experiments cytokine production by cells activated in non-polarizing conditions (ThN), Th1, or Th2 conditions was determined on day 5 following restimulation and intracellular cytokine staining as above. Inducible T regulatory (iTreg) differentiation was assessed by CD25 and Foxp3 upregulation on day 3-post activation using a commercially available Foxp3 intracellular staining kit (eBioscience).

Primers and Probes for Q-PCR

Q-PCR was performed using the Roche LightCycle UPR system, using the following primers and probes:

(SEQ ID NO: _)

| | |
|---|---|
| Col1A1: probe 15, primer 1: | catgttcagctttgtggacct |

(SEQ ID NO: _)

| | |
|---|---|
| Col1A1: probe 15, primer 2: | gcagctgacttcagggatgt |

(SEQ ID NO: _)

| | |
|---|---|
| Col1A2: probe 46, primer 1: | gcaggttcacctactctgtcct |

(SEQ ID NO: _)

| | |
|---|---|
| Col1A2: probe 46, primer 2: | cttgccccattcatttgtct |

(SEQ ID NO: _)

| | |
|---|---|
| S100A4: probe 56, primer 1: | ggagctgcctagcttcctg |

(SEQ ID NO: _)

| | |
|---|---|
| S100A4: probe 56, primer 2: | tcctggaagtcaacttcattgtc |

(SEQ ID NO: _)

| | |
|---|---|
| CHOP: probe 21, primer 1: | gcgacagagccagaataaca |

(SEQ ID NO: _)

| | |
|---|---|
| CHOP: probe 21, primer 2: | gatgcacttccttctggaaca |

(SEQ ID NO: _)

| | |
|---|---|
| TBP: probe 107, primer 1: | ggcggtttggctaggttt |

(SEQ ID NO: _)

| | |
|---|---|
| TBP: probe 107, primer 2: | gggttatcttcacacaccatga |

(SEQ ID NO: _)

| | |
|---|---|
| Tubb5: probe 16, primer 1: | ctgagtaccagcagtaccaggat |

(SEQ ID NO: _)

| | |
|---|---|
| Tubb5: probe 16, primer 2: | ctctctgccttaggcctcct |

Probe and primers were designed using the ProbeFinder software (www.roche-applied-science.com/sis/rpcr/upl/index.jsp?id=uplct_{—}030000).

Anti-HF ELISA

Polyclonal anti-HF antibody was raised by immunizing rabbits with KLH coupled to an HF derivative (MAZ1356) containing a linker attached to the quinazolinone and terminated by a primary amino group. Crude antibody was affinity purified using MAZ1356 linked to NHS-agarose. For the ELISA assay, MAZ1356 was coupled to 96-well Reacti-bind Plates (Pierce). After binding, plates were blocked with 10% goat serum in PBS/0.2% Tween-20 (PBST). In preliminary experiments, a range of concentrations of MAZ1356 coupling and anti-HF antibody were tested to determine concentrations that yielded optimally sensitive and linear detection of HF in the 10-100 nM range. To establish a standard curve for HF concentration, MAZ1356-bound plates were incubated with affinity purified anti-HF antibody in 10% goat serum/PBST for 2 hours at room temperature in the presence of known concentrations of HF, then washed 5 times with PBST and incubated with Goat anti-rabbit HRP in 10% goat serum/PBST. Captured antibody was quantitated using TMB based colorimetric detection (Pierce), and a standard curve for HF concentration fitted from the colorimetric data. To assay HF concentration in cells, mouse embryo fibroblasts were incubated with indicated concentrations of HF for 2 hours, and lysed in 200 μl 1% NP40 buffer. Bulk protein was precipitated by incubation with 0.1 M acetic acid and centrifugation for 10′. Cleared lysates were neutralized with Tris pH 7.5. To determine HF concentration in cells, 8 μl of cleared lysate (or control buffer) was incubated with anti-HF antibody in MAZ1356 bound wells, and captured antibody detected and quantitated as described above. HF concentration was then determined by fitting colorimetry results with cell samples to a standard curve of HF concentration. Data in FIG. 24C are plotted as “arbitrary units” because the precise intracellular volume of the cells lysed is not known, and therefore the absolute concentration of HF in cells can only be estimated. Estimating the total packed cell volume of 5×10⁵ mouse embryo fibroblasts as 4 μl (Pierce Nebr.—PER kit) yields an absolute value of ˜800 nM HF inside cells that have been incubated with 20 nM HF. These data suggest that there is substantial concentration of HF from the medium.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

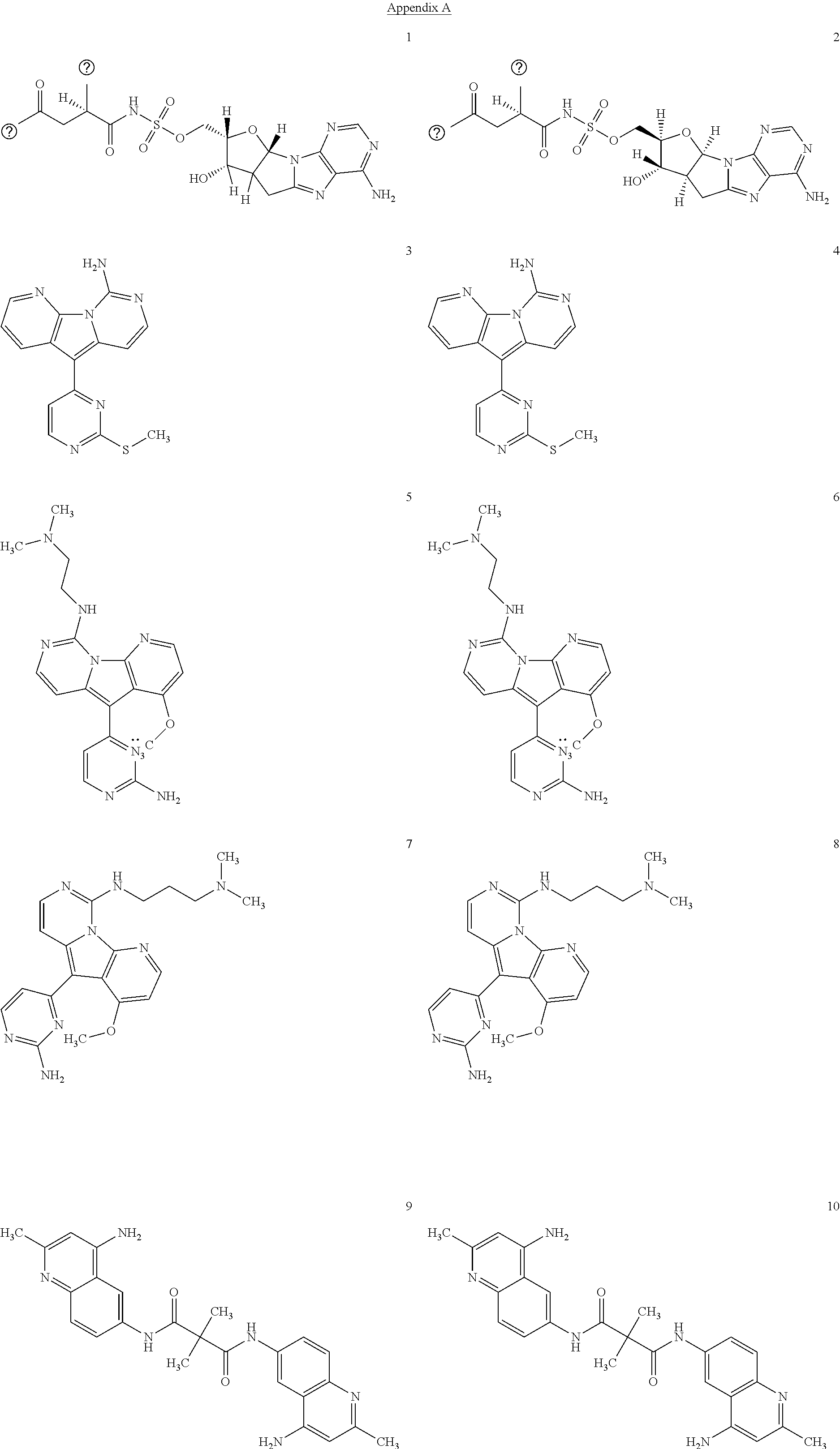

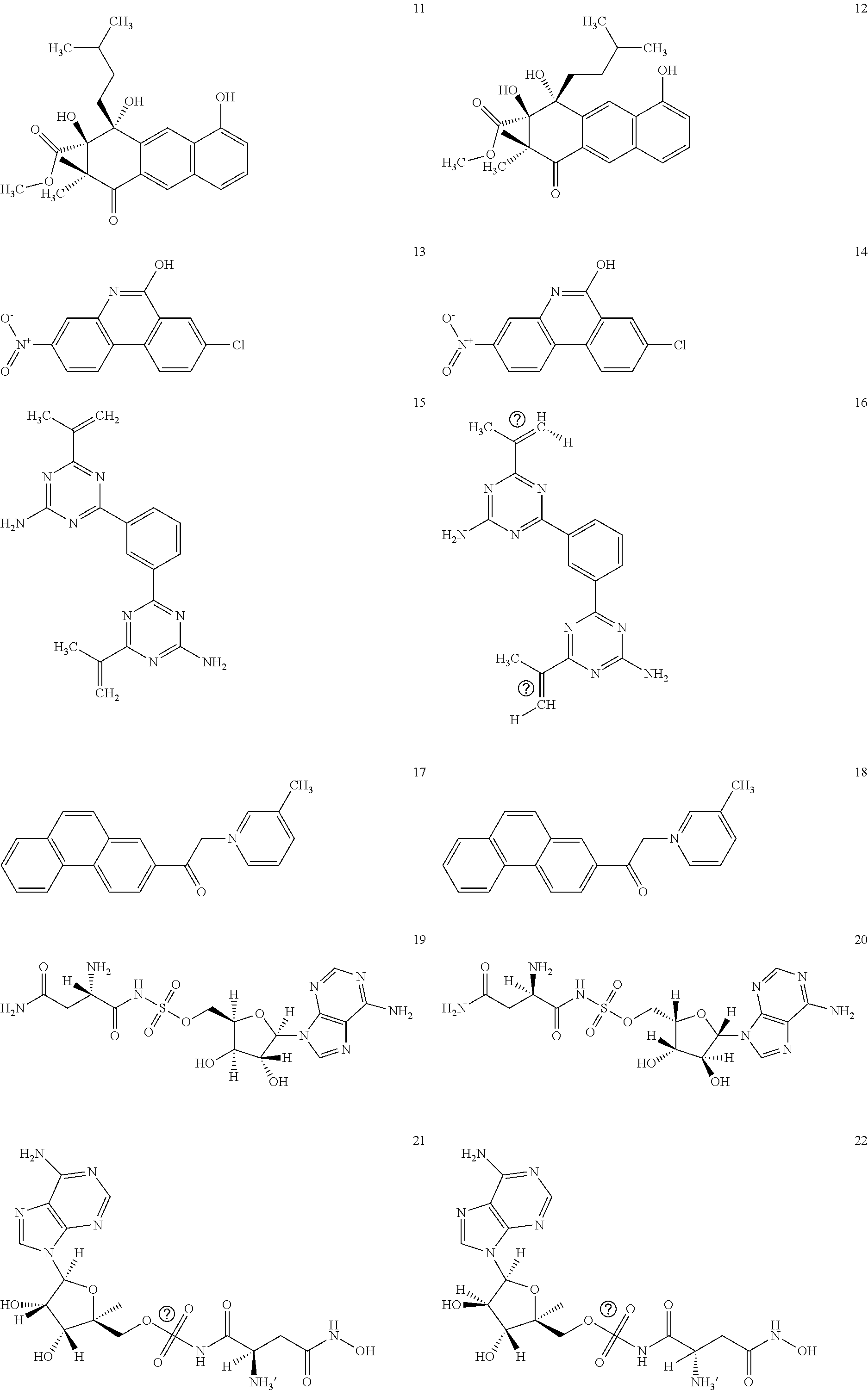

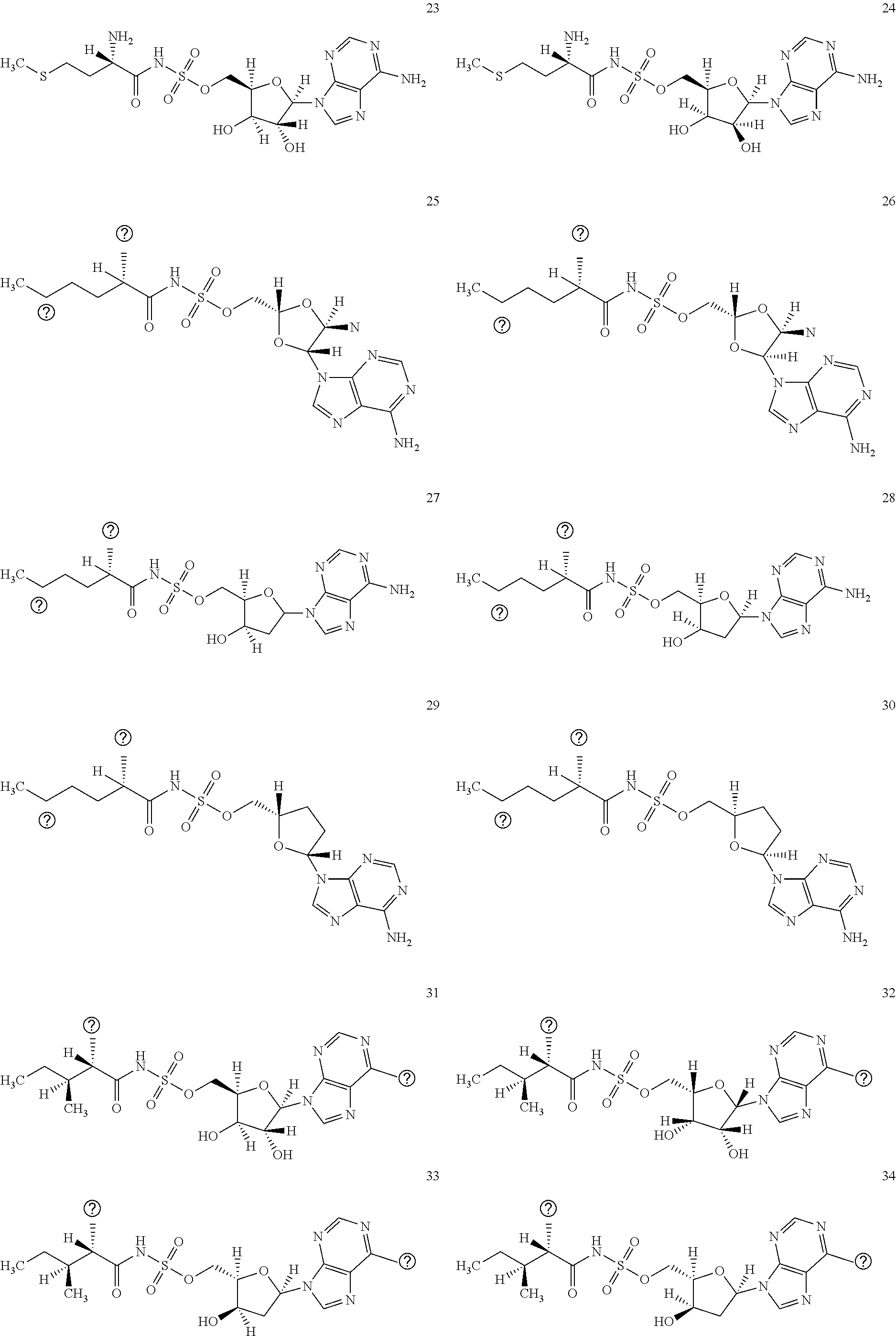

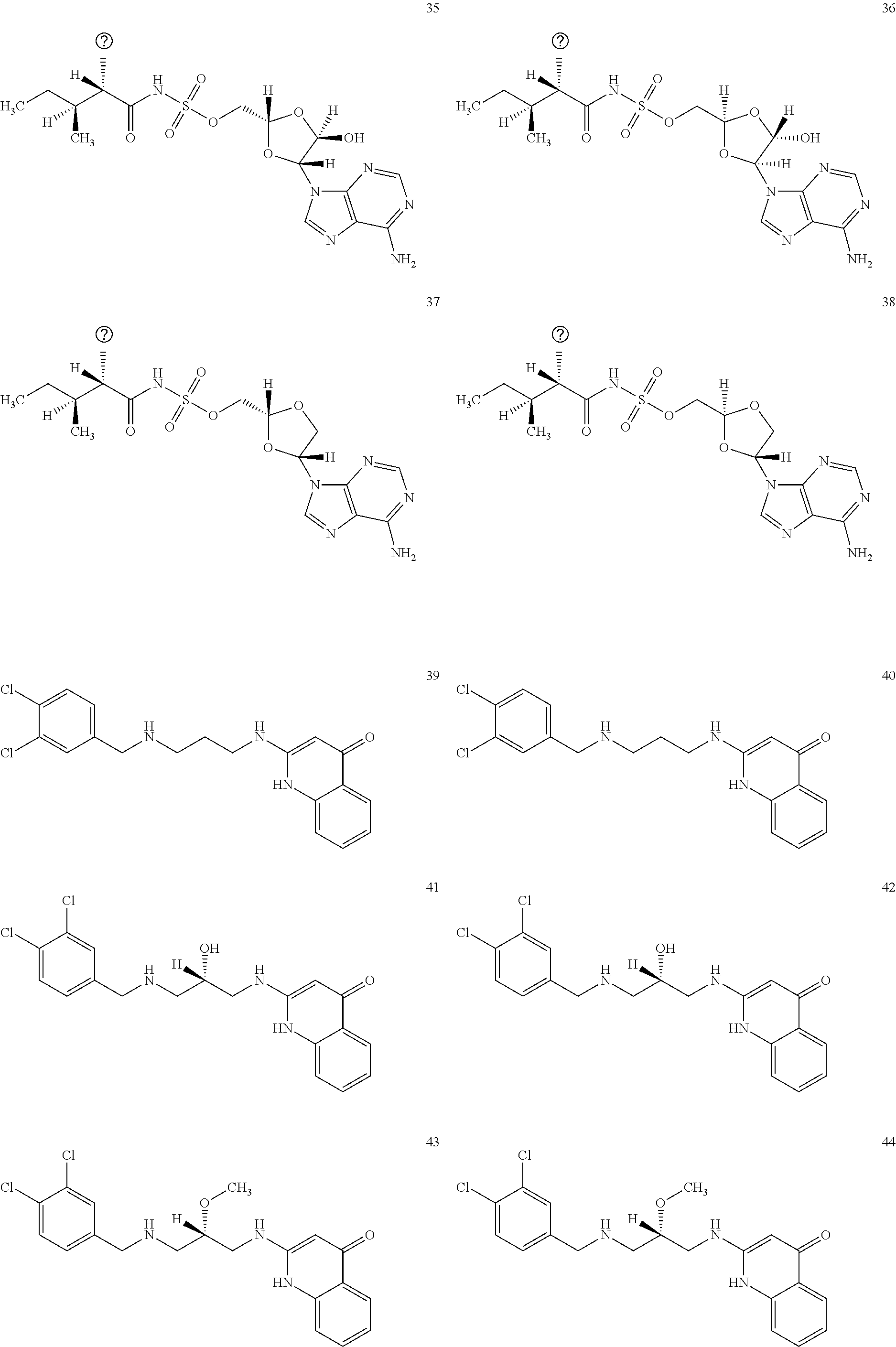

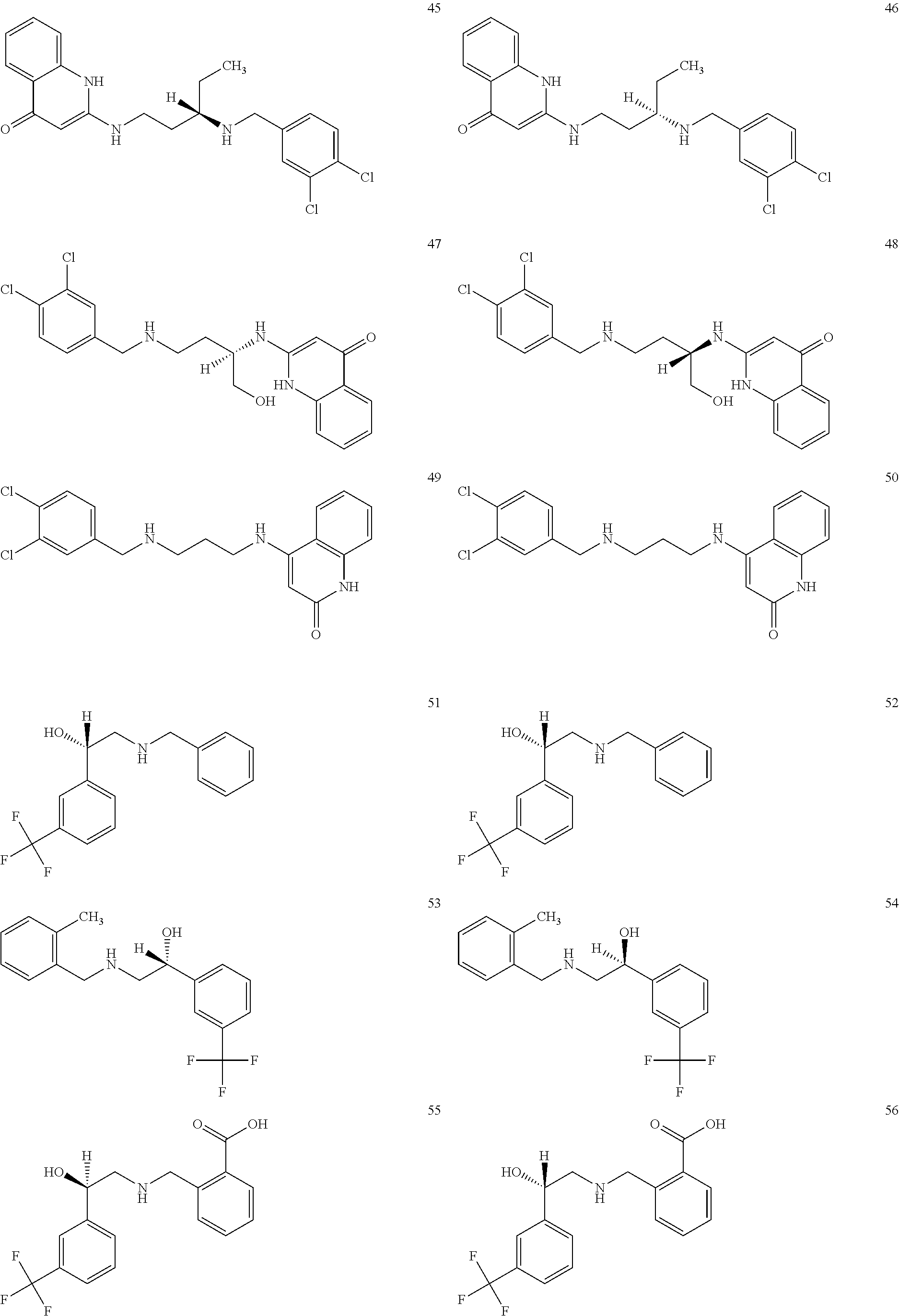

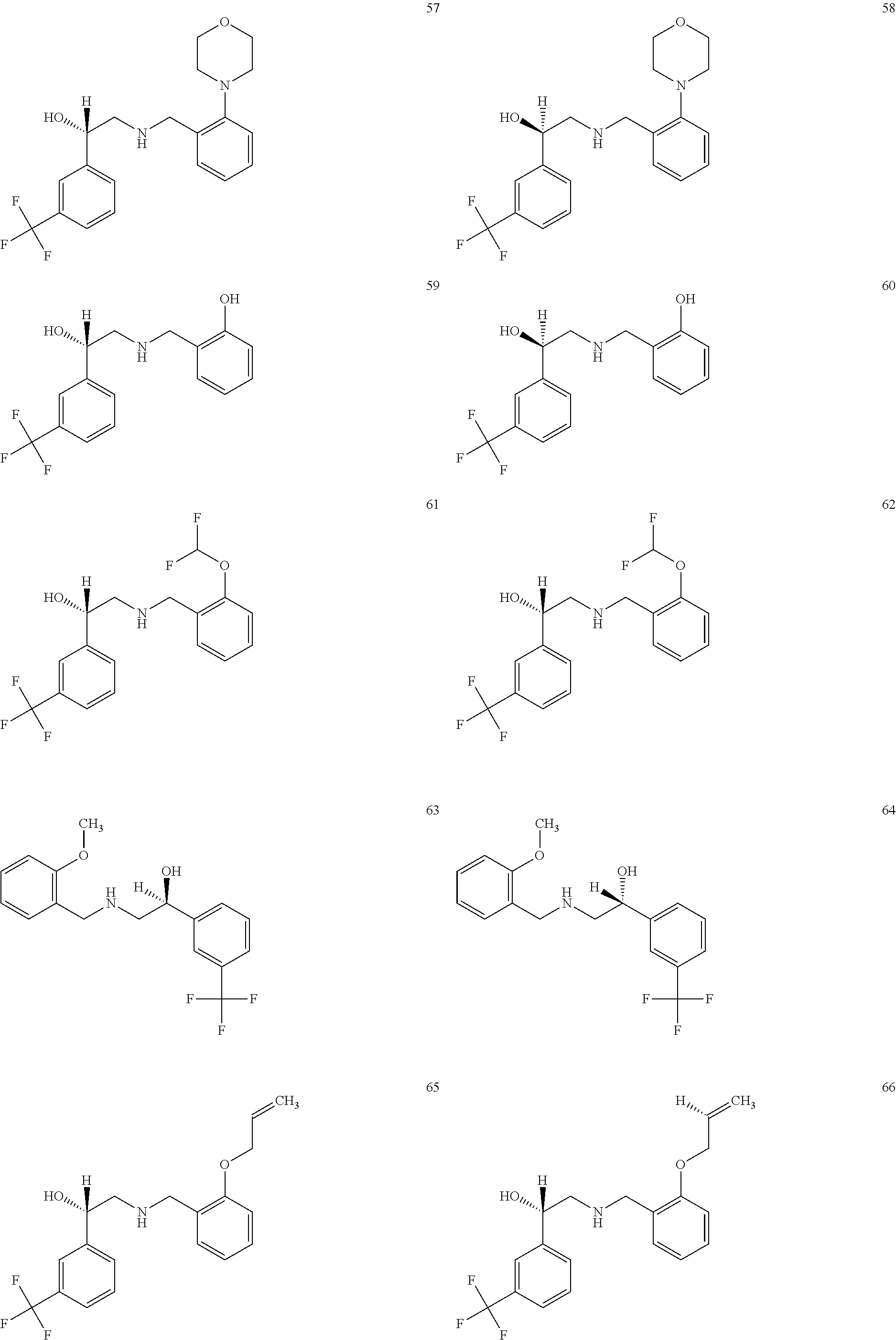

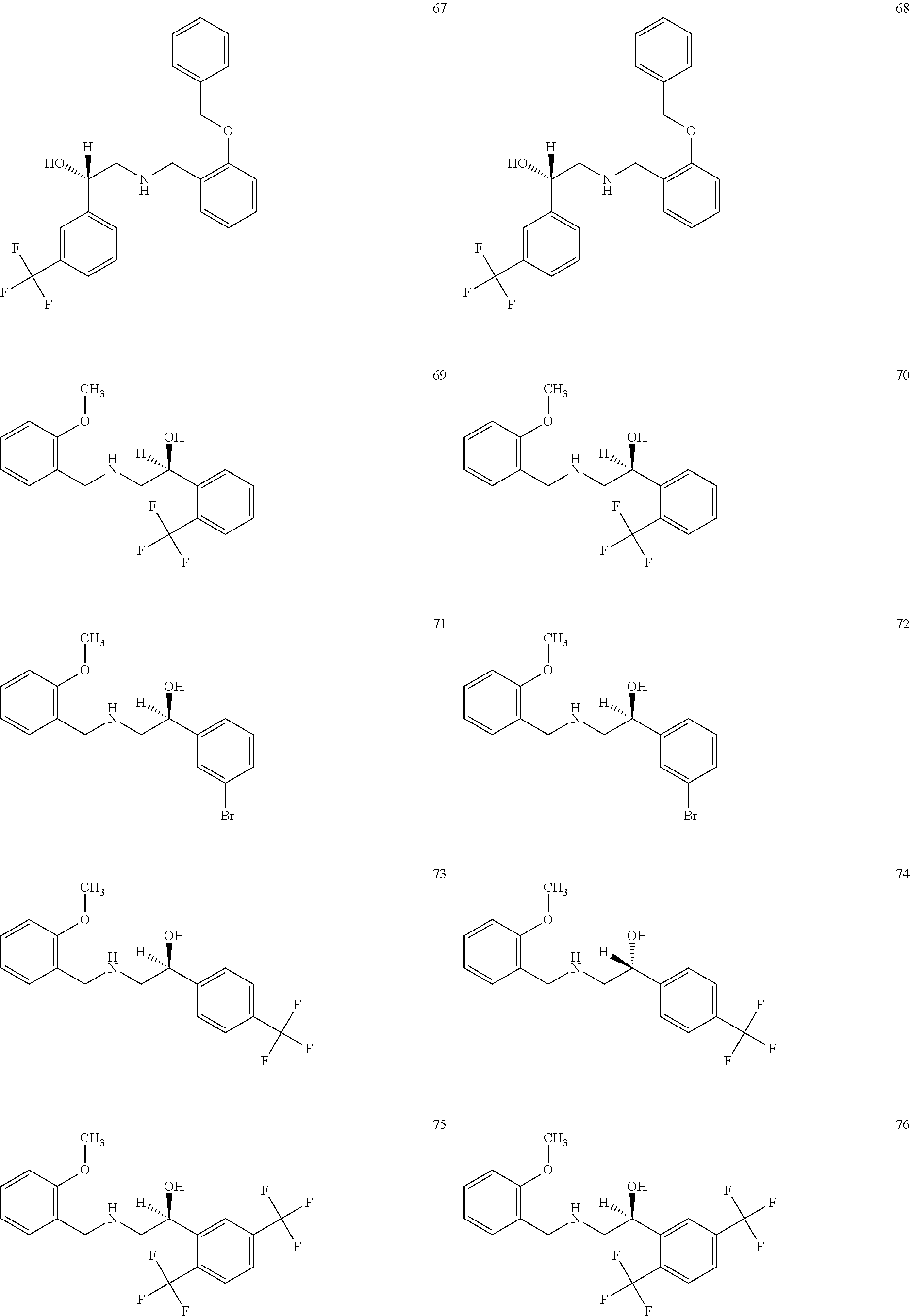

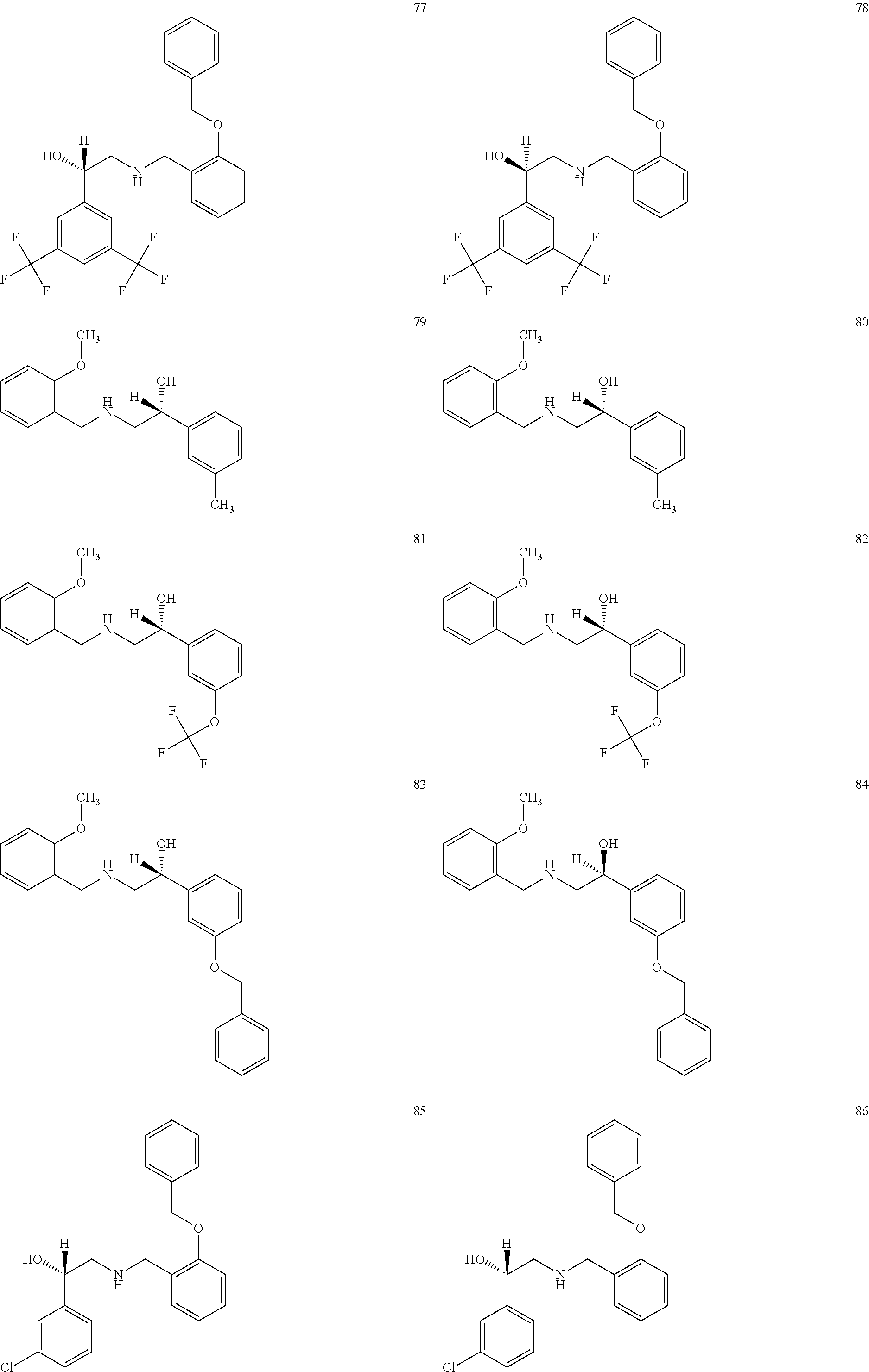

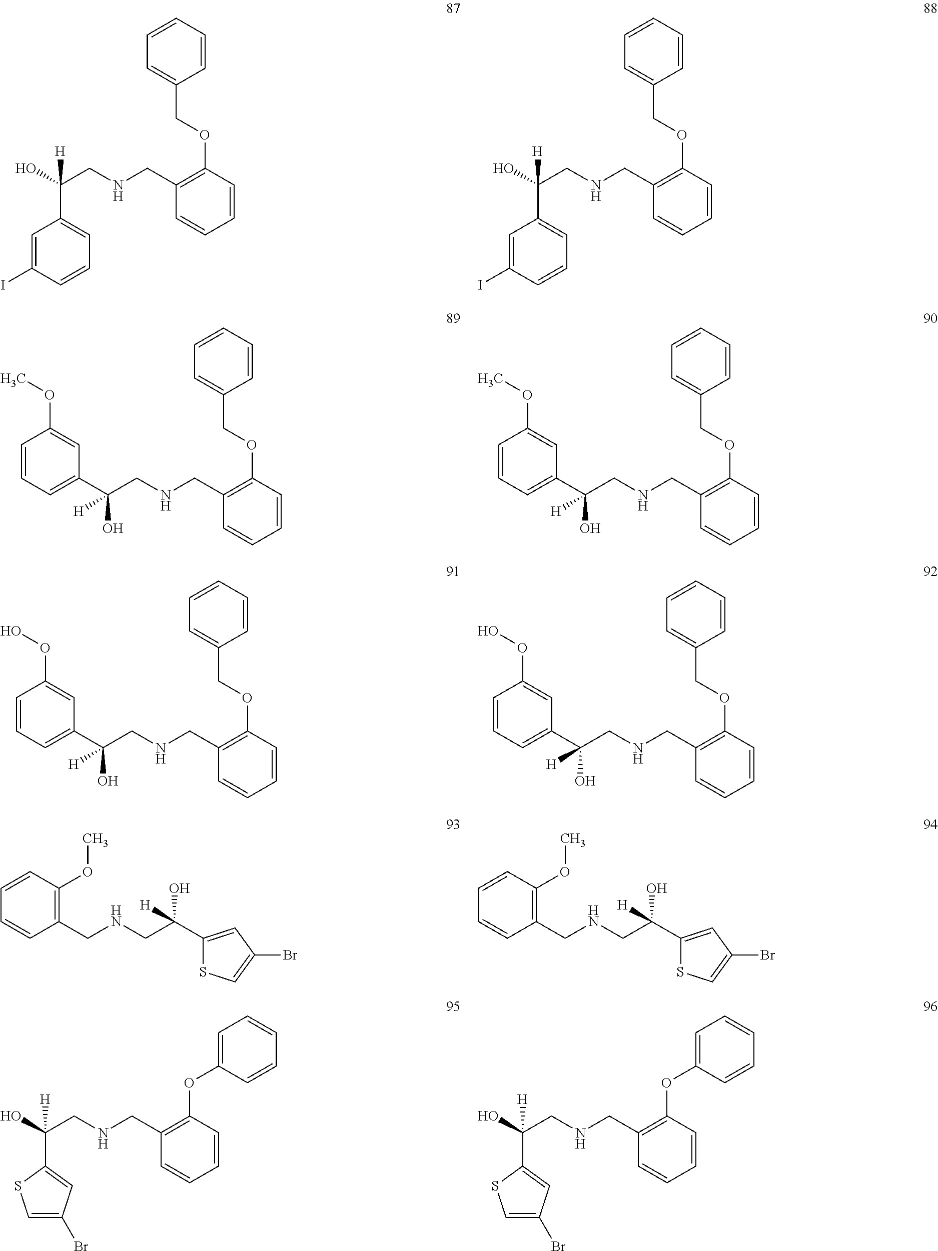

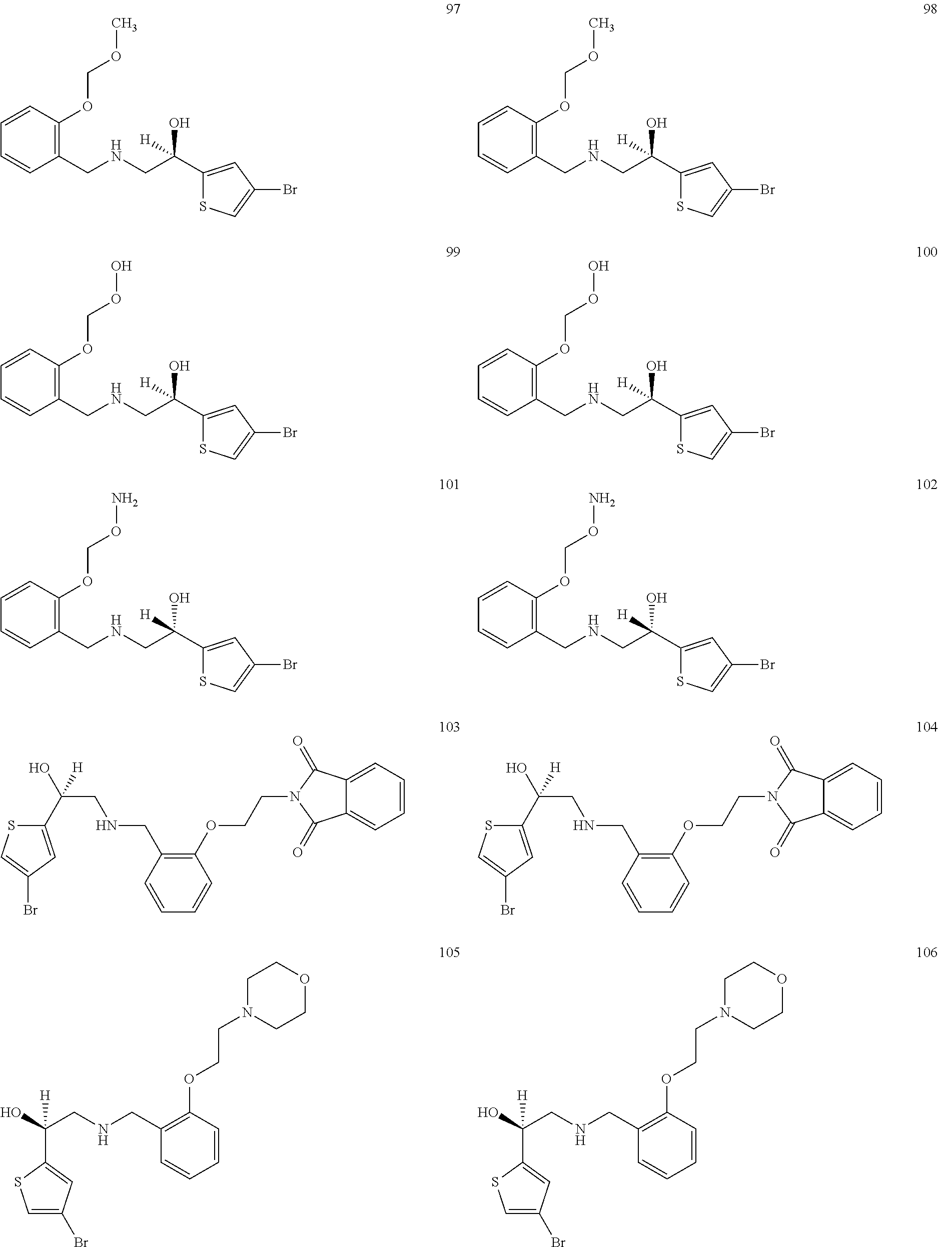

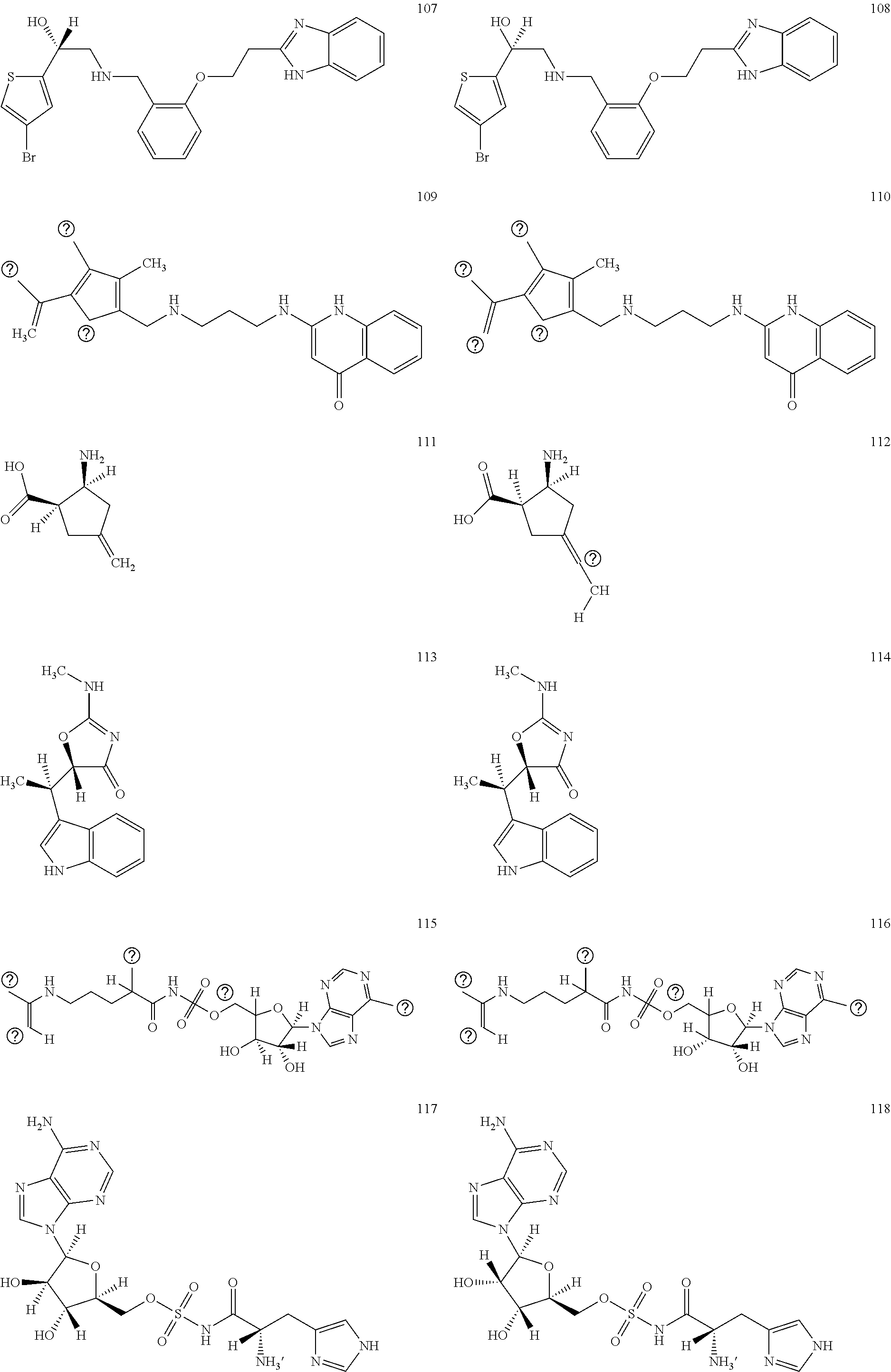

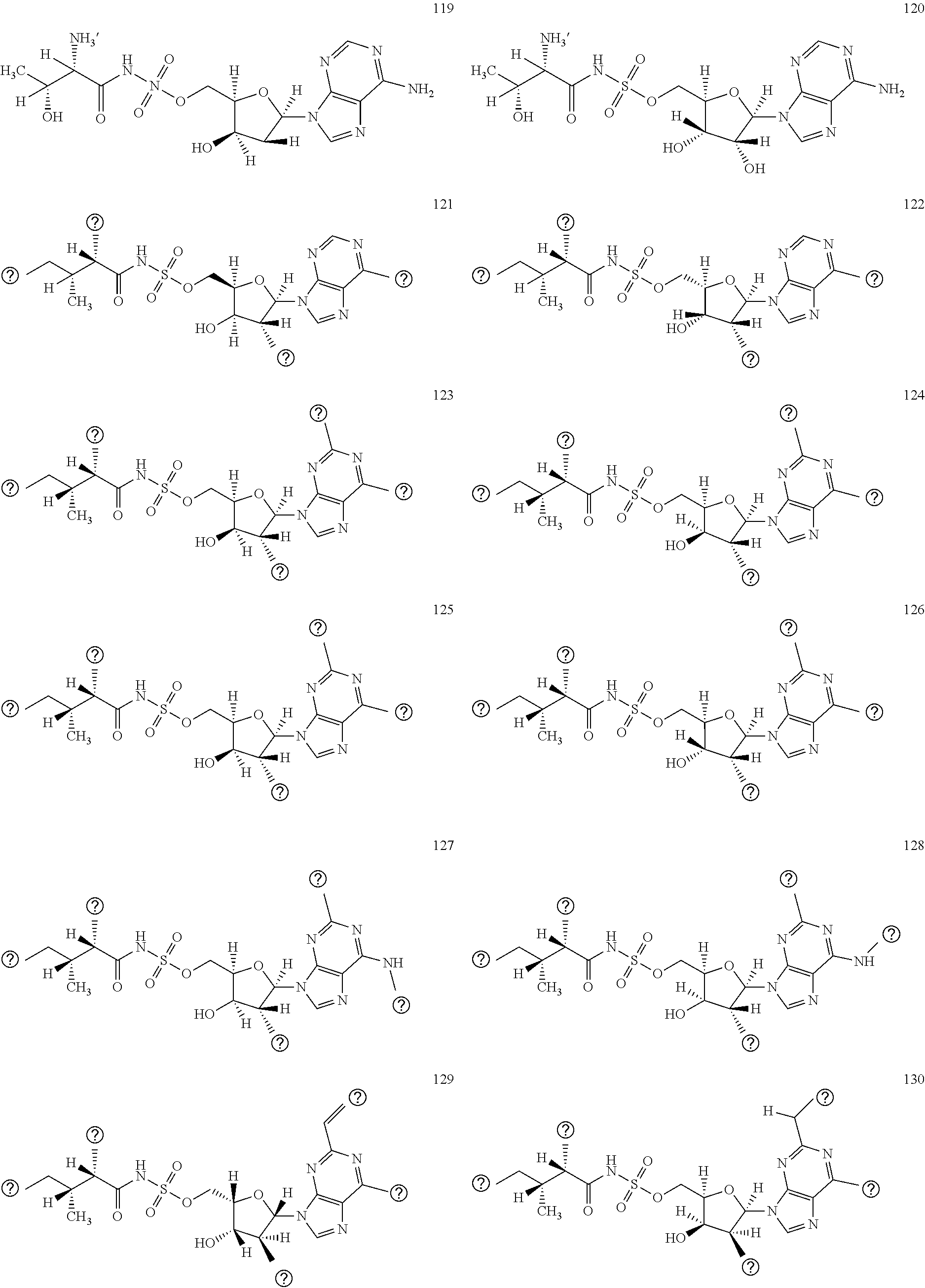

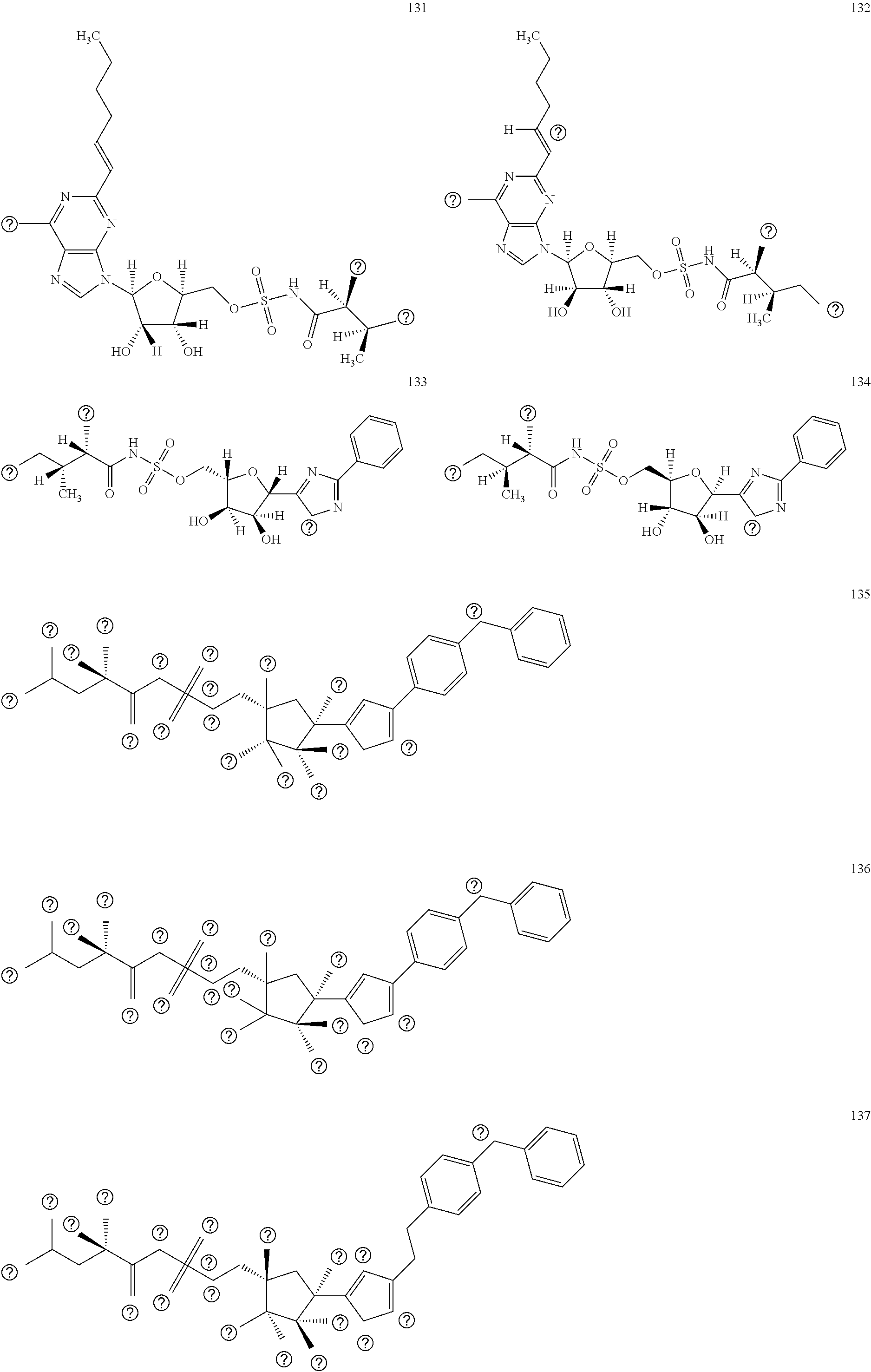

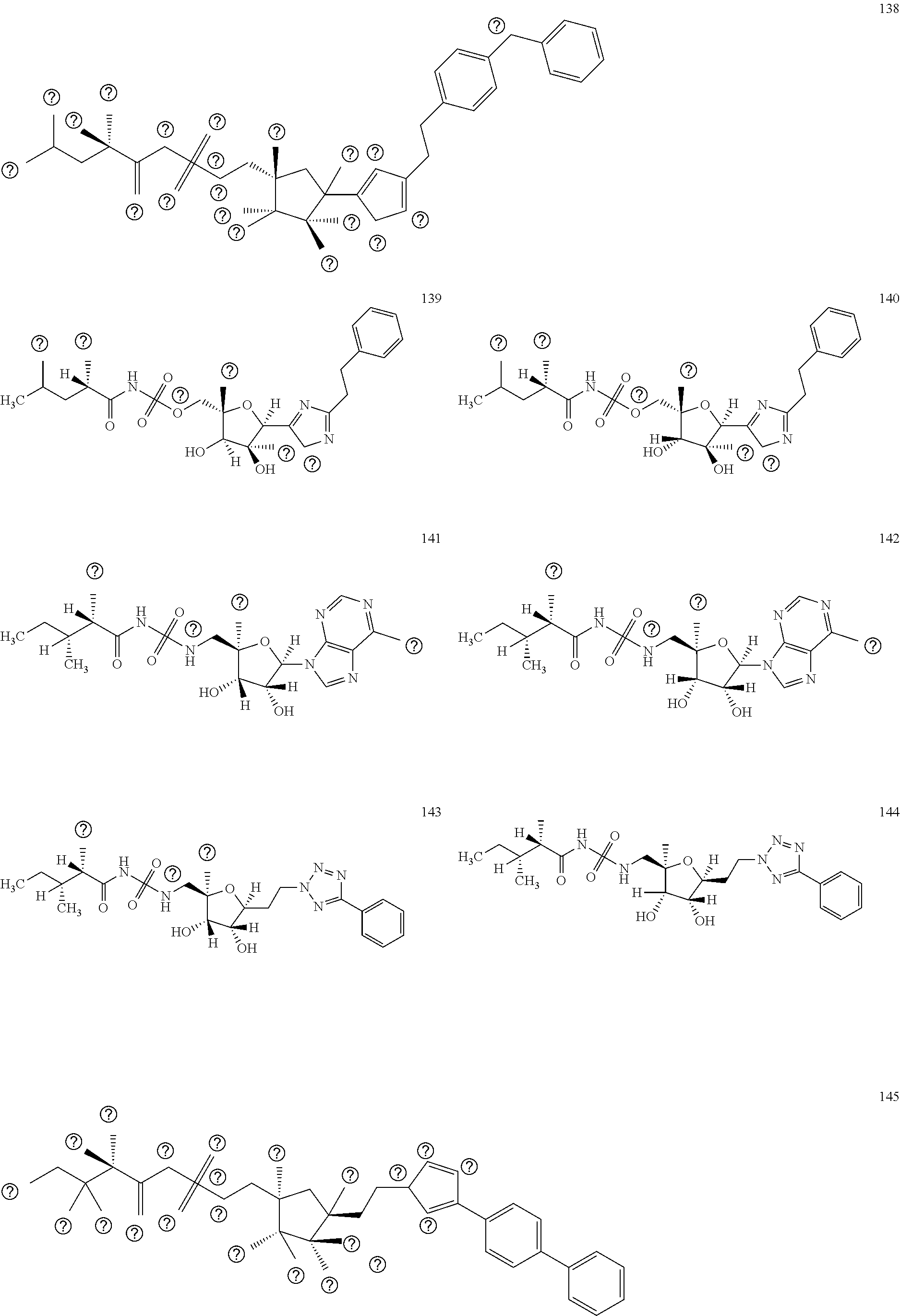

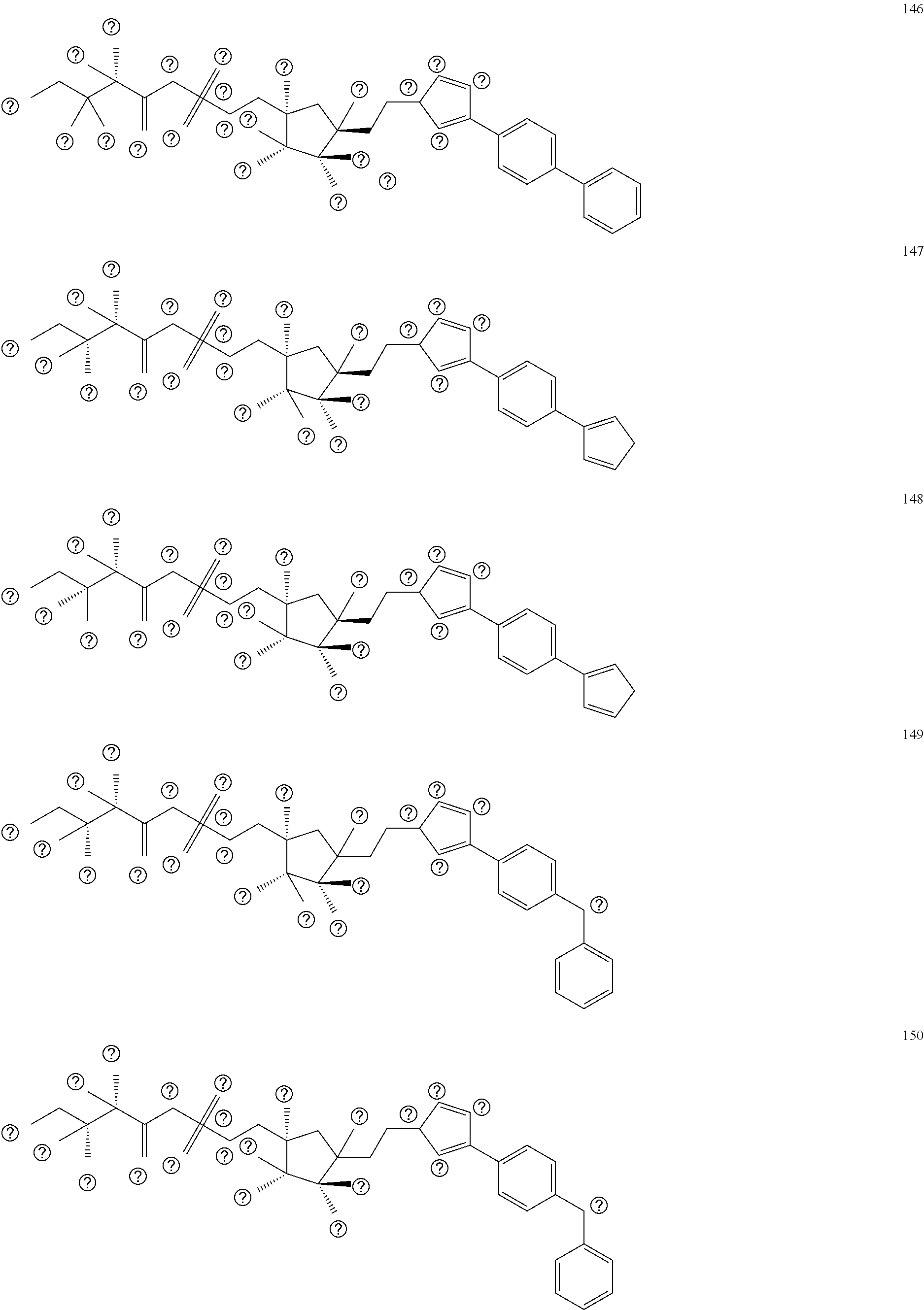

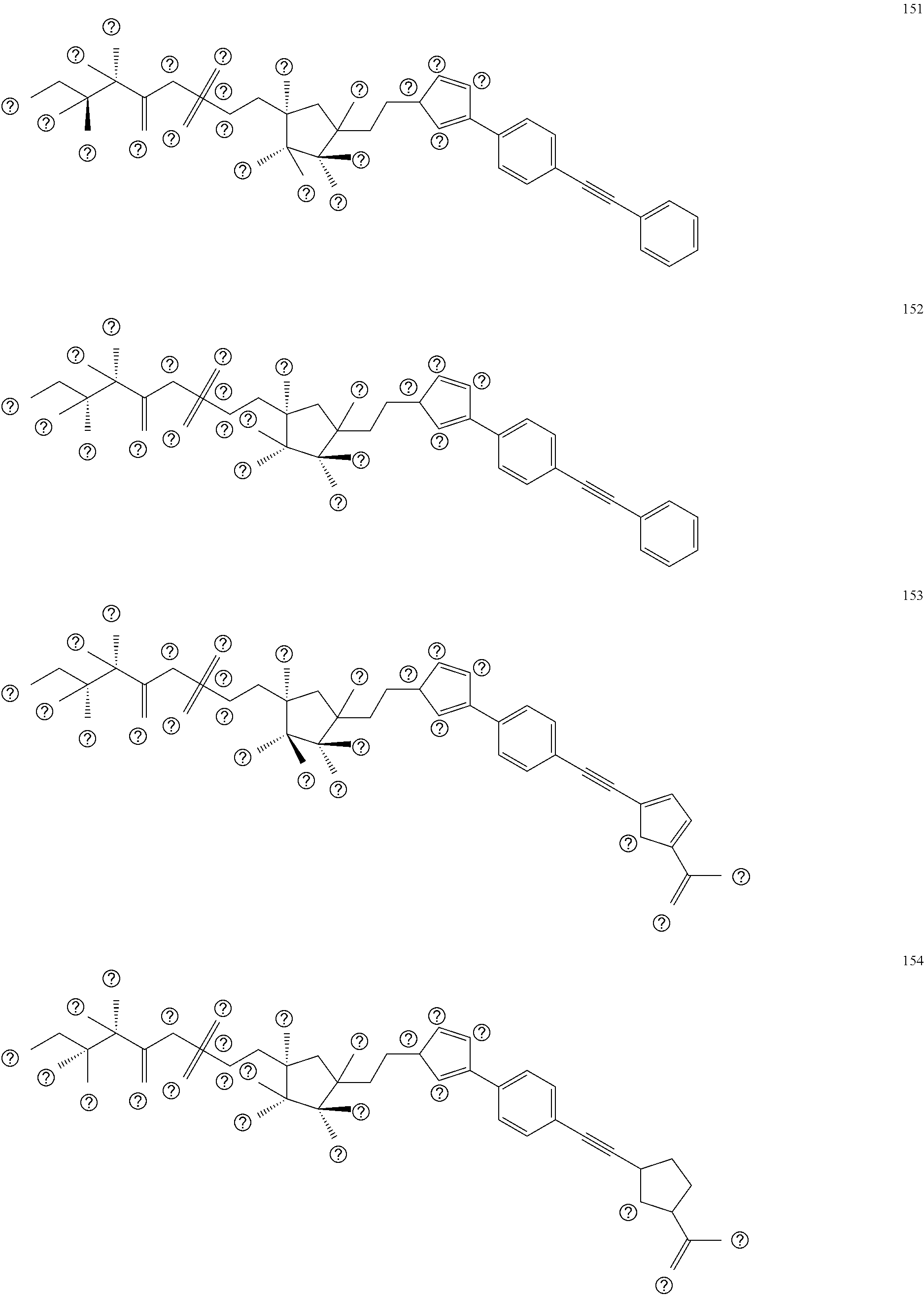

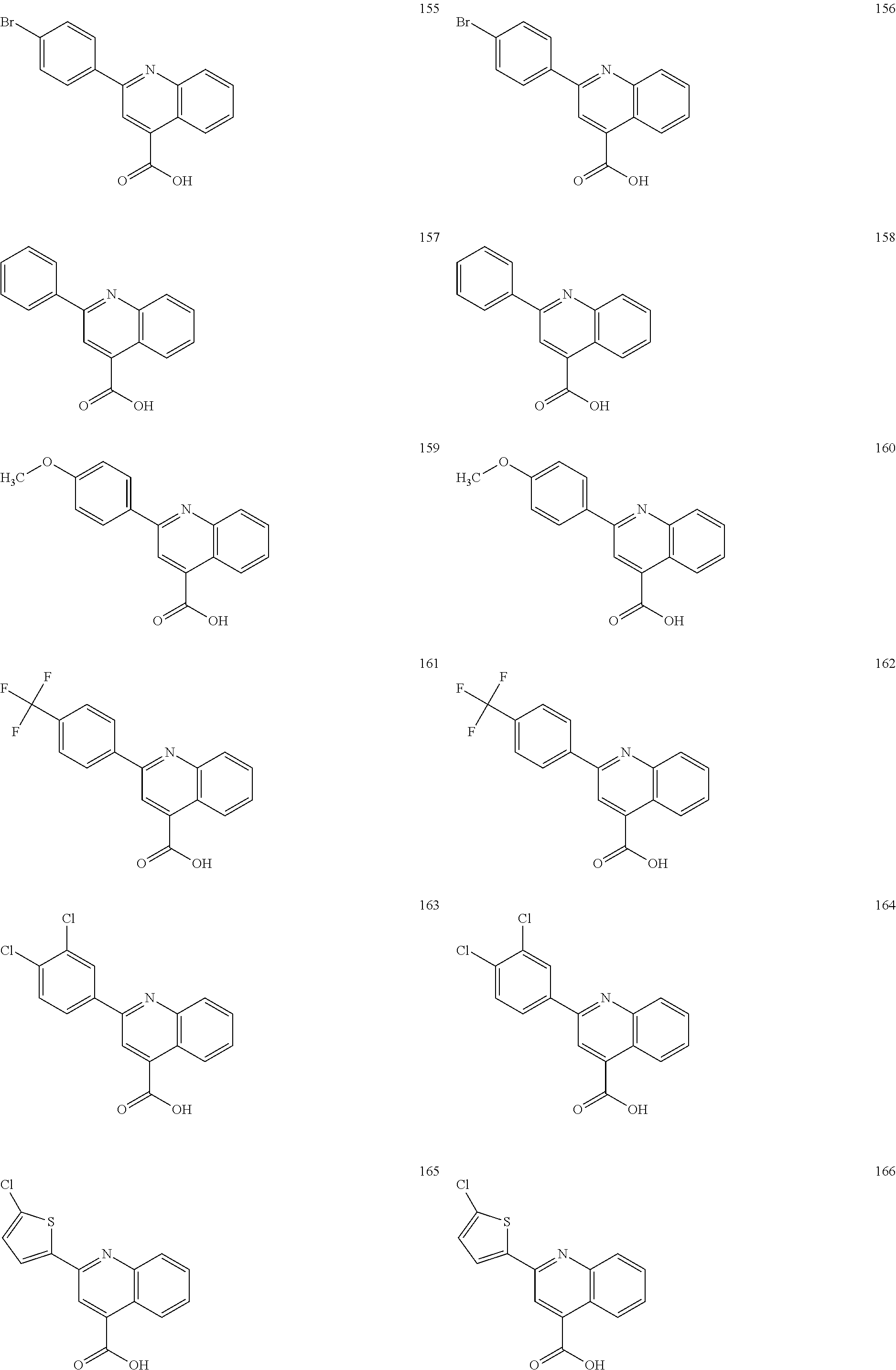

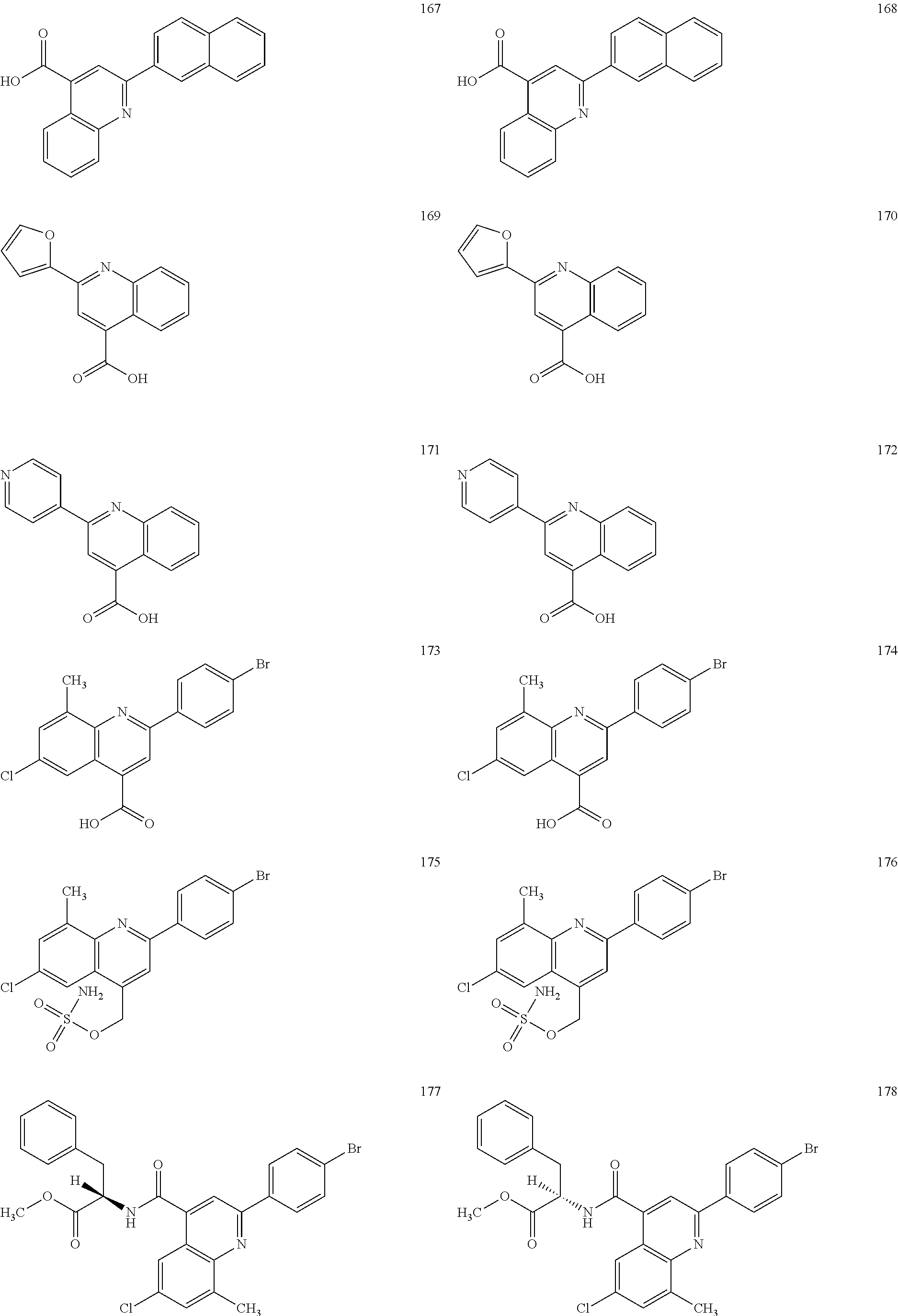

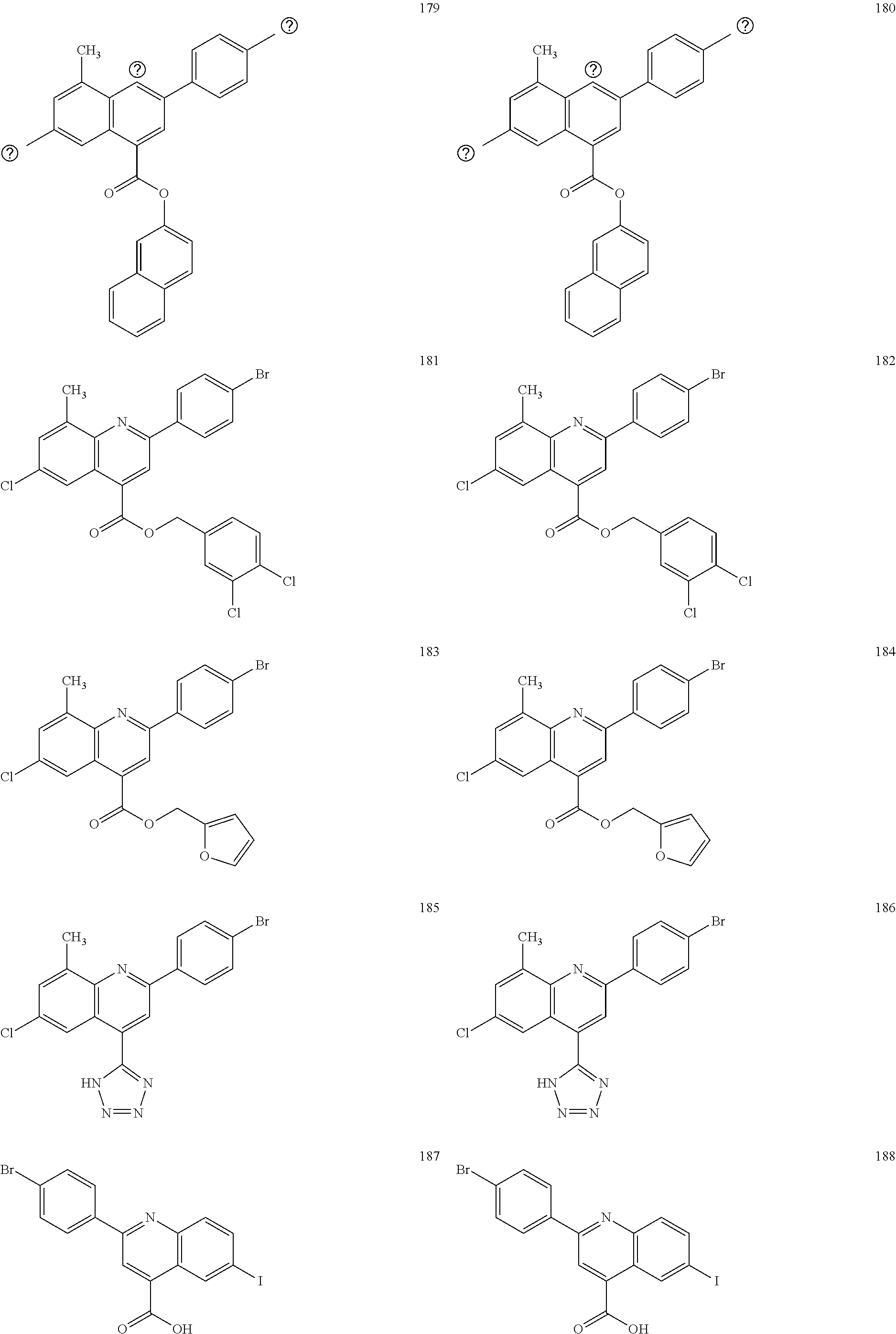

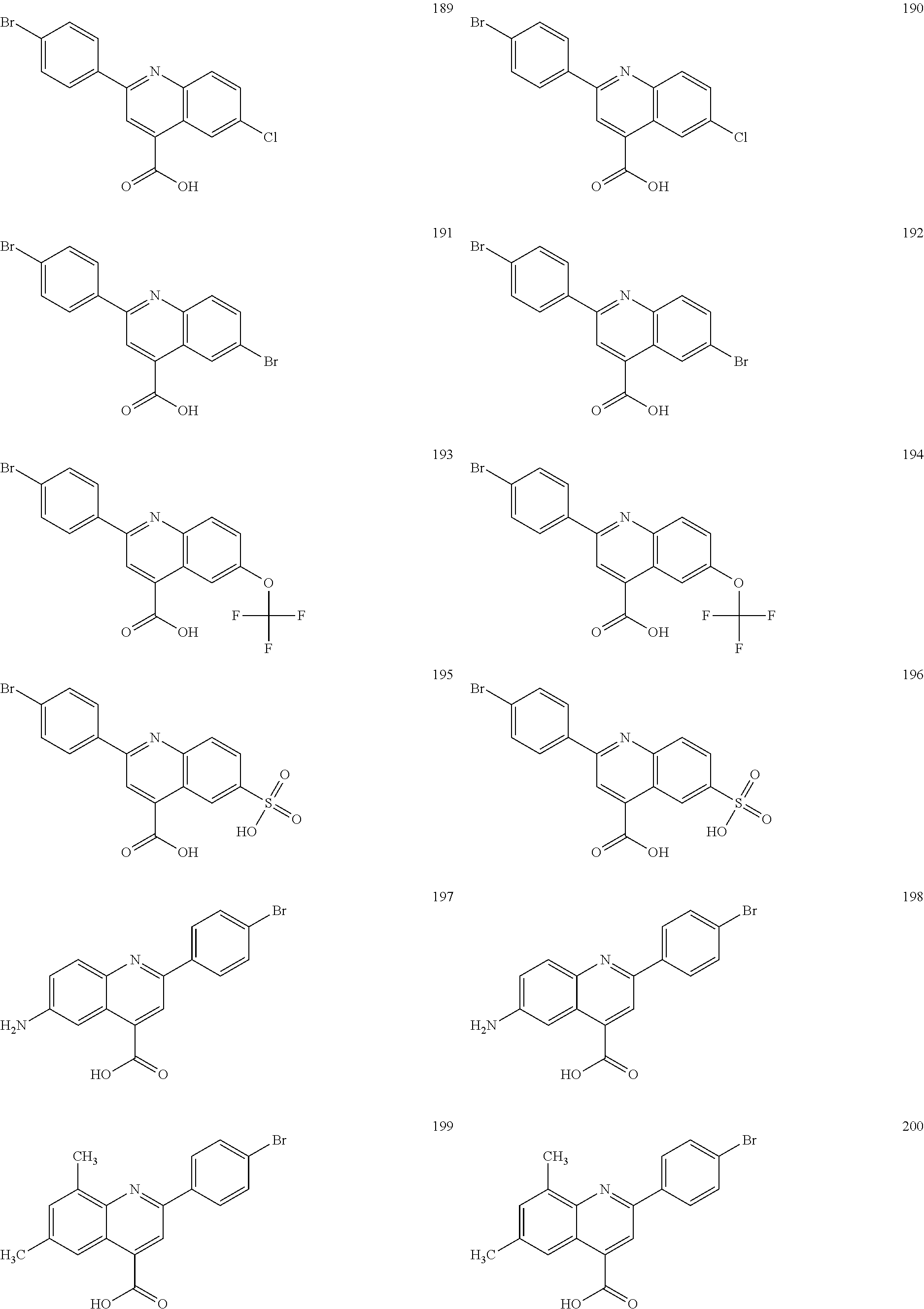

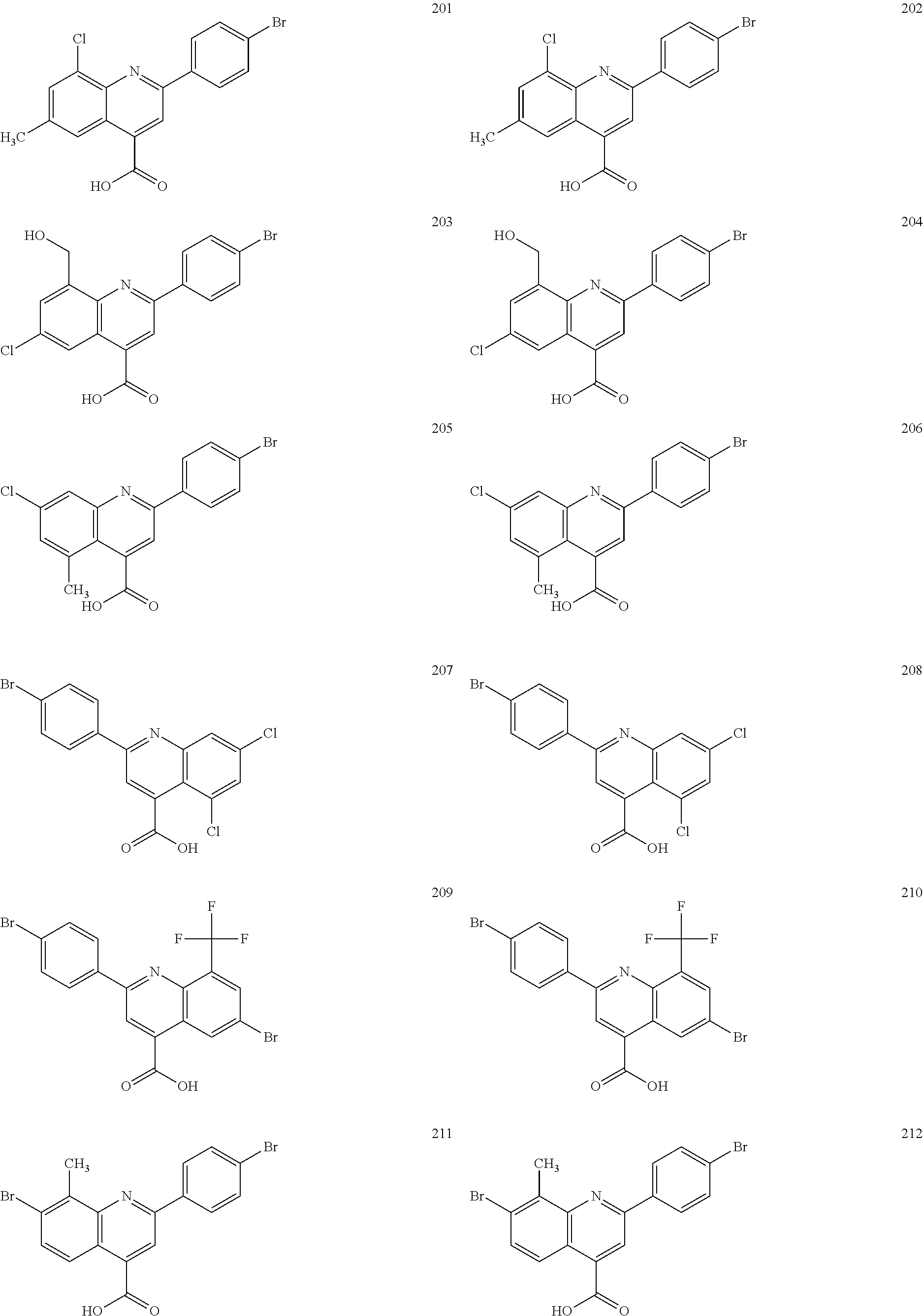

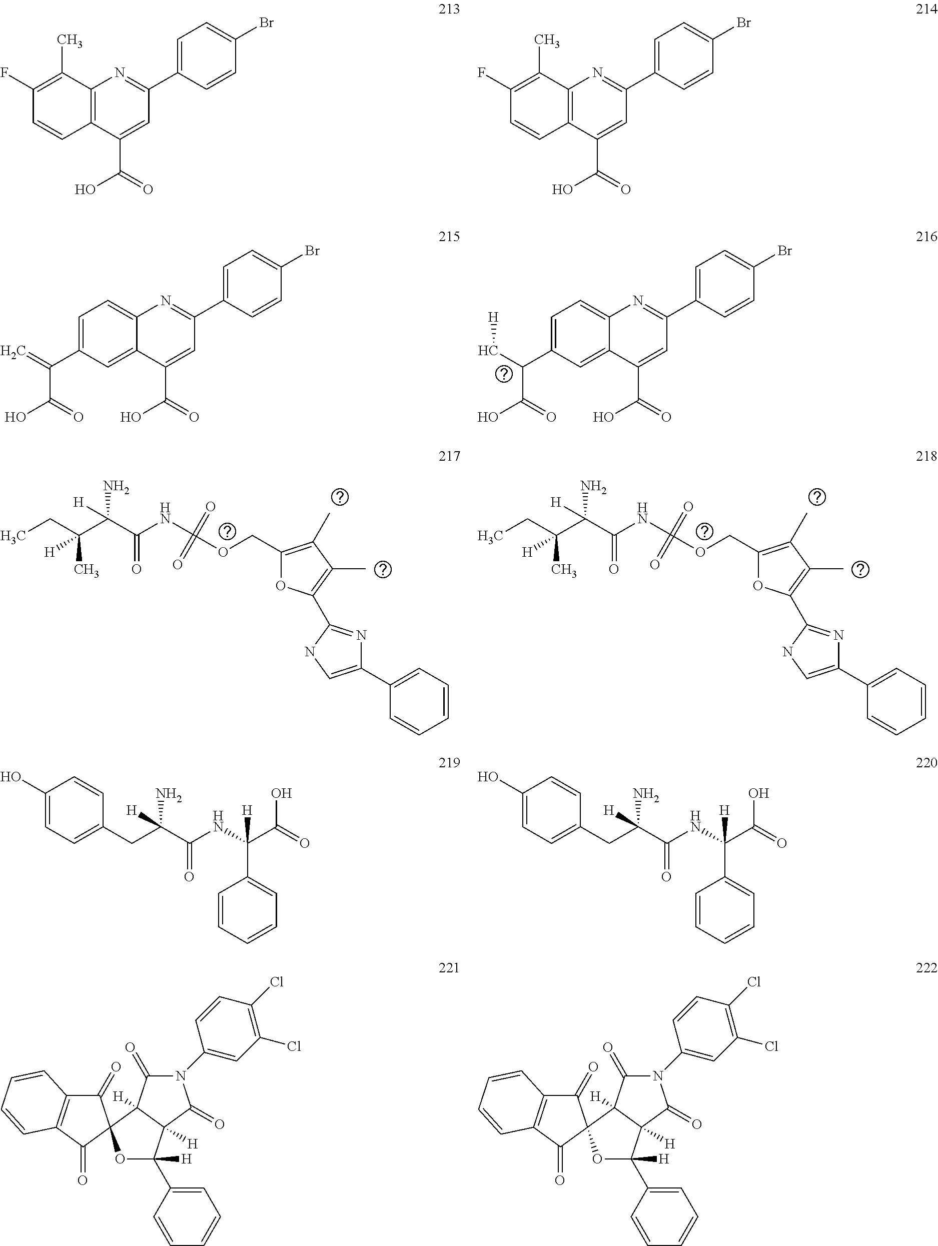

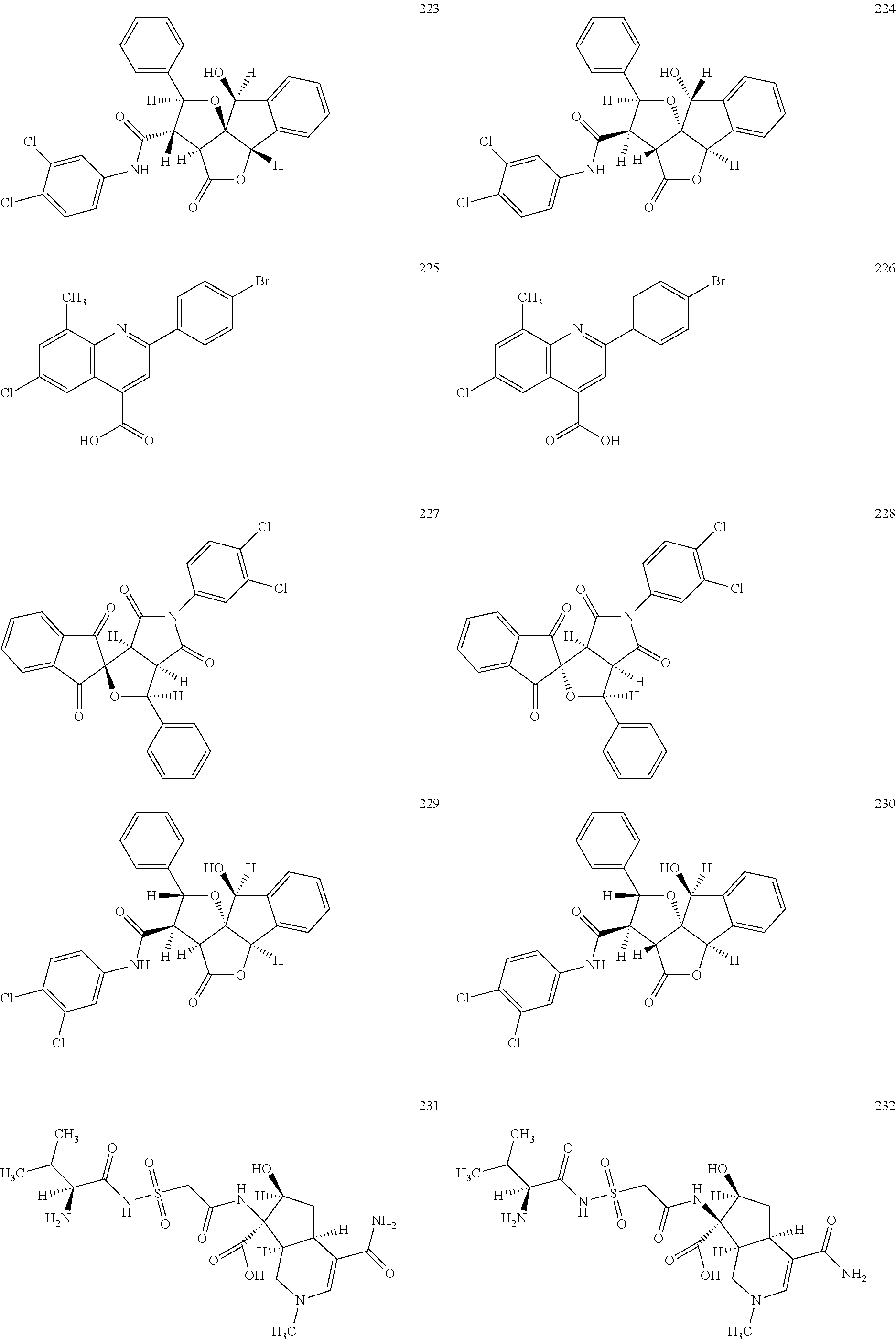

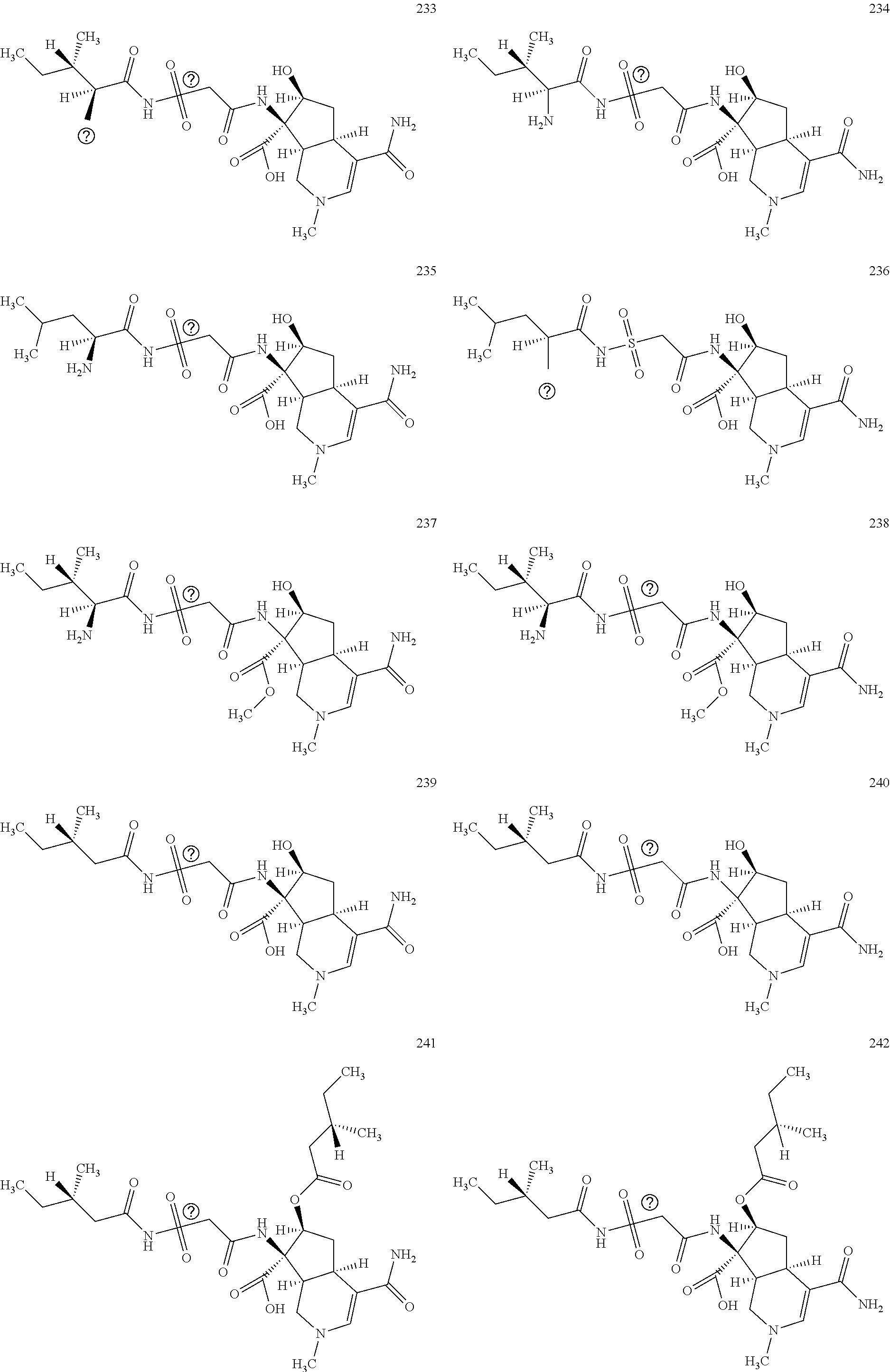

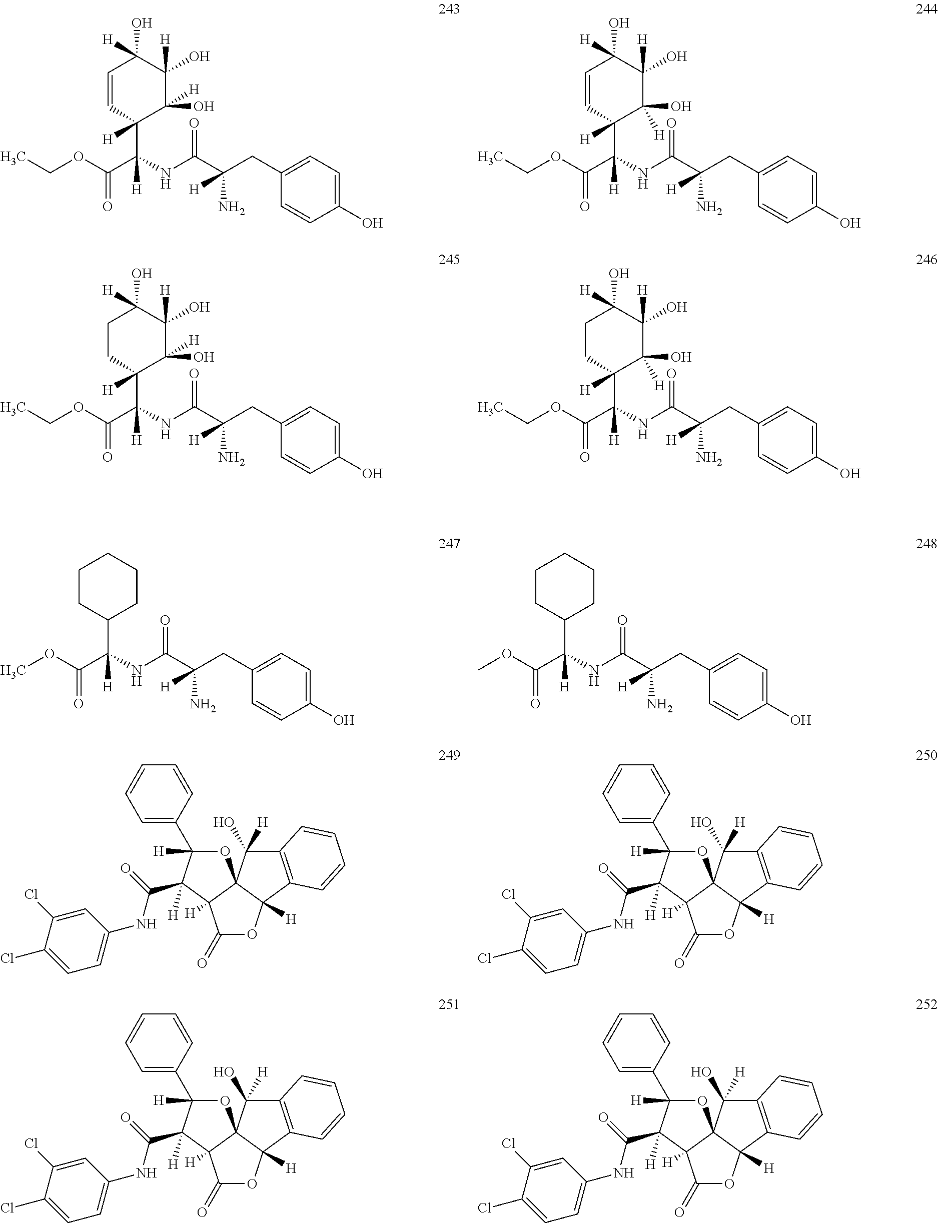

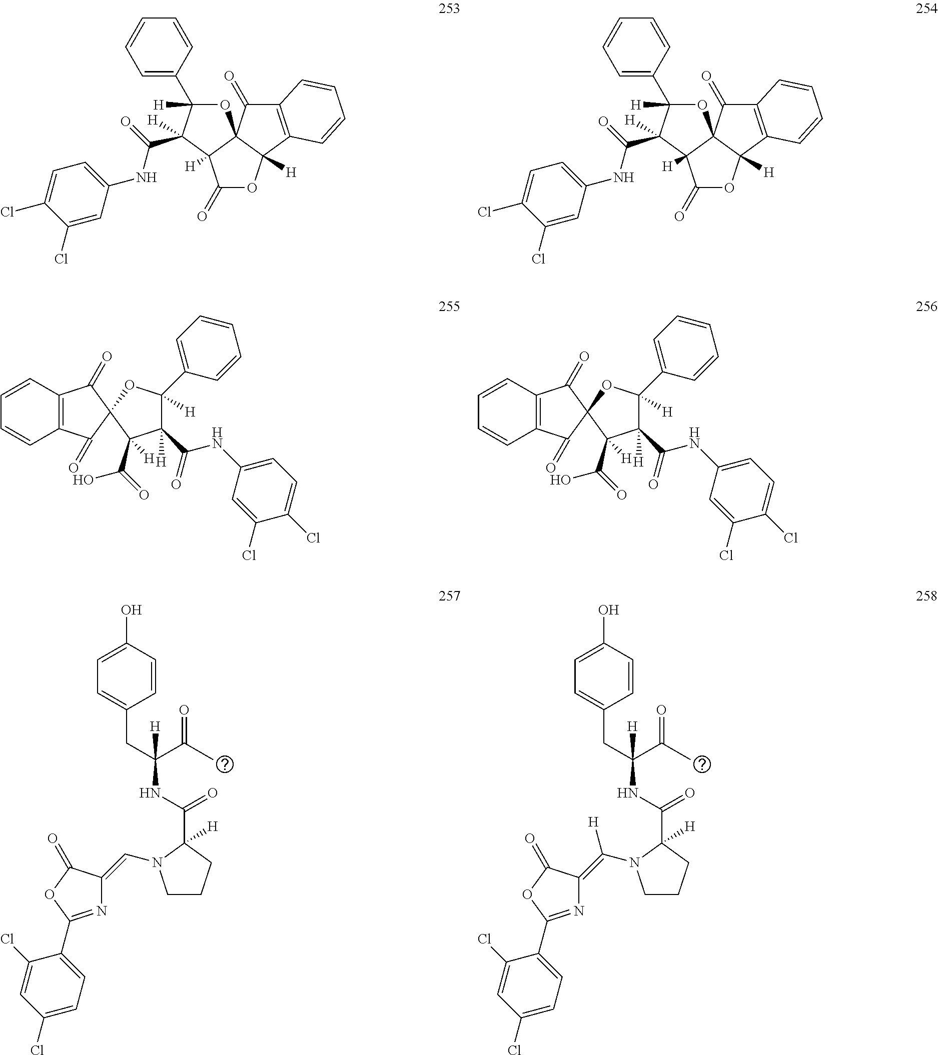

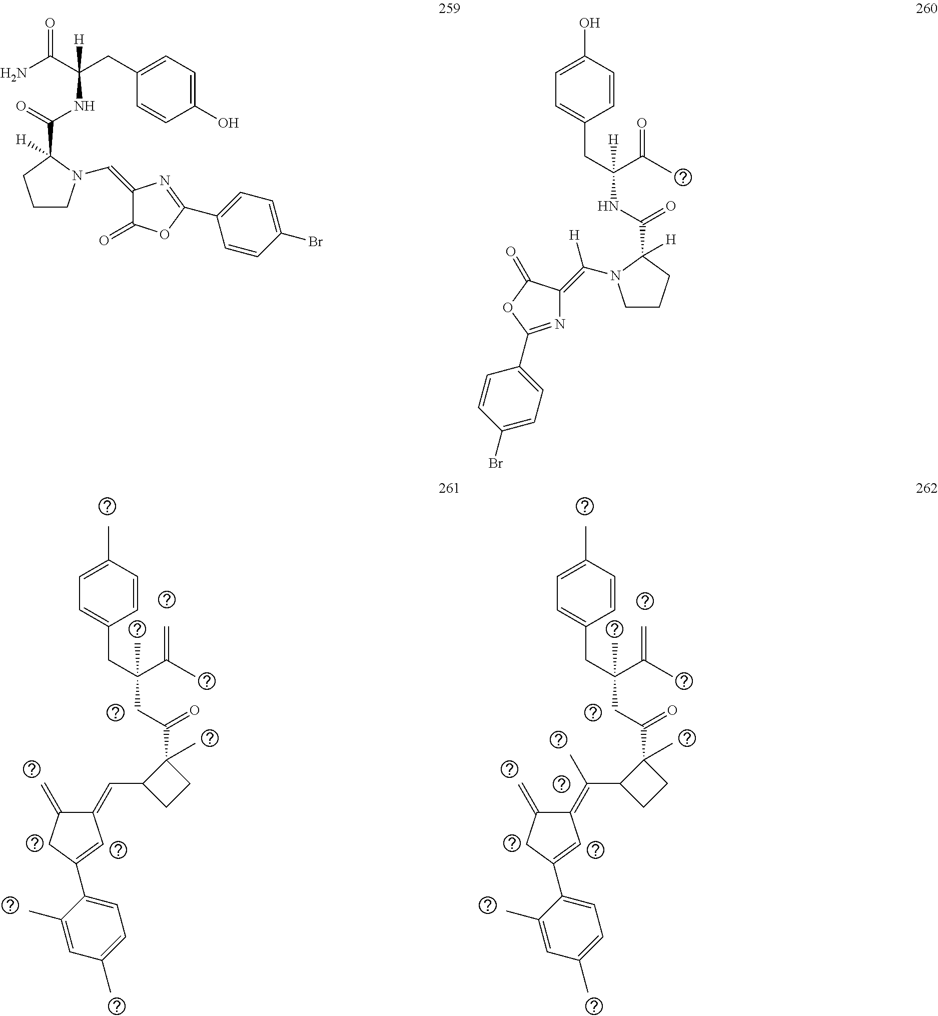

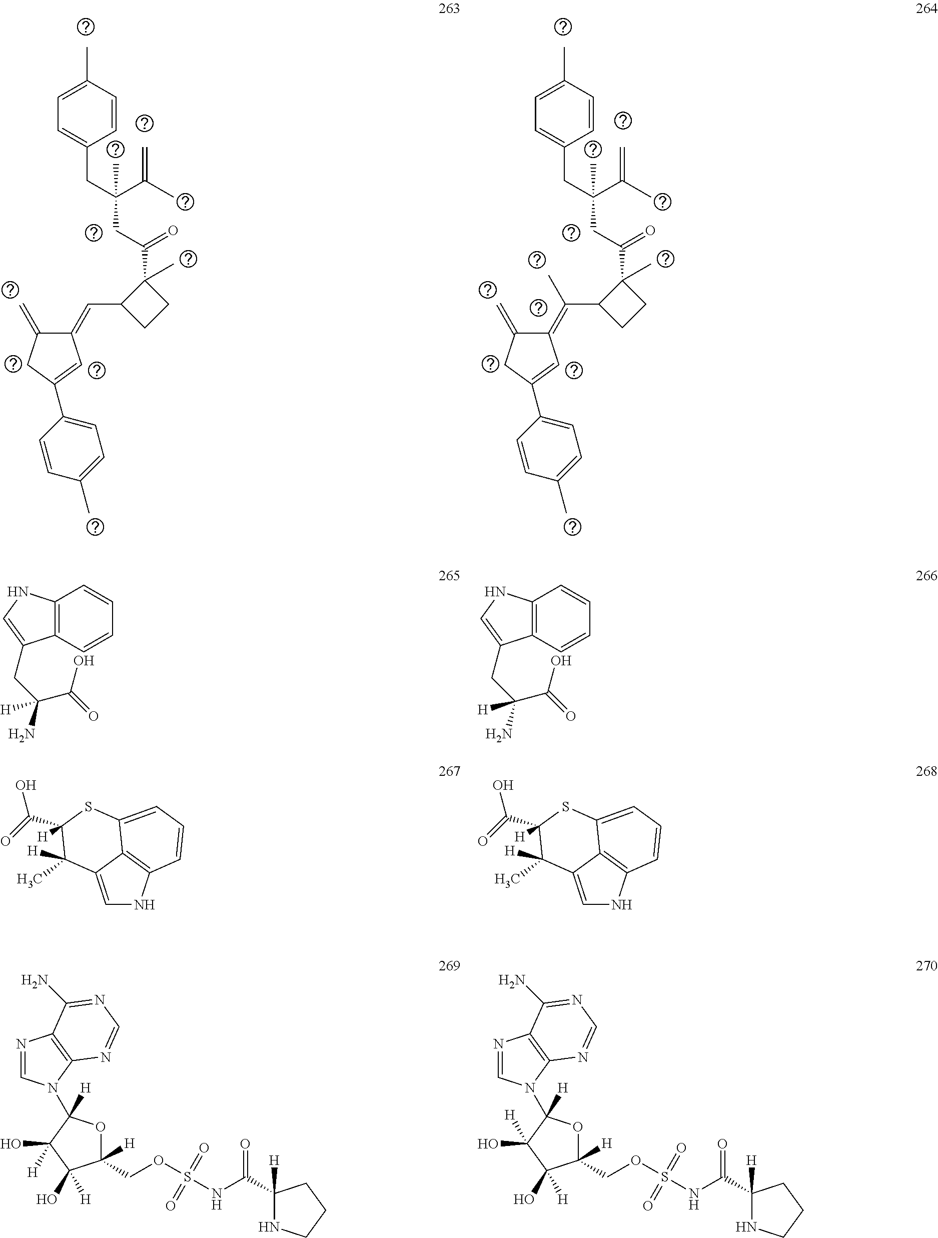

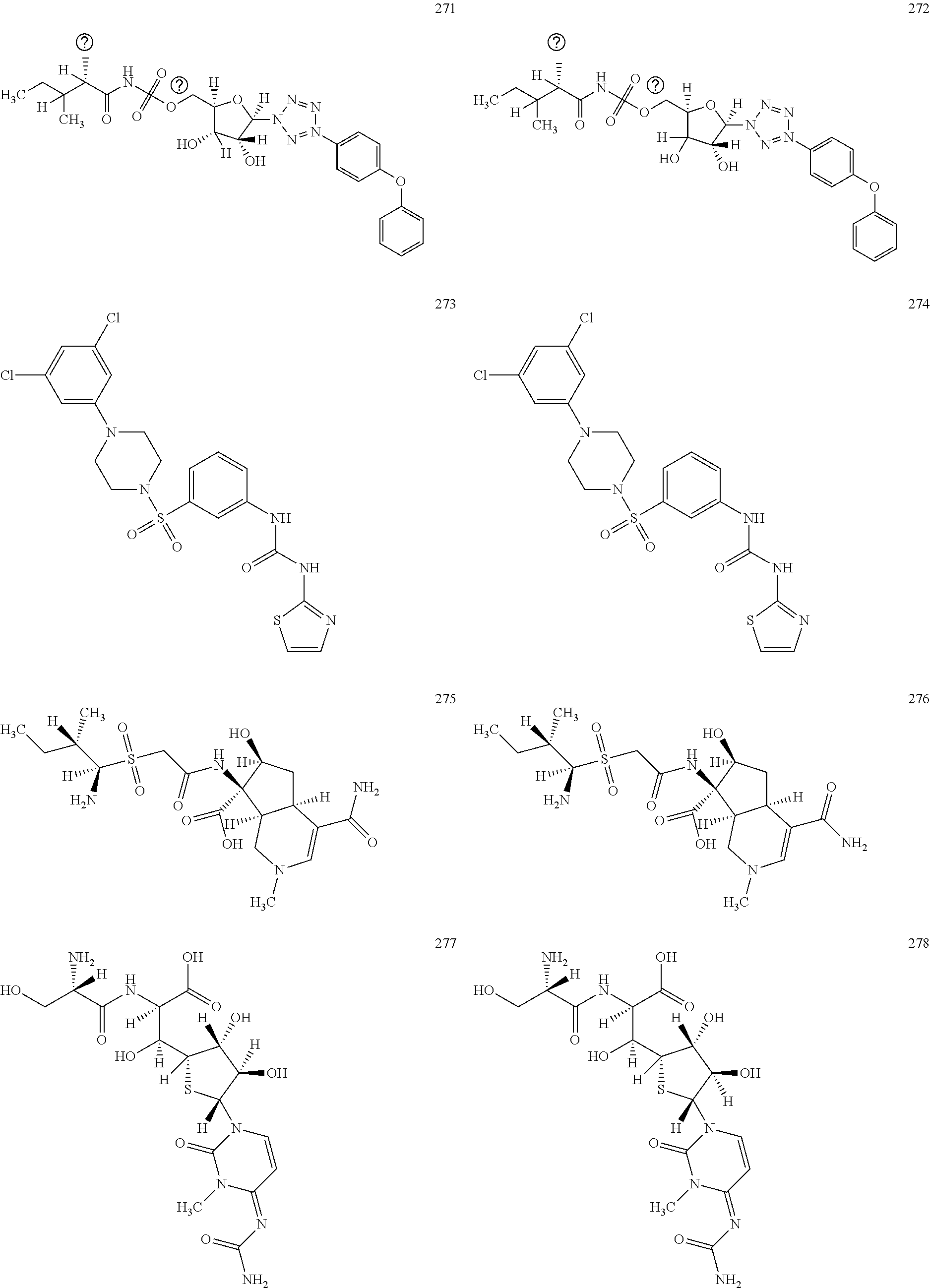

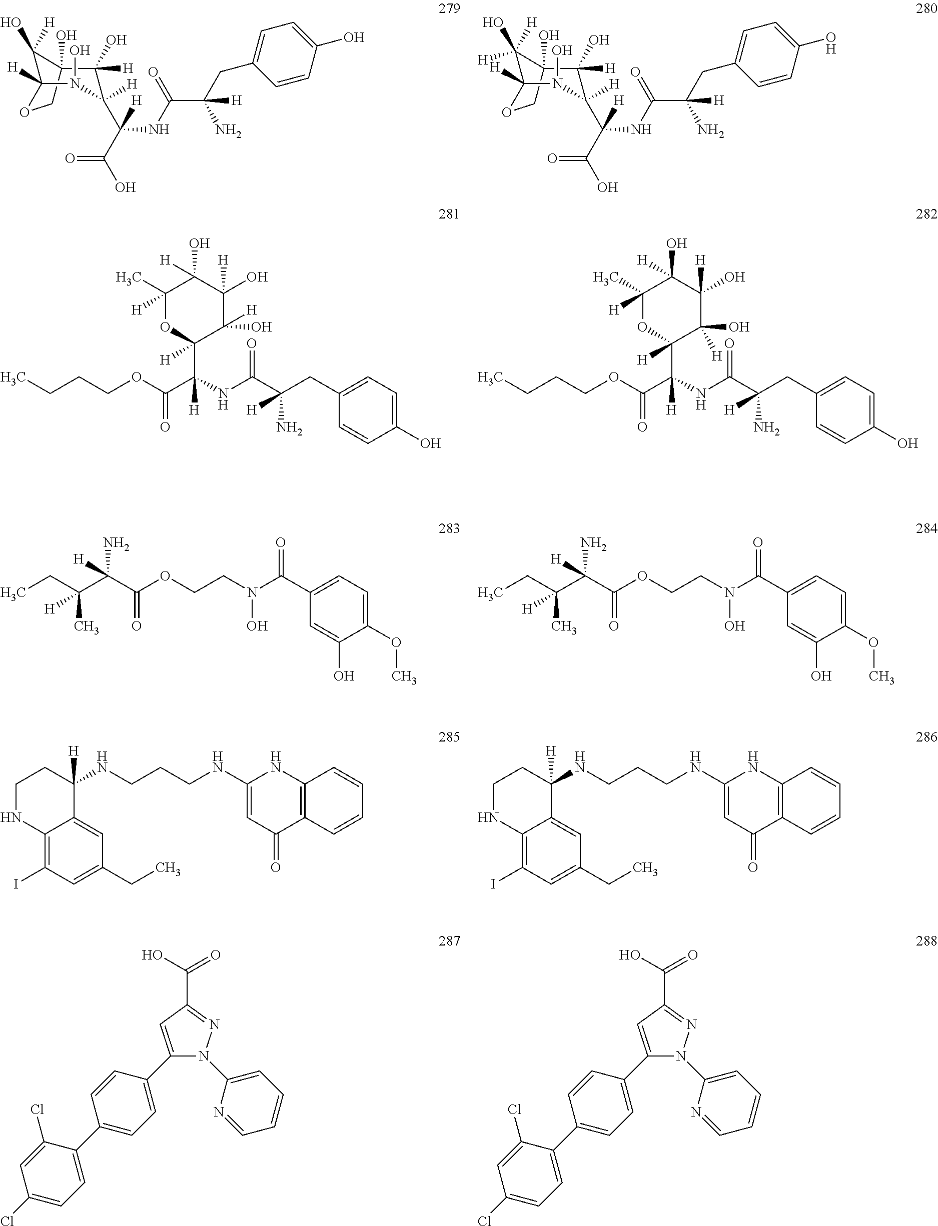

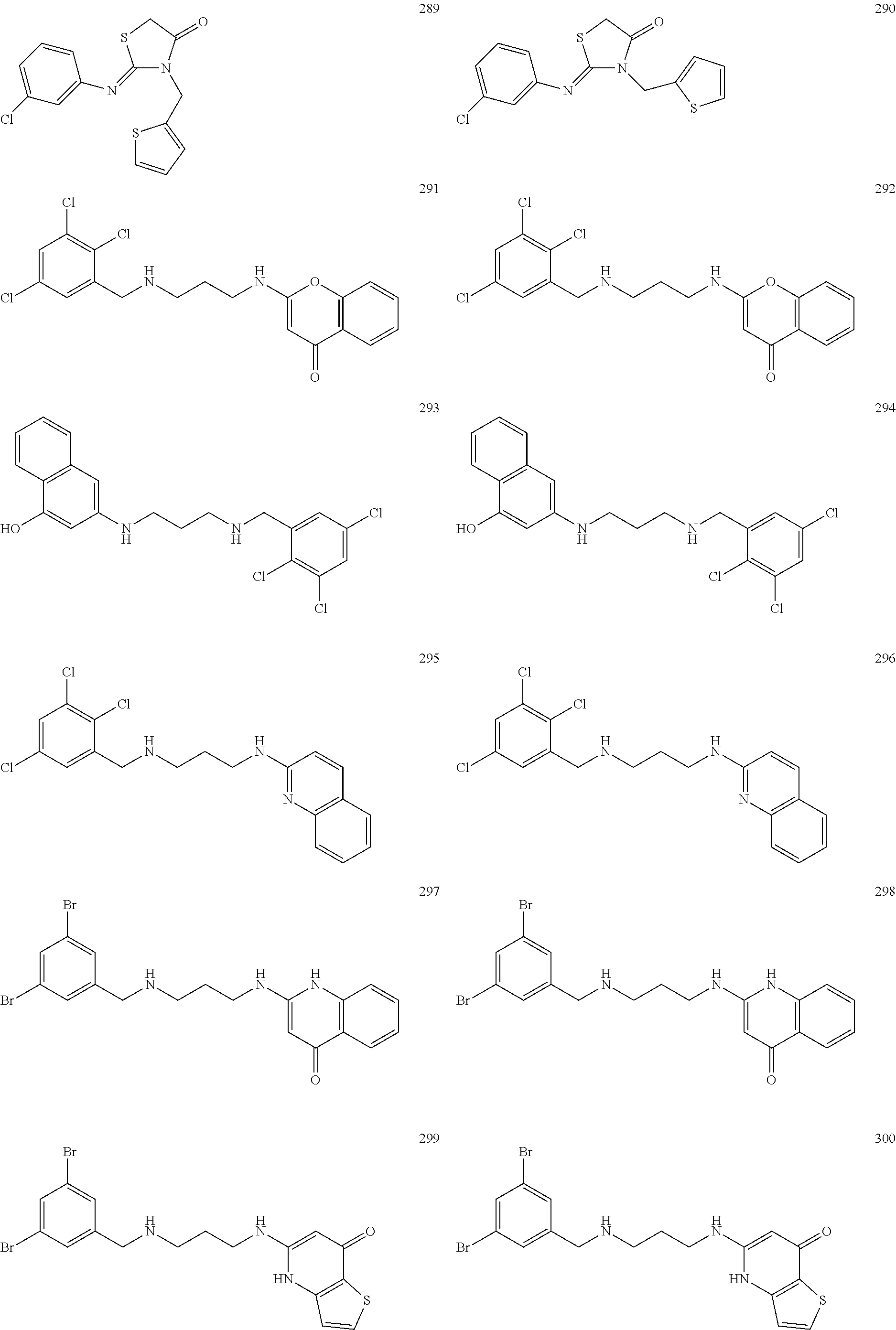

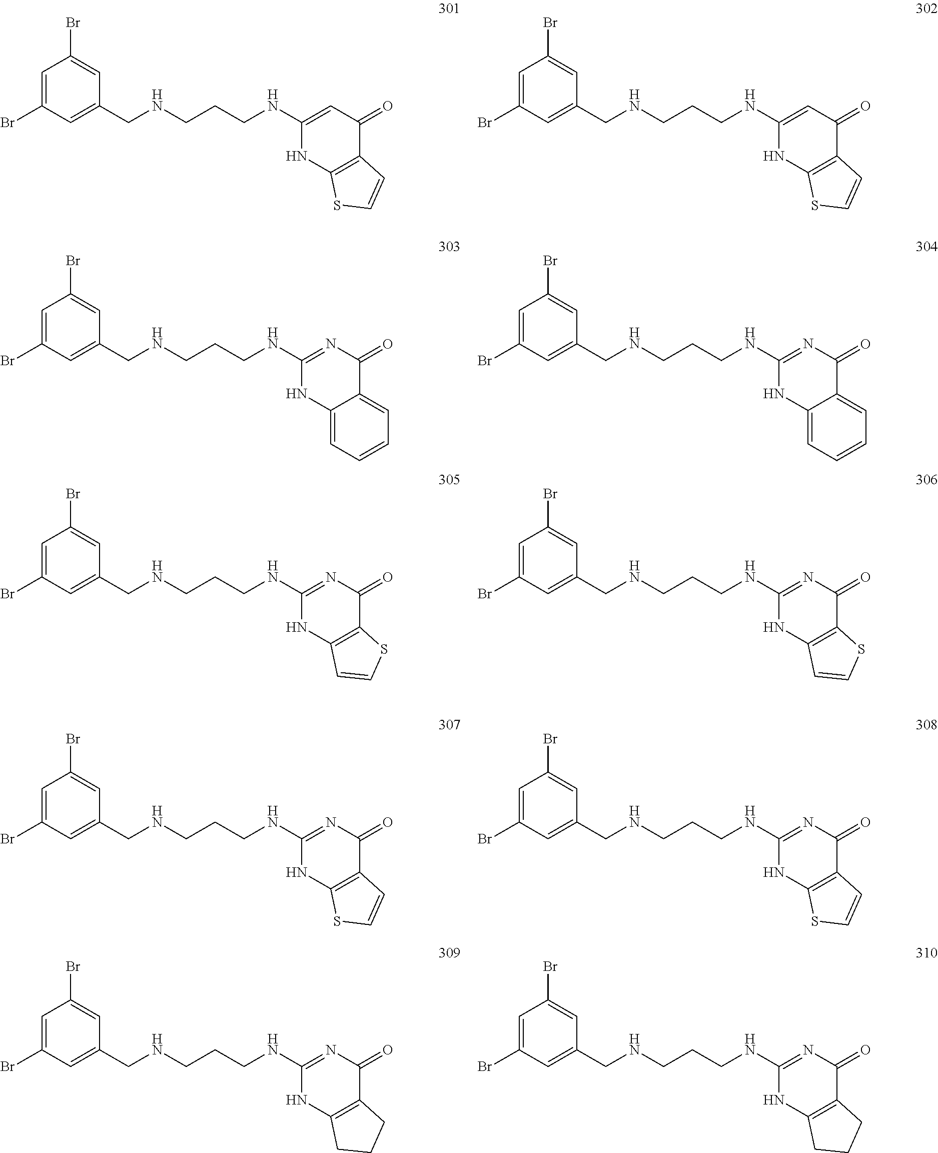

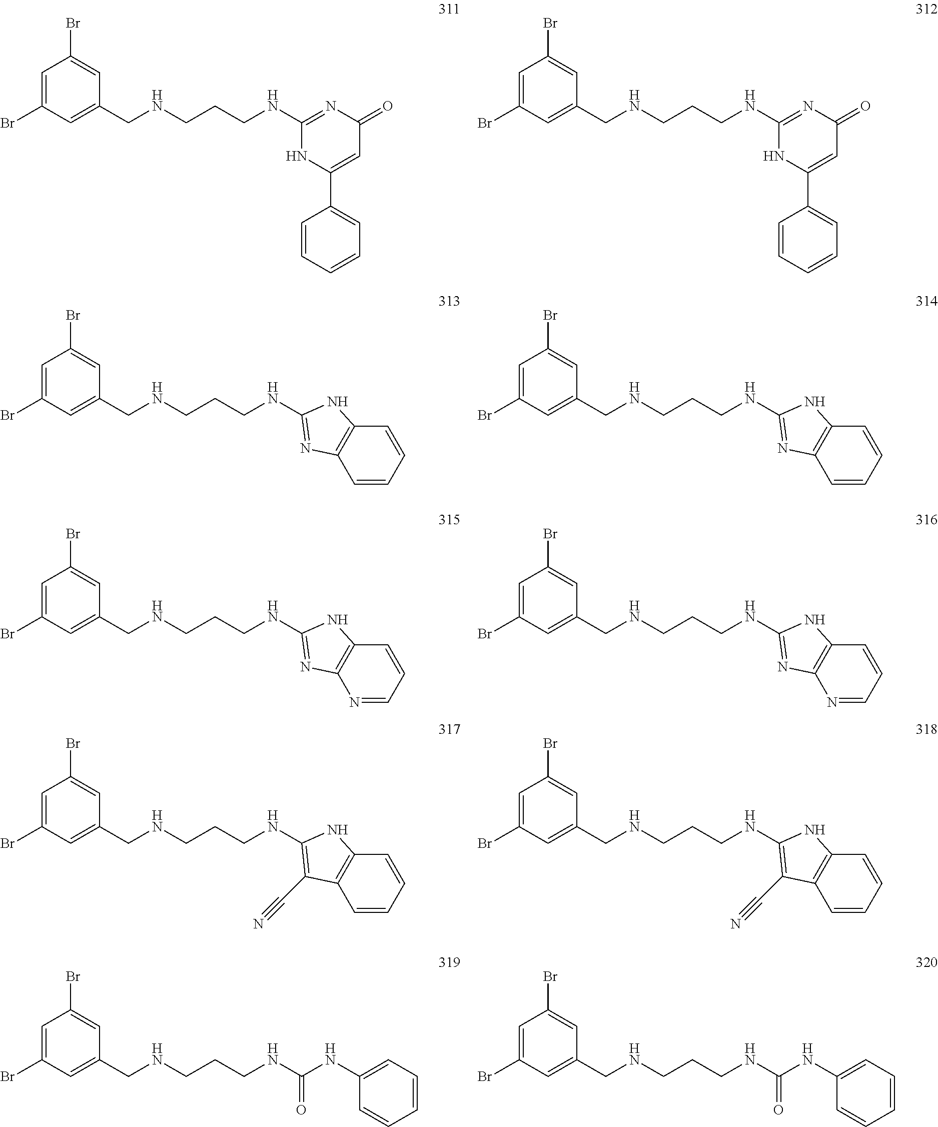

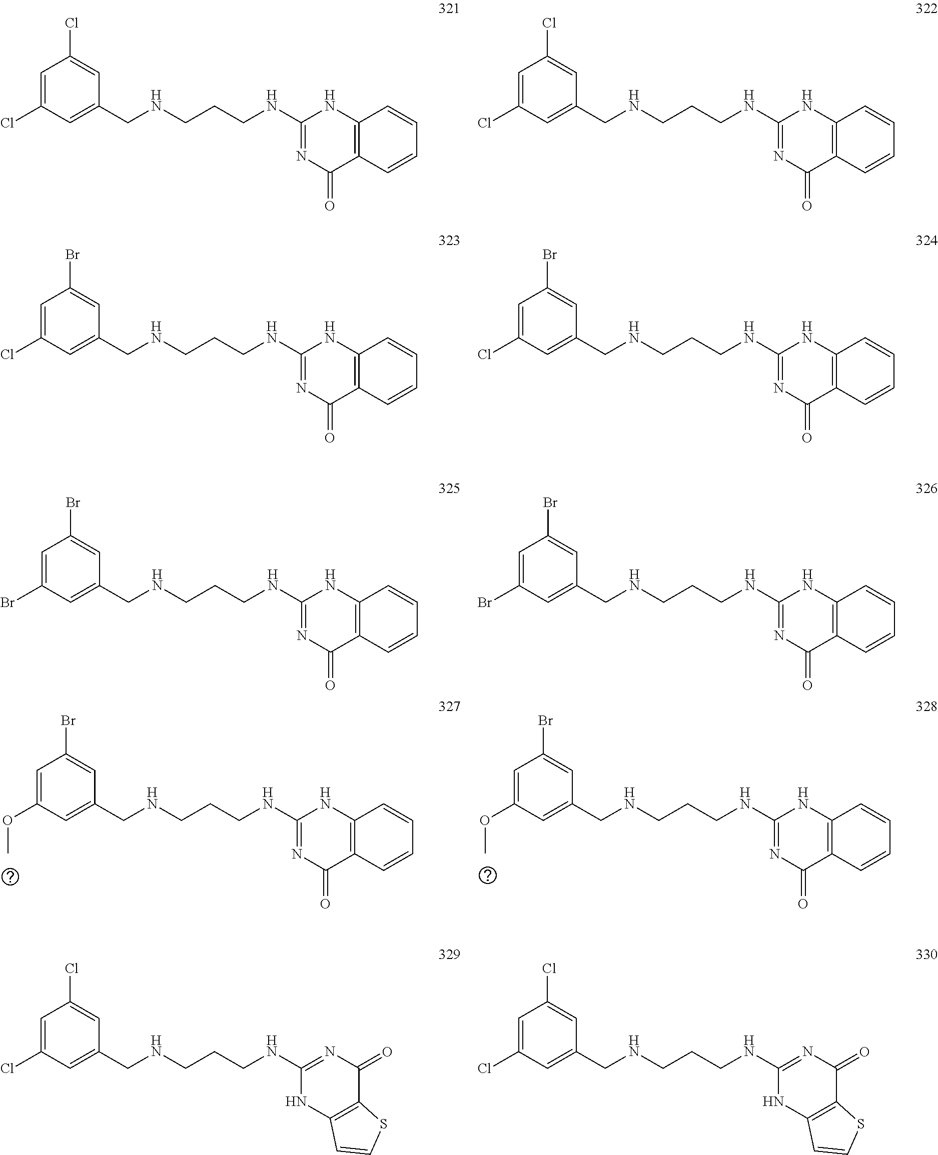

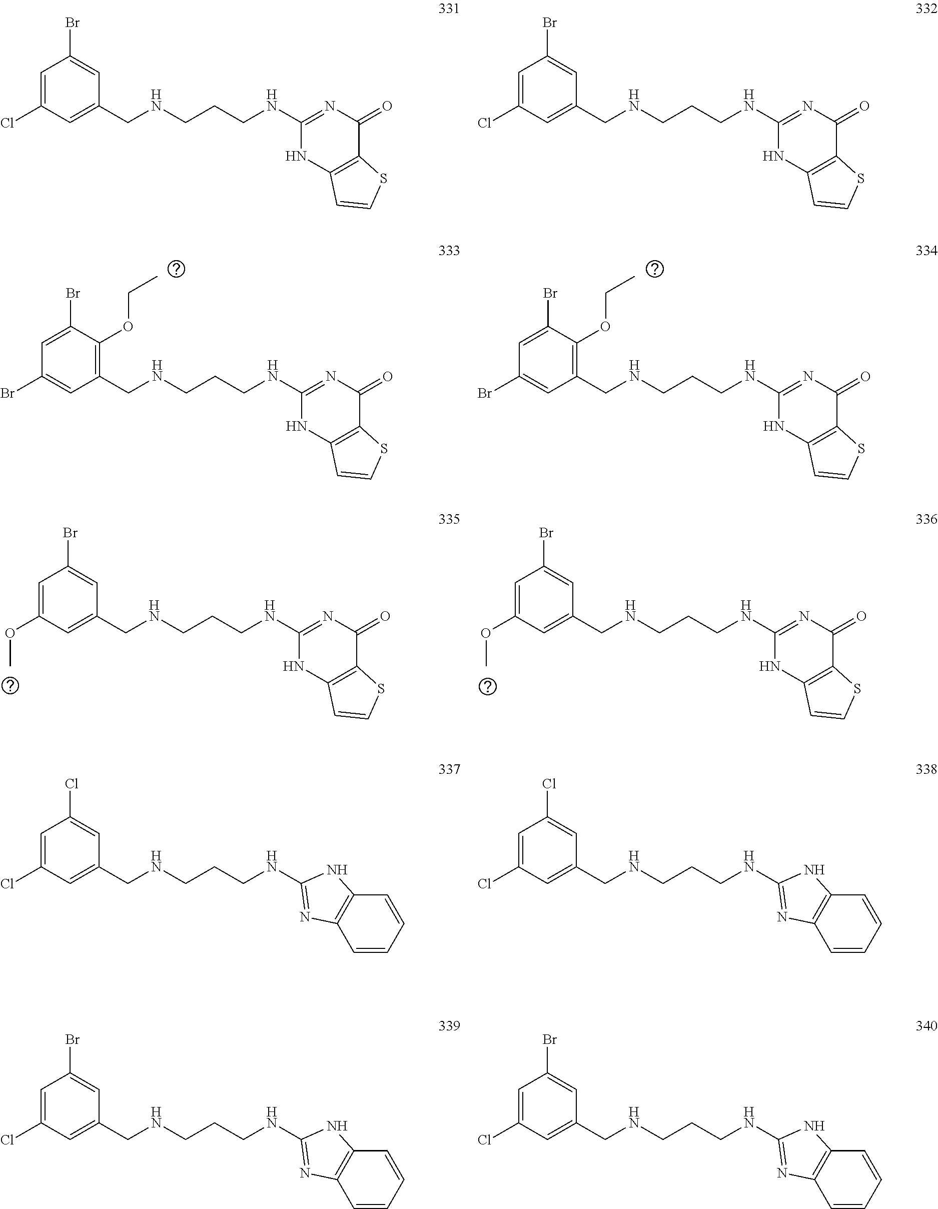

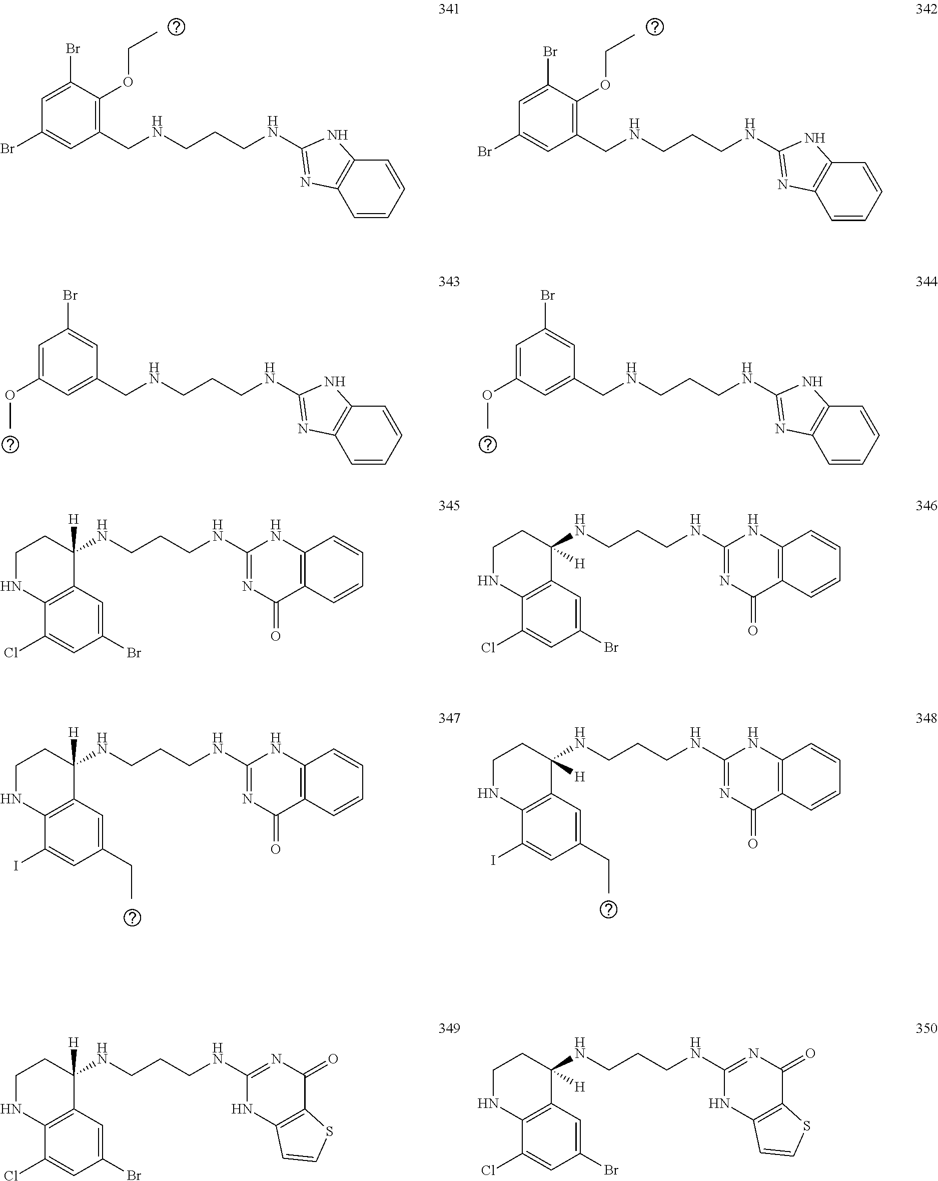

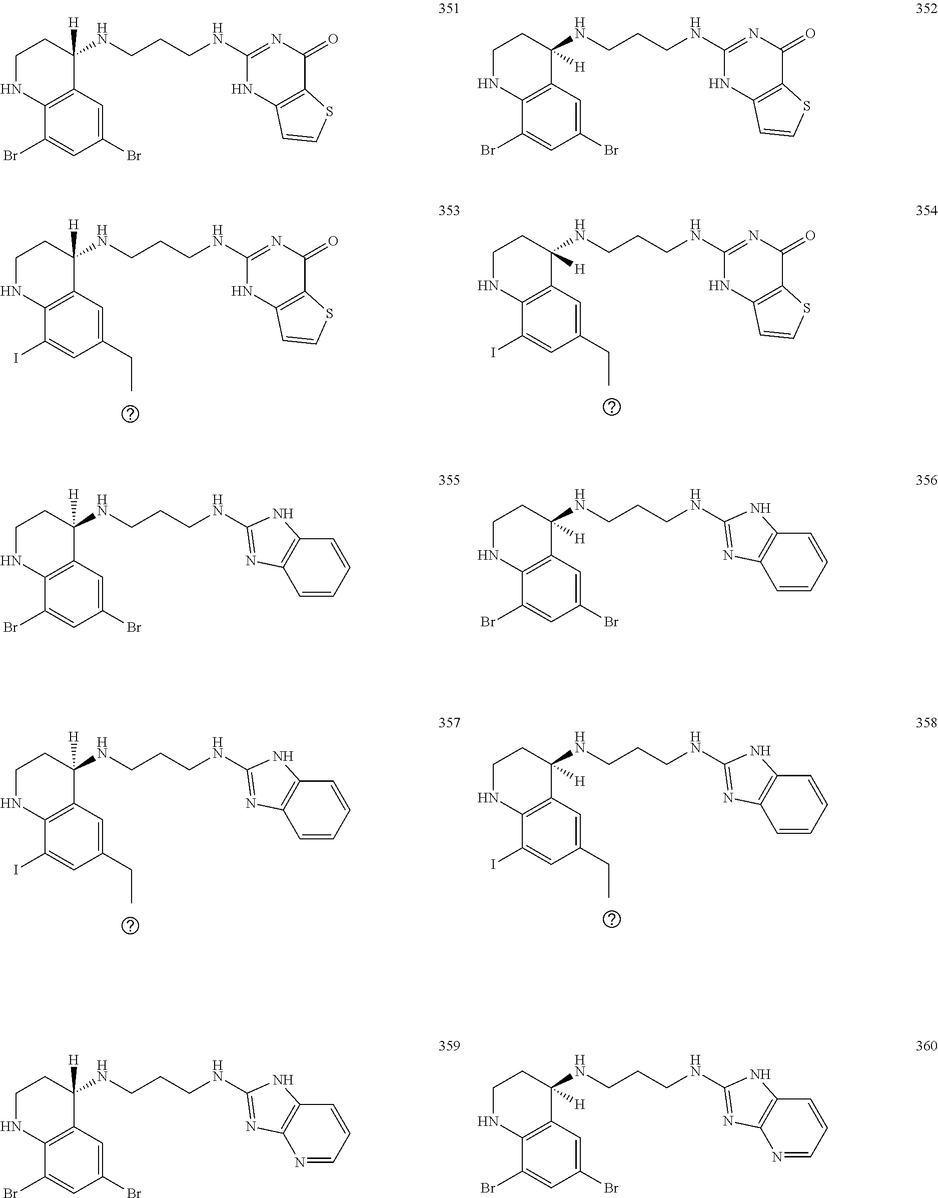

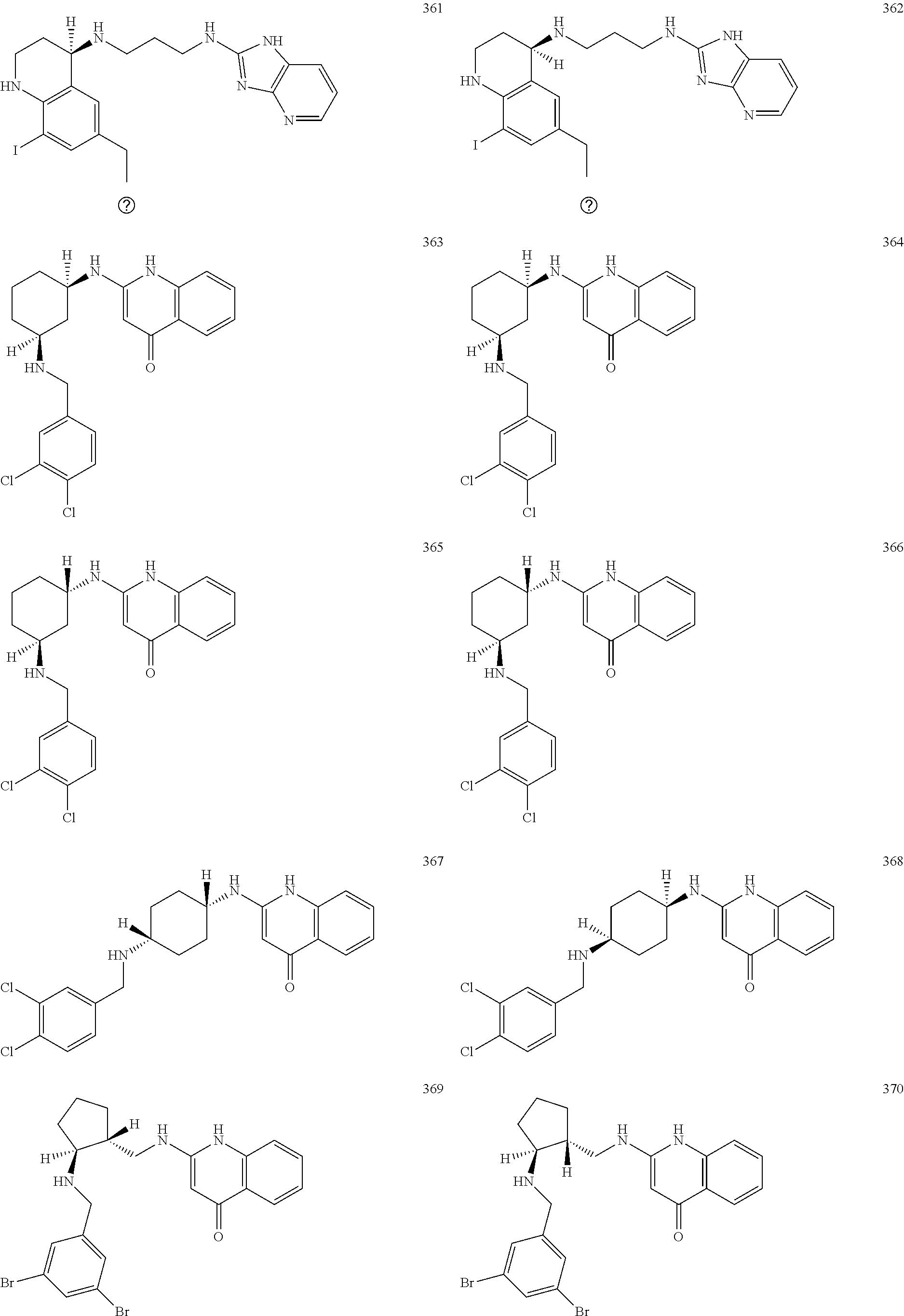

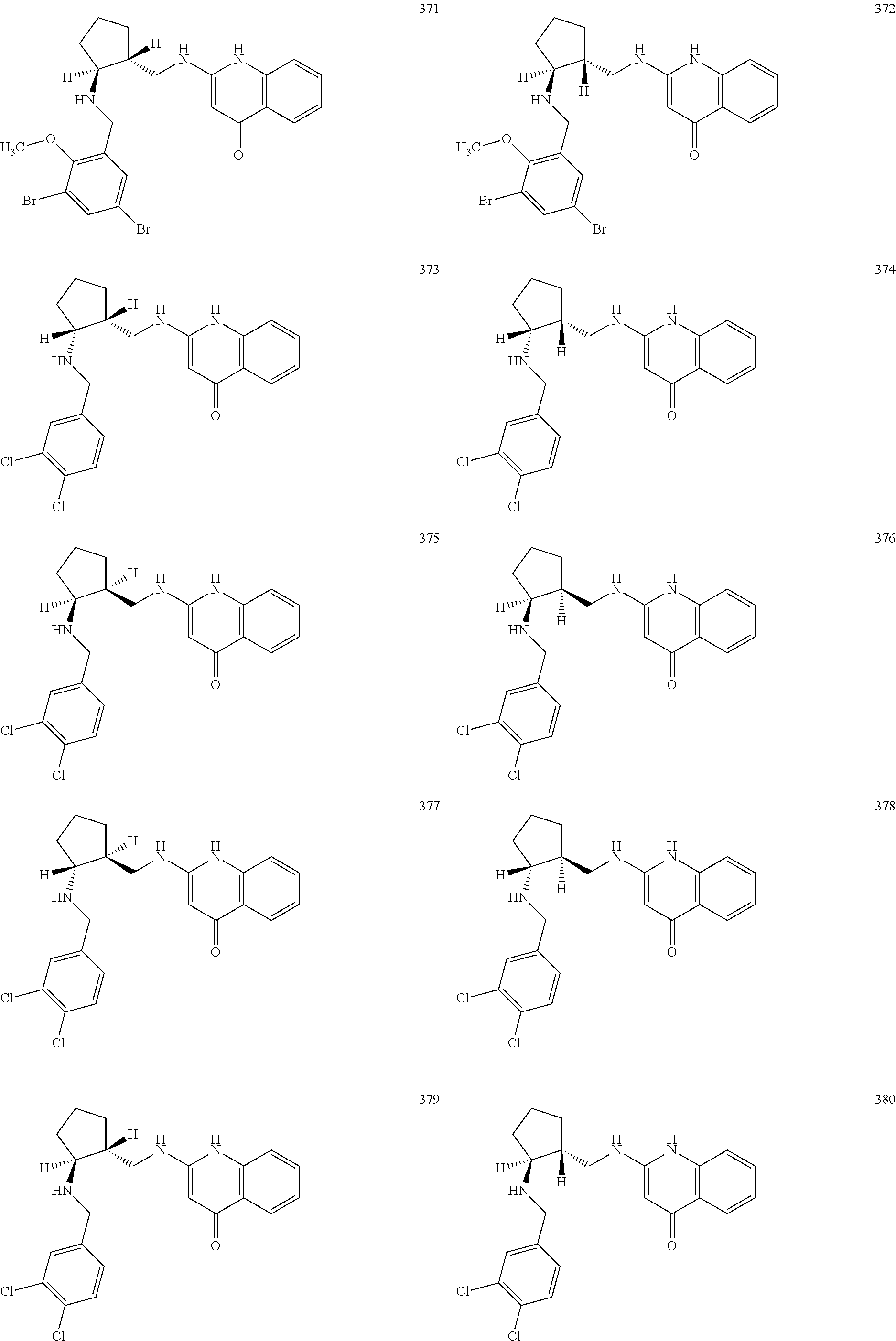

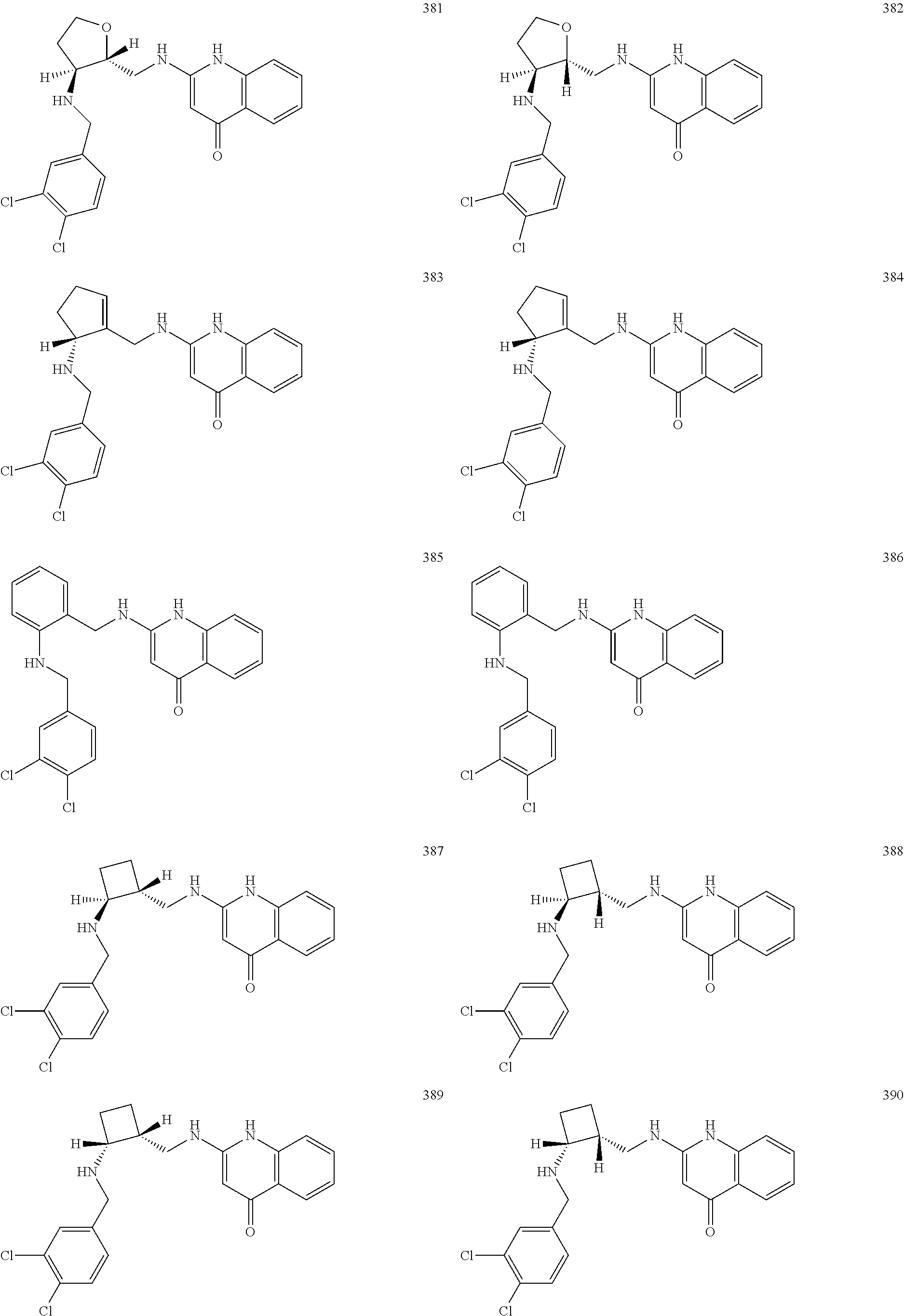

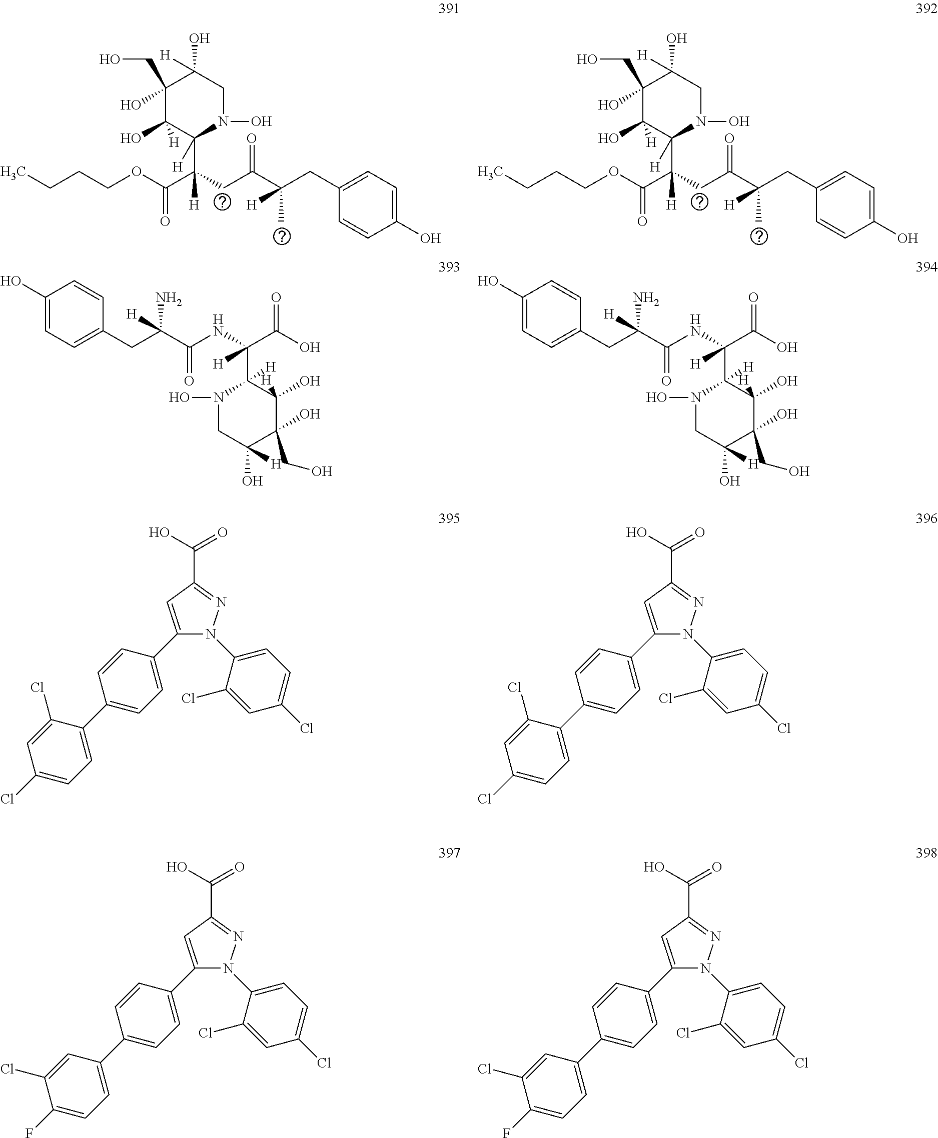

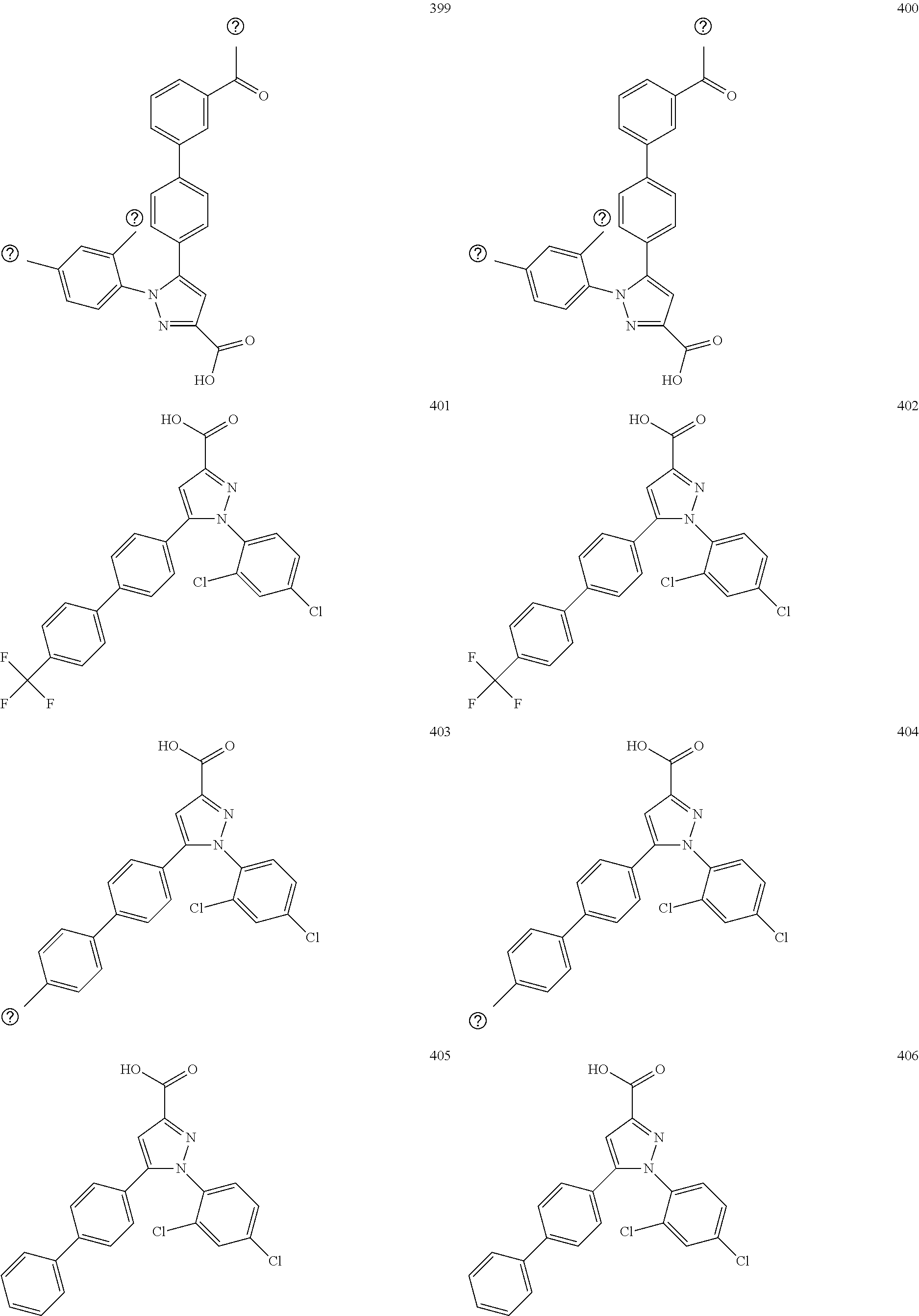

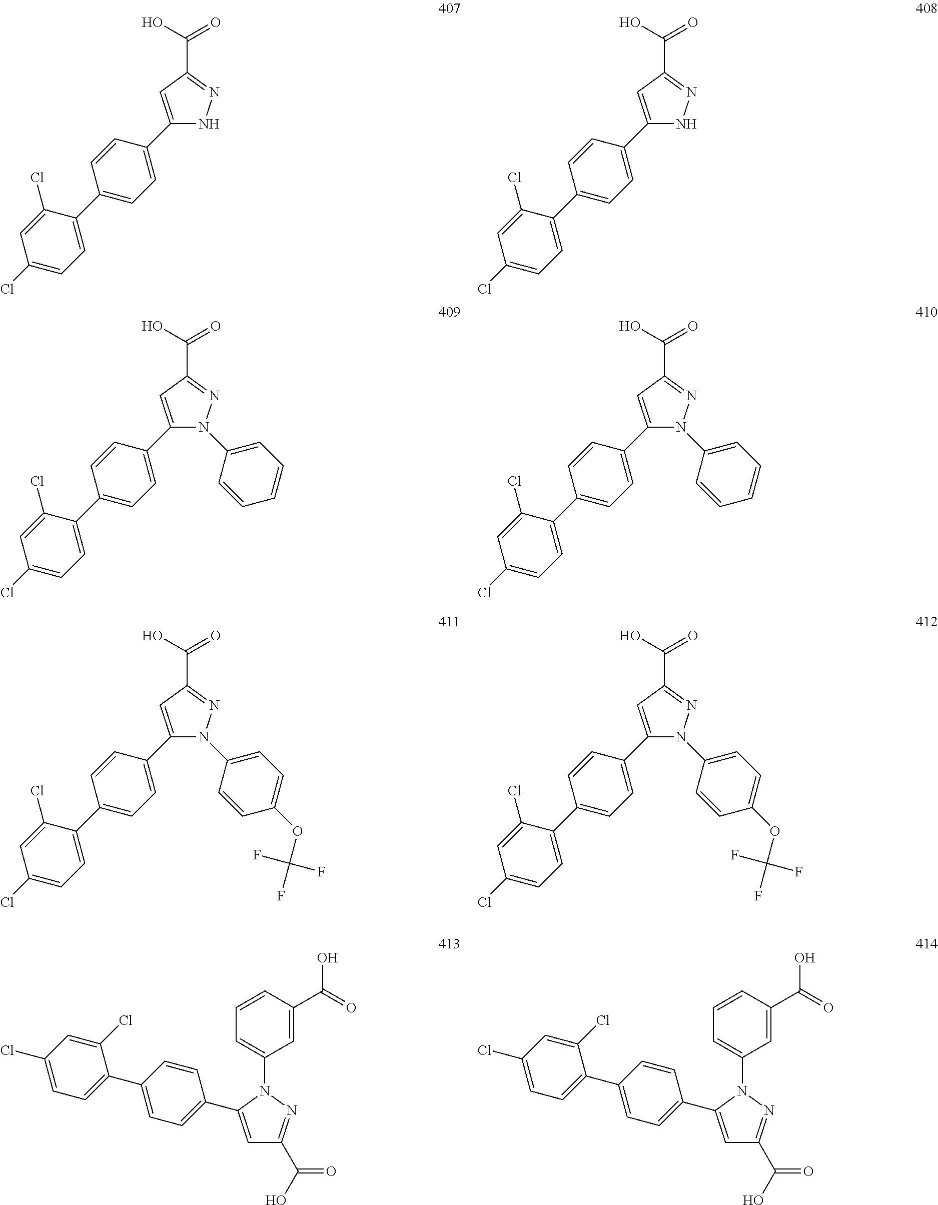

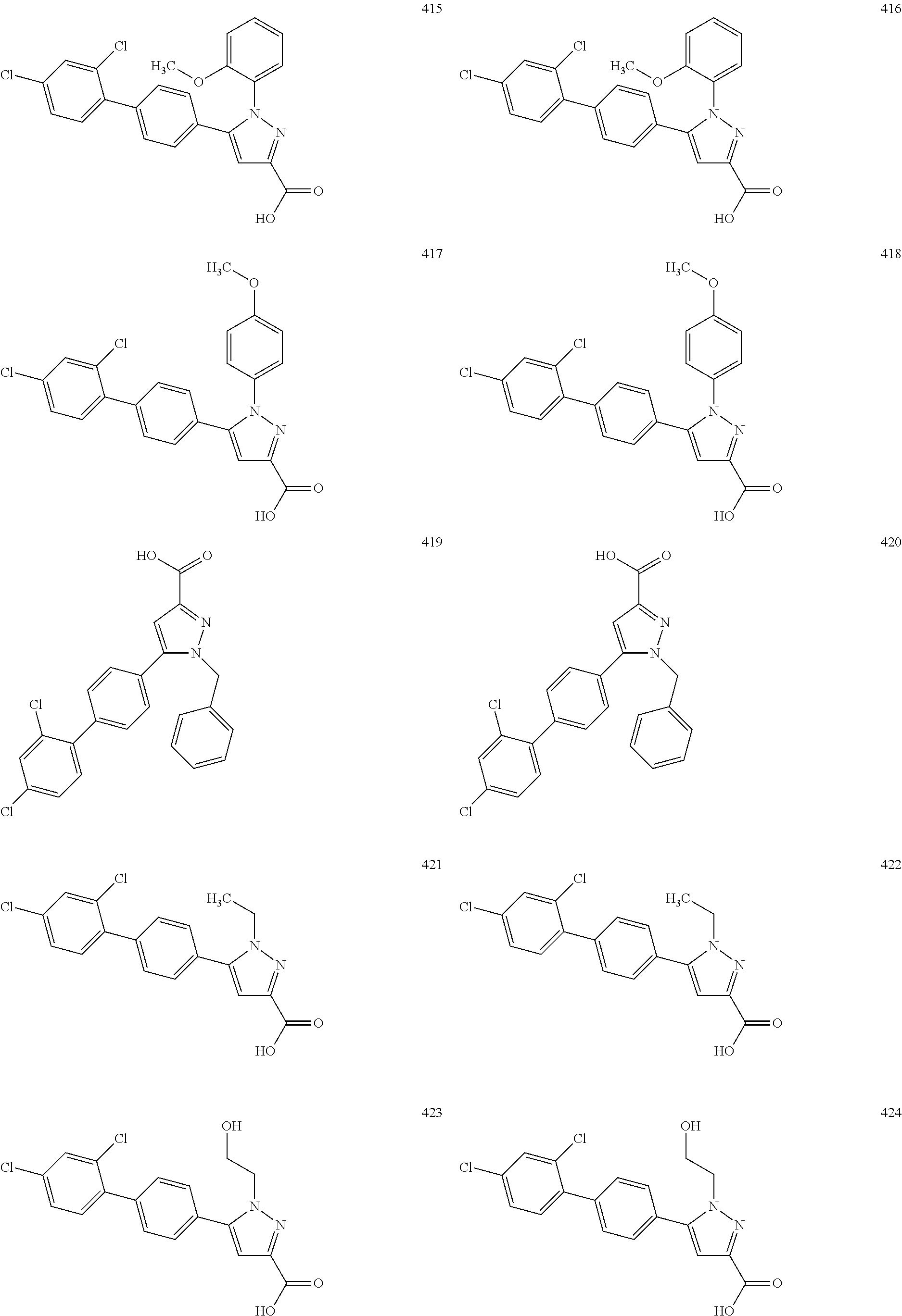

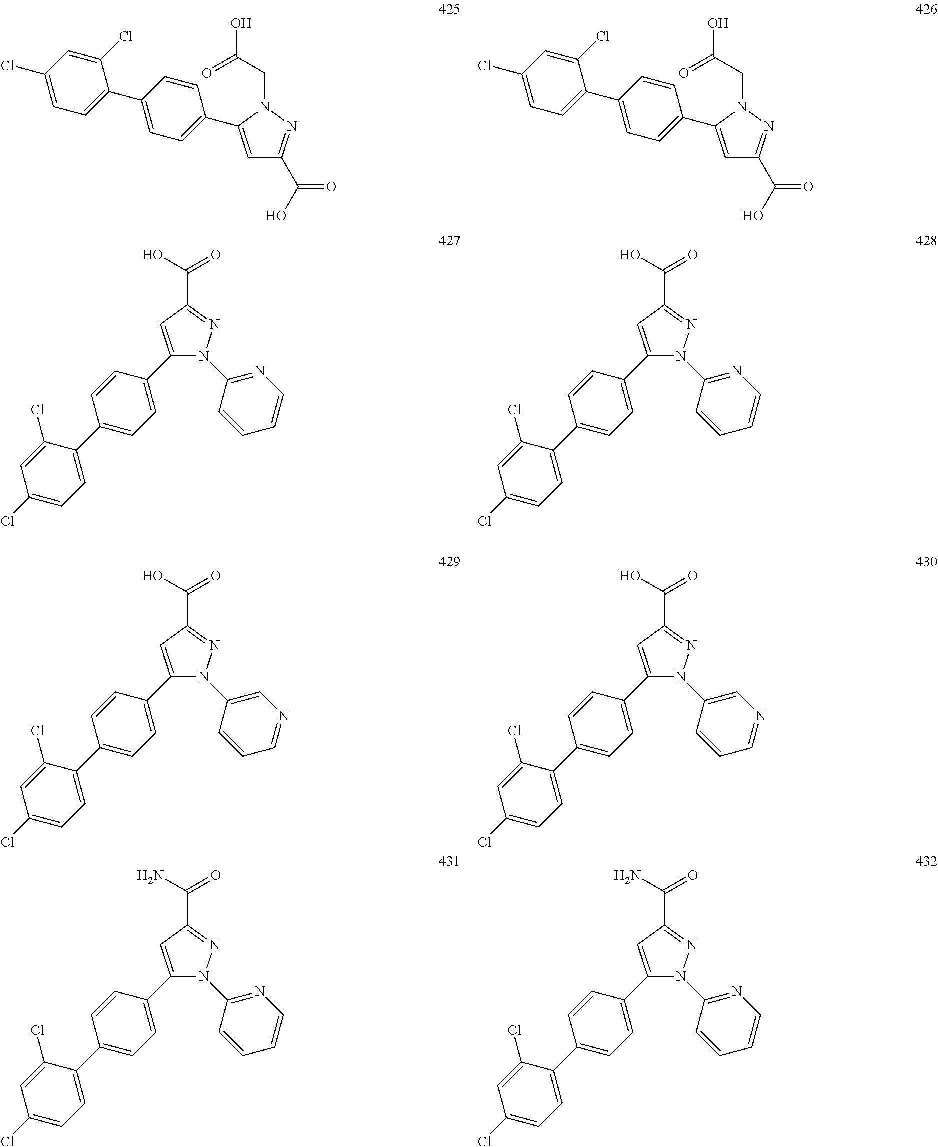

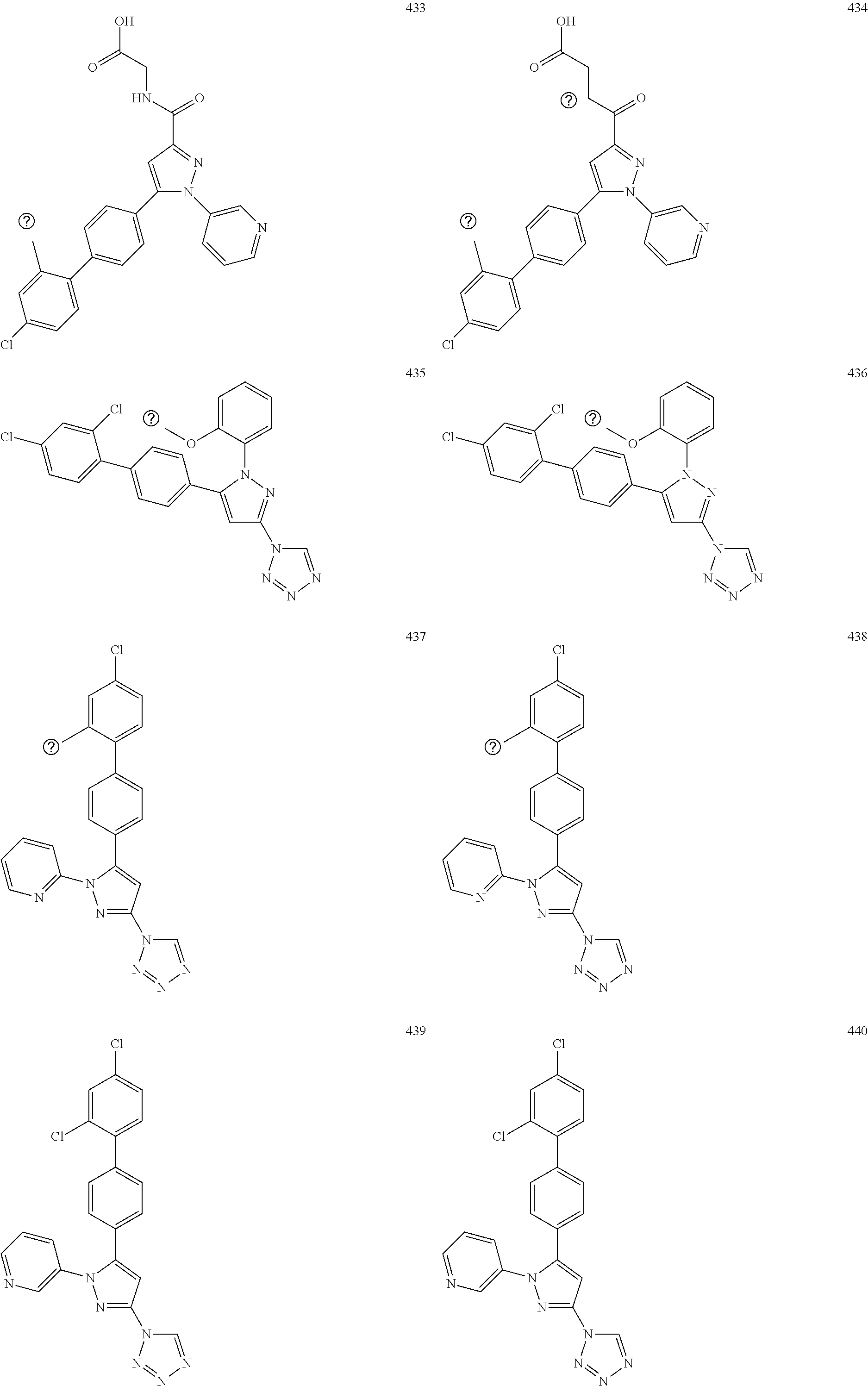

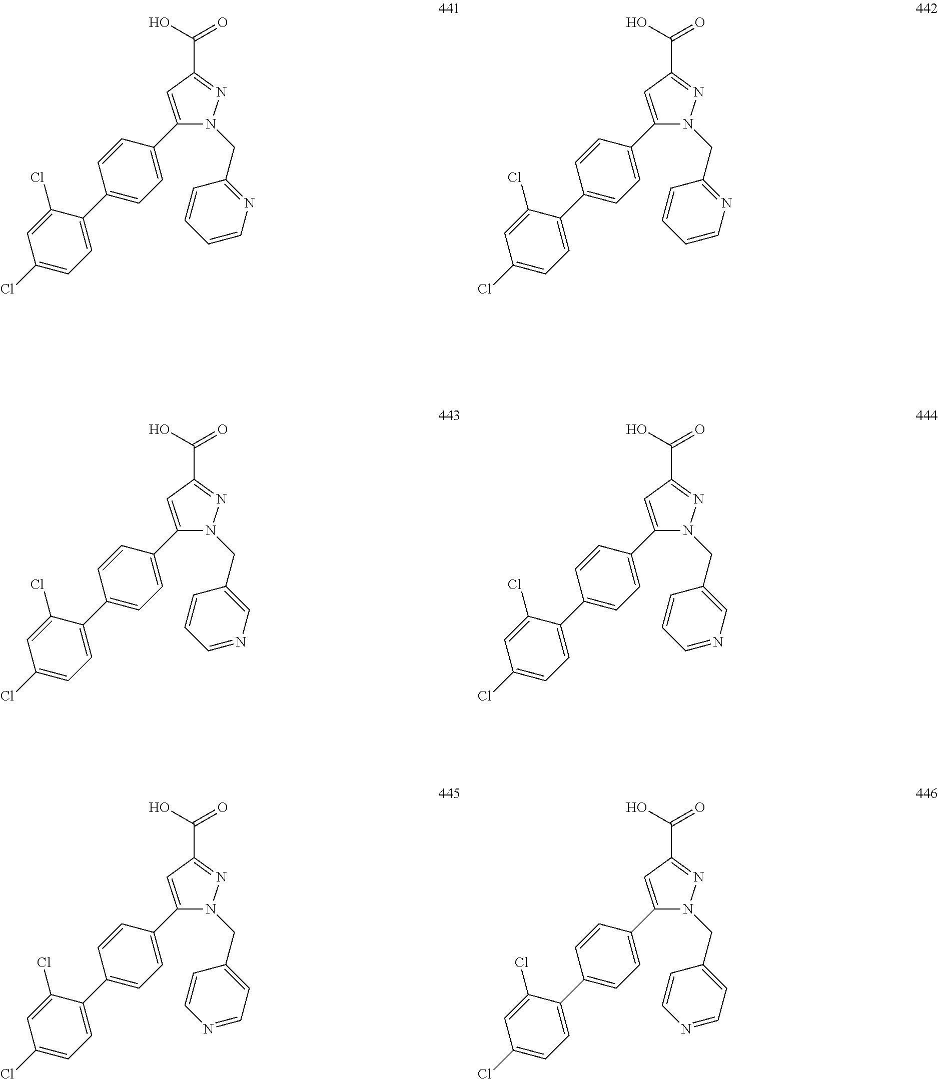

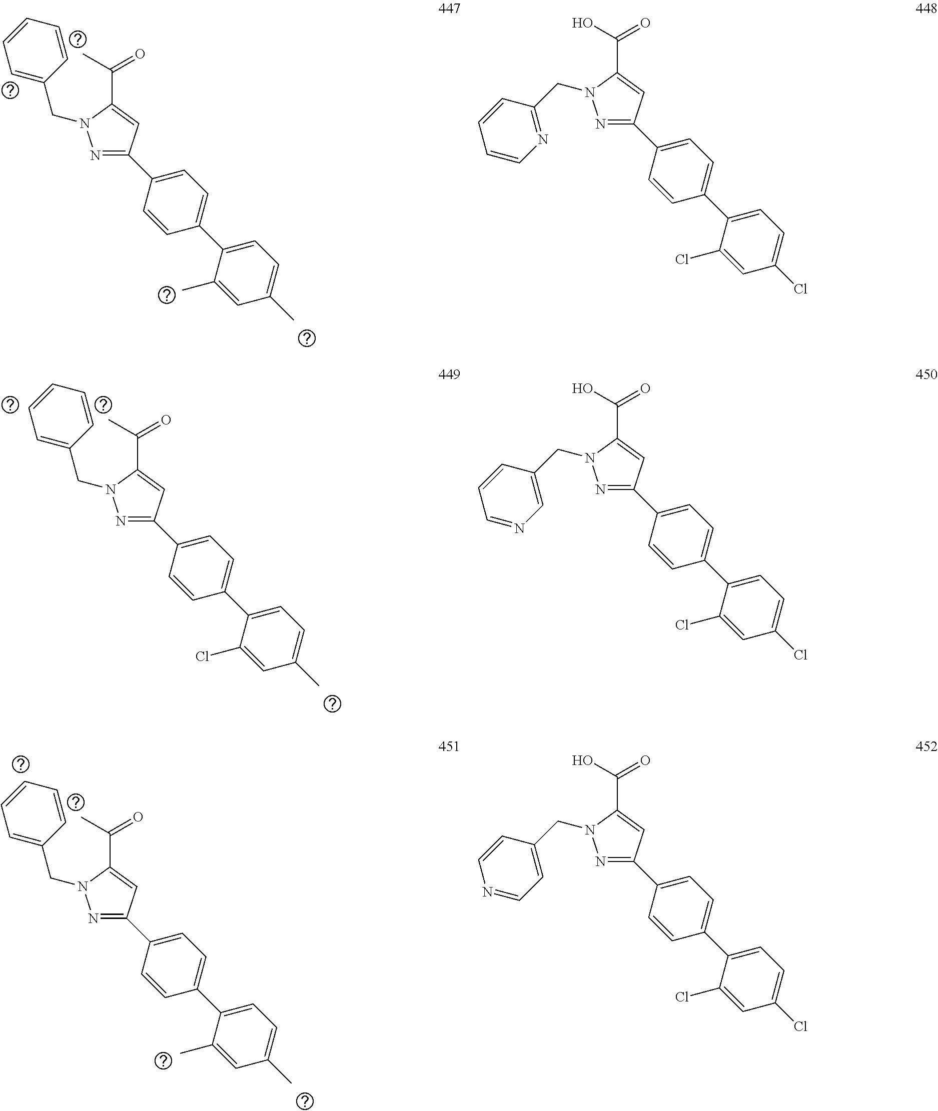

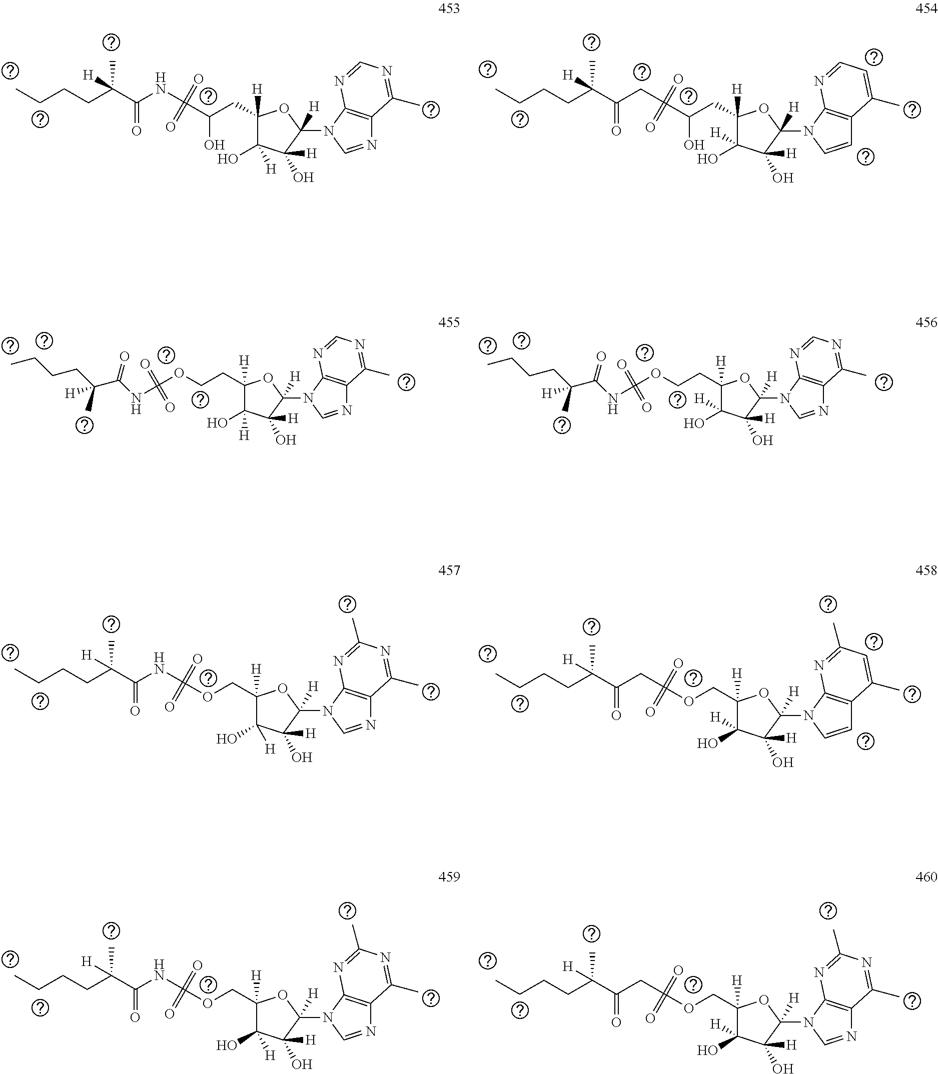

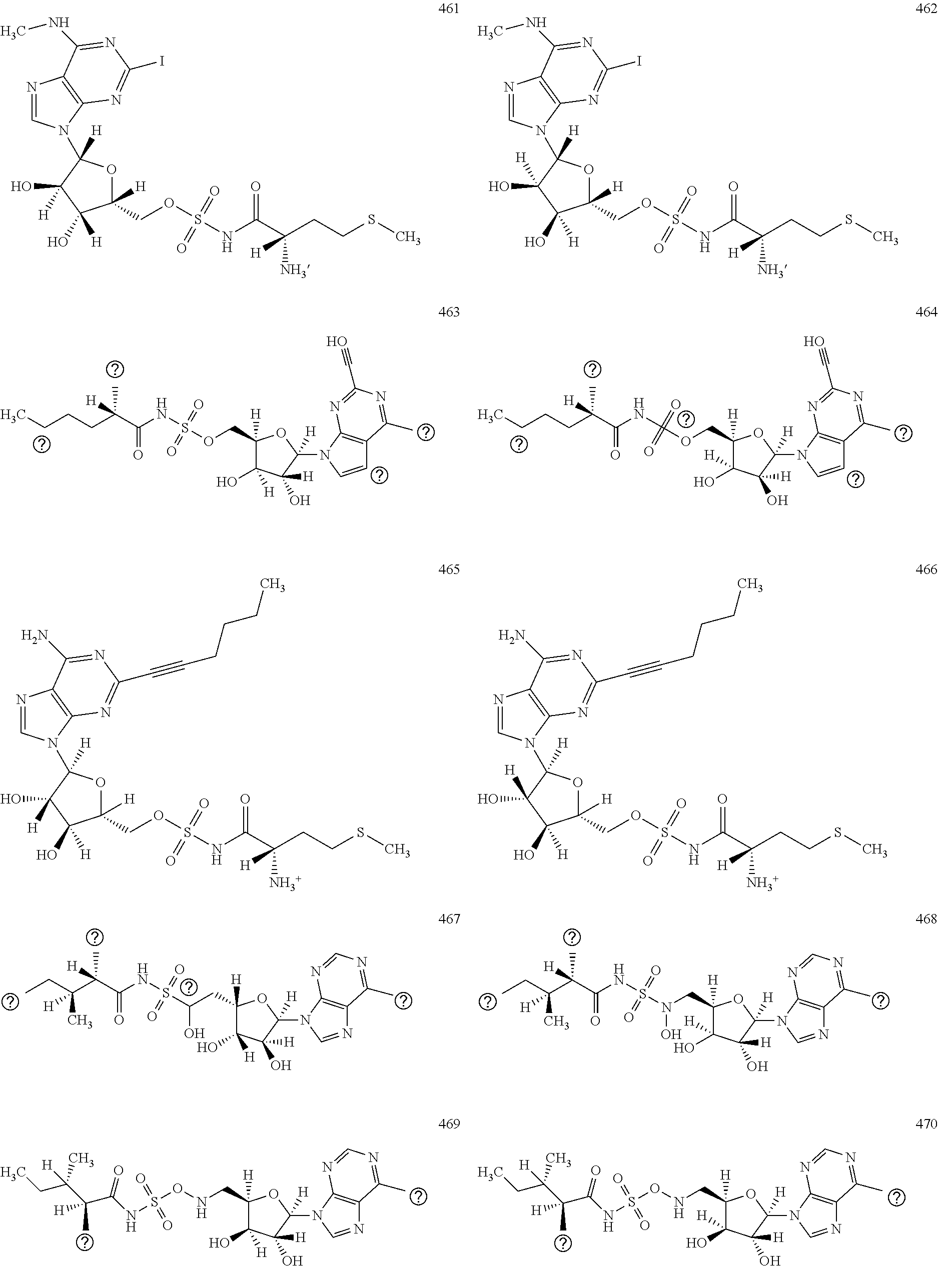

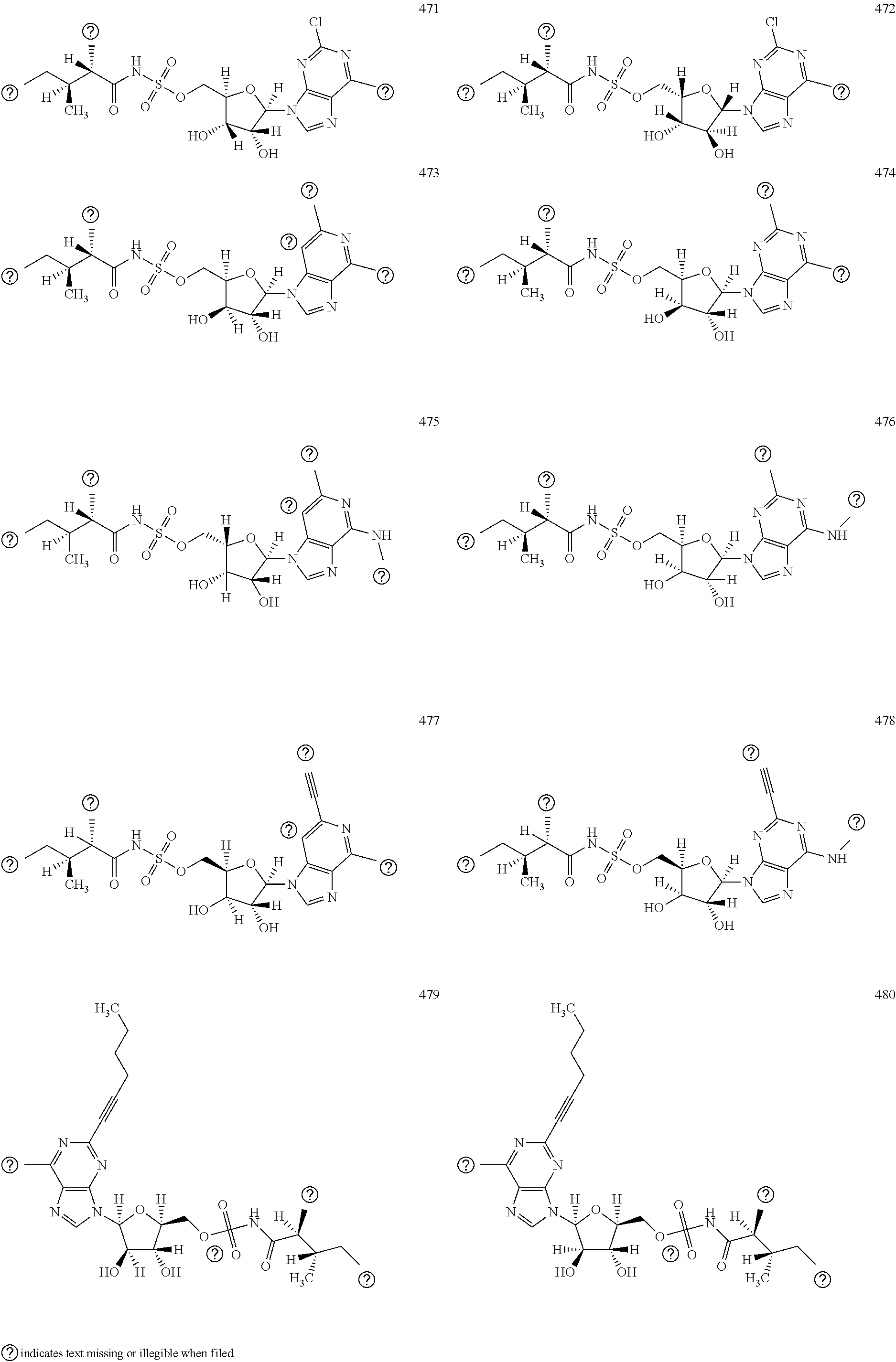

1. A method of inhibiting an immune response mediated by IL-17 expressing T cells in a subject, the method comprising administering to the subject an agent that inhibits a eukaryotic aminoacyl tRNA synthetase, wherein the agent is administered in an amount effective to inhibit the aminoacyl tRNA synthetase in T cells in the subject.

2. The method of claim 1, wherein the agent induces an amino acid starvation response (AAR) in T cells of the subject.

3. The method of claim 1, wherein the agent is an agent that inhibits Th17 differentiation in vitro.

4-5. (canceled)

6. The method of claim 1, wherein the agent inhibits a eukaryotic aminoacyl tRNA synthetase selected from: a prolyl tRNA synthetase, a cysteinyl tRNA synthetase, a methionyl tRNA synthetase, a leucyl tRNA synthetase, a tryptophanyl tRNA synthetase, a glycyl tRNA synthetase, an alanyl tRNA synthetase, a valyl tRNA synthetase, an isoleucyl tRNA synthetase, an aspartyl tRNA synthetase, a glutamyl tRNA synthetase, an asparagyl tRNA synthetase, a glutaminyl tRNA synthetase, a seryl tRNA synthetase, a threonyl tRNA synthetase, a lysyl tRNA synthetase, an arginyl tRNA synthetase, a histidyl tRNA synthetase, a phenylalanyl tRNA synthetase, a tyrosyl tRNA synthetase, and a glutamyl-prolyl-tRNA synthetase (EPRS).

7. The method of claim 6, wherein the agent inhibits a eukaryotic aminoacyl tRNA synthetase of an essential amino acid.

8. The method of claim 6, wherein the agent inhibits a eukaryotic aminoacyl tRNA synthetase of a non-essential amino acid.

9. The method of claim 8, wherein the agent inhibits a eukaryotic aminoacyl tRNA synthetase selected from a prolyl tRNA synthetase, a cysteinyl tRNA synthetase, a glycyl tRNA synthetase, an alanyl tRNA synthetase, an aspartyl tRNA synthetase, a glutamyl tRNA synthetase, an asparagyl tRNA synthetase, a glutaminyl tRNA synthetase, a seryl tRNA synthetase, an arginyl tRNA synthetase, a histidyl tRNA synthetase, a tyrosyl tRNA synthetase, and a glutamyl-prolyl-tRNA synthetase (EPRS).

10-12. (canceled)

13. The method of claim 6, wherein the agent comprises a compound shown in FIG. 1 or Appendix A.

14. The method of claim 1, further comprising administering to the subject a second agent that inhibits a second eukaryotic tRNA synthetase.

15. The method of claim 1, further comprising administering to the subject a second agent, wherein the second agent is an agent that inhibits expression or activity of one or more of IL-6, IL-21, TNFα, IFNγ, GM-CSF, MIP-2, IL-12, IL-1α, IL-Iβ, and IL-23.

16. (canceled)

17. The method of claim 1, wherein the agent inhibits an activity of IL-17-expressing T cells in the subject.

18. The method of claim 17, wherein the agent inhibits proliferation of IL-17-expressing T cells in the subject.

19. The method of claim 1, wherein the agent inhibits production of a cytokine in cells of the subject, wherein the cytokine is selected from IL-17, IL-6, IL-21, TNFα, and GM-CSF.

20. The method of claim 1, wherein the subject is a subject at risk for, or suffering from, an IL-17-mediated disorder.

21. The method of claim 20, wherein the IL-17-mediated disorder is an autoimmune disease, an infectious disease, graft rejection, graft versus host disease, asthma, chronic inflammation, or inflammation associated with a microbial infection.

22-31. (canceled)

32. The method of claim 1, wherein the method comprises identifying the subject as at risk for, or suffering from an IL-17-mediated disorder, prior to the administering.

33. A method of inhibiting one or more of fibrosis, angiogenesis, scar formation, cellulite formation or cellulite progression in a subject, the method comprising administering to the subject an agent that inhibits a eukaryotic aminoacyl tRNA synthetase, wherein the agent is administered in an amount effective to inhibit the aminoacyl tRNA synthetase in the subject.

34-47. (canceled)

48. A method of modulating differentiation of a T cell, the method comprising:
contacting a T cell with an agent that inhibits a eukaryotic tRNA synthetase under conditions in which differentiation occurs, thereby modulating differentiation of the T cell.

49-55. (canceled)

56. A method of identifying an agent that modulates T cell differentiation, the method comprising:
(a) contacting a T cell with an inhibitor of a eukaryotic aminoacyl tRNA synthetase under conditions in which T cell differentiation occurs, and
(b) evaluating a marker of T cell differentiation, wherein a change in the marker of T cell differentiation, relative to a control, indicates that the inhibitor of the aminoacyl tRNA synthetase is an agent that modulates T cell differentiation.

57-61. (canceled)

62. A pharmaceutical composition comprising an agent that inhibits a eukaryotic aminoacyl tRNA synthetase in a pharmaceutically acceptable carrier.

63-65. (canceled)
